(19) United States
(12) Patent Application Publication
(10) Pub. No.: US 2007/0166788 A1
(43) Pub. Date: Jul. 19, 2007

(54) Methods for production of receptor and ligand isoforms

(76) Inventors: Pei Jin, (US); H. Michael Shepard, (US); Cornelia M. Gorman, (US); Juan Zhang, (US)

Correspondence Address: MORRISON & FOERSTER LLP, 755 PAGE MILL RD, PALO ALTO, CA, 94304-1018, US

(21) Appl. No.: 11/591,229

(22) Filed: Oct. 31, 2006

Related U.S. Application Data
(60) Provisional application No. 60/736,134, filed on Nov. 10, 2005.

(51) Int. Cl. A61K 48/00; C12P 21/06; C12N 9/12; C07H 21/04; C12N 5/06

(52) U.S. Cl. 435/69.1; 435/194; 435/320.1; 435/325; 536/23.2; 435/364; 435/366; 435/349; 435/353; 435/354; 435/358; 514/44

(57) ABSTRACT

Provided are methods for production of cell surface receptor (CSR) and ligand isoforms. In particular, isoform fusions that a precursor sequence for secretion, processing and intracellular trafficking are provided. Nucleic acid molecules encoding the fusions are expressed in a host cell and the encoded and partially or completely processed encoded CSR or ligand isoforms is produced in the cell culture medium. The resulting polypeptide optionally includes an epitope tag for the detection and/or purification thereof.

### RELATED APPLICATIONS

Benefit of priority is claimed to U.S. provisional application Ser. No. 60/736,134, filed Nov. 10, 2005, entitled “METHODS FOR PRODUCTION OF RECEPTOR AND LIGAND ISOFORMS,” to Pei Jin, H. Michael Shepard, Cornelia Gorman and Juan Zhang.

This application is related to International PCT Application Ser. No. (Attorney Docket No. 17118-041W01/2822PC), filed the same day herewith, entitled “METHODS FOR PRODUCTION OF RECEPTOR AND LIGAND ISOFORMS,” to Receptor Biologix, Inc., Pei Jin, H. Michael Shepard, Cornelia Gorman and Juan Zhang, which also claims priority to U.S. Provisional Application Ser. No. 60/736,134.

This application is related to U.S. application Ser. No. 10/846,113, filed May 14, 2004, and to corresponding International PCT application No. WO 05/016966, published Feb. 24, 2005, entitled “INTRON FUSION PROTEINS, AND METHODS OF IDENTIFYING AND USING SAME.” This application also is related to U.S. application Ser. No. 11/129,740, filed May 13, 2005, and to corresponding International PCT application No. US2005/17051, filed May 13, 2005, entitled “CELL SURFACE RECEPTOR ISOFORMS AND METHODS OF IDENTIFYING AND USING THE SAME.” The application also is related to U.S. Provisional application No. 60/678,076, entitled “ISOFORMS OF RECEPTOR FOR ADVANCED GLYCATION END PRODUCTS (RAGE) AND METHODS OF IDENTIFYING AND USING SAME”, filed May 04, 2005. This application also is related to U.S. application No. (Attorney Docket No. 17118-045001/2824) and to International application No. (Attorney Dockety No. 17118-045W01/2824PC), entitled “HEPATOCYTE GROWTH FACTOR INTRON FUSION PROTEINS,” filed the same day herewith, which each claim priority to U.S. Provisional Application No. 60/735,609 filed Nov. 10, 2005.

The subject matter of each of the above-noted applications, provisional applications and international applications as well as any applications noted throughout the disclosure herein is incorporated herein by reference thereto.

An electronic version on compact disc (CD) ROM of the Sequence Listing is filed herewith in duplicate, the contents of which are incorporated by reference in their entirety. The computer-readable file on each of the aforementioned duplicate compact discs created on Oct. 31, 2006, is identical, 1,589 kilobytes in size, and is entitled 2822SEQ.001.txt.

### FIELD OF THE INVENTION

Provided are methods for production of cell surface receptor (CSR) and ligand isoforms. In particular, isoform fusions that contain a precursor sequence for secretion, processing and intracellular trafficking are provided. Nucleic acid molecules encoding the fusions are expressed in a host cell and the encoded and partially or completely processed CSR or ligand isoform is produced in the cell culture medium. The resulting polypeptide optionally includes an epitope tag for the detection and/or purification thereof.

### BACKGROUND

Cell signaling pathways involve a network of molecules including polypeptides and small molecules that interact to transmit extracellular, intercellular and intracellular signals. Such pathways interact like a relay, handing off signals from one member of the pathway to the next. Modulation of the activity of one member of the pathway can be transmitted through the signal transduction pathway, resulting in modulation of activities of other pathway members and modulation of the outcomes of such signal transduction, such as affecting phenotypes and responses of a cell or organism to a signal. Diseases and disorders can involve misregulation, or changes in modulation, of signal transduction pathways. A goal of drug development is to target such misregulated pathways to restore more normal regulation in the signal transduction pathway.

Receptor tyrosine kinases (RTKs) are among the polypeptides involved in many signal transduction pathways. RTKs play a role in a variety of cellular processes, including cell division, proliferation, differentiation, migration and metabolism. RTKs can be activated by ligands. Such activation in turn activates events in a signal transduction pathway, such as by triggering autocrine or paracrine cellular signaling pathways, for example, activation of second messengers, which results in specific biological effects. Ligands for RTKs specifically bind to the cognate receptors.

RTKs have been implicated in a number of diseases including cancers such as breast and colorectal cancers, gastric carcinoma, gliomas and mesodermal-derived tumors. Disregulation of RTKs has been noted in several cancers. For example, breast cancer can be associated with amplified expression of p185-HER2. RTKs also have been associated with diseases of the eye, including diabetic retinopathies and macular degeneration. RTKs also are associated with regulating pathways involved in angiogenesis, including physiologic and tumor blood vessel formation. RTKs also are implicated in the regulation of cell proliferation, migration and survival.

The human epidermal growth factor receptor 2 gene (HER-2; also referred to as ErbB2) encodes a receptor tyrosine kinase that has been implicated as an oncogene. HER-2 has a major mRNA transcript of 4.5 Kb that encodes a polypeptide of about 185 kDa (p185HER2). P185HER2 contains an extracellular domain, a transmembrane domain and an intracellular domain with tyrosine kinase activity. Several polypeptide forms are produced from the HER-2 gene and include polypeptides generated by proteolytic processing and forms generated from alternatively spliced RNAs. Herstatins and fragments thereof are HER-2 binding proteins, encoded by the HER-2 gene. Herstatins (also referred to as p68HER-2) are encoded by alternatively spliced variants of the gene encoding thep 185-HER2receptor. For example, one herstatin occurs in fetal kidney and liver, and includes a 79 amino acid intron-encoded insert, relative to the membrane-localized receptor, at the C terminus (see U.S. Pat. No. 6,414,130 and U.S. Published Application No. 20040022785). Several herstatin variants have been identified (see, e.g., U.S. Pat. No. 6,414,130; U.S. Published Application No. 20040022785, U.S. application Ser. No. 09/234,208; U.S. application Ser. No.09/506,079; published international application Nos. WO0044403 and WO0161356). Herstatins lack an epidermal growth factor (EGF) homology domain and contain part of the extracellular domain, typically the first 340 amino acids, of p185-HER2. Herstatins contain subdomains I and II of the human epidermal growth factor receptor, the HER-2 extracellular domain and a C-terminal domain encoded by an intron. The resulting herstatin polypeptides typically contain 419 amino acids (340 amino acids from subdomains I and II, plus 79 amino acids from intron 8). The herstatin proteins lack extracellular domain IV, as well as the transmembrane domain and kinase domain.

In contrast, positive acting EGFR ligands, such as the epidermal growth factor and transforming growth factor-alpha, possess such domains. Additionally, binding of a herstatin does not activate the receptor. Herstatins can inhibit members of the EGF-family of receptor tyrosine kinases as well as the insulin-like growth factor-1 (IGF-1) receptor and other receptors. Herstatins prevent the formation of productive receptor dimers (homodimers and heterodimers) required for transphosphorylation and receptor activation. Alternatively or additionally, herstatin can compete with a ligand for binding to the receptor terminus (see, U.S. Pat. No. 6,414,130; U.S. Published Application No. 20040022785, U.S. application Ser. No. 09/234,208; U.S. application Ser. No.09/506,079; published international application Nos. WO0044403 and WO0161356).

The tumor necrosis factor family of receptors (TNFRs) is another example of a family of receptors involved in signal transduction and regulation. The TNF ligand and receptor family regulate a variety of signal transduction pathways including those involved in cell differentiation, activation, and viability. TNFRs contain an extracellular domain, including a ligand binding domain, a transmembrane domain and an intracellular domain that participates in signal transduction. Additionally, TNFRs are typically trimeric proteins that trimerize at the cell surface. TNFRs play a role in inflammatory diseases, central nervous system diseases, autoimmune diseases, airway hyper-responsiveness conditions such as asthma, rheumatoid arthritis and inflammatory bowel disease. TNFRs also play a role in infectious diseases, such as viral infection.

The TNF family of receptors (TNFR) exhibit homology among the extracellular domains. Some of these receptors initiate apoptosis, some initiate cell proliferation and some initiate both activities. Signaling by this family requires clustering of the receptors by a trimeric ligand and subsequent association of proteins with the cytoplasmic region of the receptors. The TNFR family contains a sub family with homologous 80-amino-acid cytoplasmic domains. This domain is referred to as a death domain (DD), so named because proteins that contain this domain are involved in apoptosis. The distinction between members of the TNFR family is exemplified by two TNFRs coded by distinct genes. TNFR1 (55 kDa) signals the initiation of apoptosis and the activation of the transcription factor NFκB. TNFR2 (75 kDa) functions also to induce signal activation of NFκB but not the initiation of apoptosis. TNFR1 contains a DD; TNFR2 does not.

In some cases, accumulations of altered molecules can be causative of pathological conditions and disease. In other cases, a disease or condition can result in altered molecule metabolism and lead to the accumulations of particular molecules in altered form and/or amount. One example is the accumulation of proteins and lipids as glycated products. The products, referred to as advanced glycation end products (AGEs), are the result of nonenzymatic glycation and oxidation of proteins and lipids in the presence of aldose sugars. Initial early products are formed as reversible Schiff bases and Amadori products. Molecular rearrangements result in irreversible modifications to form AGEs. AGEs accumulate during the normal aging process in humans. AGE accumulation can be accelerated in particular diseases and conditions.

The accumulation of AGEs impact cell and tissue metabolism and signal transduction through their interactions with cellular binding proteins. One such binding protein is the receptor for advanced glycation end products (RAGE). RAGE interaction with AGEs is implicated in induction of cellular oxidant stress responses, including the RAS-MAP kinase pathway and NF-κB activation.

RAGE also binds to other molecules, including small molecules and proteins. S100A12 (also known as EN-RAGE, p6 and calgranulin C) is a calcium binding protein that can act as a ligand for RAGE. RAGE also can interact with β-sheet fibrilar materials including amyloid β-peptides, Aβ, amylin, serum amyloid A and prion-derived peptides. Amphoterin, a heparin-binding neurite outgrowth promoting protein also is a ligand for RAGE. Each of these ligand interactions can affect signal transduction pathways. Binding of these ligands to RAGE leads to cellular activation mediated by receptor-dependent signaling to thereby mediate or participate in a variety of disease processes. These include diabetic complications, amyloidoses, inflammatory/immune disorders and tumors.

Because of their involvement in a variety of diseases and conditions, cell surface receptors (CSRs) such as RTKs, RAGE and TNFRs and their ligands are targets for therapeutic intervention. Among therapeutic proteins of interest are isoforms of cell surface receptors (CSR), and isoforms of ligands of CSRs, that modulate an activity of a CSR involved in a variety of diseases and conditions, including cancers, angiogenesis, and other diseases involving undesirable cell proliferation and inflammatory reactions (see, e.g., copending U.S. application Ser. No. 10/846,113 and corresponding International PCT published application No. WO 05/016,966; U.S. application Ser. No. 11/129,740 and corresponding International PCT published application No. WO 05/113,596; U.S. Provisional application No. 60/678,076 and corresponding U.S. application No. 11/429,090 and International application No. PCTUS2006/17786; and U.S. Provisional application No. 60/735,609 and corresponding U.S. application No. (Attorney Docket No. 17118-045001/2824) and International application No. (Attorney Dockety No. 17118-045WO1/2824PC). These therapeutic proteins target diseases and disorders that involve disregulation of and/or changes in the modulation of signal transduction pathways

To permit effective use of such therapeutic molecules, it is important to optimize methods for production. While such molecules are known and available, a need exists to produce large quantities for widespread dissemination and use thereof . Accordingly, among the objects herein, it is an object to provide methods for production of such therapeutic isoforms as well as nucleic acid molecules that encode fusions of the therapeutic molecules with polypeptides that improve the secretion, expression, and/or purification thereof.

### SUMMARY

Provided are methods and products for production of therapeutic isoforms of CSRs and ligands and nucleic acid molecules that encode fusions of the therapeutic isoforms, that improve the secretion, expression, and/or purification. The isoforms can additionally can include additional functional moieties, such as multimerization domains, including Fc domains.

Provided herein are polypeptides of receptor tyrosine kinase (RTK) isoforms, including intron fusion proteins, operatively linked to a heterologous precursor sequence sufficient to effect secretion and/or trafficking of the RTK isoform. The RTK isoforms provided herein for operative linkage include those that contain an endogenous signal sequence and those that do not contain an endogenous signal sequence.

Provided herein are RTK isoform polypeptides operatively linked to a tissue plasminogen activator (tPA) precursor sequence (tPA pre/prosequence), or a sufficient portion of a tPA pre/prosequence to effect secretion of the RTK isoform. Included are RTK isoform polypeptides operatively linked to a tPA pre/prosequence having a sequence of amino acids set forth in SEQ ID NO:2, or allelic variants thereof.

Provided herein are RTK isoform polypeptides including any one of RTK that is an isoform of a VEGFR, FGFR, PDGFR, MET, EPH, TIE, DDR, or HER polypeptide including isoforms of a DDR1, EphA1, EphA2, EphB1, EphB4, EGFR, HER2, ErbB3, FGFR-1, FGFR-2, FGFR-4, MET, RON, CSF1R, KIT, PDGFR-A, PDGFR-B, TEK, Tie-1, VEGFR-1, VEGFR-2, or VEGFR-3 operatively linked to a tPA pre/prosequence.

Provided herein are RTK isoforms having a sequence of amino acids set forth in any one of SEQ ID NOS: 140, 142, 143, 145, 147, 149, 150,152, 153, 155, 157, 159, 161-168, 170, 172, 174, 176, 178, 180,181, 183, 185, 186, 188, 190, 192, 194, 196, 198, 200, 202, 204, 206, 208, 210, 212, 214, 216, 217, 219, 221, 223, 225, 227, 229-231, 233, 245, 247-251, 253, 255, 257, 259, 261, 263-270, 274-280, 282, 284, 286, 288, 289-303, or an active portion thereof operatively linked to all or a portion of a tPA pre/prosequence sufficient to effect secretion of the isoform.

Provided herein are RTK isoforms, including intron fusion proteins, operatively linked to a tPA pre/prosequence by a linker, including a restriction enzyme linker. Included are polypeptides of RTK isoforms wherein the restriction enzyme linker is joined between the isoform and all or a portion of a tPA pre/prosequence to effect secretion of the isoform. Also included are polypeptides of RTK isoforms optionally including a tag that facilitates polypeptide purification and/or detection. The tag can be joined between the restriction enzyme linker and all or a portion of a tPA pre/prosequence to effect secretion of the polypeptide. Alternatively, the tag can be joined between the restriction enzyme linker and the isoform. The tag can be a myc tag or a Poly His tag.

Provided herein are isoform polypeptides of a VEGFR-1, FGFR-2, FGFR-4, TEK, RON, or MET operatively linked to all or a portion of a tPA pre/prosequence containing a restriction enzyme linker and also optionally a myc tag. The tPA-isoform fusions, including tPA-intron fusion protein fusions, have a sequence of amino acids set forth in any one of SEQ ID NOS: 32, 34, 36, 40, 42, 46, or 48.

Provided herein are isoform polypeptides of a HER2, including intron fusion proteins, operatively linked to all or a portion of a tPA pre/prosequence containing a restriction enzyme linker and also optionally a Poly-His tag. The tPA-HER2 isoform- has a sequence of amino acids set forth in SEQ ID NO:38.

Provided herein are polypeptides of receptor for advanced glycation endproducts (RAGE) isoforms, including intron fusion proteins, operatively linked to a heterologous precursor sequence sufficient to effect secretion and/or trafficking of the RAGE isoform. The RAGE isoforms provided herein for operative linkage include those that contain an endogenous signal sequence and those that do not contain an endogenous signal sequence.

Provided herein are RAGE isoform polypeptides operatively linked to a tissue plasminogen activator (tPA) precursor sequence (tPA pre/prosequence), or a sufficient portion of a tPA pre/prosequence to effect secretion of the RAGE isoform. Included are RAGE isoform polypeptides operatively linked to a tPA pre/prosequence having a sequence of amino acids set forth in SEQ ID NO:2, or allelic variants thereof.

Provided herein are a RAGE isoforms having a sequence of amino acids set forth in any one of SEQ ID NOS: 235, 237, 239, 241, 243 or an active portion thereof operatively linked to all or a portion of a tPA pre/prosequence sufficient to effect secretion of the isoform.

Provided herein are RAGE isoforms, including intron fusion proteins, operatively linked to a tPA pre/prosequence by a linker, including a restriction enzyme linker. Included are polypeptides of RAGE isoform intron fusion proteins wherein the restriction enzyme linker is joined between the isoform and all or a portion of a tPA pre/prosequence to effect secretion of the isoform. Also included are polypeptides of RTK isoforms optionally including a tag that facilitates polypeptide purification and/or detection. The tag can be joined between the restriction enzyme linker and all or a portion of a tPA pre/prosequence to effect secretion of the polypeptide. The tag can be a myc tag.

Provided herein are isoform polypeptides of a RAGE operatively linked to all or a portion of a tPA pre/prosequence containing a restriction enzyme linker and also optionally a myc tag. The tPA-RAGE isoform has a sequence of amino acids set forth in SEQ ID NO: 44.

Provided herein are polypeptides of tumor necrosis factor receptor (TNFR) isoforms, including intron fusion proteins, operatively linked to a heterologous precursor sequence sufficient to effect secretion and/or trafficking of the TNFR isoform. The TNFR isoforms provided herein for operative linkage include those that contain an endogenous signal sequence and those that do not contain an endogenous signal sequence.

Provided herein are TNFR isoform polypeptides operatively linked to a tissue plasminogen activator (tPA) precursor sequence (tPA pre/prosequence), or a sufficient portion of a tPA pre/prosequence to effect secretion of the TNFR isoform. Included are TNFR isoform polypeptides operatively linked to a tPA pre/prosequence having a sequence of amino acids set forth in SEQ ID NO:2, or allelic variants thereof.

Provided herein are TNFR isoform polypeptides including an isoform of a TNFR1 or TNFR2 operatively linked to a tPA pre/prosequence.

Provided herein are a TNFR2 isoforms having a sequence of amino acids set forth in any one of SEQ ID NO: 272, or an active portion thereof operatively linked to all or a portion of a tPA pre/prosequence sufficient to effect secretion of the isoform.

Provided herein are TNFR isoform polypeptides, including intron fusion proteins, operatively linked to a tPA pre/prosequence by a linker, including a restriction enzyme linker. Included are polypeptides of TNFR isoforms wherein the restriction enzyme linker is joined between the isoform and all or a portion of a tPA pre/prosequence to effect secretion of the isoform. Also included are polypeptides of TNFR isoforms optionally including a tag that facilitates polypeptide purification and/or detection. The tag can be joined between the restriction enzyme linker and all or a portion of a tPA pre/prosequence to effect secretion of the polypeptide. The tag can be a myc tag.

Provided herein are polypeptides of hepatocyte growth factor (HGF) isoforms, including intron fusion proteins, operatively linked to a heterologous precursor sequence sufficient to effect secretion and/or trafficking of the HGF isoforms. The HGF isoforms provided herein for operative linkage include those that contain an endogenous signal sequence and those that do not contain an endogenous signal sequence.

Provided herein are HGF isoform polypeptides operatively linked to a tissue plasminogen activator (tPA) precursor sequence (tPA pre/prosequence), or a sufficient portion of a tPA pre/prosequence to effect secretion of the HGF isoform. Included are HGF isoform polypeptides operatively linked to a tPA pre/prosequence having a sequence of amino acids set forth in SEQ ID NO:2, or allelic variants thereof.

Provided herein are HGF isoforms having a sequence of amino acids set forth in any one of SEQ ID NO: 350, 352, or 354, or an active portion thereof operatively linked to all or a portion of a tPA pre/prosequence sufficient to effect secretion of the isoform.

Provided herein are HGF isoform polypeptides, including intron fusion proteins, operatively linked to a tPA pre/prosequence by a linker, including a restriction enzyme linker. Included are polypeptides of HGF isoforms wherein the restriction enzyme linker is joined between the isoform and all or a portion of a tPA pre/prosequence to effect secretion of the isoform. Also included are polypeptides of HGF isoforms optionally including a tag that facilitates polypeptide purification and/or detection. The tag can be joined between the restriction enzyme linker and all or a portion of a tPA pre/prosequence to effect secretion of the polypeptide. The tag can be a myc tag.

Also encompassed are polypeptides that are allelic variants, species variants, or variants having at least 80%, 85%, 90%, 95%, 96%, 97%, 98%, 99%, or more sequence identity to any of the polypeptide isoforms provided herein and that retain an activity as compared to an isoform of a polypeptide provided herein.

Provided herein are DNA constructs containing nucleic acid molecules encoding CSR isoforms, including isoforms of RTK, TNFR, or RAGE operatively linked to a heterologous precursor sequence. Included among these are nucleic acids of the tPA pre/prosequence isoform polypeptide fusions. Provided herein are nucleic acid molecules having a sequence of nucleic acids set forth in SEQ ID NOS. 31, 33, 35, 37, 39, 41, 43, 45, or 47, and allelic variants thereof.

Provided herein are vectors containing the nucleic acid molecules. Vectors include mammalian vectors. Included among mammalian vectors are a pDrive vector, pCI vector, or pcDNA 3.1 vector. Vectors also can include an adenovirus vector, an adeno-associated virus vector, EBV, SV40, cytomegalovirus vector, vaccinia virus vector, herpesvirus vector, a retrovirus vector, a lentivirus vector, or an artificial chromosome. Vectors can be those that remain episomal or integrate into the chromosome of a cell into which they are introduced.

Also provided are cells containing a vector as described herein. Cells include mammalian cells. Included among mammalian cells are mouse, rat, human, monkey, chicken, or hamster cells, including CHO, Balb/3T3, HeLa, MT2, mouse NS0 and other myeloma cell lines, hybridoma and heterohybridoma cell lines, lymphocytes, fibroblasts, Sp2/0, COS, NIH3T3, HEK293, 293T, S93S, 2B8, HKB, or EBNA-l cells.

Provided herein are methods of producing an isoform by culturing any of the cells described herein to effect the secretion of an isoform. The secreted isoform can be further purified from the cell culture. An epitope tag expressed by the secreted isoform can facilitate protein purification. Also provided herein are methods by which the secreted isoform is treated with an exoprotease, including a plasmin-like exoprotease.

Provided herein are methods of producing an isoform by introducing a cell with a DNA construct to effect the secretion of the isoform from the cells. Exemplary DNA constructs include any described herein encoding a polypeptide of an isoform operatively linked to a heterologous precursor sequence such as a tPA pre/prosequence. The DNA construct can be introduced into a mammalian cell including mouse, rat, human, monkey, chicken, or hamster cells, including CHO, Balb/3T3, HeLa, MT2, mouse NS0 and other myeloma cell lines, hybridoma and heterohybridoma cell lines, lymphocytes, fibroblasts, Sp2/0, COS, NIH3T3, HEK293, 293T, S93S, 2B8, HKB, or EBNA-1 cells. Introduction of a DNA construct can be by transfection, electroporation, or nuclear microinjection. Exemplary methods of introducing a DNA construct into a cell include using calcium phosphate, a cationic lipid reagent, or a polycation. Examples of cationic lipid compounds include, but are not limited to: Lipofectin (Life Technologies, Inc., Burlington, Ont.)(1:1 (w/w) formulation of the cationic lipid N-[1-(2,3-dioleyloxy)propyl]-N,N,N-trimethylammonium chloride (DOTMA) and dioleoyl-phosphatidyl-ethanol-amine (DOPE)); LipofectAMINE (Life Technologies, Burlington, Ont., see U.S. Pat. No. 5,334,761) (3:1 (w/w) formulation of polycationic lipid 2,3-dioleyloxy-N-[2(spermine-carboxamido)ethyl]-N,N-dimethyl-1-propanaminiumtrifluoroacetate (DOSPA) and dioleoyl phosphatidyi-ethanolamine (DOPE)), LipofectAMINE PLUS (Life Technologies, Burlington, Ont. see U.S. Pat. Nos. 5,334,761 and 5,736,392; see, also U.S. Pat. No. 6,051,429) (LipofectAmine and Plus reagent), LipofectAMINE 2000 (Life Technologies, Burlington, Ont.; see also International PCT application No. WO 00/27795). Further provided herein, are methods of purifying the isoform from the cell culture. Purification can be facilitated by expression of an epitope tag by the isoform. Also provided herein are methods by which the secreted isoform is treated with an exoprotease, including a plasmin-like exoprotease.

Provided herein are polypeptides of cell surface receptor or ligand isoforms, including intron fusion protein iso forms, that lack an endogenous precursor sequence and further contain additional amino acids at its N-terminus. The endogenous precursor sequence that the polypeptide lacks can be a signal sequence or can be a signal sequence and one additional amino acid. Exemplary iso forms include isoforms of CSRs including isoforms of an RTK, TNFR, or RAGE receptor. Isoforms also can include ligand isoforms such as an HGF isoform. The iso forms provided herein as polypeptides lacking a precursor sequence have a sequence of amino acids set forth in any one of SEQ ID NOS: 140, 142, 143, 145, 147, 149, 150, 152, 153, 155, 157, 159, 161-168, 170, 172, 174, 176, 178, 180, 181, 183, 185, 186, 188,190, 192, 194,196, 198,200,202, 204,206, 208, 210, 212, 214, 216, 217, 219, 221, 223, 225, 227, 229-231, 233, 235, 237, 239, 241, 243, 245, 247-251, 253, 255, 257, 259, 261, 263-270, 272, 274-280, 282, 284, 286, 288, 289-303, 350, 352, or 354, or an active portion thereof. The one or more additional amino acids included at the N-terminus of a polypeptide of an isoform provided herein can include a restriction enzyme linker sequence, a portion of a prosequence of tPA, or an epitope tag. Included among sequences that can be included at the N-terminus of an isoform polypeptide include GAR, SR, LE, or combinations thereof including GARSR or GARLE. Also provided are pharmaceutical compositions containing the polypeptide isoforms that contain one or more additional amino acids at their N-terminus.

Provided herein are methods of treating a disease or condition by administering any of the pharmaceutical compositions, described herein. Diseases or conditions treated include inflammatory diseases, cancer, angiogenesis-mediated diseases, or hyperproliferative diseases. Exemplary diseases include, but are not limited to, ocular disease, atherosclerosis, diabetes, rheumatoid arthritis, hemangioma, wound healing, Alzheimer's disease, Creutzfeldt-Jakob disease, Huntington's disease, smooth muscle proliferative-related disease, multiple sclerosis, cardiovascular disease, and kidney disease.

Exemplary of cancers are carcinoma, lymphoma, blastoma, sarcoma, leukemia, lymphoid malignancies, squamous cell cancer, lung cancer including small-cell lung cancer, non-small-cell lung cancer, adenocarcinoma of the lung and squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial/uterine carcinoma, salivary gland carcinoma, kidney or renal cancer, prostate cancer, vulval cancer, thyroid cancer, hepatic carcinoma, anal carcinoma, penile carcinoma, and head and neck cancer.

### DETAILED DESCRIPTION

Outline

A. DEFINITIONS
B. CELL SURFACE RECEPTOR AND LIGAND ISOFORMS
   1. CELL SURFACE RECEPTOR ISOFORMS
   2. LIGAND ISOFORMS
   3. ALLELIC AND SPECIES VARIANTS OF ISOFORMS AND MUTATIONS
C. ISOFORM FUSION PROTEIN PRODUCTION
   1. SECRETION
   2. PURIFICATION AND/OR DETECTION
D. ISOFORM FUSIONS
   1. EXEMPLARY tPA SECRETORY SEQUENCE
   2. tPA-INTRON FUSION PROTEIN AND OTHER CSR FUSIONS
      a. FGFR-2 tPA-intron fusion protein Fusion
      b. FGFR4-tPA intron fusion protein Fusion
      c. VEGFR-1-tPA intron fusion protein Fusion
      d. tPA-MET intron fusion protein FUSION
      e. tPA-RON intron fusion protein FUSION
      f. tPA-HER2 intron fusion protein FUSION
      g. tPA-RAGE intron fusion protein FUSION
      h. tPA-TEK intron fusion protein FUSION
E. METHODS FOR PRODUCING NUCLEIC ACID ENCODING ISOFORM FUSION POLYPEPTIDES
   1. SYNTHETIC GENES AND POLYPEPTIDES
   2. METHODS OF CLONING AND ISOLATING ISOFORMS AND ISOFORM FUSIONS
   3. METHODS OF GENERATING AND CLONING intron fusion protein FUSIONS
   4. EXPRESSION SYSTEMS
      a. PROKARYOTIC EXPRESSION
      b. YEAST
      c. INSECT CELLS
      d. MAMMALIAN CELLS
      e. PLANTS
   5. METHODS OF TRANSFECTION AND TRANSFORMATION
   6. PRODUCTION AND PURIFICATION
   7. SYNTHETIC ISOFORMS
   8. FORMATION OF MULTIMERS
      a. PEPTIDE LINKERS
      b. POLYPEPTIDE MULTIMERIZATION DOMAINS
         i. IMMUNOGLOBULIN DOMAIN
            (A) FC DOMAIN
            (B) PROTUBERANCES-INTO-CAVITY (I.E. KNOBS AND HOLES)
         ii. LEUCINE ZIPPER
            (A) FOS AND JUN
            (B) GCN4
         iii. OTHER MULTIMERIZATION DOMAINS
            R/PKA-AD/AKAP
F. ASSAYS TO ASSESS ACTIVITY OF AN ISOFORM
   1. KINASE ASSAYS
   2. COMPLEXATION
   3. LIGAND BINDING
   4. RECEPTOR BINDING
   5. CELL PROLIFERATION ASSAYS
   6. MOTOGENIC ASSAYS
   7. APOPTOTIC ASSAYS
   8. CELL DISEASE MODEL ASSAYS
   9. ANIMAL MODELS
G. PREPARATION, FORMULATION AND ADMINISTRATION OF CSR AND LIGAND ISOFORMS AND CSR AND LIGAND ISOFORM COMPOSITIONS
H. IN VIVO EXPRESSION OF CSR AND LIGAND ISOFORMS AND GENE THERAPY
   1. DELIVERY OF NUCLEIC ACIDS
      a. VECTORS—EPISOMAL AND INTEGRATING
      b. ARTIFICIAL CHROMOSOMES AND OTHER NON-VIRAL VECTOR DELIVERY METHODS
      c. LIPOSOMES AND OTHER ENCAPSULATED FORMS AND ADMINISTRATION OF CELLS CONTAINING THE NUCLEIC ACIDS
   2. IN VITRO AND EX VIVO DELIVERY
   3. SYSTEMIC, LOCAL AND TOPICAL DELIVERY
I. EXEMPLARY TREATMENTS AND STUDIES WITH CSR ISOFORMS
   1. ANGIOGENESIS-RELATED CONDITIONS
   2. ANGIOGENESIS-RELATED ATHEROSCLEROSIS
   3. ANGIOGENESIS-RELATED DIABETES
      a. VASCULAR DISEASE
      b. PERIODONTAL DISEASE
   4. ADDITIONAL ANGIOGENESIS-RELATED TREATMENTS
   5. CANCERS
   6. ALZHEIMER'S DISEASE
   7. SMOOTH MUSCLE PROLIFERATIVE-RELATED DISEASES AND CONDITIONS
   8. INFLAMMATORY DISEASES
   9. CARDIOVASCULAR DISEASE
   10. KIDNEY DISEASE
J. COMBINATION THERAPIES
K. EXAMPLES
   A. DEFINITIONS

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, a cell surface receptor (CSR) is a protein that is expressed on the surface of a cell and typically includes a transmembrane domain or other moiety that anchors it to the surface of a cell. As a receptor it binds to ligands that mediate or participate in an activity of the cell surface receptor, such as signal transduction or ligand internalization. Cell surface receptors include, but are not limited to, single transmembrane receptors and G-protein coupled receptors. Receptor tyrosine kinases, such as growth factor receptors, also are among such cell surface receptors.

As used herein, a receptor tyrosine kinase (RTK) refers to a protein, typically a glycoprotein, that is a member of the growth factor receptor family of proteins. Growth factor receptors are typically involved in cellular processes including cell growth, cell division, differentiation, metabolism and cell migration. RTKs also are known to be involved in cell proliferation, differentiation and determination of cell fate as well as tumor growth. RTKs have a conserved domain structure including an extracellular domain, a membrane-spanning (transmembrane) domain and an intracellular tyrosine kinase domain. Typically, the extracellular domain binds to a polypeptide growth factor or a cell membrane-associated molecule or other ligand. The tyrosine kinase domain is involved in positive and negative regulation of the receptor.

Receptor tyrosine kinases are grouped into families based on, for example, structural arrangements of sequence motifs in their extracellular domains. Structural motifs include, but are not limited to, repeats of regions of: immunoglobulin, fibronectin, cadherin, epidermal growth factor and kringle repeats. Classification by structural motifs has identified greater than 16 families of RTKs, each with a conserved tyrosine kinase domain. Examples of RTKs include, but are not limited to, erythropoietin-producing hepatocellular (EPH) receptors, epidermal growth factor (EGF) receptors, fibroblast growth factor (FGF) receptors, platelet-derived growth factor (PDGF) receptors, vascular endothelial growth factor (VEGF) receptor, cell adhesion RTKs (CAKs), Tie/Tek receptors, insulin-like growth factor (IGF) receptors, and insulin receptor related (IRR) receptors. Exemplary genes encoding RTKs include, but are not limited to, ErbB2, ErbB3, DDR1, DDR2, EGFR, EphA1, EphA8, FGFR-2, FGFR-4, Flt1 (fins-related tyrosine kinase 1 receptor; also known as VEGFR-1), FLK1 (also known as VEGFR-2) MET, PDGFRA, PDGFRB, and TEK (also known as TIE-2).

Dimerization of RTKs activates the catalytic tyrosine kinase domain of the receptor and tyrosine autophosphorylation. Autophosphorylation in the kinase domain maintains the tyrosine kinase domain in an activated state. Autophosphorylation in other regions of the protein influences interactions of the receptor with other cellular proteins. In some RTKs, ligand binding to the extracellular domain leads to dimerization of the receptor. In some RTKs, the receptor can dimerize in the absence of ligand. Dimerization also can be increased by receptor overexpression.

As used herein, a tumor necrosis factor receptor (TNFR) refers to a member of a family of receptors that have a characteristic repeating extracellular cysteine-rich motif such as found in TNFR1 and TNFR2. TNFRs also have a variable intracellular domain that differs between members of the TNFR family. The TNFR family of receptors includes, but is not limited to, TNFR1, TNFR2, TNFRrp, the low-affinity nerve growth factor receptor, Fas antigen, CD40, CD27, CD30, 4-1BB, OX40, DR3, DR4, DR5, and herpesvirus entry mediator (HVEM). Ligands for TNFRs include TNF-α, lymphotoxin, nerve growth factor, Fas ligand, CD40 ligand, CD27 ligand, CD30 ligand, 4-1BB ligand, OX40 ligand, APO3 ligand, TRAIL, LIGHT, and BTLA. TNFRs include an extracellular domain, including a ligand binding domain, a transmembrane domain and an intracellular domain that participates in signal transduction. TNFRs are typically trimeric proteins that trimerize at the cell surface.

As used herein, a ligand is an extracellular substance, generally a polypeptide, that binds to one or more receptors. A ligand can be soluble or can be a transmembrane protein. For purposes herein, a ligand binds to a receptor and induces signal transduction by the receptor.

As used herein, signal transduction refers to a series of sequential events, such as protein phosphorylations, consequent upon binding of a ligand by a transmembrane receptor, that transfers a signal through a series of intermediate molecules until final regulatory molecules, such as transcription factors, are modified in response to the signal. Responses triggered by signal transduction include the activation of specific genes. Gene activation leads to further effects, since genes are expressed as proteins many of which are enzymes, transcription factors, or other regulators of metabolic activity that mediate any one or more biological activities of a ligand-receptor interaction.

As used herein, an isoform refers to a protein that has an altered polypeptide structure compared to a full-length wildtype (predominant) form of the cognate protein due to a differences in the nucleic acid sequence and encoded polypeptide of the isoform compared to the corresponding protein. For purposes herein, isoforms include isoforms of a cell surface receptor (CSR) and isoforms of a ligand of a CSR. Generally an isoform provided herein lacks a domain or portion thereof (or includes insertions or both) sufficient to alter an activity, such as an enzymatic activity of a predominant form of the protein, or the structure of the protein. Reference herein to an isoform with altered activity refers to the alteration in an activity by virtue of the different structure or sequence of the isoform compared to a full-length or predominant form of the protein. With reference to an isoform, alteration of activity refers to a difference in activity between the particular isoforms and the predominant or wildtype form. Alteration of an activity includes an enhancement or a reduction of activity. In one embodiment, an alteration of an activity is a reduction in an activity; the reduction can be at least 0.1, 0.5, 1, 2, 3, 4, 5, or 10 fold compared to a wildtype and/or predominant form of the receptor. Typically, an activity is reduced 5, 10, 20, 50, 100 or 1000 fold or more. For example, a ligand can bind to a receptor and initiate or participate in signal transduction.

As used herein, a ligand isoform refers to a ligand that lacks a domain or portion of a domain or that has a disruption in a domain such as by the insertion of one or more amino acids compared to polypeptides of a wildtype or predominant form of the ligand. Typically such isoforms are encoded by alternatively spliced variants of the gene encoding the cognate ligand. Among the ligand isoforms provided herein are those that can bind to receptors but do not initiate signal transduction or initiate a reduced level of signal transduction. Such ligand isoforms act as ligand antagonists, and also process reduced activity as agonists compared to the wildtype ligand. A ligand isoform generally lacks a domain or portion thereof sufficient to alter an activity of a wildtype full-length and/or predominant form of the ligand, and/or modulates an activity of its receptor, or lacks a structural feature such as a domain. Such ligand isoforms, also include insertions and rearrangements. A ligand isoform includes those that exhibit activities that are altered from the corresponding wild-type ligand; for example, an isoform can include an alteration in a domain of the ligand so that it is unable to induce the dimerization of a receptor. In such an example, an isoform can compete for binding with a full-length wildtype ligand for its receptor, but reduce or inhibit signaling by the receptor. Generally, an activity is altered in an isoform at least 0.1, 0.5, 1, 2, 3, 4, 5, or 10 fold compared to a wildtype and/or predominant form of a ligand. Typically, an activity is altered by at least 2, 5, 10, 20, 50, 100, or 1000 fold or more. In one embodiment, alteration of an activity by a ligand isoform is a reduction in the activity compared to the predominant form of the ligand.

As used herein, a cell surface receptor (CSR) isoform, such as an isoform of a receptor tyrosine kinase, refers to a receptor that lacks a domain or portion thereof sufficient to alter or modulate an activity compared to a wildtype and/or predominant form of the receptor, or lacks a structural feature, such as a domain. A CSR isoform can include an isoform that has one or more biological activities that are altered from the receptor; for example, an isoform can include an alteration of the extracellular domain of p185-HER2, altering the isoform from a positively acting regulatory polypeptide of the receptor to a negatively acting regulatory polypeptide of the receptor, e.g. from a receptor domain into a ligand. Generally, an activity is altered in an isoform at least 0.1, 0.5, 1, 2, 3, 4, 5, or 10 fold compared to a wildtype and/or predominant form of the receptor. Typically, an activity is altered by at least 2, 5, 10, 20, 50, 100 or 1000 fold or more. In one embodiment, alteration of an activity is a reduction in the activity.

As used herein, reference to modulating the activity of a cell surface receptor means that a CSR or ligand isoform interacts in some manner with the receptor, whereby an activity, such as, but not limited to, ligand binding, dimerization and/or other signal-transduction-related activity, is altered.

As used herein, reference to a CSR isoform or ligand isoform with altered activity refers to an alteration in an activity by virtue of the different structure or sequence of the CSR or ligand isoform compared to a cognate receptor or ligand.

As used herein, an intron fusion protein refers to an isoform that lacks one or more domain(s) or portion of one or more domain(s). In addition, an intron fusion protein is encoded by nucleic acid molecules that contain one or more codons (with reference to the predominant or wildtype form of a protein), including stop codons, operatively linked to exon codons. The intron portion can be a stop codon, resulting in an intron fusion protein that ends at the exon intron junction. The activity of an intron fusion protein typically is different from the predominant form, generally by virtue of truncation(s), deletions and/or insertion of intron(s) amino acid residues. Such activities include changes in interaction with a receptor, or indirect changes that occur virtue of differences in interaction with a co-stimulating receptor or ligand, a receptor ligand or co-factor or other modulator of receptor activity. Intron fusion proteins isolated from cells or tissues or that have the sequence of such polypeptides isolated from cells or tissues, are “natural.” Those that do not occur naturally but that are synthesized or prepared by linking a molecule to an intron are referred to as “synthetic” or “recombinant” or “combinatorial”. Included among intron fusion proteins are CSR isoforms or ligand isoforms that lack one or more domain(s) or portion of one or more domain(s) resulting in an alteration of an activity of a cognate receptor or ligand by virtue of a change in the interaction between the intron fusion protein and its receptor or ligand or other interaction. Generally such isoforms are shortened compared to a wildtype or predominant form encoded by a CSR or ligand gene. They, however, can include insertions or other modifications in the exon portion and, thus, be of the same size or larger than the predominant form. Each, however, is encoded by a nucleic acid molecule that includes at least one codon (including stop codons) from an intron-encoded portion resulting either in truncation of the CSR or ligand isoform at the end of the exon or in the addition of one 2, 3, 4, 5, 8, 10, 15, 20, 25, 30, 35, 40, 45, 50, 55, 60, 65, 70, 75 and more amino acids encoded by an intron.

An intron fusion protein can be encoded by an alternatively spliced RNA and/or can be synthetically produced such as from RNA molecules identified in silico by identifying potential splice sites and then producing such molecules by recombinant methods. Typically, an intron fusion protein is shortened by the presence of one or more stop codons in an intron fusion protein-encoding RNA that are not present in the corresponding sequence of an RNA encoding a wildtype or predominant form of a corresponding polypeptide. If an intron includes an open reading frame in-frame with the exon portion, the intron encoded portion can be inserted in the polypeptide. Addition of amino acids and/or a stop codon results in an intron fusion protein that differs in size and sequence from a wildtype or predominant form of a polypeptide.

Intron fusion proteins for purposes herein include natural, combinatorial and synthetic intron fusion proteins. A natural intron fusion protein refers to a polypeptide that is encoded by an alternatively spliced RNA molecule that contains one or more amino acids encoded by an intron linked to one or more portions of the polypeptide encoded by one or more exons of a gene. Alternatively spliced mRNA is isolated or can be prepared synthetically by joining splice donor and acceptor sites in a gene. A natural intron fusion protein contains one or more amino acids or is truncated at the exon-intron junction because the intron contains a stop codon as the first codon. The natural intron fusion proteins generally occur in cells and/or tissues. Intron fusion proteins can be produced synthetically, for example based upon the sequence encoded by a gene by identifying splice donor and acceptor sites and identifying possible encoded spliced variants. A combinatorial intron fusion protein refers to a polypeptide that is shortened compared to a wildtype or predominant form of a polypeptide. Typically, the shortening removes one or more domains or a portion thereof from a polypeptide such that an activity is altered. Combinatorial intron fusion proteins often mimic a natural intron fusion protein in that one or more domains or a portion thereof is/are deleted in a natural intron fusion protein derived from the same gene or derived from a gene in a related gene family. Those that do not occur naturally but that are synthesized or prepared by linking a molecule to an intron such that the resulting construct modulates the activity of a CSR are “synthetic.”

As used herein, natural with reference to intron fusion protein or a CSR or ligand isoform, refers to any protein, polypeptide or peptide or fragment thereof (by virtue of the presence of the appropriate splice acceptor/donor sites) that is encoded within the genome of an animal and/or is produced or generated in an animal or that could be produced from a gene. Natural intron fusion proteins include allelic variants and species variants. Intron fusion proteins can be modified post-translationally.

As used herein, an exon refers to a nucleic acid molecule containing a sequence of nucleotides that is transcribed into RNA and is represented in a mature form of RNA, such as MRNA (messenger RNA), after splicing and other RNA processing. An mRNA contains one or more exons operatively linked. Exons can encode polypeptides or a portion of a polypeptide. Exons also can contain non-translated sequences for example, translational regulatory sequences. Exon sequences are often conserved and exhibit homology among gene family members.

As used herein, an intron refers to a sequence of nucleotides that is transcribed into RNA and is then typically removed from the RNA by splicing to create a mature form of an RNA, for example, an MRNA. Typically, nucleotide sequences of introns are not incorporated into mature RNAs, nor are intron sequences or a portion thereof typically translated and incorporated into a polypeptide. Splice signal sequences such as splice donors and acceptors are used by the splicing machinery of a cell to remove introns from RNA. It is noteworthy that an intron in one splice variant can be an exon (i.e., present in the spliced transcript) in another variant. Hence, spliced mRNA encoding an intron fusion protein can include an exon(s) and introns.

As used herein, splicing refers to a process of RNA maturation where introns in the mRNA are removed and exons are operatively linked to create a messenger RNA (mRNA).

As used herein, alternative splicing refers to the process of producing multiple mRNAs from a gene. Alternate splicing can include operatively linking less than all the exons of a gene, and/or operatively linking one or more alternate exons that are not present in all transcripts derived from a gene.

As used herein, exon deletion refers to an event of alternative RNA splicing that produces a nucleic acid molecule that lacks at least one exon compared to an RNA molecule encoding a wildtype or predominant form of a polypeptide. An RNA molecule that has a deleted exon can be produced by such alternative splicing or by any other method, such as an in vitro method to delete the exon.

As used herein, exon insertion, refers to an event of alternative RNA splicing that produces a nucleic acid molecule that contains at least one exon not typically present in an RNA molecule encoding a wildtype or predominant form of a polypeptide. An RNA molecule that has an inserted exon can be produced by such alternative splicing or by any other method, such as an in vitro method to add or insert the exon.

As used herein, exon extension refers to an event of alternative RNA splicing that produces a nucleic acid molecule that contains at least one exon that is greater in length (number of nucleotides contained in the exon) than the corresponding exon in an RNA encoding a wildtype or predominant form of a polypeptide. An RNA molecule that has an extended exon can be produced by such alternative splicing or by any other method, such as an in vitro method to extend the exon. In some instances, as described herein, an mRNA produced by exon extension encodes an intron fusion protein.

As used herein, exon truncation refers to an event of alternative RNA splicing that produces a nucleic acid molecule that contains a truncation or shortening of one or more exons such that the one or more exons are shorter in length (number of nucleotides) compared to a corresponding exon in an RNA molecule encoding a wildtype or predominant form of a polypeptide. An RNA molecule that has a truncated exon can be produced by such alternative splicing or by any other method, such as an in vitro method to truncate the exon.

As used herein intron retention refers to an event of alternative RNA splicing that produces a nucleic acid molecule that contains an intron or a portion thereof operatively linked to one or more exons. An RNA molecule that retains an intron or portion thereof can be produced by such alternative splicing or by any other method, such as an in vitro method to produce an RNA molecule with a retained exon. In some cases, as described herein, an MRNA molecule produced by intron retention encodes an intron fusion protein.

As used herein, a gene, also referred to as a gene sequence, refers to a sequence of nucleotides transcribed into RNA (introns and exons), including a nucleotide sequence that encodes at least one polypeptide. A gene includes sequences of nucleotides that regulate transcription and processing of RNA. A gene also includes regulatory sequences of nucleotides such as promoters and enhancers, and translation regulation sequences.

As used herein, a splice site refers to one or more nucleotides within the gene that participate in the removal of an intron and/or the joining of an exon. Splice sites include splice acceptor sites and splice donor sites.

As used herein, an open reading frame refers to a sequence of nucleotides that encodes a functional polypeptide or a portion thereof, typically at least about fifty amino acids. An open reading frame can encode a full-length polypeptide or a portion thereof. An open reading frame can be generated by operatively linking one or more exons or an exon and intron, when the stop codon is in the intron and all or a portion of the intron is in a transcribed mRNA.

As used herein, a polypeptide refers to two or more amino acids covalently joined. The terms “polypeptide” and “protein” are used interchangeably herein.

As used herein, truncation or shortening with reference to the shortening of a nucleic acid molecule or protein, refers to a sequence of nucleotides or amino acids that is less than full-length compared to a wildtype or predominant form of the protein or nucleic acid molecule.

As used herein, a reference gene refers to a gene that can be used to map introns and exons within a gene. A reference gene can be genomic DNA or a portion thereof, that can be compared with, for example, an expressed gene sequence, to map introns and exons in the gene. A reference gene also can be a gene encoding a wildtype or predominant form of a polypeptide.

As used herein, a family or related family of proteins or genes refers to a group of proteins or genes, respectively that have homology and/or structural similarity and/or functional similarity with each other.

As used herein, a premature stop codon is a stop codon occurring in the open reading frame of a sequence before the stop codon used to produce or create a full-length form of a protein, such as a wildtype or predominant form of a polypeptide. The occurrence of a premature stop codon can be the result of, for example, alternative splicing and mutation.

As used herein, an expressed gene sequence refers to any sequence of nucleotides transcribed or predicted to be transcribed from a gene. Expressed gene sequences include, but are not limited to, cDNAs, ESTs, and in silico predictions of expressed sequences, for example, based on splice site predictions and in silico generation of spliced sequences.

As used herein, an expressed sequence tag (EST) is a sequence of nucleotides generated from an expressed gene sequence. ESTs are generated by using a population of MRNA to produce cDNA. The cDNA molecules can be produced for example, by priming from the polyA tail present on mRNAs. cDNA molecules also can be produced by random priming using one or more oligonucleotides which prime cDNA synthesis internally in mRNAs. The generated cDNA molecules are sequenced and the sequences are typically stored in a database. An example of an EST database is dbEST found online at ncbi.nlm.nih.gov/dbEST. Each EST sequence is typically assigned a unique identifier and information such as the nucleotide sequence, length, tissue type where expressed, and other associated data is associated with the identifier.

As used herein, cognate receptor with reference to the isoforms provided herein refers to the receptor that is encoded by the same gene as the particular isoform. Generally, the cognate receptor also is a predominant form in a particular cell or tissue. For example, herstatin is encoded by a splice variant of the pre-mRNA which encodes p185-HER2 (ErbB2 receptor). Thus, p185-HER2 is the cognate receptor for herstatin.

As used herein, a cognate ligand with reference to the isoforms provided herein refers to the ligand that is encoded by the same gene as the particular isoform. Generally, the cognate ligand also is a predominant form in a particular cell or tissue.

As used herein, a wildtype form, for example, a wildtype form of a polypeptide, refers to a polypeptide that is encoded by a gene. Typically a wildtype form refers to a gene (or RNA or protein derived therefrom) without mutations or other modifications that alter function or structure; wildtype forms include allelic variation among and between species.

As used herein, a predominant form, for example, a predominant form of a polypeptide, refers to a polypeptide that is the major polypeptide produced from a gene. A “predominant form” varies from source to source. For example, different cells or tissue types can produce different forms of polypeptides, for example, by alternative splicing and/or by alternative protein processing. In each cell or tissue type, a different polypeptide can be a “predominant form”.

As used herein, a domain refers to a portion (typically a sequence of three or more, generally 5 or 7 or more amino acids) of a polypeptide chain that can form an independently folded structure within a protein made up of one or more structural motifs (e.g. combinations of alpha helices and/or beta strands connected by loop regions) and/or that is recognized by virtue of a functional activity, such as kinase activity. A protein can have one, or more than one, distinct domain. For example, a domain can be identified, defined or distinguished by homology of the sequence therein to related family members, such as homology of motifs that define an extracellular domain. In another example, a domain can be distinguished by its function, such as by enzymatic activity, e.g. kinase activity, or an ability to interact with a biomolecule, such as DNA binding, ligand binding, and dimerization. A domain independently can exhibit a biological function or activity such that the domain independently or fused to another molecule can perform an activity, such as, for example proteolytic activity or ligand binding. A domain can be a linear sequence of amino acids or a non-linear sequence of amino acids from the polypeptide. Many polypeptides contain a plurality of domains. For example, receptor tyrosine kinases typically include, an extracellular domain, a membrane-spanning (transmembrane) domain and an intracellular tyrosine kinase domain.

As used herein, a polypeptide lacking all or a portion of a domain refers a polypeptide that has a deletion of one or more amino acids or all of the amino acids of a domain compared to a cognate polypeptide. Amino acids deleted in a polypeptide lacking all or part of a domain need not be contiguous amino acids within the domain of the cognate polypeptide. Polypeptides that lack all or a part of a domain can exhibit a loss or reduction of an activity of the polypeptide compared to the activity of a cognate polypeptide or loss of a structure in the polypeptide.

For example, if a cognate protein has a transmembrane domain, then an isoform polypeptide lacking all or a part of the transmembrane domain can have a deletion of 1, 2, 3, 4, 5, 6, 7, 8, 9, 10, 11, 12, 13, 14, 15, 16, 17, 18, 19, 20 or more amino acids of the amino acids corresponding to the same amino acid positions in the cognate polypeptide.

As used herein, a polypeptide that contains a domain refers to a polypeptide that contains a complete domain with reference to the corresponding domain of a cognate protein. A complete domain is determined with reference to the definition of that particular domain within a cognate polypeptide. For example, an isoform comprising a domain refers to an isoform that contains a domain corresponding to the complete domain as found in the cognate protein. If a cognate protein, for example, contains a transmembrane domain of 21 amino acids between amino acid positions 400-420, then a receptor isoform that comprises such a transmembrane domain, contains a 21 amino acid domain that has substantial identity with the 21 amino acid domain of the cognate protein. Substantial identity refers to a domain that can contain allelic variation and conservative substitutions compared to the domain of the cognate protein. Domains that are substantially identical do not have deletions, non-conservative substitutions or insertions of amino acids compared to the domain of the cognate protein.

Such domains are known to those of skill in the art who can identify such. Domains (i.e., a furin domain, an Ig-like domain) often are identified by virtue of structural and/or sequence homology to domains in particular proteins. For exemplification herein, definitions are provided, but it is understood that it is well within the skill in the art to recognize particular domains by name. If needed appropriate software can be employed to identify domains. Further, reference to the amino acids positions of a domain herein are for exemplification purposes only. Since interactions are dynamic, amino acid positions noted are for reference and exemplification. The noted positions reflects a range of loci that vary by 2, 3, 4, 5 or more amino acids. Variations also exist among allelic variants and species variants. Those of skill in the art can identify corresponding sequences by visual comparison or other comparisons including readily available algorithms and software.

As used herein, an extracellular domain is a portion of a cell surface receptor that occurs on the surface of the receptor and includes the ligand binding site(s). In one example, a receptor L domain (RLD) (also called an EGFR-like domain), such as for example in HER2, is an example of a domain that includes a ligand binding site. Each L domain contains a single-stranded right hand beta-helix that can associate with a second L domain to form a three-dimensional bilobal structure surrounding a central space of sufficient size to accommodate a ligand molecule.

As used herein, a furin domain is a domain recognized as such by those of skill in the art and is a cysteine rich region. Furin is a type 1 transmembrane serine protease. A furin domain can function as a cleavage site for a furin protease.

As used herein a Sema domain is a domain recognized as such by those of skill in the art and is a receptor recognition and binding module. The Sema domain is characterized by a conserved set of cysteine residues, which form four disulfide bonds to stabilize the structure. The Sema domain fold is a variation of a β propeller topology, with seven blades radially arranged around a central axis. Each blade contains a four-stranded antiparallel β sheet. The Sema domain uses a ‘loop and hook’ system to close the circle between the first and the last blades. The blades are constructed sequentially with an N-terminal β-strand closing the circle by providing the outermost strand of the seventh (C-terminal) blade. The β-propeller is further stabilized by an extension of the N-terminus, providing an additional, fifth β-strand on the outer edge of blade 6.

As used herein, a plexin domain is a domain recognized as such by those of skill in the art and contains a cysteine rich repeat. Plexins are receptors that as a complex interact with membrane-bound semaphorins. The plexins contain three domains with homology to c-met, the receptor for scatter factor-induced motility, but they lack the intrinsic tyrosine kinase activity of c-met. Intracellullarly, invariant arginines identify a plexin domain with homology to guanosine triphosphatase-activating proteins. A protein can contain one, or more than one, plexin domain. As described herein, the MET receptor contains a single plexin domain.

As used herein an Ig-like domain is a domain recognized as such by those of skill in the art and is a domain containing folds of beta strands forming a compact folded structure of two beta sheets stabilized by hydrophobic interactions and sandwiched together by an intra-chain disulfide bond. Ig domains differ in the number of strands in the beta sheets and are typically grouped into four types, Ig-like V-type, Ig-like C1-type, Ig-like C2-type, and I-set. In one example, an Ig-like C-type domain contains seven beta strands arranged as four-strand plus three-strand so that four beta strands form one beta sheet and three beta strands form the second beta sheet. In another example, an Ig-like V-type domain contains nine beta strands arranged as four beta strands plus five beta strands (Janeway C. A. et al. (eds): Immunobiology-the immune system in health and disease, 5th edn. New York, Garland Publishing, 2001.). In addition, some Ig-like domains cannot be classified into one of the above groups and are sometimes simply called Ig-like.

As used herein, the immunoglobulin superfamily is a heterogenic group of proteins containing immunoglobulin-like domains. Proteins of the immunoglobulin superfamily include proteins involved in the immune system such as immunoglobulins and the T cell receptors, proteins involved in cell-cell recognition in the nervous system and other tissues, and other proteins.

As used herein, a fibronectin type-III (FN3) domain is a domain recognized as such by those of skill in the art and contains a conserved β sandwich fold with one β sheet containing four strands and the other sheet containing three strands. The folded structure of an FN3 domain and an Ig-like domain are topologically very similar except the FN3 domain lacks a conserved disulfide bond. The portion of the polypeptide encoding an FN3 domain also is characterized by a short stretch of amino acids containing an Arg-Gly-Asp (RGD) that mediates interactions with cell adhesion molecules to modulate thrombosis, inflammation, and tumor metastasis.

As used herein, an IPT/TIG domain is a domain recognized as such by those of skill in the art has an immunoglobulin fold-like domain. Proteins contain one, or more than one, IPT/TIG domain. IPT/TIG domains are found in plexins, transcription factors, and extracellular regions of receptor proteins, such as for example the cell surface receptors MET and RON as described herein, that appear to regulate cell proliferation and cellular adhesion (Johnson C A et al, Journal of Medical Genetics, 40:311-319, (2003)).

As used herein, an EGF domain is a domain recognized as such by those of skill in the art and contains a repeat pattern involving a number of conserved cysteine residues which is important to the three-dimensional structure of the protein, and hence its recognition by receptors and other molecules. The EGF domain as described herein contains six cysteine residues which are involved in forming disulfide bonds. An EGF domain forms a two-stranded β sheet followed by a loop to a C-terminal short two-stranded sheet. Subdomains between the conserved cysteines vary in length. Repeats of EGF domains are typically found in the extracellular domain of membrane-bound proteins, such as for example in TEK as described herein. A variation of the EGF domain is the laminin (Lam) EGF domain which, as described herein, has eight instead of six conserved cysteines and therefore is longer than the average EGF module and contains a further disulfide bond C-terminal of the EGF-like region.

As used herein, a transmembrane domain spans the plasma membrane anchoring the receptor and generally includes hydrophobic residues.

As used herein, a cytoplasmic domain is a domain that participates in signal transduction and occurs in the cytoplasmic portion of a transmembrane cell surface receptor. In one example, the cytoplasmic domain can include a protein kinase (PK) domain. A PK domain is recognized as such by those of skill in the art and is a domain that contains a conserved catalytic core. The conserved catalytic core is recognized to have a glycine-rich stretch of residues in the vicinity of a lysine residue in the N-terminal extremity of the domain, which has been shown to be involved in ATP binding, and an aspartic acid residue in the central part of the catalytic domain, which is important for the catalytic activity of the enzyme. Typically, the PK domain can be a serine/threonine protein kinase or a tyrosine protein kinase domain depending on the substrate specificity of the receptor domain such that, for example, a protein containing a tyrosine kinase domain phosphorylates substrate proteins on tyrosine residues whereas, for example, a protein containing a serine/threonine protein kinase domain phosphorylates substrate proteins on serine or threonine residues.

As used herein, a kinase is a protein that is able to phosphorylate a molecule, typically a biomolecule, including macromolecules and small molecules. For example, the molecule can be a small molecule, or a protein. Phosphorylation includes auto- phosphorylation. Some kinases have constitutive kinase activity. Other kinases require activation. For example, many kinases that participate in signal transduction are phosphorylated. Phosphorylation activates their kinase activity on another biomolecule in a pathway. Some kinases are modulated by a change in protein structure and/or interaction with another molecule. For example, complexation of a protein or binding of a molecule to a kinase can activate or inhibit kinase activity.

As used herein, designated refers to the selection of a molecule or portion thereof as a point of reference or comparison. For example, a domain can be selected as a designated domain for the purpose of constructing polypeptides that are modified within the selected domain. In another example, an intron can be selected as a designated intron for the purpose of identifying RNA transcripts that include or exclude the selected intron.

As used herein, production with reference to a polypeptide refers to expression and recovery of an expressed protein (or recoverable or isolatable expressed protein). Factors that can influence the production of a protein include the expression system and host cell chosen, the cell culture conditions, the secretion of the protein by the host cell, and ability to detect a protein for purification purposes. Production of a protein can be monitored by assessing the secretion of a protein, such as for example, into cell culture medium.

As used herein, “improved production” refers to an increase in the production of a polypeptide compared to the production of a control polypeptide. For example, production of an isoform fusion protein is compared to a corresponding isoform that is not a fusion protein or that contains a different fusion. For example, the production of an isoform containing a tPA pre/prosequence can be compared to an isoform containing its endogenous signal sequence. Generally, production of a protein can be improved more than, about or at least 1, 1.5, 2, 3, 4, 5, 6, 7, 8, 9 10 fold and more. Typically, production of a protein can be improved by 5, 10, 20, 30, 40, 50 fold or more compared to a corresponding isoform that is not an isoform fusion or does not contain the same fusion.

As used herein, secretion refers to the process by which a protein is transported into the external cellular environment or, in the case of gram-negative bacteria, into the periplasmic space. Generally, secretion occurs through a secretory pathway in a cell, for example, in eukaryotic cells this involves the endoplasmic reticulum and golgi apparatus.

As used herein, a “precursor sequence” or “precursor peptide” or “precursor polypeptide” refers to a sequence of amino acids, that is processed, and that occurs at a terminus, typically at the amino terminus, of a polypeptide prior to processing or cleavage. The precursor sequence includes sequences of amino acids that effect secretion and/or trafficking of the linked polypeptide. The precursor sequence can include one or more functional portions. For example, it can include a presequence (a signal polypeptide) and/or a pro sequence. Processing of a polypeptide into a mature polypeptide results in the cleavage of a precursor sequence from a polypeptide. The precursor sequence, when it includes a presequence and a prosequence also can be referred to as a pre/prosequence.

As used herein, a “presequence”, “signal sequence”, “signal peptide”, “leader sequence” or “leader peptide” refers to a sequence of amino acids at the amino terminus of nascent polypeptides, which target proteins to the secretory pathway and are cleaved from the nascent chain once translocated in the endoplasmic reticulum membrane.

As used herein, a prosequence refers to a sequence encoding a propeptide which when it is linked to a polypeptide can exhibit diverse regulatory functions including, but not limited to, contributing to the correct folding and formation of disulfide bonds of a mature polypeptide, contributing to the activation of a polypeptide upon cleavage of the pro-peptide, and/or contributing as recognition sites. Generally, a pro-sequence is cleaved off within the cell before secretion, although it can also be cleaved extracellularly by exoproteases. In some examples, a pro-sequence is autocatalytically cleaved while in other examples another polypeptide protease cleaves a pro-sequence.

As used herein, homologous refers to a molecule, such as a nucleic acid molecule or polypeptide, from different species that correspond to each other and that are identical or very similar to each other (i.e., are homologs).

As used herein, heterologous refers to a molecule, such as a nucleic acid or polypeptide, that is unique in activity or sequence. A heterologous molecule can be derived from a separate genetic source or species. For purposes herein, a heterologous molecule is a protein or polypeptide, regardless of origin, other than a CSR or ligand isoform, or allelic variants thereof. Thus, molecules heterologous to a CSR or ligand isoform include any molecule containing a sequence that is not derived from, endogenous to, or homologous to the sequence of a CSR or ligand isoform. Examples of heterologous molecules of interest herein include secretion signals from a different polypeptide of the same or different species, a tag such as a fusion tag or label, or all or part of any other molecule that is not homologous to and whose sequence is not the same as that of a CSR isoform or ligand. A heterologous molecule can be fused to a nucleic acid or polypeptide sequence of interest for the generation of a fusion or chimeric molecule.

As used herein, a heterologous secretion signal refers to the a signal sequence from a polypeptide, from the same or different species, that is different in sequence from the signal sequence of a CSR or ligand isoform. A heterologous secretion signal can be used in a host cell from which it is derived or it can be used host cells that differ from the cells from which the signal sequence is derived.

As used herein, an endogenous precursor sequence or endogenous signal sequence refers to the naturally occurring signal sequence associated with all or part of a polypeptide. The approximate location of exemplary signal sequence of various CSR and ligand isoforms, based on their corresponding cognate receptor or ligand signal sequence, are provided such as in Table 3 and 4. The C-terminal boundary of a signal peptide may vary, however, typically by no more than about 5 amino acids on either side of the signal peptide C-terminal boundary. Algorithms are available and known to one of skill in the art to identify signal sequences and predict their cleavage site (see e.g., Chou et al., (2001), Proteins 42:136; McGeoch et al., (1985) Virus Res. 3:271; von Heijne et al., (1986) Nucleic Acids Res. 14:4683).

As used herein, tissue plasminogen activator (tPA) refers to an extrinsic (tissue-type) plasminogen activator having fibrinolytic activity and typically having a structure with five domains (finger, growth factor, kringle-1, kringle-2, and protease domains). Mammalian t-PA includes t-PA from any animal, including humans. Other species include, but are not limited to, rabbit, rat, porcine, non human primate, equine, murine, dog, cat, bovine and ovine tPA. Nucleic acid encoding tPA including the precursor polypeptide(s) from human and non-human species is known in the art.

As used herein, a tPA precursor sequence refers to a sequence of amino residues that includes the presequence and prosequence from tPA (i.e., is a pre/prosequence, see e.g., U.S. Pat. Nos. 6,693,181 and 4,766,075). This polypeptide is naturally associated with tPA and acts to direct the secretion of a tPA from a cell. An exemplary precursor sequence for tPA is set forth in SEQ ID NO:2 and encoded by a nucleic acid sequence set forth in SEQ ID NO: 1. The precursor sequence includes the signal sequence (amino acids 1-23) and a prosequence (amino acids 24-35). The prosequence includes two protease cleavage sites: one after residue 32 and another after residue 35. Exemplary species variants of precursor sequences are forth in any one of SEQ ID NOS: 52-59; exemplary nucleotide and amino acid allelic variants are set forth in SEQ ID NOS:5 and 6.

As used herein, all or a portion of a tPA precursor sequence refers to any contiguous portion of amino acids of a tPA precursor sequence sufficient to direct processing and/or secretion of tPA from a cell. All or a portion of a precursor sequence can include all or a portion of a wildtype or predominant tPA precursor sequence such as set forth in SEQ ID NO:2 and encoded by SEQ ID NO: 1, allelic variants thereof set forth in SEQ ID NO: 6, or species variants set forth in SEQ ID NOS:52-59. For example, for the exemplary tPA precursor sequence set forth in SEQ ID NO:2, a portion of a tPA precursor sequence can include amino acids 1-23, or amino acids 24-35, 24-32, or amino acids 33-35, or any other contiguous sequence of amino acids 1-35 set forth in SEQ ID NO:2.

As used herein, an active portion of a polypeptide, such as with reference to an active portion of an isoform, refers to a portion of polypeptide that has an activity.

As used herein, purification of a protein refers to the process of isolating a protein, such as from a homogenate, which can contain cell and tissue components, including DNA, cell membrane and other proteins. Proteins can be purified in any of a variety of ways known to those of skill in the art, such as for example, according to their isoelectric points by running them through a pH graded gel or an ion exchange column, according to their size or molecular weight via size exclusion chromatography or by SDS-PAGE (sodium dodecyl sulfate-polyacrylamide gel electrophoresis) analysis, or according to their hydrophobicity. Other purification techniques include, but are not limited to, precipitation or affinity chromatography, including immuno-affinity chromatography, and other techniques and methods that include a combination of any of these methods. Furthermore, purification can be facilitated by including a tag on the molecule, such as a his tag for affinity purification or a detectable marker for identification.

As used herein, detection includes methods that permit visualization (by eye or equipment) of a protein. A protein can be visualized using an antibody specific to the protein. Detection of a protein can also be facilitated by fusion of a protein with a tag including an epitope tag or label.

As used herein, a “tag” refers to a sequence of amino acids, typically added to the N- or C-terminus of a polypeptide. The inclusion of tags fused to a polypeptide can facilitate polypeptide purification and/or detection.

As used herein, an epitope tag includes a sequence of amino acids that has enough residues to provide an epitope against which an antibody can be made, yet short enough so that it does not interfere with an activity of the polypeptide to which it is fused. Suitable tag polypeptides generally have at least 6 amino acid residues and usually between about 8 and 50 amino acid residues.

As used herein, a label refers to a detectable compound or composition which is conjugated directly or indirectly to an isoform so as to generate a labeled isoform. The label can be detectable by itself (e.g., radioisotope labels or fluorescent labels) or, in the case of an enzymatic label, can catalyze chemical alteration of a substrate compound composition which is then detectable. Non-limiting examples of labels included fluorogenic moieties, green fluorescent protein, or luciferase.

As used herein, a fusion tagged polypeptide refers to a chimeric polypeptide containing an isoform polypeptide fused to a tag polypeptide.

As used herein, expression refers to the process by which a gene's coded information is converted into the structures present and operating in the cell. Expressed genes include those that are transcribed into mRNA and then translated into protein and those that are transcribed into RNA but not translated into protein (e.g., transfer and ribosomal RNA). For purposes herein, a protein that is expressed can be retained inside the cells, such as in the cytoplasm, or can be secreted from the cell.

As used herein, a fusion construct refers to a nucleic acid sequence containing a coding sequence from one nucleic acid molecule and the coding sequence from another nucleic acid molecule in which the coding sequences are in the same reading frame such that when the fusion construct is transcribed and translated in a host cell, the protein is produced containing the two proteins. The two molecules can be adjacent in the construct or separated by a linker polypeptide that contains, 1, 2, 3, or more, but typically fewer than 10, 9, 8, 7, 6 amino acids. The protein product encoded by a fusion construct is referred to as a fusion polypeptide.

As used herein, a restriction enzyme linker is a linker that is encoded by a sequence of nucleotides recognized by one or more restriction enzymes As used herein, an isoform fusion protein or an isoform fusion polypeptide refers to a polypeptide encoded by a nucleic acid molecule that contains a coding sequence from an isoform, with or without an intron sequence, and a coding sequence that encodes another polypeptide, such as a precursor sequence or an epitope tag. The nucleic acids are operatively linked such that when the isoform fusion construct is transcribed and translated, an isoform fusion polypeptide is produced in which the isoform polypeptide is joined directly or via a linker to another peptide. An isoform polypeptide, typically is linked at the N-, or C-terminus, or both, to one or more other peptides.

As used herein, an allelic variant or allelic variation references to a polypeptide encoded by a gene that differs from a reference form of a gene (i.e. is encoded by an allele) among a population. Typically the reference form of the gene encodes a wildtype form and/or predominant form of a polypeptide from a population or single reference member of a species. Typically, allelic variants, have at least 80%, 90%, 95% or greater amino acid identity with a wildtype and/or predominant form from the same species.

As used herein, species variants refers to variants of the same polypeptide between and among species. Generally, interpecies allelic variants have at least about 60%, 70%, 80%, 85%, 90% or 95% identity or greater with a wildtype and/or predominant form of another species, including 96%, 97%, 98%, 99% or greater identity with a wildtype and/or predominant form of a polypeptide.

As used herein, modification refers to modification of a sequence of amino acids of a polypeptide or a sequence of nucleotides in a nucleic acid molecule and includes deletions, insertions, and replacements of amino acids and nucleotides, respectively.

As used herein, modulate and modulation refer to a change of an activity of a molecule, such as a protein. Exemplary activities include, but are not limited to, activities such as signal transduction and protein phosphorylation. Modulation can include an increase in the activity (i.e., up-regulation of an activity) a decrease in activity (i.e., down-regulation or inhibition) or any other alteration in an activity (such as in the periodicity, frequency, duration and kinetics). Modulation can be context dependent and typically modulation is compared to a designated state, for example, the wildtype protein, the protein in a constitutive state, or the protein as expressed in a designated cell type or condition.

As used herein, inhibit and inhibition refer to a reduction in an activity, such as a biological activity, relative to the uninhibited activity.

As used herein, a therapeutic protein refers to a protein used for the treatment of a condition, disease, or disorder.

As used herein, treatment means any manner in which the symptoms of a condition, disorder or disease or other indication, are ameliorated or otherwise beneficially altered.

As used herein, a disease or disorder mediated by a cognate receptor or ligand refers to any disease in which an cognate receptor or ligand plays a role, whereby modulation of its activity would effect treatment of the disease or symptom of the disease. Exemplary of cognate receptors of ligands are any provided herein including any CSR, such as RTK, a RAGE, or a TNF receptor, or a ligand such as HGF. Exemplary diseases or disorders for which a cognate receptor or ligand plays a role, such as a cognate receptor or ligand for any isoform provided herein, include but are not limited to angiogenesis-related diseases and conditions including ocular diseases, atherosclerosis, cancer and vascular injuries; neurodegenerative diseases, including Alzheimer's disease; inflammatory diseases and conditions, including atherosclerosis and Rhematoid Arthritis; diseases and conditions associated with cell proliferation including cancers, and smooth muscle cell-associated conditions; and various autoimmune diseases.

As used herein therapeutic effect means an effect resulting from treatment of a subject that alters, typically improves or ameliorates, the symptoms of a disease or condition or that cures a disease or condition. A therapeutically effective amount refers to the amount of a composition, molecule or compound which results in a therapeutic effect following administration to a subject.

As used herein, the term “subject” refers to animals, including mammals, such as human beings.

As used herein, a patient refers to a human subject.

As used herein, an activity refers to a function or functioning or changes in or interactions of a biomolecule, such as polypeptide. Exemplary, but not limiting of such activities are: complexation, dimerization, multimerization, receptor-associated kinase activity or other enzymatic or catalytic activity, receptor-associated protease activity, phosphorylation, dephosphorylation, autophosphorylation, ability to form complexes with other molecules, ligand binding, catalytic or enzymatic activity, activation including auto-activation and activation of other polypeptides, inhibition or modulation of another molecule's function, stimulation or inhibition of signal transduction and/or cellular responses such as cell proliferation, migration, differentiation, and growth, degradation, membrane localization, membrane binding, and oncogenesis. An activity can be assessed by assays described herein and by any suitable assays known to those of skill in the art, including, but not limited to, in vitro assays, including cell-based assays, in vivo assays, including assays in animal models for particular diseases. Biological activities refer to activities exhibited in vivo. For purposes herein, biological activity refers to any of the activities exhibited by a polypeptide provided herein.

As used herein, angiogenic diseases (or angiogenesis-related diseases) are diseases in which the balance of angiogenesis is altered or the timing thereof is altered. Angiogenic diseases include those in which an alteration of angiogenesis, such as undesirable vascularization, occurs. Such diseases include, but are not limited to cell proliferative disorders, including cancers, diabetic retinopathies and other diabetic complications, inflammatory diseases, endometriosis and other diseases in which excessive vascularization is part of the disease process, including those noted above.

As used herein, complexation refers to the interaction of two or more molecules such as two molecules of a protein to form a complex. The interaction can be by noncovalent and/or covalent bonds and includes, but is not limited to, hydrophobic and electrostatic interactions, Van der Waals forces and hydrogen bonds. Generally, protein- protein interactions involve hydrophobic interactions and hydrogen bonds. Complexation can be influenced by environmental conditions such as temperature, pH, ionic strength and pressure, as well as protein concentrations.

As used herein, dimerization refers to the interaction of two molecules of the same type, such as two molecules of a receptor. Dimerization includes homodimerization where two identical molecules interact. Dimerization also includes heterodimerization of two different molecules, such as two subunits of a receptor and dimerization of two different receptor molecules. Typically, dimerization involves two molecules that interact with each other through interaction of a dimerization domain contained in each molecule.

As used herein, a ligand antagonist refers to the activity of a CSR or ligand isoform that antagonizes an activity that results from ligand interaction with a CSR.

As used herein, in silico refers to research and experiments performed using a computer. In silico methods include, but are not limited to, molecular modeling studies, biomolecular docking experiments, and virtual representations of molecular structures and/or processes, such as molecular interactions.

As used herein, biological sample refers to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or a sample that is processed For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. Also included are soil and water samples and other environmental samples, viruses, bacteria, fungi, algae, protozoa and components thereof.

As used herein, the term “nucleic acid” refers to single-stranded and/or double-stranded polynucleotides such as deoxyribonucleic acid (DNA), and ribonucleic acid (RNA) as well as analogs or derivatives of either RNA or DNA. Also included in the term “nucleic acid” are analogs of nucleic acids such as peptide nucleic acid (PNA), phosphorothioate DNA, and other such analogs and derivatives or combinations thereof. Nucleic acid can refer to polynucleotides such as deoxyribonucleic acid (DNA) and ribonucleic acid (RNA). The term also includes, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine.

As used herein, “nucleic acid molecule encoding” refers to a nucleic acid molecule which directs the expression of a specific protein or peptide. The nucleic acid sequences include both the DNA strand sequence that is transcribed into RNA and the RNA sequence that is translated into protein or peptide. The nucleic acid molecule includes both the full length nucleic acid sequences as well as non-full length sequences derived from the full length mature polypeptide, such as for example a full length polypeptide lacking a precursor sequence. For purposes herein, a nucleic acid sequence also includes the degenerate codons of the native sequence or sequences which may be introduced to provide codon preference in a specific host.

As used herein, the term “polynucleotide” refers to an oligomer or polymer containing at least two linked nucleotides or nucleotide derivatives, including a deoxyribonucleic acid (DNA), a ribonucleic acid (RNA), and a DNA or RNA derivative containing, for example, a nucleotide analog or a “backbone” bond other than a phosphodiester bond, for example, a phosphotriester bond, a phosphoramidate bond, a phophorothioate bond, a thioester bond, or a peptide bond (peptide nucleic acid). The term “oligonucleotide” also is used herein essentially synonymously with “polynucleotide,” although those in the art recognize that oligonucleotides, for example, PCR primers, generally are less than about fifty to one hundred nucleotides in length.

Polynucleotides can include nucleotide analogs, including, for example, mass modified nucleotides, which allow for mass differentiation of polynucleotides; nucleotides containing a detectable label such as a fluorescent, radioactive, luminescent or chemiluminescent label, which allow for detection of a polynucleotide; or nucleotides containing a reactive group such as biotin or a thiol group, which facilitates immobilization of a polynucleotide to a solid support. A polynucleotide also can contain one or more backbone bonds that are selectively cleavable, for example, chemically, enzymatically or photolytically. For example, a polynucleotide can include one or more deoxyribonucleotides, followed by one or more ribonucleotides, which can be followed by one or more deoxyribonucleotides, such a sequence being cleavable at the ribonucleotide sequence by base hydrolysis. A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well-known methods (see, for example, Weiler et al. Nucleic acids Res. 25: 2792-2799 (1997)).

As used herein, synthetic, in the context of a synthetic sequence and synthetic gene refers to a nucleic acid molecule that is produced by recombinant methods and/or by chemical synthesis methods.

As used herein, oligonucleotides refer to polymers that include DNA, RNA, nucleic acid analogues, such as PNA, and combinations thereof. For purposes herein, primers and probes are single-stranded oligonucleotides or are partially single-stranded oligonucleotides.

As used herein, primer refers to an oligonucleotide containing two or more deoxyribonucleotides or ribonucleotides, generally more than three, from which synthesis of a primer extension product can be initiated. Experimental conditions conducive to synthesis include the presence of nucleoside triphosphates and an agent for polymerization and extension, such as DNA polymerase, and a suitable buffer, temperature and pH.

As used herein, production by recombinant means by using recombinant DNA methods, refers to the use of the well-known methods of molecular biology for expressing proteins encoded by cloned DNA.

As used herein, “isolated,” with reference to a molecule, such as a nucleic acid molecule, oligonucleotide, polypeptide or antibody, indicates that the molecule has been altered by the hand of man from how it is found in its natural environment. For example, a molecule produced by and/or contained within a recombinant host cell is considered “isolated”. Likewise, a molecule that has been purified, partially or substantially, from a native source or recombinant host cell, or produced by synthetic methods, is considered “isolated.” Depending on the intended application, an isolated molecule can be present in any form, such as in an animal, cell or extract thereof; dehydrated, in vapor, solution or suspension; or immobilized on a solid support.

As used herein, the term “vector” refers to a nucleic acid molecule capable of transporting another nucleic acid to which it has been linked. For example, a vector refers to viral expression systems, autonomous self-replicating circular DNA (plasmids), and includes expression and nonexpression plasmids. One type of vector also can be an episome, i.e., a nucleic acid capable of extra chromosomal replication. Vectors include those capable of autonomous replication and/or expression of nucleic acids to which they are linked. Vectors capable of directing the expression of genes to which they are operatively linked are referred to herein as “expression vectors”. In general, expression vectors are often in the form of “plasmids”, which are generally circular double stranded DNA loops that, in their vector form, are not bound to the chromosome. “Plasmid” and “vector” are used interchangeably as the plasmid is the most commonly used form of vector. Other forms of expression vectors include those that serve equivalent functions and that become known in the art subsequently hereto. Where a recombinant microorganism or cell is described as hosting an “expression vector”, this includes both extrachromosomal circular DNA and DNA that has been incorporated into the host chromosome(s). Where a vector is being maintained by a host cell, the vector may either be stably replicated by the cells during mitosis as an autonomous structure, or the vector may be incorporated within the host's genome.

As used herein, a reporter gene construct is a nucleic acid molecule that includes a nucleic acid encoding a reporter operatively linked to transcriptional control sequences. Transcription of the reporter gene is controlled by these sequences. The activity of at least one or more of these control sequences is directly or indirectly regulated by another molecule such as a cell surface protein, a protein or small molecule involved in signal transduction within the cell. The transcriptional control sequences include the promoter and other regulatory regions, such as enhancer sequences, that modulate the activity of the promoter, or control sequences that modulate the activity or efficiency of the RNA polymerase. Such sequences are herein collectively referred to as transcriptional control elements or sequences. In addition, the construct can include sequences of nucleotides that alter translation of the resulting mRNA, thereby altering the amount of reporter gene product.

As used herein, “reporter” or “reporter moiety” refers to any moiety that allows for the detection of a molecule of interest, such as a protein expressed by a cell, or a biological particle. Typical reporter moieties include, for example, fluorescent proteins, such as red, blue and green fluorescent proteins (see, e.g., U.S. Pat. No. 6,232,107, which provides GFPs from Renilla species and other species), the lacZ gene from E. coli, alkaline phosphatase, chloramphenicol acetyl transferase (CAT) and other such well-known genes. For expression in cells, nucleic acid encoding the reporter moiety, referred to herein as a “reporter gene”, can be expressed as a fusion protein with a protein of interest or under to the control of a promoter of interest.

As used herein, the phrase “operatively linked” with reference to sequences of nucleic acids means the nucleic acid molecules or segments thereof are covalently joined into one piece of nucleic acid such as DNA or RNA, whether in single or double stranded form. The segments are not necessarily contiguous, rather two or more components are juxtaposed so that the components are in a relationship permitting them to function in their intended manner. For example, segments of RNA (exons) can be operatively linked such as by splicing, to form a single RNA molecule. In another example, DNA segments can be operatively linked, whereby control or regulatory sequences on one segment permit expression or replication or other such control of other segments. Thus, in the case of a regulatory region operatively linked to a reporter or any other polynucleotide, or a reporter or any polynucleotide operatively linked to a regulatory region, expression of the polynucleotide/reporter is influenced or controlled (e.g., modulated or altered, such as increased or decreased) by the regulatory region. For gene expression, a sequence of nucleotides and a regulatory sequence(s) are connected in such a way to control or permit gene expression when the appropriate molecular signal, such as transcriptional activator proteins, are bound to the regulatory sequence(s). Operative linkage of heterologous nucleic acid, such as DNA, to regulatory and effector sequences of nucleotides, such as promoters, enhancers, transcriptional and translational stop sites, and other signal sequences, refers to the relationship between such DNA and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA in reading frame.

As used herein, the term “operatively linked” with reference to amino acids in polypeptides refers to covalent linkage (direct or indirect) of the amino acids. For example, when used in the context of the phrase “at least one domain of a cell surface receptor operatively linked to at least one amino acid encoded by an intron of a gene encoding a cell surface receptor”, means that the amino acids of a domain from a cell surface receptor are covalently joined to amino acids encoded by an intron from a cell surface receptor gene. Such linkage, typically direct via peptide bonds, also can be effected indirectly, such as via a linker or via non-peptidic linkage. Hence, a polypeptide that contains at least one domain of a cell surface receptor operatively linked to at least one amino acid encoded by an intron of a gene encoding a cell surface receptor can be an intron fusion protein. It contains one or more amino acids that are not found in a predominant form of the receptor, but rather, contains a portion that is encoded by an intron of the gene that encodes the predominant form. These one or more amino acids are encoded by an intron sequence of the gene encoding the cell surface receptor. Nucleic acids encoding such polypeptides can be produced when an intron sequence is spliced or otherwise covalently joined in-frame to an exon sequence that encodes a domain of a cell surface receptor. Translation of the nucleic acid molecule produces a polypeptide where the amino acid(s) of the intron sequence are covalently joined to a domain of the cell surface receptor. They also can be produced synthetically by linking a portion containing an exon to a portion containing an intron, including chimeric intron fusion proteins in which the exon is encoded by a gene for a different cell surface receptor isoform from the intron portion.

As used herein, the phrase “generated from a nucleic acid” in reference to the generating of a polypeptide, such as an isoform and intron fusion protein, includes the literal generation of a polypeptide molecule and the generation of an amino acid sequence of a polypeptide from translation of the nucleic acid sequence into a sequence of amino acids.

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences can be cis acting or can be responsive to trans acting factors. Promoters, depending upon the nature of the regulation, can be constitutive or regulated.

As used herein, regulatory region means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3 of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, the “amino acids,” which occur in the various amino acid sequences appearing herein, are identified according to their well-known, three-letter or one-letter abbreviations (see Table 1). The nucleotides, which occur in the various DNA fragments, are designated with the standard single-letter designations used routinely in the art.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH2 refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§ 1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | Asparagines |
| B | Asx | Asn and/or Asp |
| C | Cys | Cysteine |
| X | Xaa | Unknown or other |

All sequences of amino acid residues represented herein by a formula have a left to right orientation in the conventional direction of amino-terminus to carboxyl-terminus. In addition, the phrase “amino acid residue” is defined to include the amino acids listed in the Table of Correspondence modified, non-natural and unusual amino acids. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Curmmings Pub. co., p.224).

Such substitutions may be made in accordance with those set forth in TABLE 2 as follows:

TABLE 2

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative or non-conservative substitutions.

As used herein, “similarity” between two proteins or nucleic acids refers to the relatedness between the sequence of amino acids of the proteins or the nucleotide sequences of the nucleic acids. Similarity can be based on the degree of identity and/or homology of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

As used herein, the terms “homology” and “identity” are used interchangeably, but homology for proteins can include conservative amino acid changes. In general, to identify corresponding positions the sequences of amino acids are aligned so that the highest order match is obtained (see, e.g. Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48:1073).

As use herein, “sequence identity” refers to the number of identical amino acids (or nucleotide bases) in a comparison between a test and a reference polypeptide or polynucleotide. Homologous polypeptides refer to a pre-determined number of identical or homologous amino acid residues. Homology includes conservative amino acid substitutions as well identical residues. Sequence identity can be determined by standard alignment algorithm programs used with default gap penalties established by each supplier. Homologous nucleic acid molecules refer to a pre-determined number of identical or homologous nucleotides. Homology includes substitutions that do not change the encoded amino acid (i.e., “silent substitutions”) as well identical residues. Substantially homologous nucleic acid molecules hybridize typically at moderate stringency or at high stringency all along the length of the nucleic acid or along at least about 70%, 80% or 90% of the fill-length nucleic acid molecule of interest. Also contemplated are nucleic acid molecules that contain degenerate codons in place of codons in the hybridizing nucleic acid molecule. (For determination of homology of proteins, conservative amino acids can be aligned as well as identical amino acids; in this case, percentage of identity and percentage homology vary). Whether any two nucleic acid molecules have nucleotide sequences (or any two polypeptides have amino acid sequences) that are at least 60%, 70%, 80%, 85%, 90%, 95%, 96%, 97%, 98% or 99% “identical” can be determined using known computer algorithms such as the “FAST A” program, using for example, the default parameters as in Pearson et al. Proc. Natl. Acad. Sci. USA 85: 2444 (1988) (other programs include the GCG program package (Devereux, J., et al., Nucleic Acids Research 12(I): 387 (1984)), BLASTP, BLASTN, FASTA (Altschul, S. F., et al., J Molec. Biol. 215:403 (1990); Guide to Huge Computers, Martin J. Bishop, ed., Academic Press, San Diego (1994), and Carrillo et al. SIAM J Applied Math 48: 1073 (1988)). For example, the BLAST function of the National Center for Biotechnology Information database can be used to determine identity. Other commercially or publicly available programs include, DNAStar “MegAlign” program (Madison, WI) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. J Mol. Biol. 48: 443 (1970), as revised by Smith and Waterman (Adv. Appl. Math. 2: 482 (1981)). Briefly, a GAP program defines similarity as the number of aligned symbols (i.e., nucleotides or amino acids) which are similar, divided by the total number of symbols in the shorter of the two sequences. Default parameters for the GAP program can include: (1) a unary comparison matrix (containing a value of 1 for identities and 0 for non identities) and the weighted comparison matrix of Gribskov et al. Nucl. Acids Res. 14: 6745 (1986), as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps. Therefore, as used herein, the term “identity” represents a comparison between a test and a reference polypeptide or polynucleotide. In one non-limiting example, “at least 90% identical to” refers to percent identities from 90 to 100% relative to the reference polypeptides. Identity at a level of 90% or more is indicative of the fact that, assuming for exemplification purposes a test and reference polynucleotide length of 100 amino acids are compared, no more than 10% (i.e., 10 out of 100) of amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often without relying on software.

As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, “primer” refers to a nucleic acid molecule that can act as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. It will be appreciated that certain nucleic acid molecules can serve as a “probe” and as a “primer.” A primer, however, has a 3′ hydroxyl group for extension. A primer can be used in a variety of methods, including, for example, polymerase chain reaction (PCR), reverse-transcriptase (RT)-PCR, RNA PCR, LCR, multiplex PCR, panhandle PCR, capture PCR, expression PCR, 3′and 5′RACE, in situ PCR, ligation-mediated PCR and other amplification protocols.

As used herein, “primer pair” refers to a set of primers that includes a 5′ (upstream) primer that hybridizes with the 5′ end of a sequence to be amplified (e.g. by PCR) and a 3′ (downstream) primer that hybridizes with the complement of the 3′ end of the sequence to be amplified.

As used herein, “specifically hybridizes” refers to annealing, by complementary base-pairing, of a nucleic acid molecule (e.g. an oligonucleotide) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application.

B. Cell Surface Receptor and Ligand Isoforms

Provided herein are nucleic acids encoding cell surface receptor (CSR) isoforms or ligand isoforms fused to another nucleic acid that alters the production of a CSR isoform, such as by altering secretion, expression, and/or purification of a CSR or ligand isoform. The isoform fusion results in a polypeptide that has improved secretion and expression compared to an isoform that is not a fusion with another nucleic acid sequence. Also provided herein are expression vectors containing nucleic acid encoding an isoform as provided herein, and cells containing such vectors.

The isoforms exemplified herein represent variants of a predominant or wildtype gene that can be generated by alternate splicing or by recombinant or synthetic (e.g., in silico and/or chemical synthesis) methods. The isoforms are described in related applications (copending U.S. application Ser. No. 10/846,113 and corresponding International PCT application No. WO 05/016966, U.S. application Ser. No. 11/129,740, U.S. Provisional application No. 60/678,076, and U.S. application No. (Attorney Docket No. 17118-045P01/P2824), which, as all such documents, are incorporated by reference in their entirety). Typically, an isoform produced from an alternatively spliced RNA is not a predominant form of a polypeptide encoded by a gene. In some instances, an isoform can be a tissue-specific or developmental stage-specific polypeptide or disease-specific (i.e., can be expressed at a different level from tissue-to-tissue or stage-to-stage or in a diseased state compared to a non-diseased state or only can be expressed in the tissue, at the stage or during the disease process or progress). Alternatively spliced RNA forms that can encode isoforms include, but are not limited to, exon deletion, exon retention, exon extension, exon truncation, and intron retention alternatively spliced RNAs. Generally, an isoform provided herein is generated by intron modification.

Isoforms generated by alternative splicing of encoding nucleic molecules include intron fusion proteins, whereby one or more codons (including stop codons) from one or more introns is/are retained compared to an mRNA transcript encoding a wildtype or predominant form of an isoform. The retention of one or more intron codons can generate transcripts encoding isoforms that are shortened compared to a wildtype or predominant form of an isoform. A retained intron sequence can introduce a stop codon in the transcript and thus prematurely terminate the encoded polypeptide. A retained intron sequence also can introduce additional amino acids into an isoform polypeptide, such as the insertion of one or more codons into a transcript such that one or more amino acids are inserted into a domain of an isoform. Intron retention includes the inclusion of a full or partial intron sequence into a transcript encoding an isoform. The retained intron sequence can introduce nucleotide sequence with codons in-frame to the surrounding exons or it can introduce a frame shift into the transcript.

1. Cell Surface Receptor Isoforms

Isoforms that are cell surface receptor isoforms can be linked to a signal sequence or to a precursor sequence as described herein or can be produced by expression of a nucleic acid construct that encodes an isoform operatively linked to a prescursor or signal sequence. CSR isoforms can contain a new domain and/or exhibit a new or different biological function compared to a wildtype and/or predominant form of the receptor. For example, intron-encoded amino acids can introduce a new domain or portion thereof into an isoform. Biological activities that can be altered include, but are not limited to, protein-protein interactions such as dimerization, multimerization and complex formation, specificity and/or affinity for ligand, cellular localization and relocalization, membrane anchoring, enzymatic activity such as kinase activity, response to regulatory molecules including regulatory proteins, cofactors, and other signaling molecules, such as in a signal transduction pathway. Generally, a biological activity is altered in an isoform at least 0.1, 0.5, 1, 2, 3, 4, 5, or 10 fold compared to a wildtype and/or predominant form of the receptor. Typically, a biological activity is altered 10, 20, 50, 100 or 1000 fold or more. For example, an isoform can be reduced in a biological activity.

CSR isoforms also can modulate an activity of a wildtype and/or predominant form of the receptor. For example, a CSR isoform can interact directly or indirectly with a CSR isoform and modulate a biological activity of the receptor. Biological activities that can be altered include, but are not limited to, protein-protein interactions such as dimerization, multimerization and complex formation, specificity and/or affinity for ligand, cellular localization and relocalization, membrane anchoring, enzymatic activity such as kinase activity, response to regulatory molecules including regulatory proteins, cofactors, and other signaling molecules, such as in a signal transduction pathway.

A CSR isoform can interact directly or indirectly with a cell surface receptor to cause or participate in a biological effect, such as by modulating a biological activity of the cell surface receptor. A CSR isoform also can interact independently of a cell surface receptor to cause a biological effect, such as by initiating or inhibiting a signal transduction pathway. For example, a CSR isoform can initiate a signal transduction pathway and enhance or promote cell growth. In another example, a CSR isoform can interact with the cell surface receptor as a ligand causing a biological effect, for example by inhibiting a signal transduction pathway that can impede or inhibit cell growth. Hence, the isoforms provided herein can function as cell surface receptor ligands in that they interact with the targeted receptor in the same manner that a cognate ligand interacts with and alters receptor activity. The isoforms can bind as a ligand, but not necessarily, to a ligand binding site and serve to block receptor dimerization. They act as ligands in that they interact with the receptor. The CSR isoforms also can act by binding to ligands for the receptor and/or by preventing receptor activities, such as dimerization.

For example, a CSR isoform can compete with a CSR for ligand binding. A CSR isoform, when it binds to a receptor, can be a negative effector ligand, which results in inhibition of receptor function. It also is possible that some CSR isoforms bind a cognate receptor, resulting in activation of the receptor. A CSR isoform can act as a competitive inhibitor of a CSR, for example, by complexing with a CSR isoform and altering the ability of the CSR to multimerize (e.g. dimerize or trimerize) with other CSRs. A CSR isoform can compete with a CSR for interactions with other polypeptides and cofactors in a signal transduction pathway. The cell surface isoforms and families of isoforms provided herein include, but are not limited to, isoforms of receptor tyrosine kinases (also referred to herein as RTK isoforms) and isoforms of other families of CSRs, such as TNFs and other G-protein-coupled receptors. In one example, a CSR isoform is a soluble polypeptide. For example, a CSR isoform lacks at least part or all of a transmembrane domain. Soluble isoforms can modulate a biological activity of a wildtype or predominant form of a receptor (see for example, Kendall et al. (1993) PNAS 90: 10705, Werner et al. (1992) Molec. Cell Biol. 12: 82, Heaney et al. (1995) PNAS 92: 2365, Fukunaga et al. (1990) PNAS 87:8702, Wypych et al. (1995) Blood 85: 66-73, Barron et al. (1994) Gene 147:263, Cheng et al. (1994) Science 263: 1759, Dastot et al. (1996) PNAS 93:10723, Abramovich et al. (1994) FEBS Lett 338:295, Diamant et al. (1997) FEBS Lett 412:379, Ku et al. (1996) Blood 88:4124, Heaney ML and Golde DW (1998), J Leukocyte Biol. 64:135-146).

Exemplary CSR isoforms, including receptor tyrosine kinases (RTKs) or tumor necrosis factor receptors (TNFRs) or RAGE isoforms, include CSR intron fusion proteins provided herein and known to those of skill in the art including any described in copending U.S. application Ser. No. 10/846,113 and corresponding International PCT application No. WO 05/016966, U.S. application Ser. No. 11/129,740, U.S. Provisional application No. 60/678,076, and U.S. application No. (Attorney Docket No. 17118-045P01/P2824).

Generally, CSR intron fusion proteins are encoded by nucleic acid molecules that are generated by alternative splicing of a gene encoding a cognate cell surface receptor. Typically, a CSR isoform polypeptide contains at least one domain of a cell surface receptor either truncated at the end of an exon or linked to at least one amino acid encoded by an intron of a gene encoding a cognate cell surface receptor. CSRs include all cell surface receptors, such as receptor tyrosine kinases (RTKs), TNFRs, and RAGE receptors.

Examples of RTKs include, but are not limited to, erythropoietin-producing hepatocellular (EPH) receptors (also referred to as ephrin receptors), epidermal growth factor (EGF) receptors, fibroblast growth factor (FGF) receptors, platelet-derived growth factor (PDGF) receptors, vascular endothelial growth factor (VEGF) receptors, cell adhesion RTKs (CAKs), TIE/Tek receptors, hepatocyte growth factor (HGF) receptors (termed MET), discoidin domain receptors (DDR), insulin growth factor (IGF) receptors, insulin receptor-related (IRR) receptors and others, such as Tyro3/Axl. Examples of TNFRs include, but are not limited to TNFR1, TNFR2, TNFRrp, the low-affinity nerve growth factor receptor, Fas antigen, CD40, CD27, CD30, 4-1BB, OX40, DR3, DR4, DR5, and herpesvirus entry mediator (HVEM). Exemplary genes encoding RTKs or TNFRs include any listed in Table 3 including, but are not limited to, ErbB2, ErbB3, DDR1, DDR2, EGFR, EphA1, EphA2, EphA3, EphA 4, EphA 5, EphA 6, EphA 7, EphA8, EphB1, EphB2, EphB3, EphB4, EphB5, EphB6, FGFR-1, FGFR-2, FGFR-3, FGFR-4, Flt1(also known as VEGFR-1), VEGFR-2, VEGFR-3 (also known as VEGFRC), MET, RON, PDGFR-A, PDGFR-B, CSF1R, Flt3, KIT, TIE-1, TEK (also known as TIE-2), HER-2, RAGE, TNFR2, and genes encoding the RTKs and TNFRs noted above and not set forth. Table 3 provides non-limiting examples of exemplary CSR intron fusion proteins, including SEQ ID NOS for exemplary polypeptide sequences and the encoding nucleic acid sequences. Typically, one of skill in the art can determine the presence or absence of structural motifs of an isoform, including a precursor or signal sequence or other protein domain(s), compared to a cognate full-length receptor of an isoform. For example, alignment of an isoform with a full-length cognate receptor can be made to determine the presence or absence of a signal sequence and/or other domains known to exist for a cognate receptor. Using such alignments, amino acid residues contained in a signal sequence of exemplary CSR isoforms are listed in Table 3. In another example, an isoform can be tested for an activity, such as for example secretion or ligand binding, to determine if an activity of a domain is reduced or eliminated and/or a structure is altered compared to a full-length cognate receptor. CSR isoforms, such as those described below in Table 3, can be used in a fusion protein to improve the production, such as by secretion, of a CSR isoform.

TABLE 3
Exemplary CSR Intron Fusion Proteins

| | | | Signal | SEQ ID NO: | SEQ ID NO: |
|---|---|---|---|---|---|
| Gene | ID # | AA length | Sequence | (nucleic acid) | (amino acid) |
| DDR1 | SR005A11 | 286 | 1-18 | 139 | 140 |
| DDR1 | SR005A10 | 243 | 1-18 | 141 | 142 |
| DDR1.h | | 444 | 1-18 | n/a | 143 |
| EphA1 | SR004G03 | 474 | 1-23 | 144 | 145 |
| EphA1 | SR004G07 | 311 | 1-23 | 146 | 147 |
| EphA1 | SR004H03 | 490 | 1-23 | 148 | 149 |
| EphA1.b | | 166 | | n/a | 150 |
| EphA2 | SR016E12 | 497 | 1-24 | 151 | 152 |
| EphA8.b | | 495 | 1-30 | n/a | 153 |
| EphB1 | SR005D06 | 242 | 1-17 | 154 | 155 |
| EphB4 | SR012C08 | 306 | 1-15 | 156 | 157 |
| EphB4 | SR012D11 | 516 | 1-15 | 158 | 159 |
| EphB4 | SR012E11 | 414 | 1-15 | 160 | 161 |
| EGFR.a | | 405 | 1-24 | n/a | 162 |
| ErbB2 | herstatin | 419 | 1-22 | n/a | 289 |
| ErbB2.1.d | | 680 | 1-24 | n/a | 163 |
| ErbB2.1.e | | 633 | 1-22 | n/a | 164 |
| ErbB2.1.f | | 575 | 1-22 | n/a | 165 |
| ErbB2.a | | 90 | 1-22 | n/a | 166 |
| ErbB2.c 31 | | 419 | 1-22 | n/a | 167 |
| ErbB3.d 31 | | 331 | 1-19 | n/a | 168 |
| FGFR-1 | SR001E12 | 228 | 1-21 | 169 | 170 |
| FGFR-1 | SR022C02 | 320 | 1-21 | 171 | 172 |
| FGFR-2 | SR022C10 | 266 | 1-21 | 173 | 174 |
| FGFR-2 | SR022C11 | 317 | 1-21 | 175 | 176 |
| FGFR-2 | SR022D04 | 281 | 1-21 | 177 | 178 |
| FGFR-2 | SR022D06 | 396 | 1-21 | 179 | 180 |
| FGFR-2.b 31 | | 366 | 1-21 | n/a | 181 |
| FGFR-4 | SR002A11 | 72 | 1-24 | 182 | 183 |
| FGFR-4 | SR002A10 | 446 | 1-24 | 184 | 185 |
| FGFR-4.d 31 | | 209 | | n/a | 186 |
| MET | SR020C10 | 413 | 1-24 | 187 | 188 |
| MET | SR020C12 | 468 | 1-24 | 189 | 190 |
| MET | SR020D04 | 518 | 1-24 | 191 | 192 |
| MET | SR020D07 | 596 | 1-24 | 193 | 194 |
| MET | SR020D11 | 408 | 1-24 | 195 | 196 |
| MET | SR020E11 | 621 | 1-24 | 197 | 198 |
| MET | SR020F08 | 664 | 1-24 | 199 | 200 |
| MET | SR020F11 | 719 | 1-24 | 201 | 202 |
| MET | SR020F12 | 697 | 1-24 | 203 | 204 |
| MET | SR020G03 | 691 | 1-24 | 205 | 206 |
| MET | SR020G07 | 661 | 1-24 | 207 | 208 |
| MET | SR020H03 | 755 | 1-24 | 209 | 210 |
| MET | SR020H06 | 823 | 1-24 | 211 | 212 |
| MET | SR020H07 | 877 | 1-24 | 213 | 214 |
| MET | SR020H08 | 764 | 1-24 | 215 | 216 |
| MET 34 | | 934 | 1-24 | | 217 |
| RON | SR004C11 | 495 | 1-24 | 218 | 219 |
| RON | SR014C01 | 541 | 1-24 | 220 | 221 |
| RON | SR014C09 | 908 | 1-24 | 222 | 223 |
| RON | SR014E12 | 647 | 1-24 | 224 | 225 |
| CSF1R | SR00SA06 | 306 | 1-19 | 226 | 227 |
| KIT | SR002H01 | 413 | 1-22 | 228 | 229 |
| PDGFR-A.b 31 | | 217 | 1-23 | n/a | 230 |
| PDGFR-A.c 34 | | 218 | 1-23 | n/a | 231 |
| PDGFR-B | SR007C09 | 336 | 1-32 | 232 | 233 |
| RAGE | SR021A05 | 146 | 1-22 | 234 | 235 |
| RAGE | SR021C02 | 266 | 1-22 | 236 | 237 |
| RAGE | SR021C06 | 387 | 1-22 | 238 | 239 |
| RAGE | SR021C08 | 173 | 1-22 | 240 | 241 |
| RAGE | SR021F06 | 172 | 1-22 | 242 | 243 |
| TEK | SR007G02 | 367 | 1-18 | 244 | 245 |
| TEK | SR007H03 | 468 | 1-18 | 246 | 247 |
| TEKc | | 864 | 1-18 | n/a | 248 |
| TEKc 31 | | 798 | | n/a | 249 |
| TEKc 34 | | 821 | 1-18 | n/a | 250 |
| Tie-1 | | 786 | 1-21 | n/a | 251 |
| Tie-1 | SR006A04 | 251 | 1-21 | 252 | 253 |
| Tie-1 | SR006B07 | 379 | 1-21 | 254 | 255 |
| Tie-1 | SR006B06 | 161 | 1-21 | 256 | 257 |
| Tie-1 | SR006B12 | 414 | 1-21 | 258 | 259 |
| Tie-1 | SR006B10 | 317 | 1-21 | 260 | 261 |
| Tie-1 | SR016G03 | 751 | 1-21 | 262 | 263 |
| Tie-1 | | 838 | 1-21 | n/a | 264 |
| Tie-1 | | 632 | 1-21 | n/a | 265 |
| Tie-1 | | 533 | 1-21 | n/a | 266 |
| Tie-1 | | 428 | 1-21 | n/a | 267 |
| Tie-1 | | 344 | 1-21 | n/a | 268 |
| Tie-1 | | 255 | 1-21 | n/a | 269 |
| Tie-1 | | 197 | 1-21 | n/a | 270 |
| TNFR2 (TNFR1B) | SR003H02 | 155 | 1-22 | 271 | 272 |
| VEGFR-1 | SR004C05 | 174 | 1-26 | 273 | 274 |
| VEGFR-1 (FLT1.c 31) | | 479 | 1-26 | n/a | 275 |
| VEGFR-1 (FLT1.c 32) | | 523 | 1-26 | n/a | 276 |
| VEGFR-1 (FLT1.c 33) | | 436 | 1-26 | n/a | 277 |
| VEGFR-1 (FLT1.c 34) | | 365 | 1-26 | n/a | 278 |
| VEGFR-1 (FLT1.c) | SR018C02 | 541 | 1-26 | n/a | 279 |
| VEGFR-1 (FLT1.d 31) | | 687 | 1-26 | n/a | 280 |
| VEGFR-2 | SR01SF01 | 712 | 1-19 | 281 | 282 |
| VEGFR-3 | SR01SG09 | 765 | 1-22 | 283 | 284 |
| VEGFR-3 | SR007E10 | 227 | 1-22 | 285 | 286 |
| VEGFR-3 | SR007F05 | 295 | 1-22 | 287 | 288 |

2. Ligand Isoforms

Ligand isoforms are isoforms of ligands that normally interact with a receptor, such as a CSR. Ligand isoforms can contain a new domain and/or a function compared to a wildtype and/or predominant form of the ligand. The deletion, disruption and or insertion in the polypeptide sequence of a ligand isoform is sufficient to alter an activity compared to that of a wildtype or predominant form of a ligand or change the structure compared to a wildtype or predominant form of a ligand, such as by elimination of one or more domains or by addition of a domain or portion thereof, such as one encoded by an intron in the gene. One or more activities can be altered in a ligand isoform compared with a wildtype or predominant form of a ligand. Altered activities include altered interaction with one or more receptors and/or altered signal transduction that results from such interaction. For example, by virtue of such altered activity, a ligand isoform can act as an antagonist of the activity of the wild-type ligand, such as by competitively inhibiting binding to its receptor.

Generally, an activity of a ligand (i.e., receptor interaction) or a process that occurs by virtue of the activity of a ligand (i.e., signal transduction) is altered in a ligand isoform by at least 0.1, 0.5, 1, 2, 3, 4, 5, or 10 fold compared to a wildtype and/or predominant form of the ligand. Typically, an activity is altered 10, 20, 50, 100 or 1000 fold or more. For example, an isoform can exhibit a reduction in an activity compared to a wildtype and/or predominant form of the ligand. An isoform also can exhibit increased activity compared to a wildtype and/or predominant form of a ligand. Typically, a ligand isoform of a ligand that has several activities or functions will lack one or more of such activities or functions. For example, some ligands bind to receptors resulting in a cascade of events, such as signal transduction. The ligand isoform may bind to the receptor but fail to initiate the cascade of events or initiate it to a lesser extent.

Exemplary of ligand isoforms are growth factor ligand isoforms. Exemplary thereof are hepatocytes growth factor (HGF) isoforms. In one example, an HGF isoform is altered in cell surface interaction, including receptor interaction. For example, an isoform is reduced in binding affinity for one or more receptors, such as for example a MET receptor. In another example, an isoform exhibits increased affinity for one or more receptors. A ligand isoform, such as an HGF isoform, can exhibit altered binding to other cell surface molecules. In one example, isoforms can be altered in binding to glycosaminoglycans (GAGs), such as heparin or heparin sulfate. In another example, isoforms can be altered in binding to other cell surface proteins involved in angiogenesis, such as for example, endothelial ATP synthase, angiomotin, αvβ3 integrin, annexin II, and/or any one or more growth factor receptors such as MET, FGFR, or VEGFR. HGF isoforms can be altered in one or more facets of signal transduction. An isoform, compared with a wildtype or predominant form of HGF, can be altered in the modulation of one or more biological activities, including inducing, augmenting, suppressing and preventing cellular responses to a receptor. Examples of cellular responses that can be altered by an HGF isoform, include, but are not limited to, induction of mitogenic, motogenic, morphogenic and angiogenic responses, and/or the induction of signaling molecules such as those involved in a signal transductionn pathway.

Ligand isoforms, such as HGF isoforms, also can modulate an activity of another polypeptide. The modulated polypeptide can be a wildtype or predominant form of the ligand, such as HGF, or can be a wildtype or predominant form of another growth factor, such as FGF-2 or VEGF. For example, an HGF isoform also can modulate another HGF, FGF-2, or VEGF isoform, such as isoforms expressed in a disease or condition. Such HGF isoforms can act as negatively acting ligands by preventing or inhibiting one or more activities of a wildtype or predominant form of a growth factor ligand/receptor pair. A negatively acting ligand need not bind to or affect the ligand binding domain of a receptor, nor affect ligand binding of the receptor.

In one example, an HGF isoform competes with another growth factor ligand for binding to a cell surface protein necessary for mediating receptor dimerization and/or angiogenic responses of the growth factor. For example, an HGF isoform can compete with another growth factor ligand for binding to heparin or a GAG, thereby preventing the formation of a dimeric ligand required for ligand-mediated signaling of its receptor. In another example, an HGF isoform competes with another HGF form for receptor binding. Such isoforms can thus bind receptors and reduce the amount of receptor available to bind to other HGF polypeptides. HGF isoforms that bind and compete for one or more receptors of HGF can include HGF isoforms that do not participate in signal transduction or are reduced in their ability to participate in signal transduction compared to a cognate HGF.

Exemplary ligand isoforms, including HGF intron fusion protein isoforms, include ligand isoforms provided herein and known to those of skill in the art including any described in U.S. provisional application Ser. No. 60/735,609 filed Nov. 10, 2005 and corresponding U.S. application No. (attorney docket No. 17118-045001/2824) and International application No. (attorney docket No. 17118-045WO1/2824PC) filed on the same day herewith. Generally, ligand isoforms are encoded by nucleic acid molecules that are generated by alternative splicing of a gene encoding a ligand. Typically, a ligand intron fusion protein isoform polypeptide contains at least one domain of a ligand linked to at least one amino acid encoded by an intron of a gene encoding a ligand or is truncated at the end of an exon by virtue of alternative splicing that introduces a stop codon that occurs, upon splicing, as the first codon in the intron.

Table 4 provides non-limiting examples of exemplary ligand intron fusion protein isoforms, including SEQ ID NOS for exemplary polypeptide sequences and the encoding nucleic acid sequences. One of skill in the art can determine the presence or absence of structural motifs of an isoform, including a precursor or signal sequence or other protein domain(s), compared to a cognate full-length ligand of an isoform. For example, alignment of an isoform with a full-length cognate ligand can be made to determine the presence or absence of a signal sequence and/or other domains known to exist for a cognate ligand. Using such alignments, amino acid residues contained in a signal sequence of exemplary ligand isoforms are listed in Table 4. In another example, an isoform can be tested for an activity, such as for example secretion or receptor binding, to determine if an activity of a domain is reduced or eliminated and/or a structure is altered compared to a full-length cognate ligand. Ligand isoforms, such as those described below in Table 4, can be used in a fusion protein to improve the production, such as by secretion, of a ligand isoform.

TABLE 4
Exemplary Ligand intron fusion protein isoforms

| | | AA | Signal | SEQ ID NO: | SEQ ID NO: |
|---|---|---|---|---|---|
| Gene | IFP_ID | length | Sequence | (nucleic acid) | amino acid |
| HGF | SR023A02 | 467 | 1-31 | 349 | 350 |
| HGF | SR023A08 | 472 | 1-31 | 351 | 352 |
| HGF | SR023E09 | 514 | 1-31 | 353 | 354 |

3. Allelic and Species Variants of Isoforms and Mutations

Allelic variants of CSR or ligand isoform sequences occur or can be generated or identified that differ in one or more amino acids from a particular CSR or ligand isoform. Such variation includes variations among alleles in a single population or between species.

Variations include allelic variations that occur among members of a population and species variations that occur between and among species. Variations also include mutations that occur in an animal or that are synthetically produced. For example, isoforms can be derived from different alleles of a gene; each allele can have one or more amino acid differences from the other. Such alleles can have conservative and/or non-conservative amino acid differences. Variants also include isoforms produced or identified from different subjects, such as individual subjects or animal models or other animals. Amino acid changes can result in modulation of an isoform activity. In some cases, an amino acid difference can be “silent,” having no or virtually no detectable effect on an activity. Variants of isoforms also can be generated by mutagenesis. Such mutagenesis can be random or directed. For example, allelic variant isoforms can be generated that alter amino acid sequences or a potential glycosylation site to effect a change in glycosylation of an isoform, including alternate glycosylation, such as increased or inhibited glycosylation at a site in an isoform.

Allelic and other variant isoforms can be at least 90% identical in sequence to an isoform. Generally, a variant isoform from the same species is at least 95%, 96%, 97%, 98%, 99% identical to an isoform, typically an allelic variant is 98%, 99%, 99.5% identical to an isoform. Variation between and among species for the same protein can be 60%, 70%., 80%, 85%, 90%, 95%, 96%, 97%, 98%, 99% and greater. Exemplary non-limiting polypeptide sequences, including one or more allelic variants of an isoform provided herein, are set forth in SEQ ID NOS: 290-303, 429-459, or 462. Allelic variants of CSR or ligand isoforms can be included in the fusion proteins provided herein and encoded by the nucleic acid constructs provided herein and methods for expression thereof to improve the production, such as by secretion, of a CSR or ligand isoform.

C. Isoform Fusion Protein Production

Many therapeutic proteins are produced by recombinant gene expression in appropriate prokaryotic or eukaryotic hosts. Some proteins are produced in cells and isolated therefrom. For others, the expressed protein product is isolated after secretion into the culture medium or, in the case of gram-negative bacteria, into the periplasm between the inner and outer cell membranes. For the purification of many proteins, however, the rate of secretion limits the overall yield of protein product. Production of a polypeptide can be influenced by secretion, expression, and purification of a polypeptide.

The entry of secreted proteins to the secretory pathway, in prokaryotes and eukaryotes, is directed by specific signal peptides at the N-terminus of the polypeptide chain which are cleaved off during secretion. Signal sequences are predominantly hydrophobic, a feature which may be important in directing a nascent peptide to the membrane for transfer of secretory proteins across the inner membrane of prokaryotes or the endoplasmic reticulum (ER) membrane of eukaryotes. Due to the similarity among prokaryotic and eukaryotic signal sequences, signal sequences are generally adaptable to target the secretion of diverse homologous and heterologous proteins. Secretion is, however, a multi-step process involving several elements of the cellular secretory apparatus and specific sequence elements in the signal peptide (see e.g., Miller et al., (1998) J. Biol. Chem. 273:11409). Therefore, different signal peptides vary in their efficiency with which they direct secretion depending on the particular host cell used. Similarly, different signal peptides vary in the efficiency with which they direct secretion of a heterologous protein. Thus, it is necessary to empirically determine the compatibility of a protein, signal sequence, and host cell for efficient secretion of a protein.

Methods and products for preparation of CSR isoforms and/or ligand isoforms, including intron fusion proteins, are provided. These CSR isoforms and/or ligand isoforms are produced by expression of nucleic acid molecules that encode polypeptides linked to sequences that result in improved production of a polypeptide isoform. Provided herein are isoforms fused directly or indirectly to any one or more of a precursor sequence, tag including an epitope tag, fluorescent moiety, or other tag, for improved secretion and/or purification of a polypeptide.

1. Secretion

Recombinant polypeptides expressed in host cells accumulate in one of three compartments: the cytoplasm, bacterial periplasm, or the extracellular medium. Efficient secretion of a protein into the extracellular medium provides means for the easier purification of the polypeptide for several reasons. First, there are usually fewer contaminating proteins which simplifies purification methodologies. Also, extracellular production does not require membrane disruption to recover target proteins, and therefore avoids proteolysis of the recombinant polypeptide by intracellular proteases. Finally, assuming the nucleic acid is correctly fused to a signal sequence, the N-terminal amino acid residue of the secreted polypeptide can be identical to the natural gene product after cleavage of the precursor sequence by a specific signal peptidase, endoproteinase, or exoproteinase.

Secretion requires translocation of the protein across the endoplasmic reticulum (ER) in a cotranslational translocation after the polypeptide is synthesized on a ribosome. Many polypeptides are synthesized as a preproprotein or proprotein containing a pre-and/or prosequence. In mammalian cells, a presequence, also called a signal sequence, is recognized by a 54 kDa protein of the signal recognition particle (SRP) which is believed to hold the nascent chain in a translocation-competent conformation until it contacts the ER. The SRP consists of a 7S RNA and six different polypeptides. The 7S RNA and the 54 kDa signal-sequence binding protein (SRP54) of mammalian SRP exhibit strong similarity to the 4.5S RNA and P48 protein (Ffh) of E. coli which forms the signal recognition particle in bacteria. Generally, translocation of a polypeptide across the ER occurs while it is still being translated and synthesized on a ribosome. At the ER membrane, the nascent protein is inserted into a protein channel that passes through the ER membrane. The signal sequence is immediately cleaved from the polypeptide once it has been translocated. Some polypeptides also contain one or more prosequences that can have diverse functions such as, for example, aiding in the folding of an active polypeptide thereby functioning as an intramolecular chaperone, although prosequences can exhibit other regulatory functions. Upon completion of folding, a prosequence is cleaved by endo- or exo- proteases because generally the prosequence is not necessary for the activity or stability of a mature polypeptide. The ER also contains other resident chaperones which also facilitate folding of the polypeptide protein.

Once folded, the protein is modified, such as by glycosylation, transported to the Golgi apparatus for packaging into vesicles, and secreted from the cell by exocytosis. Secretion of a polypeptide can occur constitutively, which is the default pathway in all cells, whereby transport vesicles destined for the plasma membrane leave the trans-Golgi network in a steady stream for exocytosis of a polypeptide. In some cells, such as neural or endocrine cells, secretion of a polypeptide can be regulated, such as for example by the presence of a sorting or retention signal, which targets a polypeptide to secretory vesicles for later release in response to distinct types of stimulation.

Prokaryotic cells have no organelles such as the ER, but they do have ribosomes bound to the plasma membrane which synthesize secreted proteins for secretion into the space between the plasma membrane and the cell wall (the periplasmic space) in gram negative bacteria. Such secreted proteins have similar N-terminal peptide sequences to eukaryotic secreted proteins, which are cleaved following secretion. Generally, secreted polypeptides are synthesized in the cytoplasm as premature polypeptides and are converted to a mature polypeptide upon cleavage of the signal peptide during transport out of the cytoplasm into the periplasm. Although some secreted proteins can leak from the periplasmic space into the culture medium, E. coli normally do not secrete proteins extracellularly. Rather, movement of polypeptides from the periplasm to the extracellular medium requires outer-membrane disruption. A number of methods, in addition to the presence of a precursor sequence, have been applied to promote extracellular secretion of polypeptides from E. coli including, but not limited to, hemolysin or OmpF fusion, co-expression of kil or tolA, the use of L-form cells, wall-less or wall-deficient cells, and/or coexpression of the bacteriocin release protein (BRP) (see e.g., Choi et al., (2004) Appl Microbiol Biotechnol, 64:625).

Typically, a signal sequence of a polypeptide consists of three regions: an amino-terminal region at the N-terminus of the signal peptide (n-region) containing positively charged amino acid residues, a central hydrophobic core (h-region) of more than 7-8 hydrophobic amino acid residues, and a carboxy terminal region (c-region) that includes the signal peptide cleavage site and is usually a more polar region. In eukaryotes, the characteristic charge of the n-region is supplied by a free amino group at the N-terminal amino acid, whereas in prokaryotes the N-terminal amino acid is formylated and an amino acid with a positively charged side chain is required. Further, the eukaryotic h-region is dominated by Leu with some occurrence of Val, Ala, Phe, and Ile, whereas the prokaryotic h-region is dominated by Leu and Ala in approximately equal proportions. The cleavage of the signal peptide from the mature protein occurs at a specific site in the c-region and the cleavage specificity resides in the last residue of the signal sequence. Small and neutral amino acids at position -1 and -3 of the c-region, usually an Ala, confers processing specificity. In addition to slightly different sequence preferences, eukaryotic signal peptides are somewhat shorter than gram-negative signal peptides, and markedly shorter than gram-positive signal peptides.

Various methods have been used to predict which N-terminal sequences may perform the function of a signal peptide. For example, a widely used algorithm is described in Nielsen et al., (1997) Prot. Eng. 10:1. This algorithm predicts which sequences may serve as a signal peptide with a reasonable degree of accuracy. It does not, however, predict which sequences will function most efficiently. Such methods also are only partially capable of predicting the sites of cleavage at the junction between the signal peptide and the mature protein; for example, the method of Nielsen et al., predicts correctly the site of cleavage of the signal peptide in only 89% of prokaryotic signal sequences. Indeed, some signal peptidases, although biased towards regions containing a consensus sequence following the -3, -1 rule, appear to recognize an unknown three-dimensional motif rather than a specific amino acid sequence around the cleavage site (Dev and Ray (1990) J Bioenerg Biomembr 22:271).

The efficiency of protein secretion varies depending on the host strain, signal sequence, and the type of protein to be secreted. Therefore, there is no general rule in selecting a proper signal sequence for a given recombinant protein to guarantee its successful secretion. For example, despite the similarities among signal peptides, each has a unique sequence. It is likely, therefore, that the various sequences found in different signal peptides interact in different ways with the host cell secretion apparatus. Further, a sequence encoding a signal peptide also often interacts with downstream sequences within the mature protein. For example, in prokaryotes there is a bias in the first 5 amino acids of a successfully cleaved mature protein for the amino acids Ala, Asp/Glu and Ser/Thr. Charged residues close to the N-terminus of the mature protein can negatively influence secretion (called the “charge block” effect, see e.g., Johansson et al., (1993) Mol Gen Genet. 239:256).

Consequently, the choice of signal sequence for optimizing the secretion and expression of a polypeptide is largely empirical since signal sequences widely differ in their ability to facilitate protein translocation, and this is often dependent on the polypeptide to be expressed. A fundamental reason for the variation in signal sequence function is related to the differences in efficacy between heterologous and homologous secretion signals. For example, since many proteins are regulated under physiological conditions, the use of natural endogenous regulatory signals, including signal sequences, for secretion and overexpression of a polypeptide in a homologous host system is not desirable. In another example, foreign signal sequences (e.g. mammalian signal sequences) are not always as efficient in heterologous host cells (e.g. such as insect cells). Thus, it is often, but not always, necessary to substitute an endogenous signal sequence of a foreign polypeptide with a signal sequence derived from the species of the host expression cell.

Use of a host cell for expression of an isoform fusion also can be empirically determined. Generally, a host cell is employed where a signal peptide is compatible with a host cell. A functional signal peptide promotes the extracellular secretion of the polypeptide followed by the cleavage of the signal peptide from the polypeptide. Specific endoproteinases allow the signal peptide to be cut in order to obtain the authentic target sequence. Importantly, the position at which the signal peptide is cleaved can vary according to factors such as the type of host cells employed in expressing a recombinant polypeptide, due in part to the presence of the optimum endoproteinase. Thus, in some instances, the use of a particular signal peptide in a particular host cell can result in the secretion of a polypeptide mixture having different N-terminal amino acids, resulting from cleavage of the signal peptide at more than one site.

Typically, consideration of a signal sequence to be used is dependent upon the host cell to be employed for expression, although some signal sequences are compatible with heterologous hosts. For example, for prokaryotic host cells that do not recognize and process a native intron fusion protein isoform polypeptide, a prokaryotic signal sequence such as, but not limited to, an alkaline phosphatase, penicillinase, or heat-stable enterotoxin II leaders can substitute an endogenous intron fusion protein signal sequence or can be operatively linked to an intron fusion protein that does not contain a functional signal sequence. In another example, for yeast secretion, a yeast invertase, alpha factor, or acid phosphatase signal sequence can substitute a native intron fusion protein isoform signal sequence or can be fused to an intron fusion protein that does not contain a signal sequence. Secretion and expression of an isoform polypeptide in insect cells can be facilitated by using an insect signal sequence such as, but not limited to gp67 or honeybee mellitin to substitute or provide a signal sequence for an intron fusion protein isoform. Additionally, a plant-derived signal sequence can be used to substitute or provide a signal sequence for secretion of an intron fusion protein isoform in a plant. In mammalian cell expression, although an endogenous signal sequence can be satisfactory if it is functional, other mammalian signal sequences, such as for example a tissue plasminogen activator signal sequence, can be superior particularly if secretion of an isoform is desired.

In some examples, a heterologous signal sequence is sufficient and often desired for secretion of a intron fusion protein isoform, including CSR or ligand intron fusion proteins, in a host cell. Considerations for using a cross-host secretion signal include 1) that the signal sequence confers secretion of nucleic acids of different origins (i.e. prokaryotic or eukaryotic); 2) that the functionality of the signal sequence extends beyond its original host; and 3) that the expression and secretion of a polypeptide results in a functional product of appreciable quantity. For example, a human growth hormone (hGH) signal sequence can promote the secretion and expression of recombinant proteins, including intron fusion proteins, in bacterial, insect, and mammalian host expression systems. In another example, a human serum albumin (hHSA) signal sequence can substitute for an endogenous signal sequence and/or can provide for a functional signal sequence to an intron fusion protein isoform to facilitate the expression and secretion of an isoform polypeptide in yeast, insects, and mammalian cells. Additionally, a signal sequence from tissue plasminogen activator can be used to mediate the secretion of polypeptides, including CSR and ligand intron fusion protein isoforms, in insect and mammalian cells. Exemplary signal sequences can include prokaryotic and eukaryotic signal sequences including signal sequences selected from among plant, bacterial, yeast, insect, and mammalian signal sequences.

Exemplary polypeptide precursor sequences can include a signal sequence and optionally also include a prosequence. A leader pro-peptide encoded by a pro-sequence is typically short in composition and contains specific cleavage sites for cleavage by a protease. Generally, cleavage of a pro-peptide sequence occurs within the cell before secretion, such as by an endoprotease, although some polypeptides such as for example apo A1 and prorenin, are secreted intact and cleaved by an extracellular protease or exoprotease. In some examples, a pro-sequence is cleaved both by an endoprotease and an extracellular protease. For example, the pro-sequence of tissue plasminogen activator (tPA) is cleaved by furin in the cell before secretion, and subsequently by a plasmin-like protease following secretion out of the cell. Generally, endoproteases involved in pro-peptide processing such as those with KEX or furin type activities, cleave following dibasic residues through tri and tetrabasic signals. Although many exceptions exist for cleavage requirements, generally pro-peptide cleavage sites are characterized by a basic residue at position-4. Functionally, pro-peptide sequences are diverse and can function to maintain the conformation of a polypeptide, to provide activation of a polypeptide upon the removal of a pro-peptide, and/or to provide recognition sites. Other pro-sequences, for example in tissue plasminogen activator, serve no apparent function and may be retained as an evolutionary remnant (Berg et al., (1991) Biochem Biophys Res Comm, 179: 1289). Exemplary precursor sequences are listed in Table 5.

TABLE 5
Examples of precursor sequences

| | | |
|---|---|---|
| | | SEQ |
| | | ID |
| Precursor Sequence | Amino Acid Sequence NO | |

Bacterial

| | | |
|---|---|---|
| PelB (pectate lyase B) | MKYLLPTAAAGLLLLAAQPAMA | 60 |
| from Erwinia | | |
| carotovora | | |
| OmpA (outer-membrane | MKKTAIAIAVALAGFATVAQA | 61 |
| protein A) | | |
| StII (heat-stable | MKKNIAFLLASMFVFSIATNAYA | 62 |
| enterotoxin II) | | |
| Endoxylanase from | MFKFKKKFLVGLTAAFMSISMFS | 63 |
| Bacillus sp. | ATASA | |
| PhoA (alkaline | MKQSTIALALLPLLFTPVTKA | 64 |
| phosphatase) | | |
| OmpF (outer-membrane | MMKRNILAVIVPALLVAGTANA | 65 |
| protein F) | | |
| PhoE (outer-membrane | MKKSTLALVVMGIVASASVQA | 66 |
| pore protein E) | | |
| MalE (maltose-binding | MKIKTGARILALSALTTMMFSAS | 67 |
| protein) | ALA | |
| OmpC (outer-membrane | MKVKVLSLLVPALLVAGAANA | 68 |
| protein C) | | |
| Lpp (murein lipo- | MKATKLVLGAVILGSTLLAG | 69 |
| protein) | | |
| Lipoprotein (from S. | MNRTKLVLGAVILGSHSAG | 70 |
| marcesens) | | |
| LamB (λ receptor | MMITLRKLPLAVAVAAGVMSAQA | 71 |
| protein) | MA | |
| OmpT (protease VII) | MRAKLLGIVLTTPIAISSFA | 72 |
| LTB (heat-labile | MNKVKCYVLFTALLSSLYAHG | 73 |
| enterotoxin subunit B) | | |
| RbsB (ribosome binding | MNMKKLATLVSAVALSATVSANA | 74 |
| protein) | MA | |
| Heat labile toxin | MKNITFIFFILLASPLYA | 75 |
| subunit A | | |
| β-lactamase (from S. | MKKLIFLIVIALVLSACNSNSSHA | 76 |
| Aureus) | | |
| Staphylococcal protein | MKKKNIYSIRKLGVGIASVTLGTL | 77 |
| A | LISGGVTPAANA | |
| Penicillinase | MSIQHFRVALIPFFAAFCLPVFA | 78 |
| Haemolysin | MMKKTITLLTALLPLASAV | 79 |
| Bacteriophage fd gene | MKKLLFAIPLVVPFYSHS | 80 |
| III | | |
| Yeast | | |
| α-mating factor | MRFPSIFTAVLFAASSALA | 81 |
| PHO1 (acid | MFLQNLFLGFLAVVCANA | 82 |
| phosphatase) | | |
| K. lactis killer toxin | MLVSDSSVDGGERRSS | 83 |
| invertase | MLLQAFLFLLAGFAAKISA | 84 |
| Plant | | |
| PR1b (extracellular | MGFFLFSQMPSFFLVSTLLLFLII | 85 |
| pathogenesis related | SHSSHA | |
| protein, Nichotiana | | |
| tabacum) | | |
| Insect | | |
| gp67 | MLLVNQSHQGFNKEHTSKMVSAIV | 86 |
| | LYVLLAAAAHSAFAAG | |
| Honeybee mellitin | MKFLVNVALVFMVVYISYIYA | 87 |
| EGT (ecdysteroid UDP- | MTILCWLALLSTLTAVNA | 88 |
| glucosyltransferase) | | |
| Mammalian | | |
| tPA (tissue | MDAMKRGLCCVLLLCGAVFVSPS | 89 |
| plasminogen activator) | | |
| presequence | | |
| tPA pre/prosequence | MDAMKRGLCCVLLLCGAVFVSPSQ | 2 |
| | EIHARFRRGAR | |
| pap (human placental | MLLLLLLLGLRLQLSLG | 90 |
| alkaline phosphatase) | | |
| hGH (human growth | MATGSRTSLLLAFGLLCLPWLQEG | 91 |
| hormone) | SA | |
| hHSA (human serum | MKWVTFISLLFLFSSAYS | 92 |
| albumin) | | |
| Human prostatic acid | MRAAPLLLARAASLSLGFLFLLFF | 93 |
| phosphatase | WLDRSVLA | |

2. Purification and/or Detection

Purification of a polypeptide generally is needed to produce a polypeptide in appreciable quantity for study and therapeutic use. Considerations in polypeptide purification include minimizing the existence of contaminating material in a purified preparation. Sources of contaminating material occurring during purification can include other polypeptides, nucleic acids, carbohydrates, lipids, or any other material in a starting sample. Further, a polypeptide optimally retains its biological activity following purification.

Generally, purification of a polypeptide relies on inherent similarities and differences between other polypeptides or potentially contaminating materials. For example, polypeptide similarity is used to purify a polypeptide away from other non-polypeptide contaminants. In contrast, differences in polypeptides, such as for example, differences in size, shape, charge, hydrophobicity, solubility, or biological activity, are used to purify a polypeptide away from other polypeptides. Examples of purification techniques include, but are not limited to, immuno-affinity chromatography, affinity chromatography, protein precipitation, ionic exchange chromatography, hydrophobic interaction chromatography, and size-exclusion chromatography.

Attaching a “tag” to a polypeptide can facilitate recombinant polypeptide purification and/or detection. Nucleic acids encoding a polypeptide tag can be directly fused to a nucleic acid at the carboxy or amino terminus-encoding end thereof to generate a tagged polypeptide. Generally, a coding sequence for a specific tag can be spliced in frame with the coding sequence of a nucleic acid molecule, such as one encoding an isoform, such as an intron fusion protein isoform, to produce a chimeric polypeptide in which, upon expression, the tag is fused to the isoform polypeptide. The tag can be used for detection and/or efficient purification of a polypeptide without requiring knowledge of any properties of a polypeptide or antibodies against the polypeptide or other such reagents. Certain tags encode an epitope that can be purified or detected by a specific antibody. By virtue of their properties, the tags can simplify purification of a desired polypeptide. For example, a tag can facilitate affinity purification of a polypeptide by providing a known epitope for binding to a binding matrix, such as for example a column or bead, immobilized with an affinity ligand. A polypeptide containing a tag at either its carboxy or amino terminus, can be purified in a one-step process by passing a solution, such as for example cellular medium, through an affinity column where the column matrix has a high affinity for the tag.

A tag can include short pieces of well-defined peptides (e.g., Poly-His, Flag-epitope or c-myc epitope or HA-tag) or small proteins (bacterial GST, MBP, Thioredoxin, b-Galactosidase, or VSV-Glycoprotein ). In one example, a tag can include multiple peptides creating an oligo-tag. For example, oligohistidine (Poly-His) tags can be prepared composed of a string of histidine residues, i.e. 1, 2, 3, 4, 5, 6, 7, 8, 9, 10, or more histidine residues. In one embodiment, expression of a fusion polypeptide can be monitored using a tag-specific antibody, allowing a polypeptide to be studied without generating a new, specific antibody to that polypeptide. Epitope tagging can be used to localize gene products in living cells, identify associated proteins or track movement of fusion proteins within the cell. In another embodiment, many tags have their own binding characteristics which can be exploited for purification purposes. For example, poly-His-fusion proteins can bind to Nickel-Sepharose or Nickel-HRP. GST-fusion proteins can bind to glutathione-Sepharose. GST fusion tags are particularly effective in bacterial host cell expression systems since GST isoforms are not normally found in bacteria, and thus there is no competition from endogenous bacterial proteins for binding to a glutathione purification resin. In another example, a ubiquitin tag or a SUMO tag can be employed which, besides facilitating purification, also function as chaperones promoting the correct folding of a polypeptide.

A tag can also be a label such as a luminescent or fluorescent protein and/or any other protein or enzyme that can be detected for localization and/or purification of a polypeptide. In one aspect, isoform fusions can include nucleic acid sequences encoding a luminescent and/or fluorescent protein that are operatively linked to a nucleic acid isoform, including a CSR or ligand intron fusion protein. A luminescent and/or fluorescent polypeptide facilitates the detection, purification, and/or cell localization of a polypeptide. A variety of molecules, such as proteins that emit a detectable light, including luciferins, green fluorescent protein and red fluorescent protein are contemplated herein. Any of a variety of detectable compounds can be used, and can be imaged for detection or purification of a polypeptide by any of a variety of known imaging methods such as for example by using a fluorometer, fluorescence activated cell sorter (FACS), and/or fluorescence microscopy. Exemplary fusion tags, including epitope tags, fluorescent moieies, or other moieties for the detection and/or purification of a polypeptide are listed in Table 6.

TABLE 6
Examples of Fusion Tags

| | | | |
|---|---|---|---|
| | | | SEQ |
| | | | ID |
| Tag | ACC # | Sequence | NO |

| | | | |
|---|---|---|---|
| AU1 | — | DTYRYI | 94 |
| AU5 | — | TDFYLK | 95 |
| DDDDK | — | DDDDK | 96 |
| c-myc | — | EQKLISEEDL | 97 |
| E-tag | — | GAPVPYPDPLEPR | 98 |
| HA | — | YPYDVPDYA | 99 |
| Poly-His | — | (H)n (ex. 6 X His, HHHHHH) | 100 |
| E2 tag | — | GVSSTSSDFRDR | 101 |
| HSV | — | SQPELAPEDPED | 102 |
| KT3 | — | KPPTPPPEPET | 103 |
| S-tag | — | KETAAAKFERQHMDS | 104 |
| VSV-G | — | YTDIEMNRLGK | 105 |
| T7 | — | MASMTGGQQMG | 106 |
| V5 | — | GKPIPNPLLGLDST | 107 |
| Glu-Glu | — | EYMPME | 108 |
| β-galactosidase | P00722 | — | 109 |
| Gal-4 | P04386 | — | 110 |
| Bacterial | P19908 (β chain) | — | 111 |
| luciferase | P19907 (α chain) | | 112 |
| Firefly luciferase | P08659 | —113 | |
| Maltose binding | AAB59056 | — | 114 |
| protein (MBP) | | | |
| Staphylococcal | P02976 | — | 115 |
| protein A | | | |
| Streptococcal | P06654 | — | 116 |
| protein G | | | |
| GFP | AAA27721 | — | 117 |
| Sumo | AAC50996 | — | 118 |
| Ubiquitin | P62988 | — | 119 |
| NusA | P03003 | — | 120 |
| Streptag | | AWRHPQFGG | 121 |
| thioredoxin | NP_418228 | — | 122 |
| GST | P08515 | — | 123 |
| FLAG | — | DYKDDDDK | 124 |
| Protein C | — | EDQVDPRLIDGK | 125 |
| Tag-100 | — | EETARFQPGYRS | 126 |
| T7 gene 10 | — | DLYDDDDK | 127 |

Isoform polypeptides containing one or more fusion tags can be used directly for biological studies and/or can be directly injected into animals to generate antibodies or for other in vivo uses. Among these tags is the His-tag which is relatively small (i.e. less than 10 amino acids), and therefore is less immunogenic than other larger tags. Further, because of its small size, a His-tag may not need to be removed for downstream applications of a purified polypeptide. For other purposes, such as for example therapeutic uses, and for use with some larger fusion tags that can interfere with a function of a polypeptide, a fusion tag can be removed following purification of a polypeptide by treatment with enzymes to generate tag-free recombinant polypeptide isoforms. In one example, a ubiquitin (Ub) tag can be fused to an isoform sequence and following expression and purification of an isoform polypeptide, de-ubiquitinating enzymes (DUBs) can remove Ub to produce a native polypeptide. In another example, a SUMO protease can be used to cleave a SUMO tag from an isoform polypeptide fusion. In an additional example, a fusion polypeptide can be engineered to encode a recognition site for a site-specific protease. For example, a human rhinovirus (HRV 3C) protease recognition site, LeuGluValLeuPheGln/GlyPro (SEQ ID NO: 138), can be engineered into a fusion polypeptide between the nucleic acid encoding the tag and the encoding nucleic acid of interest. A fusion polypeptide containing a tag, such as for example but not limited to, a His tag, S-tag, thioredoxin, GST, NusA, or any other fusion tag, and an HRV 3C protease recognition site, can be incubated with an HRV 3C protease once the fusion polypeptide is bound to an affinity matrix for release of the polypeptide. Other protease recognition sites, including but not limited to a thrombin (R/X or K/X; SEQ ID NO: 133), enterokinase (DDDDK/; SEQ ID NO: 134), TEV-protease (ENLYFQ/G; SEQ ID NO: 135), Factor Xa (I(D or E)GR/; SEQ ID NO: 136), Genease I (HYE or HYD; SEQ ID NO: 137) or any other protease recognition site known to one of skill in the art, can be engineered into a fusion polypeptide containing a tag for recognition by a site-specific protease and release of a tag-free polypeptide. In some instances, a protease recognition site can be engineered adjacent to a purification tag, followed by a linker between the fusion tag and a polypeptide of interest.

D. Isoform Fusions

Provided herein are nucleic acid sequences encoding intron fusion protein fusion polypeptides, including CSR and ligand isoforms, for the production of an intron fusion protein isoform and the encoded proteins. The DNA fusion constructs can include nucleic acid encoding signal and other processing sequences as well as tags and other moieties that facilitate expression and production and/or purification. The fusion constructs encoding isoform fusions can be processed intracellularly and also can be processed extracellularly.

To produce a construct, a nucleic acid encoding an intron fusion protein, such as a nucleic acid encoding all of a portion of a sequence set forth in any one of SEQ ID NOS: 140, 142, 143, 145, 147,149,150, 152,153,155,157, 159, 161, 162, 163, 164, 165, 166, 167, 168, 170, 172, 174, 176, 178, 180, 181, 183, 185, 186, 188, 190, 192, 194, 196, 198, 200,202,204, 206,208,210, 212,214,216, 217,219,221,223,225, 227,229,230,231, 233, 235, 237, 239, 241, 243, 245, 247, 248, 249, 250, 251, 253, 255, 257, 259, 261, 263, 264, 265, 266, 267, 268, 269, 270, 272, 274-280, 282, 284, 286, 288, 289, 350, 352, 354, or allelic variants thereof, can be fused to a nucleic acid encoding a homologous or heterologous precursor sequence that substitutes for and/or provides a functional secretory, processing and/or trafficking sequence. Exemplary encoded precursor sequences are set forth in any one of SEQ ID NOS:2 or 60-93. In one example, an intron fusion protein isoform containing a native or endogenous precursor sequence, such as a signal sequence, of a cognate receptor or ligand can have its precursor sequence supplemented with or replaced with a heterologous or homologous precursor sequence to direct the secretion and production of an isoform polypeptide. In another example, an intron fusion protein isoform that does not contain a precursor sequence of a cognate receptor or ligand can be provided with a heterologous or homologous precursor sequence, fused with an isoform sequence, to improve the secretion and production of an isoform polypeptide. Typically, an isoform that normally (in its native form) contains a signal sequence, does not have this sequence included in a fusion polypeptide containing a heterologous precursor sequence. The precursor sequence is generally utilized by locating it at the N-terminus of a recombinant protein to be secreted from the host cell. A nucleic acid precursor sequence can be operatively joined or linked to a nucleic acid containing the coding region of a CSR or ligand isoform in such a manner that the precursor sequence coding region is upstream of (that is, 5′ of) and in the same reading frame with the isoform coding region to provide an isoform fusion.

Nucleic acid sequences encoding polypeptide linkers can be employed in fusion proteins to link the precursor sequence to the ligand or CSR isoform. The linkage can be direct or via a linker. Such polypeptide linkers typically contain from about 2 or 2 to about 60 or 60 amino acid residues, for example from about 5 to 40, or from about I0 to 30, 2 to 6,7 or 8 amino acid residues. The linker can be used, for example, to relieve steric hindrance or to confer properties, such as altered solubility or to direct or participate in trafficking. The linker also can be used to introduce a restriction enzyme sequence that is used to facilitate direct linkage of nucleic acid sequences for the generation of fusion proteins. Such restriction enzyme linkers are described herein and known in the art. The length of linkers selected depends upon factors, such as the use for which the linker is included.

Such encoded polypeptide linkers can be used to impart advantageous properties. For example, the linker moiety can be a flexible spacer amino acid sequence, such as those used in single-chain antibodies. Examples of known linker moieties include, but are not limited to, peptides, such as (GlymSer)n and (SermGly)n, in which n is 1 to 6, including 1 to 4 and 2 to 4, and m is 1 to 6, including 1 to 4, and 2 to 4, enzyme cleavable linkers, linkers for trafficking and others.

The isoform fusion can be expressed in a host cell, such as a eukaryotic cell, to provide a fusion polypeptide that contains the precursor sequence joined, at its carboxy terminus, to a ligand or CSR isoform at its amino terminus. The fusion polypeptide can be secreted from a host cell. Typically, a precursor sequence is cleaved from the fusion polypeptide during the secretion process, resulting in the accumulation of a secreted isoform in the external cellular environment or, in some cases, in the periplasmic space.

Optionally an intron fusion protein that is a fusion nucleic acid also can include operative linkage with another nucleic acid sequence or sequences, such as a sequence that encodes a tag set forth in any one of SEQ ID NOS:94-127, that promotes the purification and/or detection of an isoform polypeptide. In other embodiments, a nucleic acid sequence of a CSR or ligand intron fusion protein can contain an endogenous signal sequence and can include fusion with a nucleic acid sequence encoding a fusion tag or tags. Many precursor sequences, including signal sequences and prosequences, and/or fusion tag sequences have been identified and are known in the art, such as, but not limited to, those provided and described herein, and are contemplated to be used in conjunction with an isoform nucleic acid molecule. A precursor sequence may be homologous or heterologous to an isoform gene or cDNA, or a precursor sequence can be chemically synthesized. In most cases, the secretion of an isoform polypeptide from a host cell via the presence of a signal peptide and/or propeptide will result in the removal of the signal peptide or propeptide from the secreted intron fusion protein polypeptide. The precursor sequence can be a component of an expression vector, or it can be part of an isoform nucleic acid sequence that is inserted into an expression vector.

Hence, expression of a fusion nucleic acid by a host cell can provide an isoform fusion protein that contains additional amino acids which do not adversely affect the secretory function of the signal peptide and/or the activity of a purified isoform protein. For example, additional amino acids can be included in the fusion protein which separate the signal peptide from the isoform protein in order to provide a favored steric configuration in the fusion protein which promotes the secretion process. The number of such additional amino acids which may serve as separators may vary, and generally do not exceed 60 amino acids. In another example, a fusion protein can contain amino acid residues encoded by a restriction enzyme linker sequence. In an additional example, an isoform fusion protein can contain selective cleavage sites at the junction or junctions between the amino acid of the signal peptide and/or epitope tag and the amino acid sequence of the isoform protein. Such selective cleavage sites may comprise one or more amino acid residues which provide a site susceptible to selective enzymatic, proteolytic, chemical, or other cleavage. For example, the additional amino acids can be a recognition site for cleavage by a site-specific protease. The fusion protein can be further processed to cleave the isoform protein therefrom; for example, if the isoform protein is required without additional amino acids.

1. Exemplary tPA Secretory Sequence

Exemplary of a signal polypeptide for linkage to an isoform is a tPA precursor sequence which, in eukaryotic cells, can direct secretion and other trafficking of linked polypeptides.

Tissue Plasminogen Activator

Tissue plasminogen activator (tPA) is a serine protease that regulates hemostasis by converting the zymogen plasminogen to its active form, plasmin. Like other serine proteases, tPA is synthesized and secreted as an inactive zymogen that is activated by proteolytic processing. Specifically, the mature partially active single chain zymogen form of tPA can be further processed into a two-chain fully active form by cleavage after Arg-310 of SEQ ID NO:4 catalyzed by plasmin, tissue kallikrein or factor Xa. tPA is secreted into the blood by endothelial cells in areas immediately surrounding blood clots, which are areas rich in fibrin. tPA regulates fibrinolysis due to its high catalytic activity for the conversion of plasminogen to plasmin, a regulator of fibrin clots. Plasmin also is a serine protease that becomes converted into a catalytically active, two-chain form upon cleavage of its zymogen form by tPA. Plasmin functions to degrade the fibrin network of blood clots by cutting the fibrin mesh at various places, leading to the production of circulating fragments that are cleared by other proteinases or by the kidney and liver.

The precursor sequence of t-PA encodes a polypeptide that includes a presequence and prosequence corresponding to amino acid residues 1-35 of a full-length tPA sequence set forth in SEQ ID NO:4 and exemplified in SEQ ID NO:2. The precursor sequence of tPA contains a signal sequence including amino acids 1-23 and also contains a prosequence including amino acids 1-35 which contains two cleavage sequences resulting in a prosequence that can include amino acids 24-35, 24-32 and 33-35 of an exemplary tPA pre/prosequences set forth in SEQ ID NO: 2 or 4. The signal sequence of tPA is cleaved co-translationally in the ER and a pro-sequence is removed in the Golgi apparatus by cleavage at a furin processing site following the sequence RFRR occurring at amino acids 29-32 of the exemplary sequences set forth in SEQ ID NO: 2 or 4. Furin cleavage of a tPA pro-sequence retains a three amino acid prosequence GAR, set forth as amino acids 33-35 of an exemplary tPA sequence set forth in SEQ ID NO: 2 or 4. The cleavage of the retained prosequence site is mediated by a plasmin-like extracellular protease to obtain a mature tPA polypeptide beginning at Ser36 set forth in SEQ ID NO:4. Inclusion of a protease inhibitor, such as for example aprotinin, in the culture medium can prevent exopeptidases cleavage and thereby retain a GAR pro-sequence in the mature polypeptide of tPA (Berg et al., (1991) Biochem Biophys Res Comm, 179:1289).

Typically, tPA is secreted by the constitutive secretory pathway, although in some cells tPA is secreted in a regulated manner. For example, in endothelial cells regulated secretion of tPA is induced following endothelial cell activation, for example, by histamine, platelet-activating factor or purine nucleotides, and requires intraendothelial Ca2+ and cAMP signaling (Knop et al., (2002) Biochem Biophys Acta 1600:162). In other cells, such as for example neural cells, specific stimuli that can induce secretion of tPA include exercise, mental stress, electroconvulsive therapy, and surgery (Parmer et al., (1997) J Biol Chem 272:1976). The mechanism mediating the regulated secretion of tPA requires signals on the tPA polypeptide itself, whereas the signal sequence of tPA efficiently mediates constitutive secretion of tPA since a GFP molecule operatively linked only to the signal sequence of tPA is constitutively secreted in the absence of carbachol stimulation (Lochner et al., (1998) Mol Biol Cell, 9:2463). In the absence of a tPA signal sequence, a tPA/GFP hybrid protein is not secreted from cells.

An exemplary tPA precursor sequence including a pre/propeptide sequence of tPA is set forth in SEQ ID NO: 2, and is encoded by a nucleic acid sequence set forth in SEQ ID NO: 1. The signal sequence of tPA includes amino acids 1-23 of SEQ ID NO:2 and the prosequence includes amino acids 24-35 of SEQ ID NO:2 whereby a furin-cleaved prosequence includes amino acids 24-32 and a plasmin-like exoprotease-cleaved prosequence includes amino acids 33-35. Allelic variants of a tPA pre/prosequence are also provided herein, such as those set forth in SEQ ID NO:6 and encoded by a nucleic acid sequence set forth in SEQ ID NO:5. Further, intron fusion protein fusion of a pre/prosequence of mammalian and non-mammalian origin of tPA are contemplated and exemplary sequences are set forth in SEQ ID NOS: 52-59.

Provided herein are nucleic acid molecules and constructs encoding tPA-intron fusion protein fusion polypeptides that contain a CSR or ligand isoform, such as an intron fusion protein, fused to a nucleic acid encoding a precursor sequence. Such intron fusion protein sequences provided herein can exhibit enhanced cellular expression and secretion of an intron fusion protein polypeptide for improved production.

2. tPA-Intron Fusion Protein and other CSR Fusions

Provided herein are nucleic acid molecules and constructs encoding tPA-intron fusion protein fusion polypeptides that contain a CSR or ligand isoform, such as an intron fusion protein. Nucleic acid sequences encoding all or a portion of an intron fusion protein or allelic variants thereof, such as encoding an isoform set forth in any one of SEQ ID NOS: 140, 142, 143, 145, 147, 149, 150, 152, 153, 155, 157, 159, 161, 162, 163, 164, 165, 166, 167, 168, 170, 172, 174, 176, 178, 180, 181, 183, 185, 186,188,190, 192, 194, 196, 198,200,202,204, 206, 208,210,212,214,216, 217, 219,221,223,225,227, 229, 230, 231, 233, 235, 237, 239, 241, 243, 245, 247, 248, 249, 250, 251, 253, 255, 257, 259, 261, 263, 264, 265, 266, 267, 268, 269, 270, 272, 274-280, 282, 284, 286, 288, 289, 350, 352, 354 or allelic variants thereof operatively linked to a tPA pre/prosequence are provided. A tPA pre/prosequence can include a tPA pre/prosequence set forth as SEQ ID NO:1 and encoding a polypeptide set forth in SEQ ID NO:2. In some examples, a tPA pre/prosequence can replace an endogenous precursor sequence of an intron fusion protein and/or provide for an optimal precursor sequence for the secretion of an intron fusion protein polypeptide.

In other embodiments, a nucleic acid encoding all or a portion of an intron fusion protein or allelic variants thereof, such as encoding an isoform set forth in any one of SEQ ID NOS: 140, 142, 143, 145, 147, 149, 150, 152, 153, 155, 157, 159, 161, 162, 163, 164, 165, 166, 167, 168, 170, 172, 174, 176, 178, 180, 181, 183, 185, 186, 188, 190, 192, 194,196, 198, 200, 202, 204, 206, 208, 210, 212, 214, 216, 217, 219, 221, 223, 225, 227, 229, 230, 231, 233, 235, 237, 239, 241, 243, 245, 247, 248, 249, 250, 251, 253, 255, 257, 259, 261, 263, 264, 265, 266, 267, 268, 269, 270, 272, 274-280, 282, 284, 286, 288, 289, 350, 352, 354, or allelic variants thereof can be operatively linked to part of a tPA pre/prosequence including the nucleic acid sequence up to the furin cleavage site of a pre/prosequence of tPA (encoded amino acids 1-32 of an exemplary tPA pre-prosequence set forth in SEQ ID NO:2), thereby excluding nucleic acids encoding amino acids GAR (encoded amino acids 33-35 of an exemplary tPA pre-prosequence set forth in SEQ ID NO:2).

Additionally, a nucleic acid sequence encoding all or a portion of an intron fusion protein or allelic variant thereof, such as encoding an isoform set forth in any one of SEQ ID NOS: 140, 142, 143, 145, 147, 149, 150, 152, 153, 155, 157, 159, 161, 162, 163, 164, 165, 166, 167, 168, 170,172, 174, 176, 178, 180, 181, 183, 185, 186,188, 190, 192,194, 196, 198, 200, 202, 204, 206, 208, 210, 212, 214, 216, 217, 219, 221, 223, 225, 227, 229, 230, 231, 233, 235, 237, 239, 241, 243, 245, 247, 248, 249, 250, 251, 253, 255, 257, 259, 261, 263, 264, 265, 266, 267, 268, 269, 270, 272, 274-280, 282, 284, 286, 288, 289, 350, 352, 354, or allelic variants thereof, can include operative linkage with allelic variants of all or part of a tPA pre/prosequence, such as encoding any allelic variant set forth in SEQ ID NOS: 5 or can include operative linkage with all or part of other tPA pre/prosequences of mammalian and non-mammalian origin, such as encoding a tPA pre/prosequence set forth in any one of SEQ ID NO:52-59. Intron fusion protein-tPA pre/pro fusion sequences provided herein can exhibit enhanced cellular expression and secretion of an intron fusion protein polypeptide for improved production.

In another embodiment, a nucleic acid sequence encoding all or a portion of an intron fusion protein or allelic variant thereof, such as encoding an isoform set forth in any one of SEQ ID NOS: 140, 142, 143, 145, 147, 149, 150, 152, 153, 155, 157, 159, 161, 162, 163, 164, 165, 166, 167, 168, 170, 172, 174, 176, 178, 180, 181, 183, 185, 186, 188, 190, 192, 194, 196, 198, 200, 202, 204, 206, 208, 210, 212, 214, 216, 217, 219, 221, 223, 225, 227, 229, 230, 231, 233, 235, 237, 239, 241, 243, 245, 247, 248, 249, 250, 251, 253, 255, 257, 259, 261, 263, 264, 265, 266, 267, 268, 269, 270, 272, 274-280, 282, 284, 286, 288, 289, 350, 352, 354, or allelic variants thereof can include operative linkage with a presequence (signal sequence) only of a tPA pre/prosequence such as an exemplary signal sequence encoding amino acids 1-23 of an exemplary tPA pre/prosequence set forth as SEQ ID NO:2. Intron fusion protein-tPA presequence fusions provided herein can exhibit enhanced cellular expression and secretion of an intron fusion protein polypeptide for improved production.

In an additional embodiment, a nucleic acid sequence encoding all or a portion of an intron fusion protein or allelic variant thereof, such as encoding any isoform set forth in any one of SEQ ID NOS: 140,142, 143, 145, 147, 149, 150, 152, 153, 155, 157, 159, 161, 162,163, 164, 165,166, 167, 168, 170, 172,174, 176, 178, 180, 181, 183, 185, 186, 188, 190, 192, 194, 196, 198, 200, 202, 204, 206, 208, 210, 212, 214, 216, 217, 219, 221, 223, 225, 227, 229, 230, 231, 233, 235, 237, 239, 241, 243, 245, 247, 248, 249, 250, 251, 253, 255, 257, 259, 261, 263, 264, 265, 266, 267, 268, 269, 270, 272, 274-280, 282, 284, 286, 288, 289, 350, 352, 354, or an allelic variant thereof that contains an endogenous signal sequence of a cognate receptor or ligand can include a fusion with a tPA prosequence where insertion of a tPA prosequence is between an intron fusion protein endogenous signal sequence and an intron fusion protein coding sequence. In one example, a tPA prosequence includes a nucleic acid sequence encoding amino acids 24-32 of an exemplary tPA pre/prosequence set forth as SEQ ID NO:2. In another example, a tPA pro-sequence includes a nucleic acid sequence encoding amino acids 33-35 of an exemplary tPA pre/prosequence set forth as SEQ ID NO:2. In an additional example, a tPA prosequence includes a nucleic acid sequence encoding amino acids 24-35 of an exemplary tPA pre/prosequence set forth as SEQ ID NO:2. Other tPA prosequences can include a nucleic acid sequence encoding amino acids 24-32, 33-35, or 24-35 of allelic variants of tPA pre/prosequences such as set forth in SEQ ID NOS:5 or species variants set forth in any one of SEQ ID NOS: 52-59. Intron fusion protein-tPA prosequence fusions provided herein can exhibit enhanced cellular expression and secretion of an intron fusion protein polypeptide for improved production.

Additionally, a nucleic acid encoding an intron fusion protein or a t-PA-intron fusion protein, such as for example, an intron fusion protein-tPA pre/prosequence fusion, intron fusion protein-tPA presequence fusion, and/or intron fusion protein-tPA prosequence fusion can optionally also include one, two, three, or more tags that facilitate the purification and/or detection of an intron fusion protein polypeptide. Generally, a coding sequence for a specific tag can be spliced in frame on the amino or carboxy ends, with or without a linker region, with a coding sequence of a nucleic acid molecule encoding an intron fusion protein polypeptide. When fusion is on an amino terminus of a sequence, a fusion tag can be placed between an endogenous or heterologous precursor sequence. In one embodiment a nucleic acid encoding a tag, such as a c-myc tag, 8× His tag, or any other fusion tag known to one of skill in the art or set forth in any one of SEQ ID NOS: 94-127, can be placed between an intron fusion protein endogenous signal sequence and an intron fusion protein coding sequence. In another embodiment, a fusion tag can be placed between a nucleic acid sequence encoding a heterologous precursor sequence, such as a tPA pre/prosequence, presequence, or prosequence set forth in SEQ ID NO:2, and an intron fusion protein coding sequence. In other embodiments, a fusion tag can be placed directly on the carboxy terminus of a nucleic acid encoding an intron fusion protein fusion polypeptide sequence. In some instances, an intron fusion protein fusion can contain a linker between an endogenous or heterologous precursor sequence and a fusion tag. Intron fusion protein fusions containing one or more fusion tag(s) provided herein, including intron fusion protein-tPA fusions, can facilitate easier detection and/or purification of an intron fusion protein polypeptide for improved production.

a. FGFR-2 tPA-Intron Fusion Protein Fusion

Provided herein are isoforms of FGFR-2 containing all or part of a pre/prosequence of tPA and optionally a c-myc fusion tag for the improved production of an FGFR-2 intron fusion protein polypeptide. FGFR-2 is a member of the fibroblast growth factor receptor family. Ligands to FGFR-2 include a number of FGF proteins, such as, but not limited to, FGF-1 (basic FGF), FGF-2 (acidic FGF), FGF-4 and FGF-7. FGF receptors are involved in cell-cell communication in tissue remodeling during development as well as cellular homeostasis in adult tissues. Overexpression of, or mutations in, FGFR-2 have been associated with hyperproliferative diseases, including a variety of human cancers, including breast, pancreatic, colorectal, bladder and cervical malignancies. FGFR-2 isoforms such as FGFR-2 intron fusion proteins can be used to treat conditions in which FGF is upregulated, including cancers.

The FGFR-2 protein (GenBank No. NP_{—}000132 set forth as SEQ ID NO:411) is characterized by a signal sequence between amino acids 1-21. FGFR-2 also contains three immunoglobulin-like domains; domain 1 between amino acids 41-125, domain 2 between amino acids 159-249, and domain 3 between amino acids 256-360. FGFR-2 also contains a transmembrane domain between amino acids 378-400 and protein kinase domain between amino acids 481-757.

Exemplary FGFR-2 isoforms include FGFR-2 isoforms set forth in SEQ ID NOS: 178 and 180. These exemplary FGFR-2 isoforms lack one or more domains or a part thereof compared to a cognate FGFR-2 such as set forth in SEQ ID NO:411. The exemplary FGFR-2 isoform set forth as SEQ ID NO: 180 contains a signal peptide at amino acids 1-21, and three immunoglobulin-like domains; domain 1 between amino acids 41-125, domain 2 between amino acids 159-249 and domain 3 between amino acids 256-360, but lacks a transmembrane and protein kinase domain. The exemplary FGFR-2 isoform set forth as SEQ ID NO: 178 contains a signal peptide at amino acids 1-21, immunoglobulin-like domain 2 between amino acids 44-134 and domain 3 between amino acids 141-245, but does not contain an immunoglobulin-like domain 1, a transmembrane domain, or a protein kinase domain.

FGFR-2 isoforms, including FGFR-2 isoforms herein, can include allelic variation in the FGFR-2 isoform polypeptide. For example, a FGFR-2 isoform can include one or more amino acid differences present in an allelic variant of the cognate FGFR-2. In one example, an allelic variant of FGFR-2 contains one or more amino acid changes compared to SEQ ID NO:41 1. For example, one or more amino acid variations can occur in the immunoglobulin domain of FGFR-2. An allelic variant can include amino acid changes at position 105 where, for example Y can be replaced by C, or at position 162 where, for example, M can be replaced by T, or at position 172 where, for example, A can be replaced by F, or at position 186 (SNP NO: 755793) where, for example, M can be replaced by T, or at position 267 where, for example, S can be replaced by P, or at position 276 where, for example, F can be replaced by V, or at position 278 where, for example, C can be replaced by F, or at position 281 where, for example, Y can be replaced by C, or at position 289 where, for example, Q can be replaced by P, or at position 290 where, for example, W can be replaced by C, or at position 315 where, for example, A can be replaced by S, or at position 338 where, for example, G can be replaced by R, or at position 340 where, for example, Y can be replaced by H, or at position 341 where, for example, T can be replaced by P, or at position 342 where, for example, C can be replaced by R, Y, S, F, or W, or at position 344 where, for example, A can be replaced by P or G, or at position 347 where, for example, S can be replaced by C, or at position 351 where, for example, S can be replaced by C, or at position 354 where, for example, S can be replaced by C. Further examples of amino acid changes can occur in the transmembrane domain. An allelic variant can include amino acid changes at position 384 where, for example, G can be replaced by R. Additional amino acid changes also can occur in the protein kinase domain. An allelic variant can include amino acid changes at position 549 where, for example, N can be replaced by H, or at position 565 where, for example, E can be replaced by G, or at position 641 where, for example, K can be replaced by R, or at position 659 where, for example, K can be replaced by N, or at position 663 where, for example, G can be replaced by E, or at position 678 where, for example, R can be replaced by G. Allelic variations also can occur at position 6 where, for example, R can be replaced by P, or at position 31 where, for example, T can be replaced by I, or at position 152 where, for example, R can be replaced by G, or at position 252 where, for example, S can be replaced by W or L, or at position 253 where, for example, P can be replaced by S or R, or at position 372 where, for example, S can be replaced by C, or at position 375 where, for example, Y can be replaced by C. An exemplary FGFR-2 allelic variant containing one or more amino acid changes described above is set forth as SEQ ID NO: 444 and an FGFR-2 isoform can include any one or more allelic variations as set forth in SEQ ID NO:444. An allelic variation in an FGFR-2 isoform can include one or more amino acid changes in the immunoglobulin domain, such as at positions 105, 162, 172, 186, 267, 276, 278, 281, 289, 290, 315, 338, 340, 341, 342, 344, 347, 351, or 354. Additional allelic variations can include one or more amino acid changes, such as at positions 6, 31, 152, 252, or 253.

FGFR-2 isoforms provided herein, or allelic variations thereof, can include a fusion with tPA, such as substitution of an endogenous signal sequence with all or part of a tPA pre/prosequence. For the exemplary FGFR-2 isoforms provided herein as SEQ ID NO: 178 or 180, amino acids 1-22 of an FGFR-2 isoform, including the endogenous signal sequence containing amino acids 1-21, can be replaced by a tPA pre/prosequence, such as for example, the exemplary tPA pre/prosequence set forth as SEQ ID NO: 2 and encoded by a tPA pre/prosequence set forth as SEQ ID NO: 1. For example, the nucleic acid sequence of an exemplary tPA-FGFR-2 intron fusion protein fusion set forth in SEQ ID NO: 39, encoding a polypeptide set forth in SEQ ID NO:40, can include the nucleic acid sequence encoding amino acids 23-281 of the FGFR-2 isoform set forth in SEQ ID NO: 178 operatively linked at the 5′ end to a sequence containing a tPA pre/prosequence (nucleotides 1-105 of SEQ ID NO:39) and a sequence containing an Xho I restriction enzyme linker site (nucleotides 136-141 of SEQ ID NO:39). Optionally, a sequence of an exemplary tPA-FGFR-2 intron fusion protein fusion set forth in SEQ ID NO:39, and encoding a polypeptide set forth in SEQ ID NO:40, also can include a myc epitope tag set forth as nucleotides 106-135 operatively fused between the tPA pre/prosequence and the Xho I linker site. In another example, the nucleic acid sequence of an exemplary tPA-FGFR-2 intron fusion protein fusion set forth in SEQ ID NO:35, encoding a polypeptide set forth in SEQ ID NO:36, can include the nucleic acid sequence encoding amino acids 23-396 of the FGFR-2 isoform set forth in SEQ ID NO: 180 operatively linked at the 5′ end to a sequence containing a tPA pre/pro sequence (nucleotides 1-1 05 of SEQ ID NO:35) and a sequence containing an Xho I restriction enzyme linker site (nucleotides 136-141 of SEQ ID NO:35). Optionally, a sequence of an exemplary tPA-FGFR-2 intron fusion protein fusion set forth in SEQ ID NO:35, and encoding a polypeptide set forth in SEQ ID NO:36, also can include a myc epitope tag set forth as nucleotides 106-135 operatively fused between the tPA pre/pro sequence and the Xho I linker site.

b. FGFR4-tPA Intron Fusion Protein Fusion

Provided herein are isoforms of FGFR-4 containing all or part of a pre/prosequence of tPA and optionally a c-myc fusion tag for the improved production of an FGFR-4 intron fusion protein polypeptide. FGFR-4 is a member of the FGF receptor tyrosine kinase family. FGFR-4 regulation is modified in some cancer cells. For example, in some adenocarcinomas FGFR-4 is down-regulated compared with expression in normal fibroblast cells. Alternate forms of FGFR-4 are expressed in some tumor cells. For example, ptd-FGFR-4 lacks a portion of the FGFR-4 extracellular domain but contains the third Ig-like domain, a transmembrane domain and a kinase domain. This isoform is found in pituitary gland tumors and is tumorigenic. FGFR-4 isoforms can be used to treat diseases and conditions in which FGFR-4 is misregulated. For example, an FGFR-4 isoform can be used to down-regulate tumorigenic FGFR-4 isoforms such as ptd-FGFR-4.

The FGFR-4 protein (GenBank No. NP_{—}002002 set forth as SEQ ID NO: 413) is characterized by a signal sequence between amino acids 1-24. FGFR-4 also contains three immunoglobulin-like domains; domain 1 between amino acids 35-113, domain 2 between amino acids 152-242, and domain 3 between amino acids 249-351. FGFR-4 also contains a transmembrane domain between amino acids 370-386 and protein kinase domain between amino acids 467-743.

Exemplary FGFR-4 isoforms lack one or more domains or a part thereof compared to a cognate FGFR-4 such as set forth in SEQ ID NO:413. The exemplary FGFR-4 isoform set forth as SEQ ID NO: 185 contains a signal peptide between amino acids 1-24, an immunoglobulin-like domain 1 between amino acids 35-113, an immunoglobulin-like domain 2 between amino acids 152-242, and an immunoglobulin-like domain 3 between amino acids 249-351, but lacks a transmembrane and protein kinase domain present in the cognate receptor (e.g., SEQ ID NO: 413).

FGFR-4 isoforms, including FGFR-4 isoforms provided herein, can include allelic variation in the FGFR-4 isoform polypeptide. For example, a FGFR-4 isoform can include one or more amino acid differences present in an allelic variant of the cognate FGFR-4. In one example, an allelic variant of FGFR-4 contains one or more amino acid changes compared to SEQ ID NO:413. For example, one or more amino acid variations can occur in the immunoglobulin domain of FGFR-4. An allelic variant can include amino acid changes at position 275 (SNP NO: 11954456) where, for example, S can be replaced by R, or at position 297 (SNP NO:1057633) where, for example, D can be replaced by V. Additional amino acid changes can occur in the protein kinase domain. An allelic variant can include an amino acid change at position 616 (SNP NO:2301344) where, for example, R can be replaced by L. Allelic variations also can occur at position 10 (SNP NO: 1966265) where, for example, V can be replaced by I, or at position 136 (SNP NO: 376618) where, for example, P can be replaced by L, or at position 388 (SNP NO: 351855) where, for example, G can be replaced by R. An exemplary FGFR-4 allelic variant containing one or more amino acid changes described above is set forth as SEQ ID NO: 446 and an FGFR-4 isoform can include any one or more allelic variations such as set forth in SEQ ID NO:446. An allelic variation in an FGFR-4 isoform can include one or more amino acid changes in an immunoglobulin domain, such as at amino acids corresponding to positions 275 or 297 of SEQ ID NO:413. Additional allelic variants of an FGFR-4 isoform can include any one or more amino acid changes, such as at amino acids corresponding to amino acid positions 10 or 136 of SEQ ID NO:413.

FGFR-4 isoforms provided herein, or allelic variations thereof, can include a fusion with tPA, such as substitution of an endogenous signal sequence with all or part of a tPA pre/prosequence. For the exemplary FGFR-4 isoform provided herein as SEQ ID NO: 185 amino acids 1-25 of the FGFR-4 isoform, including the endogenous signal sequence containing amino acids 1-24, can be replaced by a tPA pre/prosequence, such as for example, the exemplary tPA pre/prosequence set forth as SEQ ID NO: 2 and encoded by a tPA pre/prosequence set forth as SEQ ID NO: 1. For example, the nucleic acid sequence of an exemplary tPA-FGFR-4 intron fusion protein fusion set forth in SEQ ID NO:41, encoding a polypeptide set forth in SEQ ID NO:42, can include the nucleic acid sequence encoding amino acids 26-446 of the FGFR-4 isoform set forth in SEQ ID NO: 185 operatively linked at the 5′ end to a sequence containing a tPA pre/prosequence (nucleotides 1-105 of SEQ ID NO:41) and a sequence containing an Xho I restriction enzyme linker site (nucleotides 136-141 of SEQ ID NO:41). Optionally, a sequence of an exemplary tPA-FGFR-4 intron fusion protein fusion set forth in SEQ ID NO:41 also can include a myc epitope tag set forth as nucleotides 106-135 operatively fused between the tPA pre/prosequence and the Xho I linker site.

C. VEGFR-1-tPA Intron Fusion Protein Fusion

Provided herein are isoforms of VEGFR-1 containing all or part of a pre/prosequence of tPA and optionally a c-myc fusion tag for the improved production of a VEGFR-1 intron fusion protein polypeptide. VEGFR-1 (Flt-1,fms-like tyrosine kinase-1) is a member of the VEGF receptor family of tyrosine kinases. Ligands for VEGFR-1 include VEGF-A and PlGF (placental growth factor). Since VEGFR-1 and its ligands are important for angiogenesis, disregulation of these proteins have a significant impact on a variety of diseases stemming from abnormal angiogenesis, such as proliferation or metastasis of solid tumors, rheumatoid arthritis, diabetic retinopathy, retinopathy and psoriasis. VEGFR-1 also has been implicated in Kawasaki disease, a systemic vasculitis with microvascular hyperpermeability.

The VEGFR-1 polypeptide set forth as SEQ ID NO:426 (GenBank No. NP_{—}002010, SEQ ID NO:426) is characterized by a signal sequence between amino acids 1-26. The VEGFR-1 polypeptide also contains four immunoglobulin-like domains; domain 1 between amino acids 231-337, domain 2 between 332-427, domain 3 between amino acids 558-656, and domain 4 between amino acids 661-749. VEGFR-1 also contains a transmembrane domain between amino acids 764-780 and protein kinase domain between amino acids 827-1154.

The exemplary VEGFR-1 isoform set forth as SEQ ID NO: 279 contains a signal peptide between amino acids 1-26, two immunoglobulin-like domains between amino acids 231-337 and between amino acids 332-427, but does not contain immunoglobulin-like domains 2 and 3. Exemplary VEGFR-1 isoforms also can lack one or more other domains or a part thereof compared to a cognate VEGFR-1 such as set forth in SEQ ID NO:426. For example, the exemplary VEGFR-1 isoform (e.g. SEQ ID NO:279) lacks a transmembrane domain and protein kinase domain compared to a cognate VEGFR-1 (e.g. SEQ ID NO:426). VEGFR-1 isoforms, including VEGFR-1 isoforms herein, can include allelic variation in the VEGFR-l polypeptide, such as one or more amino acid changes compared to a cognate VEGFR-1 polypeptide (e.g., SEQ ID NO: 426).

In some embodiments, a VEGFR-1 polypeptide, such as set forth as SEQ ID NO:426, is described as containing seven Ig-like domains (see e.g., Wiesmann et al. (2000) J Mol Med. 78: 247-260). Such a description includes Ig-like domains that are not classified into the typical domain classifications of Ig V-type or Ig C-type. For example, the VEGFR-1 polypeptide set forth in SEQ ID NO:426 contains a signal sequence between amino acids 1-26. It also contains seven immunoglobulin-like domain including domain 1 between amino acids 38-129, domain 2 between 149-224, domain 3 between amino acids 243-329, domain 4 between amino acids 348-425, domain 5 between amino acids 439-553, domain 6 between amino acids 568-643, and domain 7 between amino acids 673-738. VEGFR-1 also contains a transmembrane domain between amino acids 770-779 and a protein kinase domain between amino acids 827-1154. Hence, based on the above description, the exemplary VEGFR-1isoform set forth as SEQ ID NO: 279 contains a signal peptide between amino acids 1-26, four immunoglobulin-like domains between amino acids 38-129, 149-224, 243-329, and 348-425. In addition, the exemplary VEGFR-1 isoform contains a partial immunoglobulin domain between amino acids 439-560 lacking amino acids 522 to 553 corresponding to the fifth Ig-like domain of a cognate VEGFR1, and does not contain the sixth and seventh Ig-like domains, a transmembrane domain and protein kinase domain compared to a cognate VEGFR-1 (e.g. SEQ ID NO:426).

A VEGFR-1 isoform provided herein, or allelic variations thereof, can include a fusion with tPA, such as substitution of an endogenous signal sequence with all or part of a tPA pre/prosequence. For the exemplary VEGFR-1 isoform provided herein as SEQ ID NO: 279, the endogenous signal sequence containing amino acids 1-26 of the VEGFR-1 isoform can be replaced by a tPA pre/prosequence, such as for example, the exemplary tPA pre/prosequence set forth as SEQ ID NO: 2 and encoded by a tPA pre/prosequence set forth as SEQ ID NO: 1. For example, the nucleic acid sequence of an exemplary tPA-VEGFR-1 intron fusion protein fusion set forth in SEQ ID NO:31, and encoding a polypeptide set forth in SEQ ID NO:32, can include the nucleic acid sequence encoding amino acids 27-541 of the VEGFR-1 isoform set forth in SEQ ID NO: 279 operatively linked at 5′ end to a sequence containing a tPA pre/prosequence (nucleotides 1-105 of SEQ ID NO:31) and a sequence containing an Xho I restriction enzyme linker site (nucleotides 136-141 of SEQ ID NO:3 1). Optionally, a sequence of an exemplary tPA-VEGFR-1 intron fusion protein fusion set forth in SEQ ID NO:31, and encoding a polypeptide set forth in SEQ ID NO:32, also can include a myc epitope tag set forth as nucleotides 106-135 operatively fused between the tPA pre/prosequence and the Xho I linker site.

d. tPA-MET Intron Fusion Protein Fusion

Provided herein are isoforms of MET containing all or part of a pre/prosequence of tPA and optionally a c-myc fusion tag for the improved production of a MET intron fusion protein polypeptide. MET is an RTK for hepatocyte growth factor (HGF), a multifunctional cytokine controlling cell growth, morphogenesis and motility. HGF, a paracrine factor produced primarily by mesenchymal cells, induces mitogenic and morphogenic changes, including rapid membrane ruffling, formation of microspikes, and increased cellular motility. Signaling through MET can increase tumorigenicity, induce cell motility and enhance invasiveness in vitro and metastasis in vivo. MET signaling also can increase the production of protease and urokinase, leading to extracellular matrix/basal membrane degradation, which are important for promoting tumor metastasis.

MET is an RTK that is highly expressed in hepatocytes. MET is comprised of two disulfide-linked subunits, a 50-kDa α subunit and a 145-kDa β subunit. In the fully processed MET protein, the a subunit is extracellular, and the β subunit has extracellular, transmembrane, and tyrosine kinase domains. The ligand for MET is hepatocyte growth factor (HGF). Signaling through FGF and MET stimulates mitogenic activity in hepatocytes and epithelial cells, including cell growth, motility and invasion. As with other RTKs, these properties link MET to oncogenic activities. In addition to a role in cancer, MEt also has been shown to be a critical factor in the development of malaria infection. Activation of MET is required to make hepatocytes susceptible to infection by malaria, thus MET is a prime target for prevention of the disease.

The MET receptor (GenBank No. NP_{—}000236 set forth as SEQ ID NO:414) is characterized by a signal sequence between amino acids 1-24 and a Sema domain between amino acids 55-500. In addition to MET, the Sema domain occurs in semaphorins, which are a large family of secreted and transmembrane proteins, some of which function as repellent signals during axon guidance. In MET, the Sema domain is involved in receptor dimerization in addition to ligand binding. The MET protein also is characterized by a plexin cysteine rich repeat between amino acids 519-562 and three IPT/TIG domains between amino acids 563-655, amino acids 657-739 and amino acids 742-836. IPT stands for Immunoglobulin-like fold shared by Plexins and Transcription factors. TIG stands for the Immunoglobulin-like domain in transcription factors (Transcription factor IG). TIG domains in MET likely play a role in mediating some of the interactions between the extracellular matrix and receptor signaling. The MET protein also is characterized by a transmembrane domain between amino acids 951-973 and a cytoplasmic protein kinase domain between amino acids 1078-1337.

Exemplary MET isoforms provided herein contain one or more domains of a wildtype or predominant form of MET receptor (e.g. set forth as SEQ ID NO:414). For example, an exemplary MET receptor isoform set forth as SEQ ID NOS: 214 contains a signal peptide between amino acids 1-26, a complete Sema domains, a complete plexin cysteine rich repeat domains, and three complete IPT/TIG domains. In addition, exemplary isoforms of MET provided herein can lack one or more domains or a part thereof compared to a cognate MET receptor such as set forth in SEQ ID NO:414. An exemplary MET receptor isoforms provided herein (e.g. SEQ ID NOS: 214) lack a transmembrane domain and a protein kinase domain.

MET isoforms, including MET isoforms herein, can include allelic variation in the MET polypeptide. For example, a MET isoform can include one or more amino acid differences present in an allelic variant of a cognate MET, such as for example, one or more amino acid changes compared to SEQ ID NO:414. For example, one or more amino acid variations can occur in the Sema domain of MET. An allelic variant can include amino acid changes at position 113 where, for example, K can be replaced by R, or at position 114 where, for example, D can be replaced by N, or at position 145 where, for example, V can be replaced by A, or at position 148 where, for example, H can be replaced by R, or at position 151 where, for example, T can be replaced by P, or at position 158 where, for example, V can be replaced by A, or at position 168 where, for example, E can be replaced by D, or at position 193 where, for example, I can be replaced by T, or at position 216 where, for example, V can be replaced by L, or at position 237 where, for example, V can be replaced by A, or at position 276 where, for example, T can be replaced by A, or at position 314 where, for example, F can be replaced by L, or at position 337 where, for example, L can be replaced by P, or at position 340 where, for example, D can be replaced by V, or at position 382 where, for example, N can be replaced by D, or at position 400 where, for example, R can be replaced by G, or at position 476 where, for example, H can be replaced by R, or at position 481 where, for example, L can be replaced by M, or at position 500 where, for example, D can be replaced by G. In a further example, one or more amino acid variation can occur in the plexin cysteine rich repeat domain of MET. An allelic variant can include amino acid changes at position 542 where, for example, H can be replaced by Y. In other examples, one or more amino acid variation can occur in the IPT/TIG domains of MET. An allelic variant can include amino acid changes at position 622 where, for example, L can be replaced by S, or at position 720 where, for example, F can be replaced by S, or at position 729 where, for example, A can be replaced by T. In an additional example, one or more amino acid variations can occur in the protein kinase domain of MET. An allelic variant can include amino acid changes at position 1094 where, for example, H can be replaced by R or at position 1100 where, for example, N can be replaced by Y or at position 1230 where, for example, Y can be replaced by C, or at position 1235 where, for example, Y can be replaced with D, or at position 1250 where, for example, M can be replaced by T. Allelic variants also can include one or more amino acid changes, such as at position 37 where, for example, V can be replaced by A, or at position 39 where, for example M can be replaced by T, or at position 42 where, for example, Q can be replaced by R, or at position 501 where, for example, Y can be replaced by H, or at position 511 where, for example, T can be replaced by A. An exemplary MET allelic variant containing one or more amino acid changes described above is set forth as SEQ ID NO: 447. A MET isoform can include one or more allelic variations as set forth in SEQ ID NO:447. An allelic variation can include one or more amino acid change in the Sema domain, such as at positions 113, 114, 145, 148, 151, 158, 168, 193, 216, 237, 276, 314, 337, 340, 382, 400, 476, 481, or 500. Allelic variations also can occur in the plexin cysteine rich repeat domain, such as at position 542. Further allelic variations also can occur in the IPT/TIG domain, such as at positions 622, 720, or 729. Allelic variations also can include other amino acid changes, such as at positions 37, 39, 42, 501, or 511.

A MET isoform provided herein, or allelic variations thereof, can include a fusion with tPA, such as substitution of an endogenous signal sequence with all or part of a tPA pre/prosequence. For the exemplary MET isoform provided herein as SEQ ID NO: 214, amino acids 1-25 of the MET isoform, including the endogenous signal sequence containing amino acids 1-24, can be replaced by a tPA pre/prosequence, such as for example, the exemplary tPA pre/prosequence set forth as SEQ ID NO: 2 and encoded by a tPA pre/prosequence set forth as SEQ ID NO: 1. For example, the nucleic acid sequence of an exemplary tPA-MET intron fusion protein fusion set forth in SEQ ID NO:33, encoding a polypeptide set forth in SEQ ID NO: 34, can include the nucleic acid sequence encoding amino acids 26-877 of the MET isoform set forth in SEQ ID NO: 214 operatively linked at the 5′ end to a sequence containing a tPA pre/prosequence (nucleotides 1-105 of SEQ ID NO:33) followed by a sequence containing an Xho I restriction enzyme linker site (nucleotides 136-141 of SEQ ID NO:33). Optionally, a sequence of an exemplary tPA-MET intron fusion protein fusion set forth in SEQ ID NO:33, encoding a polypeptide set forth in SEQ ID NO:34, also can include a myc epitope tag set forth as nucleotides 106-135 operatively fused between the tPA pre/prosequence and the Xho I linker site.

e. tPA-RON Intron Fusion Protein Fusion

Provided herein are isoforms of RON (recepteur d'origine nantais; also known as macrophage stimulating 1 receptor) containing all or part of a pre/prosequence of tPA and optionally a c-myc fusion tag for the improved production of a RON intron fusion protein polypeptide. RON is a member of the MET subfamily of RTKs. A ligand for RON is macrophage-stimulating protein (MSP). RON is expressed in cells of epithelial origin. RON plays a role in epithelial cancers including lung cancer and colon cancers. RON and MET are expressed in ovarian cancers and are suggested to confer a selective advantage to cancer cells, thus promoting cancer progression. RON also is overexpressed in certain colorectal cancers. Germline mutations in the RON gene have been linked to human tumorigenesis. RON isoforms can be used to modulate RON, such as by modulating RON activity in diseases and conditions where RON is overexpressed.

The RON protein (GenBank No. NP_{—}002438 set forth as SEQ ID NO:415) contains a signal sequence between amino acids 1-24. RON also is characterized by a Sema domain between amino acids 58-507, a plexin cysteine rich domain between amino acids 526-568, three IPT/TIG domains (between amino acids 569-671, amino acids 684-767, and amino acids 770-860), a transmembrane domain between amino acids 960-982 and a cytoplasmic protein kinase domain between amino acids 1082-1341.

Exemplary RON isoforms lack one or more domains or a part thereof compared to a cognate RON such as set forth in SEQ ID NO:415. For example, an exemplary RON isoform set forth as SEQ ID NO: 223 lacks a transmembrane domain and protein kinase domain. The exemplary RON isoform set forth in SEQ ID NO: 223 contains a complete Sema domain, plexin cysteine rich domain, and three IPT/TIG domains.

RON isoforms, including RON isoforms provided herein, can include allelic variation in the RON polypeptide. For example, a RON isoform can include one or more amino acid differences present in an allelic variant of a cognate RON, such as for example, one or more amino acid changes compared to SEQ ID NO:415. For example, one or more amino acid variations can occur in the Sema domain of RON. An allelic variant can include single nucleotide polymorphisms (SNP) at position 113 (SNP No. 3733136) where, for example, G can be replaced by S, or at position 209 where, for example, G can be replaced by A, or at position 322 (SNP No.2230593) where, for example, Q can be replaced by R, or at position 440 (SNP No.2230592) where, for example, N can be replaced by S. An amino acid variation also can occur at position 523 (SNP No.2230590) where, for example, R can be replaced by Q, or at position 946 (SNP No.13078735) where, for example V can be replaced by M. Additionally, one or more amino acid variations can occur in the protein kinase domain of RON. An allelic variant can include amino acid changes at position 1195 (SNP No.7433231) where, for example, G can be replaced by S, or at position 1335 (SNP No.1062633) where, for example, R can be replaced by G, or at position 1232 where, for example, D can be replaced by V, or at position 1254 where, for example, M can be replaced by T. An exemplary RON allelic variant containing one or more amino acid changes described above is set forth as SEQ ID NO: 448 and RON isoform can include any one or more amino acid differences in an allelic variant, such as set forth in SEQ ID NO:448. An allelic variant can include one or more amino acid changes in the SEMA domain, such as at positions 113, 209, 322, or 440. An allelic variant also can include one or more amino acid change, such as at position 523.

A RON isoform provided herein, or allelic variations thereof, can include a fusion with tPA, such as substitution of an endogenous signal sequence with all or part of a tPA pre/prosequence. For the exemplary RON isoform provided herein as SEQ ID NO:223, amino acids 1-25 of the RON isoform, including the endogenous signal sequence containing amino acids 1-24, can be replaced by a tPA pre/prosequence, such as for example, the exemplary tPA pre/prosequence set forth as SEQ ID NO: 2 and encoded by a tPA pre/prosequence set forth as SEQ ID NO: 1. For example, the nucleic acid sequence of an exemplary tPA-RON intron fusion protein fusion set forth in SEQ ID NO:47, encoding a polypeptide set forth in SEQ ID NO: 48, can include the nucleic acid sequence encoding amino acids 26-908 of the RON isoform set forth in SEQ ID NO:223 operatively linked at the 5′ end to a sequence containing a tPA pre/prosequence (nucleotides 1-105 of SEQ ID NO:47) followed by a sequence containing an Xho I restriction enzyme linker site (nucleotides 136-141 of SEQ ID NO:47). Optionally, a sequence of an exemplary tPA-RON intron fusion protein fusion set forth in SEQ ID NO:47, encoding a polypeptide set forth in SEQ ID NO:48, also can include a myc epitope tag set forth as nucleotides 106-135 operatively fused between the tPA pre/prosequence and the Xho I linker site.

f. tPA-HER2 Intron Fusion Protein Fusion

Provided herein are isoforms of HER2 containing all or part of a pre/prosequence of tPA and optionally a Poly-His fusion tag for the improved production of a HER2 intron fusion protein polypeptide. The human epidermal growth factor receptor 2 gene (HER2; also referred to as ErbB2, NEU, NGL) encodes a receptor tyrosine kinase that has been implicated as an oncogene. HER2 has a major mRNA transcript of 4.5 Kb that encodes a polypeptide of about 185 kDa (p185HER2). HER2 is a member of the human epidermal growth factor receptor (HER) family which also included HER1, HER3, and HER4. Ligands for HER1, HER3, and HER4 include HER1 itself, transforming growth factor-α, amphiregulin, betacellulin, and heregulin. A ligand for HER2 has not been identified, however, HER2 is the preferred heterodimerization partner of the other HER family members thereby enhancing their affinities for their ligands and amplifying their signals. HER2 is overexpressed in 25-30% of human breast and 8-11% of human ovarian cancers.

HER2 (GenBank # NP_{—}004439, set forth as SEQ ID NO:408), like other HER family members, is a type I RTK. The type I RTKs contain an extracellular domain, a singly hydrophobic transmembrane segment, and a cytoplasmic tyrosine kinase domain. The extracellular domains of type I RTKs, including HER2, have been divided into four domains: I (between amino acids 23-217), II (between amino acids 218-342), III (between amino acids 342-500), and IV (between amino acids 501-582). The extracellular region contains four domains arranged as a tandem repeat of a two-domain unit consisting of a ˜190-amino acid L domain referred to as EGFR-like domain since the major determinants for EGF binding lie in domain III of EGFR (domains I and III) followed by a ˜120-amino acid cysteine-rich domain or a furin-like domain (domains II and IV). Specifically, HER2 is characterized by a signal sequence between amino acids 1-22, two Receptor L domains (also called EGFR-like domains) between amino acids 52-173 and 366-486, a furin-like domain between amino acids 189-343, a transmembrane domain between amino acids 633-654, and an intracellular cytoplasmic domain between amino acids 655-1234 with a protein kinase domain between amino acids 720-987.

Several isoforms of HER2 are produced and include polypeptides generated by proteolytic processing and forms generated from alternatively spliced RNAs. Among HER2 isoforms are those designated as herstatins. Herstatins and fragments thereof are HER2 binding proteins, encoded by the HER2 gene. Herstatins (also referred to as p68HER-2) are encoded by an alternatively spliced variant of the gene encoding the p185-HER2 receptor, and retain an intron 8 portion of a HER2 gene. For example, one herstatin occurs in fetal kidney and liver, and includes a 79 amino acid intron-encoded insert, relative to the membrane-localized receptor, at the C terminus (see e.g., U.S. Pat. No. 6,414,130 and U.S. Published Application No. 20040022785). Several herstatin variants have been identified (see, e.g., U.S. Pat. No. 6,414,130; U.S. Published Application No. 20040022785, U.S. application Ser. No. 09/234,208; U.S. application Ser. No.09/506,079; published international application Nos. WO0044403 and WO0161356).

Exemplary HER2 isoforms provided herein contain one or more domains of a wildtype or predominant form of a HER2 cognate receptor (e.g. set forth as SEQ ID NO:408). For example, an exemplary HER2 herstatin isoform, set forth in SEQ ID NO:289, provided herein (for example Dimercept™ Herstatin), contains part of the extracellular domain, typically the first 340 amino acids, of HER2. Herstatins contain a signal peptide between amino acids 1-22, and subdomains I and II and part of domain III between amino acids 341 and 419 (termed IIIa subdomain) of the HER2 extracellular domain and a C-terminal domain encoded by an intron. The resulting herstatin polypeptides typically contain 419 amino acids (340 amino acids including subdomains I and II of the extracellular domain, plus 79 amino acids from intron 8). The herstatin proteins lack extracellular domain IV, as well as the transmembrane domain and kinase domain.

Herstatin binds to HER2, but does not activate the receptor. Herstatins can inhibit members of the EGF-family of receptor tyrosine kinases as well as the insulin-like growth factor-1 (IGF-1) receptor and other receptors. Herstatins prevent the formation of productive receptor dimers (homodimers and heterodimers) required for transphosphorylation and receptor activation. Alternatively or additionally, herstatin can compete with a ligand for binding to the receptor terminus (see e.g., U.S. Pat. No. 6,414,130; U.S. Published Application No. 20040022785, U.S. application Ser. No. 09/234,208; U.S. application Ser. No.09/506,079; published international application Nos. WO0044403 and WO0161356).

HER2 isoforms, including herstatin isoforms provided herein, can include allelic variation in the HER2 polypeptide. For example, a herstatin isoform can include one or more amino acid differences present in an allelic variant of a cognate HER2, such as for example, one or more amino acid changes compared to SEQ ID NO:408. For example, one or more amino acid variations can occur in a Receptor L domain of HER2. An allelic variant can include amino acid changes at position 452 where, for example, W can be replaced C. Other allelic variations can occur in the intracellular cytoplasmic domain, such as for example, at position 654 where, for example, I can be replaced by V, or at position 655 where, for example, I can be replaced by V, or at position 1170 where, for example, P can be replaced by A. An exemplary HER2 allelic variant containing one or more amino acid changes described above is set forth as SEQ ID NO: 442. A HER2 isoform, including a herstatin, can include any one or more allelic variations of a HER2, such as for example, allelic variations as set forth in SEQ ID NO:442.

Additionally, herstatin isoforms provided herein can include allelic variation in the intron 8 portion of a herstatin polypeptide such as for example, one or more amino acid changes compared to SEQ ID NO:319. For example, an allelic variant can include amino acid changes at position 2 where, for example, T can be replaced by S, or at position 5 where, for example L can be replaced by P, or at position 6, where for example, P can be replaced by L, or at position 16 where, for example, L can be replaced by Q, or at position, or at position 18, where for example M can be replaced by L or I, or at position 21, where for example G can be replaced by D, A, or V, or at position 36, where, for example L can be replaced by I, or at position 54 where, for example, P can be replaced by R, or at position 64 where, for example, P can be replaced by L, or at position 73 where, for example, D can be replaced by H or N, or at position 17 where, for example, R can be replaced by C, or at position 31 where, for example, R can be replaced by I. A herstatin variant also can include any one or more of the amino acid variations in the intron 8 portion of a herstatin as set forth above. A summary of allelic variations that can occur in a herstatin, or an intron 8 portion thereof, is set forth below in Table 7, with SEQ ID NOS: indicated in parentheses. An exemplary intron 8 containing any one or more amino acid changes as described above is set forth in SEQ ID NO:320-333, and a herstatin allelic variant containing any one or more amino acid changes in the intron 8 encoded portion is set forth in SEQ ID NO:290-303. A herstatin isoform can include any one or more amino acid variations as set forth in any one of SEQ ID NO: 290-303.

TABLE 7
Herstatin variants and intron 8 variants thereof

| Intron 8 Variant | | Herstatin Variant | |
|---|---|---|---|
| Nucleotide | Amino Acid | Nucleotide | Amino Acid |
| Prominent (334) | Prominent (319) | Prominent (304) | Prominent (289) |
| nt 4 = T (335) | aa 2 = Ser (320) | nt 1036 = T (305) | aa 342 = Ser (290) |
| nt 14 = C (336) | aa 5 = Pro (321) | nt 1046 = C (306) | aa 345 = Pro (291) |
| nt 17 = T (337) | aa 6 = Leu (322) | nt 1049 = T (307) | aa 346 = Leu (292) |
| nt 47 = A (338) | aa 16 = Gln (323) | nt 1079 = A (308) | aa 356 = Gln (293) |
| nt 49 = T (339) | aa 17 = Cys (324) | nt 1081 = T (309) | aa 357 = Cys (294) |
| nt 52 = C (340) | aa 18 = Leu (325) | nt 1084 = C (310) | aa 358 = Leu (295) |
| n 54 = A (341) | aa 18 = Ile (326) | nt 1086 = A (311) | aa 358 = Ile (296) |
| nt 62 = C, T, A (342) | aa 21 = Asp, Ala, | nt 1094 = C, T, A (312) | aa 361 = Asp, Ala, |
| | Val (327) | | Val (297) |
| nt 92 = T (343) | aa 31 = Ile (328) | nt 1124 = T (313) | aa 371 = Ile (298) |
| nt 106 = A (344) | aa 36 = Ile (329) | nt 1138 = A (314) | aa 376 = Ile (299) |
| nt 161 = G (345) | aa 54 = Arg (330) | nt 1193 = G (315) | aa 394 = Arg (300) |
| nt 191 = T (346) | aa 64 = Leu (331) | nt 1223 = T (316) | aa 404 = Leu (301) |
| nt 217 = C or A | aa 73 = His or Asn | nt 1249 = C or A | aa 413 = His or |
| (347) | (332) | (317) | Asn (302) |
| nt 17 = T and nt | aa 6 = Leu and aa | nt 1049 = T and nt | aa 346 = Leu and |
| 217 = C or A | 73 = His or Asn | 1249 = C or A | aa 413 = His or |
| (348) | (333) | (318) | Asn (303) |

A herstatin isoform provided herein, or allelic variations thereof, can include a fusion with tPA, such as substitution of an endogenous signal sequence with all or part of a tPA pre/prosequence. For the exemplary herstatin isoform provided herein as SEQ ID NO: 289 (also called Dimercept™), amino acids 1-23 of the herstatin isoform, including the endogenous signal sequence containing amino acids 1-22, can be replaced by a tPA pre/prosequence, such as for example, the exemplary tPA pre/prosequence set forth as SEQ ID NO: 2 and encoded by a tPA pre/prosequence set forth as SEQ ID NO: 1. For example, the nucleic acid sequence of an exemplary tPA-herstatin intron fusion protein fusion set forth in SEQ ID NO:37, encoding a polypeptide set forth in SEQ ID NO:38, can include the nucleic acid sequence encoding amino acids 24-419 of the herstatin isoform set forth in SEQ ID NO: 289 operatively linked at the 5′ end to a sequence containing a tPA pre/prosequence (nucleotides 1-105 of SEQ ID NO:37) followed by a sequence containing an Xba I restriction enzyme linker site (nucleotides 106-111 of SEQ ID NO:37). Optionally, a sequence of an exemplary tPA-herstatin intron fusion protein fusion set forth in SEQ ID NO:37, encoding a polypeptide set forth in SEQ ID NO:38, also can include a 8X Poly-His epitope tag set forth as nucleotides 112-135 operatively fused between the Xba I linker site and the sequence of a herstatin.

g. tPA-RAGE Intron Fusion Protein Fusion

Provided herein are isoforms of RAGE containing all or part of a pre/prosequence of tPA and optionally a c-myc fusion tag for the improved production of a RAGE intron fusion protein polypeptide. RAGE is a cell-surface receptor that is a member of the immunoglobulin family. RAGEs interact with a variety of macromolecular ligands. For example, glycated adducts of macromolecules, such as glycated proteins and lipids produced by non-enzymatic glycation interact with RAGEs. These glycated adducts, also known as advanced glycation endproducts (AGEs) accumulate in cells and tissues during the normal aging process. Enhanced and/or accelerated accumulation of AGEs occurs in sites of inflammation, in renal failure, under hyperglycemic conditions and conditions of systemic or local oxidative stress. Accumulation can occur in tissues such as vascular tissues. For example AGEs accumulate as AGE-β2-microglobulin in patients with dialysis-related amyloidosis and in vasculature and tissues of diabetes patients. RAGE can bind to additional ligands including S100/calgranulins, β-sheet fibrils, amyloid β peptide, Aβ, amylin, serum amyloid A, prion-derived peptides and amphoterin. S100/calgranulins are cytokine-like pro-inflammatory molecules. S100 proteins (S100P) participate in calcium dependent regulation and other signal transduction pathways. S100P forms S100A12 and S100B are extracellular and can bind to RAGE. S100Ps are expressed in a restricted pattern that includes expression in placental and esophageal epithelial cells. S100Ps also are expressed in cancer cells, including breast cancer, colon cancer, prostate cancer, and pancreatic adenocarcinoma. Amphoterin is a polypeptide of approximately 30 kDa, that is expressed in the nervous system. It also is expressed in transformed cells such as c6 glioma cells, HL-60 promyelocytes, U937 promonocytes, HT1080 fibrosarcoma cells and B16 melanoma cells (Hori et al. (1995) J Bio. Chem. 270:25752-61).

The RAGE polypeptide (Genbank NP_{—}001127, SEQ ID NO:421) contains a number of domains. It has a signal peptide located at the N-terminus. For example, in the exemplary full-length RAGE polypeptide set forth herein as SEQ ID NO:421 and encoded by SEQ ID NO:384, the signal peptide is located at amino acids 1-22. RAGE contains a transmembrane domain. In the exemplary full-length RAGE polypeptide set forth herein as SEQ ID NO:421, the transmembrane domain is between amino acids 343 and 363. RAGE also contains three immunoglobulin-like (Ig-like) domains on the N-terminal side from the transmembrane domain. In the exemplary full-length RAGE polypeptide set forth herein as SEQ ID NO:421, the Ig-like domains are located at amino acids 23-116, 124-221 and 227-317. The first of the Ig-like domains (amino acids 23-116 of SEQ ID NO:421) is a variable-type (V-type) Ig-like domain, whereas the other two Ig-like like domains are characterized as similar to constant regions (C-type). The V-type Ig-like domain can mediate interaction with ligands, such as AGEs (Kislinger et al. (1999(J. Biol. Chem. 274: 31740-49). The C-terminus of the RAGE protein is intracellular. In the exemplary full-length RAGE polypeptide set forth herein as SEQ ID NO:421, the C-terminus encompasses amino acids 364-404. The C-terminus participates in RAGE-mediated signal transduction (Ding et al. (2005) Neuroscience letters 373:67-72).

Exemplary RAGE isoforms provided herein lack one or more domains or parts of one or more domains of RAGE Among the RAGE isoforms provided herein is isoform C02, set forth as SEQ ID NO:237, encoded by a nucleic acid sequence set forth as SEQ ID NO:236. C02 contains 266 amino acids. This isoform includes an N-terminal signal sequence at amino acids 1-22, followed by a V-type Ig-like domain at amino acids 23-116 and one C-type Ig-like domain at amino acids 124-237. It lacks a second C-type Ig-like domain except for the first 4 amino acids (amino acids 243-246) corresponding to amino acids 227-230 of SEQ ID NO:421. In addition, the first C-type Ig-like domain included in C02 contains a disruption. An additional 16 amino acids are inserted; these 16 amino acids are positions 142-157 of SEQ ID NO:237. The insertion point for these amino acids corresponds to amino acids 141-142 of SEQ ID NO:421. C02 isoform contains an additional 20 amino acids at the C-terminus of the polypeptide, amino acids 247-266, that are not present in the cognate RAGE.

RAGE isoforms, including RAGE isoforms herein, can include allelic variation in the RAGE polypeptide. For example, a RAGE isoform can include one or more amino acid differences present in an allelic variant of a cognate RAGE, such as for example, one or more amino acid changes compared to SEQ ID NO:421. For example, one or more amino acid variations can occur in an Ig-like domain of RAGE. An allelic variant can include amino acid changes at position 77 where, for example, R is replaced by C, or at position 82 where, for example, G is replaced by S. In another example, one or more amino acid changes can occur in the C-terminus of RAGE. An allelic variant can include amino acid changes at position 369 where, for example, R can be replaced by Q, or at position 365 where, for example, R can be replaced by G, or at position 305 where, for example, H can be replaced by Q, or at position 307 where, for example, S can be replaced by C. An exemplary RAGE allelic variant containing one or more amino acid changes described above is set forth as SEQ ID NO: 453. A RAGE isoform can include one or more allelic variations as set forth in SEQ ID NO:453. An allelic variation can include one or more amino acid change in an Ig-like domain, such as at positions 77 or 82.

A RAGE isoform provided herein, or allelic variations thereof, can include a fusion with tPA, such as substitution of an endogenous signal sequence with all or part of a tPA pre/prosequence. For the exemplary RAGE isoform provided herein as SEQ ID NO: 237 amino acids 1-23 of the RAGE isoform, including the endogenous signal sequence containing amino acids 1-22, can be replaced by a tPA pre/prosequence, such as for example, the exemplary tPA pre/prosequence set forth as SEQ ID NO: 2 and encoded by a tPA pre/prosequence set forth as SEQ ID NO:1. For example, the nucleic acid sequence of an exemplary tPA-RAGE intron fusion protein fusion set forth in SEQ ID NO:43, encoding a polypeptide set forth in SEQ ID NO: 44, can include the nucleic acid sequence encoding amino acids 23-266 of the RAGE isoform set forth in SEQ ID NO: 237 operatively linked at the 5′ end to a sequence containing a tPA pre/prosequence (nucleotides 1-105 of SEQ ID NO:43) followed by a sequence containing an Xho I restriction enzyme linker site (nucleotides 136-141 of SEQ ID NO:43). Optionally, a sequence of an exemplary tPA-RAGE intron fusion protein fusion set forth in SEQ ID NO:43, encoding a polypeptide set forth in SEQ ID NO:44, also can include a myc epitope tag set forth as nucleotides 106-135 operatively fused between the tPA pre/prosequence and the Xho I linker site.

h. tPA-TEK Intron Fusion Protein Fusion

Provided herein are isoforms of TEK (also called Tie-2) containing all or part of a pre/prosequence of tPA and optionally a c-myc fusion tag for the improved production of a TEK intron fusion protein polypeptide. The known ligands for TEK include angiopoietin (Ang)-1 and Ang-2. The TIE RTKs (including Tie-1 and TEK) play important roles in the development of the embryonic vasculature and continue to be expressed in adult endothelial cells. TEK is an RTK that is expressed almost exclusively by vascular endothelium. Expression of TEK is important for the development of the embryonic vasculature. Overexpression and/or mutation of TEK has been linked to pathogenic angiogenesis, and thus tumor growth, as well as myeloid leukemia.

The TEK protein (GenBank No. NP_{—}000450 set forth as SEQ ID NO:423) contains a signal sequence between amino acids 1-18. TEK also is characterized by a laminin EGF-like domain between amino acids 219-268, three fibronectin type III domains (between amino acids 444-529, amino acids 543-626, and amino acids 639-724), a transmembrane domain between amino acids 748-770, and a cytoplasmic protein kinase domain between amino acids 824-1092.

Exemplary TEK isoforms lack one or more domains or a part thereof compared to a cognate TEK such as set forth in SEQ ID NO:423. For example, exemplary TEK isoforms, such as set forth in SEQ ID NO: 245, can lack a transmembrane domain and kinase domain. TEK isoforms also can contain other domains of a TEK cognate receptor. For example, the exemplary TEK isoform set forth as SEQ ID NO: 245 contains a signal sequence between amino acids 1-18, a laminin EGF-like domain between amino acids 219-268, but is missing the three fibronectin type III domains.

TEK isoforms, including TEK isoforms provided herein, can include allelic variation in the TEK polypeptide. For example, a TEK isoform can include one or more amino acid differences present in an allelic variant of a cognate TEK, such as for example, any one or more amino acid changes compared to SEQ ID NO:423. For example, one or more amino acid variations can occur in a fibronectin type III domain of TEK. An allelic variant can include a single nucleotide polymorphism (SNP) at position 486 (SNP No: 1334811) where, for example, V can be replaced by I, or at position 695 where, for example, I can be replaced by T, or at position 724 (SNP No. 4631561) where, for example, A can be replaced by T. An allelic variant also can occur in the protein kinase domain of TEK. An allelic variant can include amino acid changes at position 849 where, for example, R can be replaced by W. An amino acid variation also can occur at position 346 where, for example, P can be replaced by Q. An exemplary TEK allelic variant containing one or more amino acid changes described above is set forth as SEQ ID NO: 454 and a TEK isoform can include one or more amino acid differences present in an allelic variant of a cognate TEK, such as set forth in SEQ ID NO: 454. An allelic variant of a TEK isoform can include one or more amino acid changes in the fibronectin type III domain, such as at position 486 or 695. An allelic variant of a TEK isoform also can include one or more amino acid changes, such as at position 346.

A TEK isoform provided herein, or allelic variations thereof, can include a fusion with tPA, such as substitution of an endogenous signal sequence with all or part of a tPA pre/prosequence. For the exemplary TEK isoform provided herein as SEQ ID NO: 245 amino acids 1-19 of the TEK isoform, including the endogenous signal sequence containing amino acids 1-18, can be replaced by a tPA pre/prosequence, such as for example, the exemplary tPA pre/prosequence set forth as SEQ ID NO: 2 and encoded by a tPA pre/prosequence set forth as SEQ ID NO:1. For example, the nucleic acid sequence of an exemplary tPA-TEK intron fusion protein fusion set forth in SEQ ID NO:45, encoding a polypeptide set forth in SEQ ID NO: 46, can include the nucleic acid sequence encoding amino acids 20-367 of the TEK isoform set forth in SEQ ID NO: 245 operatively linked at the 5′ end to a sequence containing a tPA pre/prosequence (nucleotides 1-1 05 of SEQ ID NO:45) followed by a sequence containing an Xho I restriction enzyme linker site (nucleotides 136-141 of SEQ ID NO:45). Optionally, a sequence of an exemplary tPA-TEK intron fusion protein fusion set forth in SEQ ID NO:45, encoding a polypeptide set forth in SEQ ID NO:46, also can include a myc epitope tag set forth as nucleotides 106-135 operatively fused between the tPA pre/prosequence and the Xho I linker site.

E. Methods of Producing Nucleic Acid Encoding Isoform Fusion Polypeptides

Exemplary methods for generating isoform fusion nucleic acid molecules and polypeptides, including tPA-intron fusion protein fusions described herein, are provided. Such methods include in vitro synthesis methods for nucleic acid molecules such as PCR, synthetic gene construction and in vitro ligation of isolated and/or synthesized nucleic acid fragments. Nucleic acid molecules for CSR or ligand isoform fusions also can be isolated by cloning methods, including PCR of RNA and DNA isolated from cells and screening of nucleic acid molecule libraries by hybridization and/or expression screening methods.

CSR or ligand isoform polypeptides can be generated from CSR or ligand isoform nucleic acid molecules using in vitro and in vivo synthesis methods. Isoforms, including isoform fusions such as tPA-intron fusion protein fusions, can be expressed in any organism suitable to produce the required amounts and forms of isoform needed for administration and treatment. Expression hosts include prokaryotic and eukaryotic organisms such as E. coli, yeast, plants, insect cells, mammalian cells, including human cell lines and transgenic animals. CSR isoforms also can be isolated from cells and organisms in which they are expressed, including cells and organisms in which isoforms are produced recombinantly and those in which isoforms are synthesized without recombinant means such as genomically-encoded isoforms produced by alternative splicing events.

1. Synthetic Genes and Polypeptides

Nucleic acid molecules encoding CSR or ligand isoform polypeptides can be synthesized by methods known to one of skill in the art using synthetic gene synthesis. In such methods, a polypeptide sequence of an isoform is “back-translated” to generate one or more nucleic acid molecules encoding an isoform. The back-translated nucleic acid molecule is then synthesized as one or more DNA fragments such as by using automated DNA synthesis technology. The fragments are then operatively linked to form a nucleic acid molecule encoding an isoform. Isoform fusions can be generated by joining nucleic acid molecules encoding an isoform with additional nucleic acid molecules such as a heterologous or homologous precursor sequences, epitope or fusion tags, regulatory sequences for regulating transcription and translation, vectors, and other polypeptide-encoding nucleic acid molecules. Isoform-encoding nucleic acid molecules also can be operatively linked with other fusion tags or labels such as for tracking, including radiolabels, and fluorescent moieties.

The process of back translation uses the genetic code to obtain a nucleotide gene sequence for any polypeptide of interest, such as a CSR or ligand isoform. The genetic code is degenerate, 64 codons specify 20 amino acids and 3 stop codons. Such degeneracy permits flexibility in nucleic acid design and generation, allowing for example, the incorporation of restriction sites to facilitate the linking of nucleic acid fragments and/or the placement of unique identifier sequences within each synthesized fragment. Degeneracy of the genetic code also allows the design of nucleic acid molecules to avoid unwanted nucleotide sequences, including unwanted restriction sites, splicing donor or acceptor sites, or other nucleotide sequences potentially detrimental to efficient translation. Additionally, organisms sometimes favor particular codon usage and/or a defined ratio of GC to AT nucleotides. Thus, degeneracy of the genetic code permits design of nucleic acid molecules tailored for expression in particular organisms or groups of organisms. Additionally, nucleic acid molecules can be designed for different levels of expression based on optimizing (or non-optimizing) of the sequences.

Back-translation is performed by selecting codons that encode a polypeptide. Such processes can be performed manually using a table of the genetic code and a polypeptide sequence. Alternatively, computer programs, including publicly available software can be used to generate back-translated nucleic acid sequences.

To synthesize a back-translated nucleic acid molecule, any method available in the art for nucleic acid synthesis can be used. For example, individual oligonucleotides corresponding to fragments of a CSR or ligand isoform-encoding sequence of nucleotides are synthesized by standard automated methods and mixed together in an annealing or hybridization reaction. Such oligonucleotides are synthesized such that annealing results in the self-assembly of the gene from the oligonucleotides using overlapping single-stranded overhangs formed upon duplexing complementary sequences, generally about 100 nucleotides in length. Single nucleotide “nicks” in the duplex DNA are sealed using ligation, for example with bacteriophage T4 DNA ligase. Restriction endonuclease linker sequences can, for example, then be used to insert the synthetic gene into any one of a variety of recombinant DNA vectors suitable for protein expression. In another, similar method, a series of overlapping oligonucleotides are prepared by chemical oligonucleotide synthesis methods. Annealing of these oligonucleotides results in a gapped DNA structure. DNA synthesis catalyzed by enzymes such as DNA polymerase I can be used to fill in these gaps, and ligation is used to seal any nicks in the duplex structure. PCR and/or other DNA amplification techniques can be applied to amplify the formed linear DNA duplex.

Additional nucleotide sequences can be joined to a CSR or ligand isoform-encoding nucleic acid molecule thereby generating an isoform fusion, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to an isoform-encoding nucleic acid molecule. Examples of such sequences include, but are not limited to, promoter sequences designed to facilitate intracellular protein expression, or precursor sequences designed to facilitate protein secretion. Other examples of nucleotide sequences that can be operatively linked to an isoform-encoding nucleic acid molecule include sequences that facilitate the purification and/or detection of an isoform. For example, a fusion tag such as an epitope tag or fluorescent moiety can be fused or linked to an isoform. Additional nucleotide sequences such as sequences specifying protein binding regions also can be linked to isoform-encoding nucleic acid molecules. Such regions include, but are not limited to, sequences to facilitate uptake of an isoform into specific target cells, or otherwise enhance the pharmacokinetics of the synthetic gene.

CSR isoforms also can be synthesized using automated synthetic polypeptide synthesis. Cloned and/or in silico-generated polypeptide sequences can be synthesized in fragments and then chemically linked. Alternatively, isoforms can be synthesized as a single polypeptide. Such polypeptides then can be used in the assays and treatment administrations described herein.

2. Methods of Cloning and Isolating Isoforms and Isoform Fusions

CSR or ligand isoforms, including isoform fusions, can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Nucleic acid molecules encoding isoforms also can be isolated using library screening. For example, a nucleic acid library representing expressed RNA transcripts as cDNAs can be screened by hybridization with nucleic acid molecules encoding CSR isoforms or portions thereof. For example, an intron sequence or portion thereof from a CSR gene can be used to screen for intron retention-containing molecules based on hybridization to homologous sequences. Expression library screening can be used to isolate nucleic acid molecules encoding a CSR isoform. For example, an expression library can be screened with antibodies that recognize a specific isoform or a portion of an isoform. Antibodies can be obtained and/or prepared which specifically bind a CSR isoform or a region or peptide contained in an isoform. Antibodies which specifically bind an isoform can be used to screen an expression library containing nucleic acid molecules encoding an isoform, such as an intron fusion protein. Methods of preparing and isolating antibodies, including polyclonal and monoclonal antibodies and fragments therefrom are well known in the art. Methods of preparing and isolating recombinant and synthetic antibodies also are well known in the art. For example, such antibodies can be constructed using solid phase peptide synthesis or can be produced recombinantly, using nucleotide and amino acid sequence information of the antigen binding sites of antibodies that specifically bind a candidate polypeptide. Antibodies also can be obtained by screening combinatorial libraries containing of variable heavy chains and variable light chains, or of antigen-binding portions thereof. Methods of preparing, isolating and using polyclonal, monoclonal and non-natural antibodies are reviewed, for example, in Kontermann and Dubel, eds. (2001) “Antibody Engineering” Springer Verlag; Howard and Bethell, eds. (2001) “Basic Methods in Antibody Production and Characterization” CRC Press; and O'Brien and Aitkin, eds. (2001) “Antibody Phage Display” Humana Press. Such antibodies also can be used to screen for the presence of an isoform polypeptide, for example, to detect the expression of a CSR isoform in a cell, tissue or extract.

Methods for amplification of nucleic acids can be used to isolate nucleic acid molecules encoding an isoform, including for example, polymerase chain reaction (PCR) methods. A nucleic acid containing material can be used as a starting material from which an isoform-encoding nucleic acid molecule can be isolated. For example, DNA and mRNA preparations, cell extracts, tissue extracts, fluid samples (e.g. blood, serum and saliva), samples from healthy and/or diseased subjects can be used in amplification methods. Nucleic acid libraries also can be used as a source of starting material. Primers can be designed to amplify an isoform. For example, primers can be designed based on expressed sequences from which an isoform is generated. Primers can be designed based on back-translation of an isoform amino acid sequence. Nucleic acid molecules generated by amplification can be sequenced and confirmed to encode an isoform.

3. Methods of Generating and Cloning Intron Fusion Protein Fusions

The methods by which DNA sequences can be obtained and linked to provide the DNA sequence encoding the fusion protein are well known in the field of recombinant DNA technology. DNA for a precursor sequence, such as DNA encoding a signal peptide, can be generated by various methods including: synthesis using an oligonucleotide synthesizer; isolation from a target DNA such as from an organism, cell, or vector containing the precursor sequence, by appropriate restriction enzyme digestion; or can be obtained from a target source by PCR of genomic DNA with the appropriate primers. Likewise, the DNA encoding an isoform fusion protein, epitope tag, or other protein to be fused to an isoform can be synthesized using an oligonucleotide synthesizer, isolated from the DNA of a parent cell which produces the protein by appropriate restriction enzyme digestion, or obtained from a target source, such as a cell, tissue, vector, or other target source, by PCR of genomic DNA with appropriate primers. Additionally, a small epitope tag, such as a myc tag, His tag, or other small epitope tag, and/or any other additional DNA sequence such as a restriction enzyme linker sequence or a protease cleavage site sequence can be engineered into a PCR primer sequence for incorporation into a nucleic acid sequence encoding another protein upon PCR amplification for incorporation into the DNA encoding the fusion protein.

In one example, intron fusion protein fusion sequences can be generated by successive rounds of ligating DNA target sequences, amplified by PCR, into a vector at engineered recombination site. For example, a nucleic acid sequence for an intron fusion protein isoform, fusion tag, and/or a homologous or heterologous precursor sequence can be PCR amplified using primers that hybridize to opposite strands and flank the region of interest in a target DNA. Cells or tissues or other sources known to express a target DNA molecule, or a vector containing a sequence for a target DNA molecule, can be used as a starting product for PCR amplification events. The PCR amplified product can be subcloned into a vector for further recombinant manipulation of a sequence, such as to create a fusion with another nucleic acid sequence already contained within a vector, or for the expression of a target molecule.

PCR primers used in the PCR amplification also can be engineered to facilitate the operative linkage of nucleic acid sequences. For example, non-template complementary 5′ extension can be added to primers to allow for a variety of post-amplification manipulations of the PCR product without significant effect on the amplification itself. For example, these 5′ extensions can include restriction sites, promoter sequences, sequences for epitope tags, etc. In one example, for the purpose of creating a fusion sequence, sequences that can be incorporated into a primer include, for example, a sequence encoding a myc epitope tag or other small epitope tag, such that the amplified PCR product effectively contains a fusion of a nucleic acid sequence of interest with an epitope tag.

In another example, incorporation of restriction enzyme sites into a primer can facilitate subdloning of the amplification product into a vector that contains a compatible restriction site, such as by providing sticky ends for ligation of a nucleic acid sequence. Subcloning of multiple PCR amplified products into a single vector can be used as a strategy to operatively link or fuse different nucleic acid sequences. Examples of restriction enzyme sites that can be incorporated into a primer sequence can include, but are not limited to, an Xho I restriction site (CTCGAG, SEQ ID NO:128), an Nhe I restriction site (GCTAGC, SEQ ID NO:130), a Not I restriction site (GCGGCCGC, SEQ ID NO: 131), an EcoR I restriction site (GAATTC, SEQ ID NO:132), or an Xba I restriction site (TCTAGA, SEQ ID NO:129). Other methods for subcloning of PCR products into vectors include blunt end cloning, TA cloning, ligation independent cloning, and in vivo cloning.

The creation of an effective restriction enzyme site into a primer facilitates the digestion of the PCR fragment with a compatible restriction enzyme to expose sticky ends, or for some restriction enzyme sites, blunt ends, for subsequent subcloning. There are several factors to consider in engineering a restriction enzyme site into a primer so that it retains its compatibility for a restriction enzyme. First, the addition of 2-6 extra bases upstream of an engineered restriction site in a PCR primer can greatly increase the efficiency of digestion of the amplification product. Other methods that can be used to improve digestion of a restriction enzyme site by a restriction enzyme include proteinase K treatment to remove any thermostable polymerase that can block the DNA, end-polishing with Klenow or T4 DNA polymerase, and/or the addition of spermidine. An alternative method for improving digestion efficiency of PCR products also can include concatamerization of the fragments after amplification. This is achieved by first treating the cleaned up PCR product with T4 polynucleotide kinase (if the primers have not already been phosphorylated). The ends may already be blunt if a proofreading thermostable polymerase such as Pfu was used or the amplified PCR product can be treated with T4 DNA polymerase to polish the ends if a non-proofreading enzyme such as Taq is used. The PCR products can be ligated with T4 DNA ligase. This effectively moves the restriction enzyme site away from the end of the fragments and allows for efficient digestion.

Prior to subcloning of a PCR product containing exposed restriction enzyme sites into a vector, such as for creating a fusion with a sequence of interest, it is sometimes necessary to resolve a digested PCR product from those that remain uncut. In such examples, the addition of fluorescent tags at the 5′ end of a primer can be added prior to PCR. This allows for identification of digested products since those that have been digested successfully will have lost the fluorescent label upon digestion.

In some instances, the use of amplified PCR products containing restriction sites for subsequent subcloning into a vector for the generation of a fusion sequence can result in the incorporation of restriction enzyme linker sequences in the fusion protein product. Generally such linker sequences are short and do not impair the function of a polypeptide so long as the sequences are operatively linked.

The nucleic acid molecule encoding an isoform fusion protein can be provided in the form of a vector which comprises the nucleic acid molecule. One example of such a vector is a plasmid. Many expression vectors are available and known to those of skill in the art and can be used for expression of CSR or ligand isoform, including isoform fusions. The choice of expression vector can be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

4. Expression Systems

CSR and ligand isoforms, including natural and combinatorial intron fusion proteins and isoform fusions provided herein, can be produced by any method known to those of skill in the art including in vivo and in vitro methods. CSR and ligand isoforms and fusion isoforms can be expressed in any organism suitable to produce the required amounts and form of isoform needed for administration and treatment. Generally, any cell type that can be engineered to express heterologous DNA and has a secretory pathway is suitable. Expression hosts include prokaryotic and eukaryotic organisms such as E. coli, yeast, plants, insect cells and mammalian cells, including human cell lines and transgenic animals. Expression hosts can differ in their protein production levels as well as the types of post-translational modifications that are present on the expressed proteins. Further, the choice of expression hosts is often, but not always, dependent on the choice of precursor sequence utilized. For example, many heterologous signal sequences can only be expressed in a host cell of the same species (i.e., an insect cell signal sequence is optimally expressed in an insect cell). In contrast, other signal sequences can be used in heterologous hosts such as, for example, the human serum albumin (hHSA) signal sequence which works well in yeast, insect, or mammalian host cells and the tissue plasminogen activator pre/pro sequence which has been demonstrated to be functional in insect and mammalian cells (Tan et al., (2002) Protein Eng. 15:337). The choice of expression host can be made based on these and other factors, such as regulatory and safety considerations, production costs and the need and methods for purification.

a. Prokaryotic Expression

Prokaryotes, especially E. coli, provide a system for producing large amounts of proteins such as isoforms and isoform fusions provided herein. Other microbial strains may also be used, such as bacilli, for example Bacillus subtilus, various species of Pseudomonas, or other bacterial strains. Transformation of bacteria, including E. coli, is a simple and rapid technique well known to those of skill in the art. In such prokaryotic systems, plasmid vectors which contain replication sites and control sequences derived from a species compatible with the host are often used. For example, common vectors for E. coli include pBR322, pUC18, pBAD, and their derivatives. Commonly used prokaryotic control sequences, which contain promoters for transcription initiation, optionally with an operator, along with ribosome binding-site sequences, include such commonly used promoters as the beta-lactamase (penicillinase) and lactose (lac) promoter systems, the tryptophan (trp) promoter system, the arabinose promoter, and the lambda-derived Pl promoter and N-gene ribosome binding site. Any available promoter system compatible with prokaryotes, however, can be used. Expression vectors for E. coli can contain inducible promoters. Such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated λPL promoter.

Isoforms can be expressed in the cytoplasmic environment of E. coli. The cytoplasm is a reducing environment and for some molecules, this can result in the formation of insoluble inclusion bodies. Reducing agents such as dithiothreotol and β-mercaptoethanol and denaturants, such as guanidine-HCl and urea can be used to resolubilize the proteins. An alternative approach is the expression of CSR or ligand isoforms, including isoform fusions, in the periplasmic space of bacteria which provides an oxidizing environment and chaperonin-like and disulfide isomerases and can lead to the production of soluble protein. Typically, a precursor sequence, such as but not limited to precursor sequences described herein for use in bacteria including an OmpA, OmpF, PelB, or other precursor sequence, is fused to the protein to be expressed, such as by replacing an endogenous precursor sequence, which directs the protein to the periplasm. The leader peptide is then removed by signal peptidases inside the periplasm. Examples of periplasmic-targeting precursor or leader sequences include the PelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce aglycosylated proteins. Thus, if proteins require glycosylation for function, glycosylation can be added in vitro after purification from host cells.

b. Yeast

Yeasts such as Saccharomyces cerevisae, Schizosaccharomyces pombe, Yarrowia lipolytica, Kluyveromyces lactis and Pichia pastoris are well known yeast expression hosts that can be used for production of CSR isoforms. Yeast can be transformed with episomal replicating vectors or by stable chromosomal integration by homologous recombination. Typically, inducible promoters are used to regulate gene expression. Examples of such promoters include GAL 1, GAL7 and GAL5 and metallothionein promoters, such as CUP1, AOX1 or other Pichia or other yeast promoter. Other yeast promoters include promoters for synthesis of glycolytic enzymes, e.g., those for 3-phosphoglycerate kinase, or those from the enolase gene or the Leu2 gene obtained from Yep13. Expression vectors often include a selectable marker such as LEU2, TRP1, HIS3 and URA3 for selection and maintenance of the transformed DNA. An exemplary expression vector system for use in yeast is the POT1 vector system (see e.g., U.S. Pat. No. 4,931,373), which allows transformed cells to be selected by growth in glucose-containing media. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase, or any other heterologous or homologous precursor sequence that promotes the secretion of a polypeptide in yeast. A protease cleavage site such as, for example, the Kex-2 protease, can be engineered to remove the fused sequences from the expressed polypeptides as they exit the secretion pathway. Yeast also are capable of glycosylation at Asn-X-Ser/Thr motifs.

c. Insect Cells

Insect cells, particularly using baculovirus expression, are useful for expressing polypeptides such as CSR or ligand isoforms, including isoform fusions. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the Bombyx mori nuclear polyhedrosis virus (BmNPV) and an insect cell line such as Sf9 derived from Spodoptera frugiperda, Pseudaletia unipuncta (A7S) and Danaus plexippus (DpN1). For high-level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. For example, a mammalian tissue plasminogen activator precursor sequence facilitates expression and secretion of proteins by insect cells. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schnieder 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian Cells

Mammalian expression systems can be used to express CSR or ligand isoforms, including isoform fusions provided herein. Expression constructs can be transferred to mammalian cells by viral infection such as by using an adenovirus vector or by direct DNA transfer such as by conventional transfection methods involving liposomes, calcium phosphate, DEAE-dextran and by physical means such as electroporation and microinjection. Exemplary expression vectors include, fore example pCI expression plasmid (Promega, SEQ ID NO:50), or the pcDNA3.1 expression plasmid (Invitrogen, SEQ ID NO:51). Expression vectors for mammalian cells typically include an mRNA cap site, a TATA box, a translational initiation sequence (Kozak consensus sequence) and polyadenylation elements. Such vectors often include transcriptional promoter-enhancers for high-level expression, for example the SV40 promoter-enhancer, the human cytomegalovirus (CMV) promoter, such as the hCMV-MIE promoter-enhancer, and the long terminal repeat of Rous sarcoma virus (RSV), or other viral promoters such as those derived from polyoma, adenovirus II, bovine papilloma virus or avian sarcoma viruses. Additional suitable mammalian promoters include the β-actin promoter-enhancer and the human metallothionein II promoter. These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha-1-antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase and thymidine kinase. Fusion with cell surface signaling molecules such as TCR-ζ and Fc_{ε}RI-γ can direct expression of the proteins in an active state on the cell surface.

Many cell lines are available for mammalian expression including mouse, rat human, monkey, chicken and hamster cells. Exemplary cell lines include, but are not limited to, CHO, Balb/3T3, HeLa, MT2, mouse NS0 (nonsecreting) and other myeloma cell lines, hybridoma and heterohybridoma cell lines, lymphocytes, fibroblasts, Sp2/0, COS, NIH3T3, HEK293, 293T, 293S, 2B8, and HKB cells. Cell lines also are available adapted to serum-free media which facilitates purification of secreted proteins from the cell culture media. One such example is the serum free EBNA-1 cell line (Pham et al., (2003) Biotechnol. Bioeng. 84:332-42.)

e. Plants

Transgenic plant cells and plants can be used to express CSR isoforms. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline synthase promoter, the ribose bisphosphate carboxylase promoter and the ubiquitin and UBQ3 promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce CSR isoforms (see for example, Mayfield et al. (2003) PNAS 100:438-442). Because plants have different glycosylation patterns than mammalian cells, this can influence the choice of CSR isoforms produced in these hosts.

5. Methods of Transfection and Transformation

Transformation or transfection of host cells is accomplished using standard techniques suitable to the chosen host cells. Methods of transfection are known to one of skill in the art, for example, calcium phosphate and electroporation, as well as the use of commercially available cationic lipid reagents, such as Lipofectamine™ Lipofectamine™ 2000, or Lipofectin® (Invitrogen, Carlsbad Calif.), which facilitate transfection. Depending on the host cell used, transformation is performed using standard techniques appropriate to such cells. Calcium treatment, employing calcium chloride for example, or electroporation is generally used for prokaryotes or other cells that contain substantial cell-wall barriers. Infection with Agrobacterium tumefaciens is used for transformation of certain plant cells. For mammalian cells without such cell walls, calcium phosphate precipitation can be employed. General aspects of transformation are described for plant cells (see e.g., Shaw et al., (1983) Gene, 23:315, WO89/05859), mammalian cells (see e.g., U.S. Pat. No. 4,399,216, Keown et al., Methods in Enzymolog., (1990) 185:527; Mansour et al., (1988) Nature 336:348), or yeast cells (see e.g. Val Solingen et al., (1977) J Bact (1977) 130:946, Hsiao et al., (1979) Proc. Natl. Acad. Sci., 76:3829). Other methods for introducing DNA into a host cell include, but are not limited to, nuclear microinjection, electroporation, bacterial protoplast fusion with intact cells, or using polycations such as polybrene or polyornithine.

6. Production and Purification

The cells containing the expression vectors are cultured under conditions appropriate for production of the fusion polypeptide, and the fusion polypeptide or the cleaved mature recombinant protein (that is, the expressed protein with or without the precursor peptide sequence) is then recovered and purified. In general the protein that will be recovered is the isoform fusion polypeptide (for example containing fusion with an epitope tag or other fusion sequence) or the isoform (after cleavage of the precursor peptide), or both. It will be apparent that when the fusion polypeptide is secreted and the precursor peptide is cleaved during the process, the protein that will be recovered will be the isoform protein, or a modified form thereof. In some cases, the fusion polypeptide will be designed such that there can be additional amino acids present between the precursor peptide sequence and the isoform protein, such as for example, a restriction enzyme linker site. In these instances, cleavage of the precursor peptide from the fusion polypeptide can produce a modified isoform polypeptide having additional amino acids at the N-terminus. Non-limiting examples of additional amino acids that can be incorporated at the N-terminus of a secreted polypeptide due to the presence of a restriction enzyme linker sequence include, for example, SR or LE. Alternatively, the fusion polypeptide may be designed such that the precursor peptide is not completely processed such that incomplete cleavage of the precursor polypeptide results. For example, for a tPA precursor sequence, incomplete cleavage can occur at the furin cleavage site or the plasmin-like cleavage site. Where incomplete cleavage occurs at the plasmin-like cleavage site a modified isoform may be produced which has an altered N-terminus including, for example, addition of amino acids GAR. In some examples, a purified isoform can be treated with a plasmin-like protease resulting in a polypeptide that does not retain a GAR sequence at its N-terminus.

Modified CSR and ligand isoforms can include one or more additional amino acids at the N-terminus. These additional amino acids can include, but are not limited to, GAR, SR, LE or combinations thereof such as GARSR (SEQ ID NO:563) or GARLE (SEQ ID NO:564). Additionally, the secreted polypeptide also can include an amino acid sequence of a tag in addition to other sequences at the N-terminus of a secreted isoform polypeptide.

An isoform fusion polypeptide can be isolated using various techniques well-known in the art. One skilled in the art can readily follow known methods for isolating polypeptides and proteins in order to obtain one of the isolated polypeptides or proteins provided herein. These include, but are not limited to, immunochromatography, HPLC, size-exclusion chromatography, and ion-exchange chromatography. Examples of ion-exchange chromatography include anion and cation exchange and include the use of DEAE Sepharose, DEAE Sephadex, CM Sepharose, SP Sepharose, or any other similar column known to one of skill in the art. Isolation of an isoform fusion polypeptide from the cell culture media or from a lysed cell can be facilitated using antibodies directed against either an epitope tag in an isoform fusion polypeptide or against the isoform polypeptide and then isolated via immunoprecipitation methods and separation via SDS-polyacrylamide gel electrophoresis (PAGE). Alternatively, an isoform fusion can be isolated via binding of a polypeptide-specific antibody to an isoform fusion polypeptide and subsequent binding of the antibody to protein-A or protein-G sepharose columns, and elution of the protein from the column. The purification of an isoform fusion protein also can include an affinity column or bead immobilized with agents which will bind to the protein, followed by one or more column steps for elution of the protein from the binding agent. Examples of affinity agents include concanavalin A-agarose, heparin-toyopearl, or Cibacrom blue 3Ga Sepharose. A protein can also be purified by hydrophobic interaction chromatography using such resins as phenyl ether, butyl ether, or propyl ether.

In some examples, an isoform fusion protein can be purified using immunoaffinity chromatography. In such examples, an isoform fusion can be expressed as a fusion protein with an epitope tag such as described herein including, but not limited to, maltose binding protein (MBP), glutathione-S-transferase (GST) or thioredoxin (TRX), or a His tag. Kits for expression and purification of such fusion proteins are commercially available from New England BioLab (Beverly, Mass.), Pharmacia (Piscataway, N.J.), Invitrogen, and others. The protein also can be fused to a tag and subsequently purified by using a specific antibody directed to such an epitope. In some examples, an affinity column or bead immobilized with an epitope tag-binding agent can be used to purify an isoform fusion. For example, binding agents can include glutathione for interaction with a GST epitope tag, immobilized metal-affinity agents such as Cu2+ or Ni2+ for interaction with a Poly-His tag, anti-epitope antibodies such as an anti-myc antibody, and/or any other agent that can be immobilized to a column or bead for purification of an isoform fusion protein.

Finally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, also can be employed to provide a substantially homogeneous isolated recombinant protein.

Prior to purification, conditioned media containing the secreted CSR or ligand isoform polypeptide, including intron fusion proteins, can be concentrated, such as for example, using tangential flow membranes or using stirred cell system filters. Various molecular weight (MW) separation cut offs can be used for the concentration process. For example, a 10,000 MW separation cutoff can be used.

7. Synthetic Isoforms

A variety of synthetic forms of the isoforms are provided. Included among them are conjugates in which the isoform or intron-encoded portion thereof is linked directly or via a linker to another agent, such as a targeting agent or to a molecule that provides the intron-encoded portion or isoform portion to a CSR or ligand isoform so that an activity of the isoform is modulated. Other synthetic forms include chimeras in which the extracellular domain portion and C-terminal portion, such as an intron-encoded portion, are from different isoforms. Also provided are “peptidomimetic” isoforms in which one or more bonds in the peptide backbone is (are) replaced by a bioisostere or other bond such that the resulting polypeptide peptidomimetic has improved properties, such as resistance to proteases, compared to the unmodified form.

Isoform Conjugates

CSR or ligand isoforms, including isoform fusions provided herein, also can be provided as conjugates between the isoform and another agent. The conjugate can be used to target to a receptor with which the isoform interacts and/or to another targeted receptor for delivery of an isoform. Such conjugates include linkage of a CSR or ligand isoform or isoform fusion to a targeted agent and/or targeting agent. Conjugates can be produced by any suitable method including chemical conjugation or chemical coupling, typically through disulfide bonds between cysteine residues present in or added to the components, or through amide bonds or other suitable bonds. Ionic or other linkages also are contemplated. Conjugates of isoforms with a targeted agent or agents also can be generated within an isoform fusion by operatively linking DNA encoding a targeted agent or targeting agent, with or without a linker region, to DNA encoding a CSR or ligand isoform or isoform fusion, such as a tPA-intron fusion protein fusion.

Pharmaceutical compositions can be prepared from CSR and ligand isoforms or isoform fusion conjugates and treatment effected by administering a therapeutically effective amount of a conjugate, for example, in a physiologically acceptable excipient. Isoform conjugates also can be used in in vivo therapy methods such as by delivering a vector containing a nucleic acid encoding a CSR or ligand isoform conjugate as a fusion protein.

Isoform conjugates can include one or more CSR or ligand isoforms linked, either directly or via a linker, to one or more targeted agents: (CSR isoform)n, (L)q, and (targeted agent)m in which at least one isoform is linked directly or via one or more linkers (L) to at least one targeted agent. Such conjugates also can be produced with any portion of an isoform sufficient to bind a target, such as a target cell type for treatment. Any suitable association among the elements of the conjugate and any number of elements where n, and m are integers greater than 1 and q is zero or any integer greater then 1, is contemplated as long as the resulting conjugates interacts with a targeted receptor or targeted cell type.

Examples of a targeted agent include drugs and other cytotoxic molecules such as toxins that act at or via the cell surface and those that act intracellularly. Examples of such moieties, include radionuclides, radioactive atoms that decay to deliver, e.g., ionizing alpha particles or beta particles, or X-rays or gamma rays, that can be targeted when coupled to an isoform. Other examples include chemotherapeutics that can be targeted by coupling with an isoform. For example, geldanamycin targets proteosomes. An isoform-geldanamycin molecule can be directed to intracellular proteosomes, degrading the targeted isoform and liberating geldanamycin at the proteosome. Other toxic molecules include toxins, such as ricin, saporin and natural products from conches or other members of phylum mollusca. Another example of a conjugate with a targeted agent is a CSR or ligand isoform coupled, for example as a protein fusion, with an antibody or antibody fragment. For example, an isoform including an isoform fusion such as, for example, a tPA-intron fusion protein fusion, can be coupled to an Fc fragment of an antibody that binds to a specific cell surface marker to induce killer T cell activity in neutrophils, natural killer cells, and macrophages. A variety of toxins are well known to those of skill in the art.

Isoform conjugates also can contain one or more CSR or ligand isoforms linked, either directly or via a linker, to one or more targeting agents: (CSR isoform)n, (L)q, and (targeting agent)m in which at least one isoform is linked directly or via one or more linkers (L) to at least one targeting agent. Any suitable association among the elements of the conjugate and any number of elements where n, and m are integer greater than 1 and q is zero or any integer greater then 1, is contemplated as long as the resulting conjugates interacts with a target, such as a targeted cell type.

Targeting agents include any molecule that targets a CSR or ligand isoform to a target such as a particular tissue or cell type or organ. Examples of targeting agents include cell surface antigens, cell surface receptors, proteins, lipids and carbohydrate moieties on the cell surface or within the cell membrane, molecules processed on the cell surface, secreted and other extracellular molecules. Molecules useful as targeting agents include, but are not limited to, an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; and other such biological materials. Exemplary molecules useful as targeting agents include ligands for receptors, such as proteinaceous and small molecule ligands, and antibodies and binding proteins, such as antigen-binding proteins.

Alternatively, a CSR or ligand isoform, which specifically interacts with a particular receptor (or receptors) is the targeting agent and it is linked to a targeted agent, such as a toxin, drug or nucleic acid molecule. The nucleic acid molecule can be transcribed and/or translated in the targeted cell or it can be regulatory nucleic acid molecule.

The CSR or ligand isoform and be linked directly to the targeted (or targeting agent) or can be linked indirectly via a linker. Linkers include peptide and non-peptide linkers and can be selected for functionality, such as to relieve or decrease steric hindrance caused by proximity of a targeted agent or targeting agent to an isoform and/or increase or alter other properties of the conjugate, such as the specificity, toxicity, solubility, serum stability and/or intracellular availability and/or to increase the flexibility of the linkage between a CSR isoform and a targeted agent or targeting agent. Examples of linkers and conjugation methods are known in the art (see, for example, WO 00/04926). Isoforms provided herein also can be targeted using liposomes and other such moieties that direct delivery of encapsulated or entrapped molecules.

8. Formation of Multimers

Also provided herein are multimers of the isoforms, including the isoforms with linked preprosequences or portions thereof . Isoform multimers can be covalently-linked, non-covalently-linked, or chemically linked multimers to form dimers, trimers, or higher ordered multimers of the isoforms. The polypeptide components of the multimer can be the same or different. Typically, the components of an isoform multimer provided herein is one or more of the isoform fusions set forth in any of SEQ ID NOS: 31-47 encoding a polypeptide set forth in any of SEQ ID NOS: 32-48. In some examples, a multimer also can be formed between a modified CSR or ligand isoforms, such as for example, any that contain one or more additional amino acids at the N-terminus. These additional amino acids can include, but are not limited to, GAR, SR, LE or combinations thereof such as GARSR (SEQ ID NO:563) or GARLE (SEQ ID NO:564). Exemplary of polypeptide and encoding nucleic acid sequences of CSR or ligand isoforms, including modified forms thereof, that can be used in the multimers are any set forth in any of SEQ ID NOS: 139-354, and variants thereof.

Multimers of isoform polypeptides can be formeded formed by dimerization, such as the interactions between Fc domains, or they can be covalently joined. Multimerization between two isoform polypeptides can be spontaneous, or can occur due to forced linkage of two or more polypeptides. In one example, multimers can be linked by disulfide bonds formed between cysteine residues on isoforms polypeptides. In an additional example, multimers can be formed between two polypeptides through chemical linkage, such as for example, by using heterobifunctional linkers.

a. Peptide Linkers

Peptide linkers can be used to produce polypeptide multimers. In one example, peptide linkers can be fused to the C-terminal end of a first polypeptide and the N-terminal end of a second polypeptide. This structure can be repeated multiples times such that at least one, preferably 2, 3, 4, or more soluble polypeptides are linked to one another via peptide linkers at their respective termini. For example, a multimer polypeptide can have a sequence Z₁-X-Z₂, where Z₁ and Z₂ are each a sequence of all or part of a cell surface polypeptide isoform and where X is a sequence of a peptide linker. In some instances, Z₁ and/or Z₂ is a all or part of an isoform polypeptide. In another example, Z₁ and Z₂ are the same or they are different. In another example, the polypeptide has a sequence of Z₁-X-Z₂(-X-Z)ₙ, where “n” is any integer, i.e. generally 1 or 2. Typically, the peptide linker is of sufficient length to so that the resulting polypeptide is a soluble Examples of peptide linkers include glycine serine polypeptides, such s -Gly-Gly-, GGGGG (SEQ ID NO:582), GGGGS (SEQ ID NO:580) or (GGGGS)n, SSSSG (SEQ ID NO:581) or (SSSSG)n

Linking moieties are described, for example, in Huston et al. (1988) PNAS 85:5879-5883, Whitlow et al. (1993) Protein Engineering 6:989-995, and Newton et al., (1996) Biochemistry 35:545-553. Other suitable peptide linkers include any of those described in U.S. Patent Nos. 4,751,180 or 4,935,233, which are hereby incorporated by reference. A polynucleotide encoding a desired peptide linker can be inserted anywhere in an isoform or at the N- or C-terminus or between the preprosequence, in frame, using any suitable conventional technique.

b. Polypeptide Multimerization Domains

Interaction of two or more polypeptides can be facilitated by their linkage, either directly or indirectly, to any moiety or other polypeptide that are themselves able to interact to form a stable structure. For example, separate encoded polypeptide chains can be joined by multimerization, whereby multimerization of the polypeptides is mediated by a multimerization domain. Typically, the multimerization domain provides for the formation of a stable protein-protein interaction between a first chimeric polypeptide and a second chimeric polypeptide. Chimeric polypeptides include, for example, linkage (directly or indirectly) of a nucleic acid encoding an isoform polypeptide with a nucleic acid encoding a multimerization domain. Homo- or heteromultimeric polypeptides can be generated from co-expression of separate chimeric polypeptides. The first and second chimeric polypeptides can be the same or different.

Generally, a multimerization domain includes any capable of forming a stable protein-protein interaction. The multimerization domains can interact via an immunoglobulin sequence, leucine zipper, a hydrophobic region, a hydrophilic region, or a free thiol which forms an intermolecular disulfide bond between the chimeric molecules of a homo- or heteromultimer. In addition, a multimerization domain can include an amino acid sequence comprising a protuberance complementary to an amino acid sequence comprising a hole, such as is described, for example, in U.S. patent application Ser. No. 08/399,106. Such a multimerization region can be engineered such that steric interactions not only promote stable interaction, but further promote the formation of heterodimers over homodimers from a mixture of chimeric monomers. Generally, protuberances are constructed by replacing small amino acid side chains from the interface of the first polypeptide with larger side chains (e.g., tyrosine or typtophan). Compensatory cavities of identical or similar size to the protuberances are optionally created on the interface of the second polypeptide by replacing large amino acid side chains with smaller ones (e.g., alanine or threonine).

A chimeric isoform polypeptide, such as for example any isoform polypeptide provided herein, can be joined anywhere, but typically via its N- or C-terminus, to the N- or C-terminus of a multimerization domain to ultimately, upon expression, form a chimeric polypeptide

The resulting chimeric polypeptides, and multimers formed therefrom, can be purified by any suitable method, such as, for example, by affinity chromatography over Protein A or Protein G columns. Where two nucleic acid molecules encoding different chimeric polypeptides are transformed into cells, formation of homo- and heterodimers will occur. Conditions for expression can be adjusted so that heterodimer formation is favored over homodimer formation.

i. Immunoglobulin Domain

Multimerization domains include those comprising a free thiol moiety capable of reacting to form an intermolecular disulfide bond with a multimerization domain of an additional amino acid sequence. For example, a multimerization domain can include a portion of an immunoglobulin molecule, such as from IgG1, IgG2, IgG3, IgG4, IgA, IgD, IgM, and IgE. Generally, such a portion is an immunoglobulin constant region (Fc). Preparations of fusion proteins containing soluble CSR extracellular domain polypeptides fused to various portions of antibody-derived polypeptides (including the Fc domain) has been described, see e.g., Ashkenazi et al. (1991) PNAS 88: 10535; Byrn et al. (1990) Nature, 344:677; and Hollenbaugh and Aruffo, (1992) “Construction of Immnoglobulin Fusion Proteins,” in Current Protocols in Immunology, Suppl. 4, pp. 10.19.1-10.19.11.

Antibodies bind to specific antigens and contain two identical heavy chains and two identical light chains covalently linked by disulfide bonds. The heavy and light chains contain variable regions, which bind the antigen, and constant (C) regions. In each chain, one domain (V) has a variable amino acid sequence depending on the antibody specificity of the molecule. The other domain (C) has a rather constant sequence common among molecules of the same class. The domains are numbered in sequence from the amino-terminal end. For example, the IgG light chain is composed of two immunoglobulin domains linked from N- to C-terminus in the order V_{L}-C_{L}, referring to the light chain variable domain and the light chain constant domain, respectively. The IgG heavy chain is composed of four immunoglobulin domains linked from the N- to C-terminus in the order V_{H}-C_{H}1-CH2-CH3, referring to the variable heavy domain, contain heavy domain 1, constant heavy domain 2, and constant heavy domain 3. The resulting antibody molecule is a four chain molecule where each heavy chain is linked to a light chain by a disulfide bond, and the two heavy chains are linked to each other by disulfide bonds. Linkage of the heavy chains is mediated by a flexible region of the heavy chain, known as the hinge region. Fragments of antibody molecules can be generated, such as for example, by enzymatic cleavage. For example, upon protease cleavage by papain, a dimer of the heavy chain constant regions, the Fc domain, is cleaved from the two Fab regions (i.e. the portions containing the variable regions).

In humans, there are five antibody isotypes classified based on their heavy chains denoted as delta (δ), gamma (γ), mu (μ), and alpha (α) and epsilon (ε), giving rise to the IgD, IgG, IgM, IgA, and IgE classes of antibodies, respectively. The IgA and IgG classes contain the subclasses IgA1, IgA2, IgG1, IgG2, IgG3, and IgG4. Sequence differences between immunoglobulin heavy chains cause the various isotypes to differ in, for example, the number of C domains, the presence of a hinge region, and the number and location of interchain disulfide bonds. For example, IgM and IgE heavy chains contain an extra C domain (C4), that replaces the hinge region. The Fc regions of IgG, IgD, and IgA pair with each other through their Cγ3, Cδ3, and Cα3 domains, whereas the Fc regions of IgM and IgE dimerize through their Cμ4 and Cε4 domains. IgM and IgA form multimeric structures with ten and four antigen-binding sites, respectively.

Immunoglobulin chimeric polypeptides provided herein include a full-length immunoglobulin polypeptide. Alternatively, the immunoglobulin polypeptide is less than full length, i.e. containing a heavy chain, light chain, Fab, Fab2, Fv, or Fc. In one example, the immunoglobulin chimeric polypeptides are assembled as monomers or hetero-or homo-multimers, and particularly as dimer or tetramers. Chains or basic units of varying structures can be utilized to assemble the monomers and hetero- and homo-multimers. For example, an isoform polypeptide can be fused to all or part of an immunoglobulin molecule, including all or part of C_{H}, C_{L}, V_{H}, or V_{L} domain of an immunoglobulin molecule (see. e.g., U.S. Pat. application No. 5,116,964). Chimeric isoform polypeptides can be readily produced and secreted by mammalian cells transformed with the appropriate nucleic acid molecule. The secreted forms include those where the isoform polypeptide is present in heavy chain dimers; light chain monomers or dimers; and heavy and light chain heterotetramers where the isoform polypeptide is fused to one or more light or heavy chains, including heterotetramers where up to and including all four variable regions analogues are substituted. In some examples, one or more than one nucleic acid fusion molecule can be transformed into host cells to produce a multimer where the isoforms portions of the multimer are the same or different. In some examples, a non-isoform polypeptide light-heavy chain variable-like domain is present, thereby producing a heterobifunctional antibody. In some examples, a chimeric polypeptide can be made fused to part of an immunoglobulin molecule lacking hinge disulfides, in which non-covalent or covalent interactions of the two polypeptides associate the molecule into a homo- or heterodimer.

(a) Fc Domain

Typically, the immunoglobulin portion of an includes the heavy chain of an immunoglobulin polypeptide, most usually the constant domains of the heavy chain. Exemplary sequences of heavy chain constant regions for human IgG sub-types are known. For example, for the exemplary heavy chain constant region set forth in SEQ ID NO:565, the CH1 domain corresponds to amino acids 1-98, the hinge region corresponds to amino acids 99-110, the CH2 domain corresponds to amino acids 111-223, and the CH3 domain corresponds to amino acids 224-330.

In one example, an immunoglobulin polypeptide chimeric protein can include the Fc region of an immunoglobulin polypeptide. Typically, such a fusion retains at least a functionally active hinge, C_{H}2 and C_{H}3 domains of the constant region of an immunoglobulin heavy chain. For example, a full-length Fc sequence of IgG1 includes amino acids 99-330 of the sequence set forth in SEQ ID NO:565. Numerous Fc domains are known, including variant Fc domains whose T-cell activity is reduced or eliminated. The precise site at which the linkage is made is not critical: particular sites are well known and can be selected in order to optimize the biological activity, secretion, or binding characteristics of the isoforn polypeptide. Exemplary sequence of Fc domains are set forth in SEQ ID NO: 566 and 567.

In addition to hIgG1 Fc, other Fc regions also can be included in the isoform polypeptides provided herein. For example, where effector functions mediated by Fc/FcγR interactions are to be minimized, fusion with IgG isotypes that poorly recruit complement or effector cells, such as for example, the Fc of IgG2 or IgG4, is contemplated. Additionally, the Fc fusions can contain immunoglobulin sequences that are substantially encoded by immunoglobulin genes belonging to any of the antibody classes, including, but not limited to IgG (including human subclasses IgG1, IgG2, IgG3, or IgG4), IgA (including human subclasses IgA1 and IgA2), IgD, IgE, and IgM classes of antibodies. Further, linkers can be used to covalently link Fc to another polypeptide to generate an Fc chimera.

Modified Fc domains also are known (see e.g. U.S. Patent Publication No. US 2006/0024298; and International Patent Publication No. WO 2005/063816 for exemplary modifications). In some examples, the Fc region is such that it has altered (i.e. more or less) effector function than the effector function of an Fc region of a wild-type immunoglobulin heavy chain. The Fc regions of an antibody interacts with a number of Fc receptors, and ligands, imparting an array of important functional capabilities referred to as effector functions. Fc effector functions include, for example, Fc receptor binding, complement fixation, and T cell depleting activity (see e.g., U.S. Pat. No. 6,136,310). Methods of assaying T cell depleting activity, Fc effector function, and antibody stability are known in the art. For example, the Fc region of an IgG molecule interacts with the FcγRs. These receptors are expressed in a variety of immune cells, including for example, monocytes, macrophages, neutrophils, dendritic cells, eosinophils, mast cells, platelets, B cells, large granular lymphocytes, Langerhans' cells, natural killer (NK) cells, and γδT cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. Recognition of and lysis of bound antibody on target cells by cytotoxic cells that express FcγRs is referred to as antibody dependent cell-mediated cytotoxicity (ADCC). Other Fc receptors for various antibody isotypes include FcFRs (IgE), FcαRs (IgA), and FcμRs (IgM).

Thus, a modified Fc domain can have altered affinity, including but not limited to, increased or low or no affinity for the Fc receptor. For example, the different IgG subclasses have different affinities for the FcγRs, with IgG1 and IgG3 typically binding substantially better to the receptors than IgG2 and IgG4. In addition, different FcγRs mediate different effector functions. FcγR1, FcγRIIa/c, and FcγRIIIa are positive regulators of immune complex triggered activation, characterized by having an intracellular domain that has an immunoreceptor tyrosine-based activation motif (ITAM). FcγRIIb, however, has an immunoreceptor tyrosine-based inhibition motif (ITIM) and is therefore inhibitory. Thus, altering the affinity of an Fc region for a receptor can modulate the effector functions induced by the Fc domain.

In one example, an Fc region is used that is modified for optimized binding to certain FcγRs to better mediate effector functions, such as for example, ADCC. Such modified Fc regions can contain modifications corresponding to any one or more of G20S, G20A, S23D, S23E, S23N, S23Q, S23T, K30H, K30Y, D33Y, R39Y, E42Y, T44H, V48I, S51E, H52D, E56Y, E56I, E56H, K58E, G65D, E67L, E67H, S82A, S82D, S88T, S108G, S108I, K110T, K110E, K110D, A111D, A114Y, A114L, A114I, I116D, I116E, I116N, I116Q, E117Y, E117A, K118T, K118F, K118A, and P180L of the exemplary Fc sequence set forth in SEQ ID NO:566, or combinations thereof. A modified Fc containing these mutations can have enhanced binding to an FcR such as, for example, the activating receptor FcγIIIa and/or can have reduced binding to the inhibitory receptor FcγRIIb (see e.g., US 2006/0024298). Fc regions modified to have increased binding to FcRs can be more effective in facilitating the destruction of cancer cells in patients, even when linked with an isoform polypeptide. There are a number of possible mechanisms by which antibodies destroy tumor cells, including anti-proliferation via blockage of need growth pathways, intracellular signaling leading to apopotosis, enhanced down-regulation and/or turnover of receptors, ADCC, and via promotion of the adaptive immune response.

In another example, a variety of Fc mutants with substitutions to reduce or ablate binding with FcγRs also are known. Such muteins are useful in instances where there is a need for reduced or eliminated effector function mediated by Fc. This is often the case where antagonism, but not killing of the cells bearing a target antigen is desired. Exemplary of such an Fc is an Fc mutein described in U.S. Pat. No. 5,457,035. An exemplary Fc mutein is set forth in SEQ ID NO: 568.

In an additional example, an Fc region can be utilized that is modified in its binding to FcRn, thereby improving the pharmacokinetics of an −Fc chimeric polypeptide. FcRn is the neonatal FcR, the binding of which recycles endocytosed antibody from the endosomes back to the bloodstream. This process, coupled with preclusion of kidney filtration due to the large size of the full length molecule, results in favorable antibody serum half-lives ranging from one to three weeks. Binding of Fc to FcRn also plays a role in antibody transport.

Typically, a polypeptide multimer is a dimer of two chimeric proteins created by linking, directly or indirectly, two of the same or different isoform polypeptide to an Fc polypeptide. In some examples, a gene fusion encoding the isoform-Fc (with the pre-prosequence as described herein) chimeric protein is inserted into an appropriate expression vector. The resulting Fc chimeric proteins can be expressed in host cells transformed with the recombinant expression vector, and allowed to assemble much like antibody molecules, where interchain disulfide bonds form between the Fc moieties to yield divalent polypeptides. Typically, a host cell and expression system is a mammalian expression system to allow for glycosylation for stabilizing the Fc proteins. Other host cells also can be used where glycosylation at this position is not a consideration.

The resulting chimeric polypeptides containing Fc moieties, and multimers formed therefrom, can be easily purified by affinity chromatography over Protein A or Protein G columns. Where two nucleic acids molecules encoding different chimeric polypeptides are transformed into cells, the formation of heterodimers must be biochemically achieved since chimeric molecules carrying the Fc-domain will be expressed as disulfide-linked homodimers as well. Thus, homodimers can be reduced under conditions that favor the disruption of inter-chain disulfides, but do no effect intra-chain disulfides. Typically, chimeric monomers with different extracellular portions are mixed in equimolar amounts and oxidized to form a mixture of homo- and heterodimers. The components of this mixture are separated by chromatographic techniques. Alternatively, the formation of this type of heterodimer can be biased by genetically engineering and expressing fusion molecules that contain a isoform polypeptide, followed by the Fc-domain of hIgG, followed by either c-jun or the c-fos leucine zippers (see below). Since the leucine zippers form predominantly heterodimers, they can be used to drive the formation of the heterodimers when desired. Chimeric polypeptides containing Fc regions also can be engineered to include a tag with metal chelates or other epitope. The tagged domain can be used for rapid purification by metal-chelate chromatography, and/or by antibodies, to allow for detection of western blots, immunoprecipitation, or activity depletion/blocking in bioassays.

(b) Protuberances-Into-Cavity (ie. Knobs and Holes)

Multimers can be engineered to contain an interface between a first chimeric polypeptide and a second chimeric polypeptide to facilitate hetero-oligomerization over homo-oligomerization. Typically, a multimerization domain of one or both of the first and second chimeric polypeptide is a modified antibody fragment such that the interface of the antibody molecule is modified to facilitate and/or promote heterodimerization. In some cases, the antibody molecule is a modified Fc region. Thus, modifications include introduction of a protuberance into a first Fc polypeptide and a cavity into a second Fc polypeptide such that the protuberance is positionable in the cavity to promote complexing of the first and second Fc-containing chimeric polypeptides.

Typically, stable interaction of a first chimeric polypeptide and a second chimeric polypeptide is via interface interactions of the same or different multimerization domain that contains a sufficient portion of a CH3 domain of an immunoglobulin constant domain. Various structural and functional data suggest that antibody heavy chain association is directed by the CH3 domain. For example, X-ray crystallography has demonstrated that the intermolecular association between human IgG1 heavy chains in the Fc region includes extensive protein/protein interaction between CH3 domain whereas the glycosylated CH2 domains interact via their carbohydrate (Deisenhofer et al. (1981) Biochem. 20: 2361). In addition, there are two inter-heavy chain disulfide bonds which are efficiently formed during antibody expression in mammalian cells unless the heavy chain is truncated to remove the CH2 and CH3 domains (King et al. (1992) Biochem. J. 281:317). Thus, heavy chain assembly appears to promote disulfide bond formation rather than vice versa. Engineering of the interface of the CH3 domain promotes formation of heteromultimers of different heavy chains and hinders the assembly of corresponding homomultimers (see e.g., U.S. Pat. No. 5,731,168; International Patent Application WO 98/50431 and WO 2005/063816; Ridgway et al. (1996) Protein Engineering, 9:617-621).

Thus, multimers provided herein can be formed between an interface of a first and second chimeric isoform polypeptide (the first and second polypeptides can be the same or different) where the multimerization domain of the first polypeptide contains at least a sufficient portion of a CH3 interface of an Fc domain that has been modified to contain a protuberance and the multimerization domain of the second polypeptide contains at least a sufficient portion of a CH3 interface of an Fc domain that has been modified to contain a cavity. All or a sufficient portion of a modified CH3 interface can be from an IgG, IgA, IgD, IgE, or IgM immunoglobulin. Interface residues targeted for modification in the CH3 domain of various immunoglobulin molecules are set forth in U.S. Pat. No. 5,731,168. Generally, the multimerization domain is all or a sufficient portion of a CH3 domain derived from an IgG antibody, such as for example, IgG1.

Amino acids targeted for replacement and/or modification to create protuberances or cavities in a polypeptide are typically interface amino acids that interact or contact with one or more amino acids in the interface of a second polypeptide. A first polypeptide that is modified to contain protuberance amino acids include replacement of a native or original amino acid with an amino acid that has at least one side chain which projects from the interface of the first polypeptide and is therefore positionable in a compensatory cavity in an adjacent interface of a second polypeptide. Most often, the replacement amino acid is one which has a larger side chain volume than the original amino acid residue. One of skill in the art can determine and/or assess the properties of amino acid residues to identify those that are ideal replacement amino acids to create a protuberance. Generally, the replacement residues for the formation of a protuberance are naturally occurring amino acid residues and include, for example, arginine (R), phenylalanine (F), tyrosine (Y), or tyrptophan (W). In some examples, the original residue identified for replacement is an amino acid residue that has a small side chain such as, for example, alanine, asparagines, aspartic acid, glycine, serine, threonine, or valine.

A second polypeptide that is modified to contain a cavity is one that includes replacement of a native or original amino acid with an amino acid that has at least one side chain that is recessed from the interface of the second polypeptide and thus is able to accommodate a corresponding protuberance from the interface of a first polypeptide. Often, the replacement amino acid is one which has a smaller side chain volume than the original amino acid residue. One of skill in the art can determine and/or assess the properties of amino acid residues to identify those that are ideal replacement residues for the formation of a cavity. Generally, the replacement residues for the formation of a cavity are naturally occurring amino acids and include, for example, alanine (A), serine (S), threonine (T) and valine (V). In some examples, the original amino acid identified for replacement is an amino acid that has a large side chain such as, for example, tyrosine, arginine, phenylalanine, or typtophan.

The CH3 interface of human IgG1, for example, involves sixteen residues on each domain located on four anti-parallel β-strands which buries 1090 Å2 from each surface (see e.g., Deisenhofer et al. (1981) Biochemistry, 20:2361-2370; Miller et al., (1990) J Mol. Biol., 216, 965-973; Ridgway et al., (1996) Prot. Engin., 9: 617-621; U.S. Pat. 5,731,168). Modifications of a CH3 domain to create protuberances or cavities are described, for example, in U.S. Pat. No. 5,731,168; International Patent Applications WO98/50431 and WO 2005/063816; and Ridgway et al., (1996) Prot. Engin., 9: 617-621. For example, modifications in a CH3 domain to create protuberances or cavities can be replacement of any amino acid corresponding to the interface amino acid Q230, V231, Y232, T233, L234, V246, S247, L248, T249, C250, L251, V252, K253, G254, F255, Y256, K275, T276, T277, P278, V279, L280, D281, G285, S286, F287, F288, L289, Y290, S291, K292, L293, T294, and V295 of the sequence set forth in SEQ ID NO:565. In some examples, modifications of a CH3 domain to create protuberances or cavities are typically targeted to residues located on the two central anti-parallel β-strands. The aim is to minimize the risk that the protuberances which are created can be accommodated by protruding into the surrounding solvent rather than being accommodated by a compensatory cavity in the partner CH3 domain. Exemplary of such modifications include, for example, replacement of any amino acid corresponding to the interface amino acid T249, L251, P278, F288, Y290, and K292. Exemplary of amino acid pairs for modification in a CH3 domain interface to create protuberances/cavity interactions include modification of T249 and Y290; and F288 and T277. For example, modifications can include T249Y and Y290T; T249W and Y290A; F288A and T277W; F288W and T277S; and Y290T and T249Y.

In some example, more than one interface interaction can be made. For example, modifications also include, for example, two or more modifications in a first polypeptide to create a protuberance and two or more medications in a second polypeptide to create a cavity. Exemplary of such modifications include, for example, modification of T249Y and F288A in a first polypeptide and modification of T277W and Y290T in a second polypeptide; modification of T277W and F288W in a first polypeptide and modification of T277S and Y290A in a second polypeptide; or modification of F288A and Y290A in a first polypeptide and T249W and T277S in a second polypeptide.

As with other multimerization domains described herein, including all or part of any immunoglobulin molecule or variant thereof, such as an Fc domain or variant thereof, an Fc variant containing CH3 protuberance/cavity modifications can be joined to an isoform polypeptide anywhere, but typically via its N- or C-terminus, to the N- or C-terminus of a first and/or second isoform polypeptide to form a chimeric polypeptide. The linkage can be direct or indirect via a linker. Also, the chimeric polypeptide can be a fusion protein or can be formed by chemical linkage, such as through covalent or non-covalent interactions. Typically, a knob and hole molecule is generated by co-expression of a first isoform polypeptide linked to an Fc variant containing CH3 protuberance modification(s) with a second isoform polypeptide linked to an Fc variant containing CH3 cavitity modification(s).

ii. Leucine Zipper

Another method for preparing multimers involves use of a leucine zipper domain. Leucine zippers are peptides that promote multimerization of the proteins in which they are found. Typically, leucine zipper is a term used to refer to a repetitive heptad motif containing four to five leucine residues present as a conserved domain in several proteins. Leucine zippers fold as short, parallel coiled coils, and can be responsible for oligomerization of the proteins of which they form a domain. Leucine zippers were originally identified in several DNA-binding proteins (see e.g., Landschulz et al. (1988) Science 240:1759), and have since been found in a variety of proteins. Among the known leucine zippers are naturally occurring peptides and derivatives thereof that dimerize or trimerize. Recombinant chimeric proteins containing an isoform polypeptide linked, directly or indirectly, to a leucine zipper peptide can be expressed in suitable host cells, and the polypeptide multimer that forms can be recovered from the culture supernatant.

Leucine zipper domains fold as short, parallel coiled coils (O'Shea et al. (1991) Science, 254:539). The general architecture of the parallel coiled coil has been characterized, with a “knobs-into-holes” packing, first proposed by Crick in 1953 (Acta Crystallogr., 6:689). The dimer formed by a leucine zipper domain is stabilized by the heptad repeat, designated (abcdefg)n (see e.g., McLachlan and Stewart (1978) J. Mol. Biol. 98:293), in which residues a and d are generally hydrophobic residues, with d being a leucine, which lines up on the same face of a helix. Oppositely-charged residues commonly occur at positions g and e. Thus, in a parallel coiled coil formed from two helical leucine zipper domains, the “knobs” formed by the hydrophobic side chains of the first helix are packed into the “holes” formed between the side chains of the second helix.

The leucine residues at position d contribute large hydrophobic stabilization energies, and are important for dimer formation (Krystek et al. (1991) Int. J. Peptide Res. 38:229). Hydrophobic stabilization energy provides the main driving force for the formation of coiled coils from helical monomers. Electrostatic interactions also contribute to the stoichiometry and geometry of coiled coils.

(a) Fos and Jun

Two nuclear transforming proteins, fos and jun, exhibit leucine zipper domains, as does the gene product of the murine proto-oncogene, c-myc. The leucine zipper domain is necessary for biological activity (DNA binding ) in these proteins. The products of the nuclear oncogenes fos and jun contain leucine zipper domains that preferentially form a heterodimer (O'Shea et al. (1989) Science, 245:646; Turner and Tijian (1989) Science, 243:1689). For example, the leucine zipper domains of the human transcription factors c-jun and c-fos have been shown to form stable heterodimers with a 1:1 stoichiometry (see e.g., Busch and Sassone-Corsi (1990) Trends Genetics, 6:36-40; Gentz et al., (1989) Science, 243:1695-1699). Although jun-jun homodimers also have been shown to form, they are about 1000-fold less stable than jun-fos heterodimers.

Thus, typically an isoform polypeptide multimer provided herein is generated using a jun-fos combination. Generally, the leucine zipper domain of either c-jun or c-fos is fused in frame at the C-terminus of an isoform of a polypeptide by genetically engineering fusion genes. Exemplary sequences of a c-jun or c-fos leucine zipper domain is set forth in SEQ ID NOS: 569 and 570, respectively. In some instances, a sequence of a leucine zipper can be modified, such as by the addition of a cysteine residue to allow formation of disulfide bonds, or the addition of a tyrosine residue at the C-terminus to facilitate measurement of peptide concentration. Exemplary sequences of a modified c-jun or c-fos leucine zipper domain are set forth in SEQ ID NOS: 571 and 572, respectively. In addition, the linkage of an isoform polypeptide with a leucine zipper can be direct or can employ a flexible linker domain, such as for example a hinge region of IgG, or other polypeptide linkers of small amino acids such as glycine, serine, threonine, or alanine at various lengths and combinations. In some instances, separation of a leucine zipper from the C-terminus of an encoded polypeptide can be effected by fusion with a sequence encoding a protease cleavage sites, such as for example, a thrombin cleavage site. Additionally, the chimeric proteins can be tagged, such as for example, by a 6×His tag, to allow rapid purification by metal chelate chromatography and/or by epitopes to which antibodies are available, such as for example a myc tag, to allow for detection on western blots, immunoprecipitation, or activity depletion/blocking bioassays.

(b) GCN4

A leucine zipper domain also occurs in a nuclear protein that functions as a transcriptional activator of a family of genes involved in the General Control of Nitrogen (GCN4) metabolism in S. cerevisiae. An exemplary sequence of the GCN4 leucine zipper domain is set forth in SEQ ID NO: 573. The protein is able to dimerize and bind promoter sequences containing the recognition sequence for GCN4, thereby activating transcription in times of nitrogen deprivation. Amino acid substitutions in the a and d residues of a synthetic peptide representing the GCN4 leucine zipper domain, change the oligomerization properties of the leucine zipper domain. For example, when all residues at position a are changed to isoleucine, the leucine zipper still forms a parallel dimer. When, in addition to this change, all leucine residues at position d also are changed to isoleucine, the resultant peptide spontaneously forms a trimeric parallel coiled coil in solution. Exemplary sequences of trimer and tetramer forms of a GCN4 leucine zipper domain are set forth in SEQ ID NOS: 574 and 575, respectively.

iii. Other Multimerization Domains

Other multimerization domains are known to those of skill in the art and are any that facilitate the protein-protein interaction of two or more polypeptides that are separately generated and expressed. Examples of other multimerization domains that can be used to provide protein-protein interactions between or among polypeptides include, but are not limited to, the barnase-barstar module (see e.g., Deyev et al., (2003) Nat. Biotechnol. 21:1486-1492); selection of particular protein domains (see e.g., Terskikh et al., (1997) PNAS 94: 1663-1668 and Muller et al., (1998) FEBS Lett. 422:259-264); selection of particular peptide motifs (see e.g., de Kruif et al., (1996) J. Biol. Chem. 271:7630-7634 and Muller et al., (1998) FEBS Lett. 432: 45-49); and the use of disulfide bridges for enhanced stability (de Kruif et al., (1996) J. Biol. Chem. 271:7630-7634 and Schmiedl et al., (2000) Protein Eng. 13:725-734). Exemplary of another type of multimerization domain is one where multimerization is facilitated by protein-protein interactions between different subunit polypeptides, such as is described below for PKA/AKAP interaction.

R/PKA-AD/AKAP

Multimeric polypeptides also can be generated utilizing protein-protein interactions between the regulatory (R) subunit of cAMP-dependent protein kinase (PKA) (see e.g., SEQ ID NO: 576 or 578) and the anchoring domains (AD) of A kinase anchor proteins (AKAPs, see e.g., Rossi et al., (2006) PNAS 103:6841-6846) (see e.g., SEQ ID NO: 577 or SEQ ID NO: 579). Two types of R subunits (RI and RII) are found in PKA, each with an α and β isoforn. The R subunits exist as dimers, and for RII, the dimerization domain resides in the 44 amino-terminal residues. AKAPs, via the interaction of their AD domain, interact with the R subunit of PKA to regulate its activity. AKAPs bind only to dimeric R subunits. For example, for human RIIα, the AD binds to a hydrophobic surface formed from the 23 amino-terminal residues.

F. Assays to Assess Activity of an Isoform

CSR and ligand isoforms such as any provided herein that contain additional amino acids as compared to a cognate CSR or ligand isoform retain their function following the production and purification of the isoform. Such modified fusion isoforms include, but are not limited to, those isoforms having additional amino acids at the N-terminus due to incomplete processing following secretion (i.e. GAR), the presence of encoded linker sequences (i.e. LE or SR), and/or the presence of an epitope tag (i.e. c-myc or His-tag). Generally, isoforms exhibit alterations in structure or in one more activities compared to a full-length, wildtype or predominant form of a cognate receptor or ligand. In addition, isoforms can alter (modulate) the activity of a cognate receptor or ligand. All such isoforms are candidate therapeutics.

Where the isoforms exhibits a difference in an activity, in vitro and in vivo assays can be used to monitor or screen isoforms. In vitro and in vivo assays also can be used to screen isoforms to identify or select those that modulate the activity of a particular receptor or pathway. Such assays are well known to those of skill in the art. One of skill in the art can test a particular purified isoform for interaction with a receptor or ligand and/or can test to assess an activity or any change in activity compared to a cognate receptor or ligand. Some such assays are exemplified herein.

Exemplary in vitro and in vivo assays are provided herein for assessing an activity of a purified isoform produced from fusion of an isoform to a precursor sequence, such as a tPA pre/prosequence, and optionally an epitope tag. The assays provided herein also can be used as a comparison of an activity of an isoform to an activity of a wildtype or predominant form of a cognate receptor or ligand. Many of the assays are applicable to RTKs or RTK isoforms, but can be used to assess other CSRs and CSR isoforms as well as other ligand isoforms that modulate the activity of a CSR. In addition, numerous assays, such as assays for kinase activities and cell proliferation activities of CSRs are known to one of skill in the art. Assays for activities of RTK isoforms and RTKs include, but are not limited to, kinase assays, homodimerization and heterodimerization assays, protein:protein interaction assays, structural assays, cell signaling assays and in vivo phenotyping assays. Assays also include employing animal models, including disease models in which an activity can be observed and/or measured or otherwise assessed. Dose response curves of a CSR or ligand isoform in such assays, such as an isoform produced from an isoform fusion, can be used to assess modulation of biological activities as well as to determine therapeutically effective amounts of an isoform for in vivo administration. Exemplary assays are described below.

1. Kinase Assays

Kinase activity can be detected and/or measured directly and indirectly. For example, antibodies against phosphotyrosine can be used to detect phosphorylation of an RTK, RTK isoform, an RTK:RTK isoform complex and phosphorylation of other proteins and signaling molecules. For example, activation of tyrosine kinase activity of an RTK can be measured in the presence of a ligand for an RTK. Transphosphorylation can be detected by anti-phosphotyrosine antibodies. Transphosphorylation can be measured and/or detected in the presence and absence of an RTK isoform, thus measuring the ability of an RTK isoform to modulate the transphosphorylation of an RTK. Briefly, cells expressing an RTK isoform or that have been exposed to an RTK isoform, are treated with ligand. Cells are lysed and protein extracts (whole cell extracts or fractionated extracts) are loaded onto a polyacrylamide gel, separated by electrophoresis and transferred to membrane, such as used for western blotting. Immunoprecipitation with anti-RTK antibodies also can be used to fractionate and isolate RTK proteins before performing gel electrophoresis and western blotting. The membranes can be probed with anti-phosphotyrosine antibodies to detect phosphorylation as well as probed with anti-RTK antibodies to detect total RTK protein. Control cells, such as cells not expressing RTK isoform and cells not exposed to ligand can be subjected to the same procedures for comparison.

Tyrosine phosphorylation also can be measured directly, such as by mass spectroscopy. For example, the effect of an RTK isoform on the phosphorylation state of an RTK can be measured, such as by treating intact cells with various concentrations of an RTK isoform and measuring the effect on activation of an RTK. The RTK can be isolated by immunoprecipitation and trypsinized to produce peptide fragments for analysis by mass spectroscopy. Peptide mass spectroscopy is a well-established method for quantitatively determining the extent of tyrosine phosphorylation for proteins; phosphorylation of tyrosine increases the mass of the peptide ion containing the phosphotyrosine, and this peptide is readily separated from the non-phosphorylated peptide by mass spectroscopy.

For example, tyrosine-1139 and tyrosine-1248 are known to be autophosphorylated in the HER2 RTK. Trypsinized peptides can be empirically determined or predicted based on polypeptide, for example by using the ExPASy-PeptideMass program. The extent of phosphorylation of tyrosine-1139 and tyrosine-1248 can be determined from the mass spectroscopy data of peptides containing these tyrosines. Such assays can be used to assess the extent of auto-phosphorylation of an RTK isoform and the ability of an RTK isoform to transphosphorylate an RTK.

2. Complexation

Complexation, such as dimerization of RTKs and RTK isoforms and trimerization of TNFRs and TNFR isoforms, can be detected and/or measured. For example, isolated polypeptides can be mixed together and subjected to gel electrophoresis and western blotting. CSRs and/or CSR isoforms also can be added to cells and cell extracts, such as whole cell or fractionated extracts, and can be subjected to gel electrophoresis and western blotting. Antibodies recognizing the polypeptides can be used to detect the presence of monomers, dimers and other complexed forms. Alternatively, labeled CSRs and/or labeled CSR isoforms can be detected in the assays.

For example, such assays can be used to compare homodimerization of an RTK or heterodimerization of two or more RTKs in the presence and absence of an RTK isoform. Assays also can be performed to assess homodimerization of an RTK isoform and/or its ability to heterodimerize with an RTK. For example, an ErbB2 RTK isoform can be assessed for its ability to heterodimerize with HER2, HER3 and HER4. Additionally, a HER2 RTK isoform can be assessed for its ability to modulate the ability of HER2 to homodimerize with itself.

3. Ligand Binding

Generally, CSRs bind to one or more ligands. Ligand binding modulates the activity of the receptor and thus modulates, for example, signaling within a signal transduction pathway. Ligand binding of a CSR isoform and ligand binding of a CSR in the presence of a CSR isoform can be measured. For example, labeled ligand such as radiolabeled ligand can be added to a purified or partially purified CSR in the presence or absence (control) of a CSR isoform. Immunoprecipitation and measurement of radioactivity can be used to quantify the amount of ligand bound to a CSR in the presence and absence of a CSR isoform. A CSR isoform also can be assessed for ligand binding such as by incubating a CSR isoform with labeled ligand and determining the amount of labeled ligand bound by a CSR isoform, for example, compared to an amount bound by a wildtype or predominant form of a corresponding CSR.

4. Receptor Binding

CSR and ligand isoforms can be assessed directly by assessing binding of an isoform to cells. For ligand isoforms, binding can be compared to binding of a cognate ligand to cells. In some examples, competitive assays can be employed with an isoform and other known ligands or isoforms for binding to cells known to express a binding receptor. For example, the ability of HGF isoforms to compete with HGF for binding to the MET receptor can be assessed. HGF and HGF isoforms can be radioiodinated by the chloramine T method (see Nakamura et al., (1997), Cancer Res. 57, 3305-3313) and specific activities of ¹²⁵I-HGF and ¹²⁵I-HGF isoforms can be measured. Cells that normally express the MET receptor are cultured in multiwell plates for the binding assay. The cells are equilibrated in an ice-cold binding buffer and incubated with various concentrations of ¹²⁵I-HGF or ¹²⁵I-HGF isoforms, with or without an excess molar ratio of unlabeled HGF or HGF isoforms. For competitive binding assays, a fixed concentration of ¹²⁵I-HGF and various concentrations of unlabelled HGF or HGF isoforms are incubated with the cells. After the incubation period, the cells are washed, solubilized, and the bound labeled proteins are measured using a γ-counter.

Binding of isoforms to cell surface molecules can be measured directly or indirectly for one or more than one cell surface molecule. For example, immunoprecipitation can be used to assess cell surface molecule binding. Cell lysates are incubated with an isoform. Antibodies against a cell surface molecule, such as a CSR including an RTK, TNFR, or other ligand receptor, can be used to immunoprecipitate the complex. The amount of isoform in the complex is quantified and/or detected using western blotting of the immunoprecipitates with anti-isoform antibodies.

5. Cell Proliferation Assays

A number of RTKs, for example VEGFRs, are involved in cell proliferation. Effects of an RTK isoform on cell proliferation can be measured. For example, ligand can be added to cells expressing an RTK. An RTK isoform can be added to such cells before, concurrently, or after ligand addition and effects on cell proliferation measured. Alternatively an RTK isoform can be expressed in such cell models, for example using an adenovirus vector. For example, a VEGFR isoform can be added to endothelial cells expressing a VEGFR. Following isoform addition, VEGF ligand is added and the cells are incubated at standard growth temperature (e.g. 37° C.) for several days. Cells are trypsinized, stained with trypan blue and viable cells are counted. Cells not exposed to a VEGFR isoform and/or ligand are used as controls for comparison. Other suitable controls can be employed.

6. Motogenic Assays

CSR or ligand isoforms, such as those produced from isoform fusions provided herein, can be assessed for their ability to interfere with ligand-induced cell motility. For example, endothelial cells are cultured in multiwell plates until firmly adhered to the culture dish surface. Fresh culture medium is then added and overlaid with light mineral oil to prevent evaporation. Medium containing HGF, HGF or MET isoforms, or a combination thereof is added and images are recorded with a digital camera and a time lapse recorder. The distance traveled is calculated from a defined number of cells from each frame.

Effects of isoforms on ligand-induced cell migration also can be assessed by an endothelial cell wounding assay. Endothelial cells are cultured on plates and grown to reach confluence. Cells are wounded with an 82-gauge needle to produce wounds of approximately 200 μm. The cells are then washed and fresh culture medium is added containing HGF or MET isoforms, HGF, or a combination thereof. Images of cell migration are recorded as described above, and migration distance over the wound front is calculated.

Cell migration can also be assessed using a modified Boyden chamber assay. Endothelial cells, such as human dermal microvascular endothelial cells, are serum starved and then plated onto the inner chamber of a Transwell plate (6.5 mm diameter polycarbonate membrane, 5 μm pore size, Costar, Cambridge, Mass.) coated with 13.4 μg/ml fibronectin. Medium containing HGF, bFGF or VEGF, or other ligand that induces cell migration, with or without a CSR or ligand isoform, is added to the outer chamber, and incubated for a period of time. The number of cells that migrate through the membrane to the under surface of the filter is quantified by counting the cells in randomly selected microscopic fields in each well.

7. Apoptotic Assays

Many ligands through signaling through specific CSRs exert antiapoptotic effects. For example, HGF exerts an antiapoptotic effect on cells treated with cytotoxic agents, such as irradiation and certain cancer therapeutics, including cisplatin, camtothesin, Adriamycin, and taxol. The ability of HGF or MET isoforms to alter the antiapoptotic effects of HGF treatment can be measured. Cells are cultured with medium containing varying concentrations of HGF or MET isoforms and/or HGF. Cells are then exposed to the cytotoxic agent for an incubation period, and cell viability is measured using a 3-(4,5-dimethylthisazol-2-yl)-2,5-diphenyl tetrazolium bromide (MTT, Sigma) assay.

Apoptotic cells show characteristic nuclear fragmentation that can be visualized by nuclear stains. Cells treated with HGF show reduced nuclear fragmentation in response to cytotoxic agents. The ability of HGF or MET isoforms to antagonize this effect of HGF can be assessed. Cells are plated onto glass slides and treated with cytotoxic agents followed by HGF and/or HGF or MET isoforms as described above. Nuclei of the cells are visualized using Hoescht 33342 stain and a fluorescent microscope at excitement wavelength of 350 nm and emission wavelength of 450 nm. Other assays to assess for effects of a CSR or ligand isoform on apoptosis can include a DNA fragmentation assay, the DNA filter elution assay, TUNEL stain, measurement of caspase-3 activity, and/or in vitro kinase activity assays for the induction of AKT.

8. Cell Disease model Assays

Cells from a disease or condition or that can be modulated to mimic a disease or condition can be used to measure/and or detect the effect of a CSR isoform. Numerous animal and in vitro disease models are known to those of skill in the art. For example, a CSR isoform is added or expressed in cells and a phenotype is measured or detected in comparison to cells not exposed to or not expressing a CSR isoform. Such assays can be used to measure effects including effects on cell proliferation, metastasis, inflammation, angiogenesis, pathogen infection and bone resorption.

For example, effects of a MET isoform can be measured using such assays. A liver cell model such as HepG2 liver cells can be used to monitor the infectivity of malaria in culture by sporozoites. An RTK isoform such as a MET isoform can be added to the cells and/or expressed in the cells. Infection of such cells with malaria sporozoites is then measured, such as by staining and counting the EEFs (exoerythrocytic forms) of the sporozoite that are produced as a result of infection (Carrolo et al. (2003) Nat Med 9(11):1363-1369). Effects of an RTK isoform can be assessed by comparing results to cells not exposed or expressing an RTK isoform and/or uninfected cells.

Effects of a CSR or ligand isoform on angiogenesis also can be measured. For example, tubule formation by endothelial cells such as human umbilical vein endothelial cells (HUVEC) in vitro can be used as an assay to measure angiogenesis and effects on angiogenesis. Addition of varying amounts of a CSR or ligand isoform to an in vitro angiogenesis assay is a method suitable for screening the effectiveness of a CSR or ligand isoform as a modulator of angiogenesis.

Bone resorption can be measured in cell culture to measure effectiveness of an CSR or ligand isoform, such as by using osteoclast cultures. Osteoclasts are highly differentiated cells of hematopoietic origin that resorb bone in the organism, and are able to resorb bone from bone slices in vitro. Methods for cell culture of osteoclasts and quantitative techniques for measuring bone resorption in osteoclast cell culture have been described in the art. For example, mononuclear cells can be isolated from human peripheral blood and cultured. Addition and/or expression of a CSR or ligand isoform can be used to assess effects on osteoclast formation such as by measuring multinucleated cells positive for tartrate-resistant acid phosphatase and resorbed area and collagen fragments released from bone slices. Dose response curves can be used to determine therapeutically effective amounts of an isoform necessary to modulate bone resorption.

9. Animal Models

Animal models can be used to assess the effect or activity of a CSR or ligand isoform, or modified form thereof containing additional amino acids. Suitable models are known to those of skill in the art. In one example, animal models of a disease can be studied to determine if introduction of an isoform affects the disease. For example, effects of CSR or ligand isoforms on tumor formation including cancer cell proliferation, migration and invasiveness can be measured. In one such assay, cancer cells such as ovarian cancer cells are infected with an adenovirus expressing an isoform, such as an isoform fusion minimally containing a tPA pre/prosequence operatively linked to a sequence of an isoform in the absence of an endogenous signal sequence. After a culturing period in vitro, cells are trypsinized, suspended in a suitable buffer and injected into mice (e.g., subcutaneously into flanks and shoulders of model mice such as Balb/c nude mice). Tumor growth is monitored over time. Control cells, not expressing a CSR or ligand isoform, can be injected into mice for comparison. Similar assays can be performed with other cell types and animal models, for example, NIH3T3 cells, murine lung carcinoma (LLC) cells, primary Pancreatic Adenocarcinoma (PANC-1) cells, TAKA-1 pancreatic ductal cells, and C57BL/6 mice and SCID mice. In a further example, effects of CSR or ligand isoforms on ocular disorders can be assessed using assays such as a corneal micropocket assay. Briefly, mice receive cells expressing an isoform fusion (or control) by injection 2-3 days before the assay. Subsequently, the mice are anesthetized, and pellets of a receptor ligand are implanted into the corneal micropocket of the eyes. Neovascularization is then measured, for example, 5 days following implantation. The effect of a CSR or ligand isoform on angiogenesis and eye phenotype compared to a control is then assessed.

In an additional example, effects of an isoform in a model of collagen type II-induced arthritis (CIA) can be assessed by intraperitoneal injection of SCID mice with splenocytes from DBA/1 mice that have been transduced with a retroviral vector containing the cDNA of a CSR or ligand isoform fusion or unmodified splenocytes. Mice that receive unmodified splenocytes develop arthritis within 11-13 days and can be used as a reference control to determine effects of isoform-expressing splenocytes on the development of arthritis as assessed, for example, by clinical, histological, or immunological (i.e. antibody levels) parameters of arthritis. In another example, disease can be induced directly in DBA/1 mice by a single intra-dermal injection of bovine type II collagen in the presence or absence of a CSR or ligand isoform, either administered in recombinant form or via gene therapy, and the onset of arthritis can be assessed over time (up to weeks) after immunization.

Effects of CSR isoforms on animal models of disease additionally can be assessed by the administration of purified or recombinant forms of a CSR or ligand isoform. For example, wound healing can be assessed in a model of impaired wound healing utilizing genetically diabetic db+/db+ mice whereby full-thickness excisional wounds are created on the backs of diabetic mice . Following treatment with an isoform, either topically or systemically, wound healing can be assessed by analyzing for wound closure, inflammatory cell infiltration at the site of the wound, and/or expression of inflammatory cytokines. The effects of isoforms on wound healing can be assessed over time and effects can be compared to mice that receive a control treatment, for example a vehicle only control. In a further example, a recombinant isoform, produced from an isoform fusion such as, for example, a tPA-intron fusion protein fusion, can be administered in a model of pulmonary fibrosis induced by bleomycin or silica to determine if lung fibrosis is reduced as assessed, for example, by analysis of histological sections for lung damage and by assaying for effects on bleomycin/silica induced increases of lung hydroxyproline content.

Animals deficient in a CSR or ligand isoform also can be used to monitor the biological activity of an isoform. For example an isoform-specific disruption can be made by creating a targeted construct whereby upstream from an IRES-LacZ cassette, translational stop codons are introduced within the appropriate reading frame to ensure that the receptor or ligand protein terminates early. Alternatively, a LoxP/Cre recombination strategy can be used. Following confirmation of the targeted disruption, the consequences of a deficiency in a CSR or ligand isoform can be established by analyzing the phenotype of the deficient mice compared to wildtype mice including the development of various organs such as, for example, lung, limbs, eyelids, anterior pituitary gland, and pancreas. In addition, by histology or isolation of specific cell populations, other parameters, such as apoptosis or cell proliferation, can be assessed to determine if there is a difference between animals or isolated cells lacking an isoform compared to wildtype CSR or ligand. Components of signaling cascades and expression of downstream genes also can be assessed to determine if the absence of a CSR isoform affects receptor signaling and gene expression.

G. Preparation, Formulation and Administration of CSR and Ligand Isoforms and CSR and Ligand Isoform Compositions

CSR and ligand isoforms and CSR and ligand isoform compositions, particularly modified CSR and ligand isoform polypeptides containing additional amino acids at the N-terminus due to incomplete processing following secretion, the presence of encoded linker sequences, or the presence of an epitope tag, can be formulated for administration by any route known to those of skill in the art including intramuscular, intravenous, intradermal, intraperitoneal, subcutaneous, epidural, nasal, oral, rectal, topical, inhalational, buccal (e.g., sublingual), and transdermal administration or any route. CSR and ligand isoforms can be administered by any convenient route, for example by infusion or bolus injection, by absorption through epithelial or mucocutaneous linings (e.g., oral mucosa, rectal and intestinal mucosa, etc.) and can be administered with other biologically active agents, either sequentially, intermittently or in the same composition. Administration can be local, topical or systemic depending upon the locus of treatment. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. Administration also can include controlled release systems including controlled release formulations and device controlled release, such as by means of a pump. The most suitable route in any given case will depend on the nature and severity of the disease or condition being treated and on the nature of the particular composition which is used.

Various delivery systems are known and can be used to administer CSR or ligand isoforms, including expressed or secreted CSR and ligand isoforms provided herein, such as but not limited to, encapsulation in liposomes, microparticles, microcapsules, recombinant cells capable of expressing the compound, receptor mediated endocytosis, and delivery of nucleic acid molecules encoding CSR isoforms such as retrovirus delivery systems.

Pharmaceutical compositions containing CSR and ligand isoforms can be prepared. Generally, pharmaceutically acceptable compositions are prepared in view of approvals by a regulatory agency or otherwise prepared in accordance with generally recognized pharmacopeia for use in animals and in humans. Pharmaceutical compositions can include carriers such as a diluent, adjuvant, excipient, or vehicle with which an isoform is administered. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polvinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrine derivatives, sorbitan monolaurate, triethanolamine sodium acetate, triethanolamine oleate, and other such agents. These compositions can take the form of solutions, suspensions, emulsion, tablets, pills, capsules, powders, and sustained release formulations. A composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and other such agents. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to the patient. The formulation should suit the mode of administration.

Formulations are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable derivatives thereof. Pharmaceutical therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. Examples of unit dose forms include ampoules and syringes and individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging.

Dosage forms or compositions containing active ingredient in the range of 0.005% to 100% with the balance made up from non-toxic carrier can be prepared. For oral administration, pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well-known in the art.

Pharmaceutical preparation also can be in liquid form, for example, solutions, syrups or suspensions, or can be presented as a drug product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

Formulations suitable for rectal administration can be provided as unit dose suppositories. These can be prepared by admixing the active compound with one or more conventional solid carriers, for example, cocoa butter, and then shaping the resulting mixture.

Formulations suitable for topical application to the skin or to the eye include ointments, creams, lotions, pastes, gels, sprays, aerosols and oils. Exemplary carriers include Vaseline, lanoline, polyethylene glycols, alcohols, and combinations of two or more thereof. The topical formulations also can contain 0.05 to 15, 20, 25 percent by weight of thickeners selected from among hydroxypropyl methyl cellulose, methyl cellulose, polyvinylpyrrolidone, polyvinyl alcohol, poly (alkylene glycols), poly/hydroxyalkyl, (meth)acrylates or poly(meth)acrylamides. A topical formulation is often applied by instillation or as an ointment into the conjunctival sac. It also can be used for irrigation or lubrication of the eye, facial sinuses, and external auditory meatus. It also can be injected into the anterior eye chamber and other places. A topical formulation in the liquid state can be also present in a hydrophilic three-dimensional polymer matrix in the form of a strip or contact lens, from which the active components are released.

For administration by inhalation, the compounds for use herein can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin, for use in an inhaler or insufflator can be formulated containing a powder mix of the compound and a suitable powder base such as lactose or starch.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

Pharmaceutical compositions of CSR and ligand isoforms can be formulated for parenteral administration by injection, e.g., by bolus injection or continuous infusion. Formulations for injection can be presented in unit dosage form, e.g., in ampoules or in multi-dose containers, with an added preservative. The compositions can be suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water or other solvents, before use.

Formulations suitable for transdermal administration can be presented as discrete patches adapted to remain in intimate contact with the epidermis of the recipient for a prolonged period of time. Such patches suitably contain the active compound as an optionally buffered aqueous solution of, for example, 0.1 to 0.2M concentration with respect to the active compound. Formulations suitable for transdermal administration also can be delivered by iontophoresis (see, e.g., Pharmaceutical Research 3(6), 318 (1986)) and typically take the form of an optionally buffered aqueous solution of the active compound.

Pharmaceutical compositions also can be administered by controlled release means and/or delivery devices (see, e.g., in U.S. Pat. Nos. 3,536,809; 3,598,123; 3,630,200; 3,845,770; 3,847,770; 3,916,899; 4,008,719; 4,687,610; 4,769,027; 5,059,595; 5,073,543; 5,120,548; 5,354,566; 5,591,767; 5,639,476; 5,674,533 and 5,733,566).

In certain embodiments, liposomes and/or nanoparticles may also be employed with CSR isoform administration. Liposomes are formed from phospholipids that are dispersed in an aqueous medium and spontaneously form multilamellar concentric bilayer vesicles, also termed multilamellar vesicles (MLVs). MLVs generally have diameters of from 25 nm to 4 μm. Sonication of MLVs results in the formation of small unilamellar vesicles (SUVs) with diameters in the range of 200 to 500 ANG., containing an aqueous solution in the core.

Phospholipids can form a variety of structures other than liposomes when dispersed in water, depending on the molar ratio of lipid to water. At low ratios, the liposomes form. Physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one may operate at the same time.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use herein, and such particles can be easily made.

Administration methods can be employed to decrease the exposure of CSR or ligand isoforms to degradative processes, such as proteolytic degradation and immunological intervention via antigenic and immunogenic responses. Examples of such methods include local administration at the site of treatment. Pegylation of therapeutics has been reported to increase resistance to proteolysis, increase plasma half-life, and decrease antigenicity and immunogenicity. Examples of pegylation methodologies are known in the art (see for example, Lu and Felix, Int. J. Peptide Protein Res., 43: 127-138, 1994; Lu and Felix, Peptide Res., 6: 142-6, 1993; Felix et al., Int. J. Peptide Res., 46: 253-64, 1995; Benhar et al., J. Biol. Chem., 269: 13398-404, 1994; Brumeanu et al., J Immunol., 154: 3088-95, 1995; see also, Caliceti et al. (2003) Adv. Drug Deliv. Rev. 55(10):1261-77 and Molineux (2003) Pharmacotherapy 23 (8 Pt 2):3S-8S). Pegylation also can be used in the delivery of nucleic acid molecules in vivo. For example, pegylation of adenovirus can increase stability and gene transfer (see, e.g., Cheng et al. (2003) Pharm. Res. 20(9): 1444-51).

Desirable blood levels can be maintained by a continuous infusion of the active agent as ascertained by plasma levels. It should be noted that the attending physician would know how, and when, to terminate, interrupt or adjust therapy to lower dosage due to toxicity, or bone marrow, liver or kidney dysfunctions. Conversely, the attending physician would also know how, and when, to adjust treatment to higher levels if the clinical response is not adequate (precluding toxic side effects). The active agent is administered, for example, by oral, pulmonary, parental (intramuscular, intraperitoneal, intravenous (IV) or subcutaneous injection), inhalation (via a fine powder formulation), transdermal, nasal, vaginal, rectal, or sublingual routes of administration and can be formulated in dosage forms appropriate for each route of administration (see, e.g., International PCT application Nos. WO 93/25221 and WO 94/17784; and European Patent Application 613,683).

A CSR or ligand isoform is included in the pharmaceutically acceptable carrier in an amount sufficient to exert a therapeutically useful effect in the absence of undesirable side effects on the patient treated. Therapeutically effective concentrations can be determined empirically by testing the compounds in known in vitro and in vivo systems, such as the assays provided herein.

The concentration-a CSR or ligand isoform in the composition will depend upon absorption, inactivation and excretion rates of the complex, the physicochemical characteristics of the complex, the dosage schedule, and amount administered as well as other factors known to those of skill in the art. The amount of a CSR or ligand isoform to be administered for the treatment of a disease or condition, for example cancer, autoimmune disease and infection can be determined by standard clinical techniques. In addition, in vitro assays and animal models can be employed to help identify optimal dosage ranges. The precise dosage, which can be determined empirically, can depend upon the route of administration and the seriousness of the disease. Suitable dosage ranges for administration can range from about 0.01 pg/kg body weight to 1 mg/kg body weight and more typically 0.05 mg/kg to 200 mg/kg CSR isoform: patient weight.

A CSR or ligand isoform can be administered at once, or can be divided into a number of smaller doses to be administered at intervals of time. CSR or ligand isoforms can be administered in one or more doses over the course of a treatment time for example over several hours, days, weeks, or months. In some cases, continuous administration is useful. It is understood that the precise dosage and duration of treatment is a function of the disease being treated and can be determined empirically using known testing protocols or by extrapolation from in vivo or in vitro test data. It is to be noted that concentrations and dosage values also can vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or use of compositions and combinations containing them.

H. In vivo Expression of CSR and Ligand Isoforms and Gene Therapy

CSR and ligand isoforms, particularly modified CSR and ligand isoforms that contain additional amino acids at their N-terminus following expression and secretion, can be delivered to cells and tissues by expression of nucleic acid molecules. CSR and ligand isoforms can be administered as nucleic acid molecules encoding a CSR or ligand isoform, including ex vivo techniques and direct in vivo expression.

1. Delivery of Nucleic Acids

Nucleic acids, such as but not limited to any set forth in any of SEQ ID NOS: 31, 33, 35, 37, 39, 41, 43, 45, or 47 can be delivered to cells and tissues by any method known to those of skill in the art.

a. Vectors—Episomal and Integrating

Methods for administering CSR and ligand isoforms by expression of encoding nucleic acid molecules include administration of recombinant vectors. The vector can be designed to remain episomal, such as by inclusion of an origin of replication or can be designed to integrate into a chromosome in the cell.

CSR and ligand isoforms also can be used in ex vivo gene expression therapy using non-viral vectors. For example, cells can be engineered to express a CSR and ligand isoform, such as by integrating a CSR and ligand isoform encoding-nucleic acid into a genomic location, either operatively linked to regulatory sequences or such that it is placed operatively linked to regulatory sequences in a genomic location. Such cells then can be administered locally or systemically to a subject, such as a patient in need of treatment.

Viral vectors, including, for example adenoviruses, herpes viruses, retroviruses and others designed for gene therapy, can be employed. The vectors can remain episomal or can integrate into chromosomes of the treated subject. A CSR or ligand isoform can be expressed by a virus, which is administered to a subject in need of treatment. Virus vectors suitable for gene therapy include adenovirus, adeno-associated virus, retroviruses, lentiviruses and others noted above. For example, adenovirus expression technology is well-known in the art and adenovirus production and administration methods also are well known. Adenovirus serotypes are available, for example, from the American Type Culture Collection (ATCC, Rockville, Md.). Adenovirus can be used ex vivo, for example, cells are isolated from a patient in need of treatment, and transduced with a CSR or ligand isoform-expressing adenovirus vector. After a suitable culturing period, the transduced cells are administered to a subject, locally and/or systemically. Alternatively, CSR or ligand isoform-expressing adenovirus particles are isolated and formulated in a pharmaceutically-acceptable carrier for delivery of a therapeutically effective amount to prevent, treat or ameliorate a disease or condition of a subject. Typically, adenovirus particles are delivered at a dose ranging from 1 particle to 1014 particles per kilogram subject weight, generally between 106 or 108 particles to 1012 particles per kilogram subject weight. In some situations it is desirable to provide a nucleic acid source with an agent that targets cells, such as an antibody specific for a cell surface membrane protein or a target cell, or a ligand for a receptor on a target cell.

b. Artificial Chromosomes and other Non-Viral Vector Delivery Methods

CSR or ligand isoforms, also can be used in ex vivo gene expression therapy using non-viral vectors. For example, cells can be engineered which express a CSR or ligand isoform, such as by integrating a CSR or ligand isoform sequence into a genomic location, either operatively linked to regulatory sequences or such that it is placed operatively linked to regulatory sequences in a genomic location. Such cells then can be administered locally or systemically to a subject, such as a patient in need of treatment.

The nucleic acid molecules can be introduced into artificial chromosomes and other non-viral vectors. Artificial chromosomes (see, e.g., U.S. Pat. No. 6,077,697 and PCT International PCT application No. WO 02/097059) can be engineered to encode and express the isoform.

c. Liposomes and Other Encapsulated Forms and Administration of Cells Containing the Nucleic Acids

The nucleic acids can be encapsulated in a vehicle, such as a liposome, or introduced into a cell, such as a bacterial cell, particularly an attenuated bacterium or introduced into a viral vector. For example, when liposomes are employed, proteins that bind to a cell surface membrane protein associated with endocytosis can be used for targeting and/or to facilitate uptake, e.g. capsid proteins or fragments thereof tropic for a particular cell type, antibodies for proteins which undergo internalization in cycling, and proteins that target intracellular localization and enhance intracellular half-life.

2. In vitro and Ex vivo Delivery

For ex vivo and in vivo methods, nucleic acid molecules encoding the CSR or ligand isoform are introduced into cells that are from a suitable donor or the subject to be treated. In vivo expression of a CSR or ligand isoform can be linked to expression of additional molecules. For example, expression of a CSR or ligand isoform can be linked with expression of a cytotoxic product such as in an engineered virus or expressed in a cytotoxic virus. Such viruses can be targeted to a particular cell type that is a target for a therapeutic effect. The expressed CSR or ligand isoform, particularly expressed and secreted modified forms of CSR and ligand isoforms containing additional amino acids at their N-terminus, can be used to enhance the cytotoxicity of the virus.

In vivo expression of a CSR or ligand isoform can include operatively linking a CSR or ligand isoform encoding nucleic acid molecule to specific regulatory sequences such as a cell-specific or tissue-specific promoter. CSR or ligand isoforms also can be expressed from vectors that specifically infect and/or replicate in target cell types and/or tissues. Inducible promoters can be use to selectively regulate CSR or ligand isoform expression.

Cells into which a nucleic acid can be introduced for purposes of therapy encompass any desired, available cell type appropriate for the disease or condition to be treated, including but not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., such as stem cells obtained from bone marrow, umbilical cord blood, peripheral blood, fetal liver, and other sources thereof. Tumor cells also can be target cells for in vivo expression of CSR or ligand isoforms. Cells used for in vivo expression of an isoform also include cells autologous to the patient. Such cells can be removed from a patient, nucleic acids for expression of a CSR or ligand isoform introduced, and then administered to a patient such as by injection or engraftment.

Techniques suitable for the transfer of nucleic acid into mammalian cells in vitro include the use of liposomes and cationic lipids (e.g., DOTMA, DOPE and DC-Chol) electroporation, microinjection, cell fusion, DEAE-dextran, and calcium phosphate precipitation methods. Methods of DNA delivery can be used to express CSR isoforms in vivo. Such methods include liposome delivery of nucleic acids and naked DNA delivery, including local and systemic delivery such as using electroporation, ultrasound and calcium-phosphate delivery. Other techniques include microinjection, cell fusion, chromosome-mediated gene transfer, microcell-mediated gene transfer and spheroplast fusion.

For ex vivo treatment, cells from a donor compatible with the subject to be treated or cells from the subject to be treated are removed, the nucleic acid is introduced into these isolated cells and the modified cells are administered to the subject.

Treatment includes direct administration, such as for, for example, encapsulated within porous membranes, which are implanted into the patient (see, e.g. U.S. Pat. Nos. 4,892,538 and 5,283,187). Techniques suitable for the transfer of nucleic acid into mammalian cells in vitro include the use of liposomes and cationic lipids (e.g., DOTMA, DOPE and DC-Chol) electroporation, microinjection, cell fusion, DEAE-dextran, and calcium phosphate precipitation methods. Methods of DNA delivery can be used to express CSR isoforms in vivo. Such methods include liposome delivery of nucleic acids and naked DNA delivery, including local and systemic delivery such as using electroporation, ultrasound and calcium-phosphate delivery. Other techniques include microinjection, cell fusion, chromosome-mediated gene transfer, microcell-mediated gene transfer and spheroplast fusion.

In vivo expression of a CSR or ligand isoform can be linked to expression of additional molecules. For example, expression of a CSR or ligand isoform can be linked with expression of a cytotoxic product such as in an engineered virus or expressed in a cytotoxic virus. Such viruses can be targeted to a particular cell type that is a target for a therapeutic effect. The expressed CSR or ligand isoform can be used to enhance the cytotoxicity of the virus.

In vivo expression of a CSR or ligand isoform can include operatively linking a CSR or ligand isoforn encoding nucleic acid molecule to specific regulatory sequences such as a cell-specific or tissue-specific promoter. CSR or ligand isoforms also can be expressed from vectors that specifically infect and/or replicate in target cell types and/or tissues. Inducible promoters can selectively regulate CSR or ligand isoform expression. Additionally, in vivo expression of CSR or ligand isoforms can include operative linkage of a CSR or ligand isoform encoding nucleic acid with a sequence, such as a precursor sequence including a tPA pre/prosequence, to effect secretion of the CSR or ligand isoform from a target cell type.

3. Systemic, Local and Topical Delivery

Nucleic acid molecules, as naked nucleic acids or in vectors, artificial chromosomes, liposomes and other vehicles can be administered to the subject by systemic administration, topical, local and other routes of administration. When systemic and in vivo, the nucleic acid molecule or vehicle containing the nucleic acid molecule can be targeted to a cell.

Administration also can be direct, such as by administration of a vector or cells that typically targets a cell or tissue. For example, tumor cells and proliferating cells can be targeted cells for in vivo expression of CSR or ligand isoforms. Cells used for in vivo expression of an isoform also include cells autologous to the patient. Such cells can be removed from a patient, nucleic acids for expression of a CSR or ligand isoform introduced, and then administered to a patient such as by injection or engraftment.

I. Exemplary Treatments and Studies with CSR Isoforms

Provided herein are methods of treatment of disease and conditions with CSR or ligand isoforms, particularly modified CSR or ligand isoforms that contain one or more additional amino acids at the N-terminus following expression and secretion of the isoform. Included among modified CSR or ligand isoforms are, for example, those isoforms having additional amino acids at the N-terminus due to incomplete processing following secretion (i.e. GAR), the presence of encoded linker sequences (i.e. LE or SR), and/or the presence of an epitope tag (i.e. c-myc or His-tag). Such CSR and ligand isoforms or nucleic acids encoding CSR and ligand isoforms, such as RTK isoforms, TNFR isoforms, RAGE isoforms, and ligand isoforms including HGF isoforms can be used in the treatment of a variety of diseases and conditions, including those described herein. Typically, treatment of a disease, disorder, or condition by a polypeptide isoform provided herein, or a nucleic acid encoding a polypeptide isoform, is one which is mediated by a cognate receptor or ligand. For example, chronic activation induced by RAGE-mediated signaling contributes to disease progression in age-related macular degeneration. Hence, treatment of age-related macular degeneration with a RAGE isoform, such as any provided herein, can be used as a treatment of age-related macular degeneration and other angiogenic conditions. Contributions of cognate CSRs and ligands to other various diseases and disorders are known to one of skill in the art, and are exemplified herein below.

Treatment can be effected by administering by suitable route formulations of the polypeptides, which can be provided in compositions as polypeptides and can be linked to targeting agents for targeted delivery or encapsulated in delivery vehicles, such as liposomes. Alternatively, nucleic acids encoding the polypeptides can be administered as naked nucleic acids or in vectors, particularly gene therapy vectors. Gene therapy can be effected by any method known to those of skill in the art. Gene therapy can be effected in vivo by directly administering the nucleic acid or vector. For example, the nucleic acids can be delivered systemically, locally, topically or by any suitable route. The vectors or nucleic acids can be targeted by including targeting agents in the delivery vehicle, such as a virus or liposome, or they can be conjugated to a targeting agent, such as an antibody. The vectors or nucleic acids can be introduced into cells ex vivo by removing cells from a subject or suitable donor, introducing the vector or nucleic acid into the cells and then introducing the modified cells into the subject.

The CSR isoforms or ligand isoforms provided herein, particularly modified isoforms containing additional amino acids at the N-terminus due to incomplete processing, the presence of an encoded linker sequence, or the presence of an epitope tag, can be used for treating a variety of disorders, particularly proliferative, immune and inflammatory disorders. Treatments include, but are not limited to, treatment of angiogenesis-related diseases and conditions including ocular diseases, atherosclerosis, cancer and vascular injuries, neurodegenerative diseases, including Alzheimer's disease, inflammatory diseases and conditions, including atherosclerosis, diseases and conditions associated with cell proliferation including cancers, and smooth muscle cell-associated conditions, and various autoimmune diseases. Exemplary treatments and preclinical studies are described for treatments and therapies with RTK isoforms, TNFR isoforms, RAGE isoforms, or HGF isoforms. Such descriptions are meant to be exemplary only and are not limited to a particular RTK, TNFR, RAGE, or HGF isoform. The particular treatment and dosage can be determined by one of skill in the art. Considerations in assessing treatment include; the disease to be treated, the severity and course of the disease, whether the molecule is administered for preventive or therapeutic purposes, previous therapy, the patient's clinical history and response to therapy, and the discretion of the attending physician.

1. Angiogenesis-Related Conditions

CSR isoforms including, but not limited to, RTK isoforms including VEGFR, PDGFR, MET, TIE/TEK, EGFR, and EphA, TNFR isoforms including TNFR1 and TNFR2, RAGE isoforms, and HGF isoforms can be used in treatment of angiogenesis-related diseases and conditions, such as ocular diseases and conditions, including ocular diseases involving neovascularization. Ocular neovascular disease is characterized by invasion of new blood vessels into the structures of the eye, such as the retina or cornea. It is the most common cause of blindness and is involved in approximately twenty eye diseases. In age-related macular degeneration, the associated visual problems are caused by an ingrowth of choroidal capillaries through defects in Bruch's membrane with proliferation of fibrovascular tissue beneath the retinal pigment epithelium. Angiogenic damage also is associated with diabetic retinopathy, retinopathy of prematurity, corneal graft rejection, neovascular glaucoma and retrolental fibroplasia. Other diseases associated with corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical bums, bacterial ulcers, fungal ulcers, Herpes simplex infections, Herpes zoster infections, protozoan infections, Karposi sarcoma, Mooren ulcer, Terrien's marginal degeneration, marginal keratolysis, rheumatoid arthritis, systemic lupus, polyarteritis, trauma, Wegeners sarcoidosis, Scleritis, Steven's Johnson disease, periphigoid radial keratotomy, and corneal graph rejection. Diseases associated with retinal/choroidal neovascularization include, but are not limited to, diabetic retinopathy, macular degeneration, sickle cell anemia, sarcoid, syphilis, pseudoxanthoma elasticum, Pagets disease, vein occlusion, artery occlusion, carotid obstructive disease, chronic uveitis/vitritis, mycobacterial infections, Lyme's disease, systemic lupus erythematosus, retinopathy of prematurity, Eales disease, Bechets disease, infections causing a retinitis or choroiditis, presumed ocular histoplasmosis, Bests disease, myopia, optic pits, Stargardt's disease, pars planitis, chronic retinal detachment, hyperviscosity syndromes, toxoplasmosis, trauma and post-laser complications. Other diseases include, but are not limited to, diseases associated with rubeosis (neovascularization of the angle) and diseases caused by the abnormal proliferation of fibrovascular or fibrous tissue including all forms of proliferative vitreoretinopathy.

The therapeutic effect of CSR and ligand isoforms, including modified forms of CSR and ligand isoforms, on angiogenesis such as in treatment of ocular diseases can be assessed in animal models, for example in cornea implants, such as described herein. For example, modulation of angiogenesis such as for an RTK can be assessed in a nude mouse model such as epidermoid A43 1 tumors in nude mice and VEGF-or PIGF-transduced rat C6 gliomas implanted in nude mice. CSR or ligand isoforms can be injected as protein locally or systemically. Alternatively cells expressing CSR isoforms can be inoculated locally or at a site remote to the tumor. Tumors can be compared between control-treated and CSR isoform-treated models to observe phenotypes of tumor inhibition including poorly vascularized and pale tumors, necrosis, reduced proliferation and increased tumor-cell apoptosis. In one such treatment, Flt-1 isoforms are used to treat ocular disease and assessed in such models.

Examples of ocular disorders that can be treated with TIE/TEK isoforms are eye diseases characterized by ocular neovascularization including, but not limited to, diabetic retinopathy (a major complication of diabetes), retinopathy of prematurity (this devastating eye condition, that frequently leads to chronic vision problems and carries a high risk of blindness, is a severe complication during the care of premature infants), neovascular glaucoma, retinoblastoma, retrolental fibroplasia, rubeosis, uveitis, macular degeneration, and corneal graft neovascularization. Other eye inflammatory diseases, ocular tumors, and diseases associated with choroidal or iris neovascularization also can be treated with TIE/TEK isoforms.

For example, CSR and ligand isoforms, including RAGE isoforms, can be used in treatment of ocular diseases and conditions, including age-related macular degeneration. Age-related macular degeneration is associated with vision loss resulting from accumulated macular drusen, extracellular deposits in Brusch's membrane, and retinal pigment epithelium (RPE) dysfunction due to degenerative cellular and molecular changes in RPE and photoreceptors overlying the macular drusen. The cellular and molecular changes occurring in the RPE, in part due to oxidative stress in the aging eye, include altered expression of genes for cytokines, matrix organization, cell adhesion, and apoptosis resulting in the possible induction of a focal inflammatory response at the RPE-Bruch's membrane border. For example, oxidative stress induces the accumulation of RAGE ligands in the RPE and photoreceptor layers in early age-related macular degeneration. The accumulated RAGE ligands stimulate RAGE-expressing RPE cells to induce a variety of inflammatory events including NFκB nuclear localization, apoptosis, and most importantly the upregulation of the RAGE receptor itself initiating a positive feedback loop sustained by continued ligand availability. The chronic activation induced by the ligand/RAGE-mediated signaling contributes to disease progression in age-related macular degeneration. Treatment of early stage age-related macular generation with CSR or ligand isoforms can ameliorate one or more symptoms of the disease.

PDGFR isoforms also can be used in the treatment of proliferative vitreoretinopathy. For example, an expression vector such as a retroviral vector is constructed containing a nucleic acid molecule encoding a PDGFR isoform. Rabbit conjunctival fibroblasts (RCFs) are produced which contain the expression vector by transfection, such for a retrovirus vector, or by transformation, such as for a plasmid or chromosomal based vector. Expression of a PDGFR isoform can be monitored in cells by means known in the art including use of an antibody which recognizes PDGFR isoforms and by use of a peptide tag (e.g a myc tag) and corresponding antibody. RCFs are injected into the vitreous part of an eye. For example, in a rabbit animal model, approximately 1×10⁵ RCFs are injected by gas vitreomy. Retrovirus expressing a PDGFR isoform, ˜2×10⁷ CFU, is injected on the same day. Effects on proliferative vitreoretinopathy can be observed, for example, 2-4 weeks following surgery, such as attenuation of the disease symptoms.

EphA isoforms can be used to treat diseases or conditions with misregulated and/or inappropriate angiogenesis, such as in eye diseases. For example, an EphA isoform can be assessed in an animal model such as a mouse corneal model for effects on ephrinA-1 induced angiogenesis. Hydron pellets containing ephrinA-1 alone or with EphA isoform protein are implanted in mouse cornea. Visual observations are taken on days following implantation to observe EphA isoform inhibition or reduction of angiogenesis. Anti-angiogenic treatments and methods such as described for VEGFR isoforms are applicable to EphA isoforms.

2. Angiogenesis Related Atherosclerosis

CSR and ligand isoforms including RTK isoforms, for example VEGFR Flt-I and TIE/TEK isoforms, can be used to treat angiogenesis conditions related to atherosclerosis such as neovascularization of atherosclerosis plaques. Plaques formed within the lumen of blood vessels have been shown to have angiogenic stimulatory activity. VEGF expression in human coronary atherosclerotic lesions is associated with the progression of human coronary atherosclerosis.

Animal models can be used to assess CSR and ligand isoforms in treatment of atherosclerosis. Apolipoprotein-E deficient mice (ApoE^{-/-}) are prone to atherosclerosis. Such mice are treated by injecting an RTK isoform, for example a VEGFR isoform, such as a Flt-1 intron fusion protein over a time course such as for 5 weeks starting at 5, 10 and 20 weeks of age. Lesions at the aortic root are assessed between control ApoE^{-/-} mice and isoform-treated ApoE^{-/-} mice to observe reduction of atherosclerotic lesions in isoform-treated mice.

3. Angiogenesis Related Diabetes

CSR and ligand isoforms, including RAGE isoforms, can be used to treat diabetes-related disease conditions such as vascular disease, periodontal disease, and autoimmune disease. Diabetes can occur by two main forms: type 1 diabetes is characterized by a progressive destruction of pancreatic β-islet cells which results in insulin deficiency; type 2 diabetes is characterized by an increased resistance and/or deficient secretion of insulin leading to hyperglycemia. Complications which result from hyperglycemia, such as myocardial infarction, stroke, and amputation of digits or limbs, can result in morbidity and mortality. Hyperglycemia results in sustained accumulation of RAGE ligands and signaling of RAGE by its ligands contributes to enhanced expression of the RAGE receptor in the diabetic tissue and chronic ligand-mediated RAGE signaling.

a. Vascular Disease

CSR and ligand isoforms, such as for example, RAGE isoforms, can be used to treat diabetes-related vascular disease, including both macrovascular and microvascular disease. Hyperglycemia occurring in type 2 diabetes results in chronic vascular injury characterized by a variety of macrovascular perturbations including the development of atherosclerotic plaques, enhanced proliferation of vascular smooth muscle, production of extracellular matrix, and vascular inflammation. Vascular inflammation can be caused and exacerbated by engagement of RAGE by its ligands leading to chronic vascular inflammation, accelerated atherosclerosis, and exaggerated restenosis after revascularization procedures. RAGE isoforms can be employed to block the ligation of RAGE by its ligands to suppress the vascular complications of diabetes. For example, in animal models of diabetes-associated hyperpermeability, treatment of animals with soluble RAGE isoform can lead to near normalization of tissue permeability. In another example of diabetes-related vascular disease, animal models of hyperlipidemia, such as ApoE−/− mice or LDL receptor−/− mice, that have been induced to develop diabetes, display increased accumulation of RAGE ligands and enhanced expression of RAGE. Treatment of diabetic mice with a soluble RAGE isoform can diminish diabetes-related atherogenesis as evidenced by reduced atherosclerotic lesion area size and decreased levels of tissue factor, VCAM-1, and NFκB compared with vehicle-treated mice. Treatment with RAGE isoforms to block diabetic atherosclerosis can be given any time during disease progression including after establishment of atherosclerotic plaques.

Diabetes-related vascular disease also can manifest in the microvasculature affecting the eyes, kidney, and peripheral nerves. Importantly, renal disease accounts for the largest percentage of mortality of any diabetes-specific complication. RAGE isoforms can be used to treat diabetes-related vascular disease, including kidney disease. For example, in a mouse model of diabetes, insulin-resistant db/db mouse, RAGE is upregulated in the glomerulus of the kidney particularly in the podocyte cells and likewise, RAGE-ligand expressing mononuclear phagocytes also are accumulated in the glomerulus. Treatment of db/db mice with a soluble RAGE isoform blocks VEGF expression, a factor known to mediate hyperpermeability and recruitment of mononuclear phagocytes into the glomerulus. Further treatment with RAGE isoforms also decrease glomerular and mesangial expansion and decrease the albumin excretion rate.

CSR and ligand isoforms, including RAGE isoforms, also can be used to treat diabetes-related vascular disease associated with wound healing. Chronic wound healing is often associated with diabetes and can lead to complications such as infection and amputation. Using the db/db mouse model of type 2 diabetes, a wound healing model can be established by performing full-thickness excisional wounds to generate chronic ulcers. In such a model, the levels of RAGE and its ligands are enhanced. Treatment of mice with a soluble RAGE isoform can increase wound closure by suppressing levels of cytokines including IL-6, TNF-α, and MMP-2, 3, and 9. This reduction in cytokine levels contributes to reduced chronic inflammation and ultimately enhances the generation of a thick, well-vascularized granulation tissue and increased levels of VEGF and PDGF-B.

b. Periodontal Disease

CSR and ligand isoforms, including RAGE isoforms, can be used to treat diabetes-related periodontal disease. Diabetes is a risk factor for the development of periodontal disease due to multiple factors including, for example, impaired host defenses upon invasion of bacterial pathogens, and exaggerated inflammatory responses once infection is established. An inappropriate immune response can lead to alveolar bone loss characteristic of periodontal disease by multiple mechanisms including, for example, impaired recruitment and function of neutrophils after infection by pathogenic bacteria, diminished generation of collagen and exaggerated collagenolytic activity, genetic predisposition, and mechanisms that lead to an enhanced inflammatory response such as, for example, sustained signaling by RAGE. RAGE and its ligands are accumulated in multiple cell types in the diabetic gingiva in patients with gingivitis-periodontitis including the endothelium and infiltrating mononuclear phagocytes. A diabetic mouse model using streptozotocin to induce diabetes, followed by inoculation of mice with the human periodontal pathogen Porphyromonas gingivalis, can be used as a model of periodontal disease. Mice treated with a RAGE isoform, such as by once daily intraperitoneal injections immediately following inoculation with P. gingivalis for 2 months, can be observed for periodontal disease by assessing the degree of alveolar bone loss. Reduction of cytokines and matrix metalloproteinases, such as IL-6, TNF-α, MMP-2, 3, 9, which are implicated in the destruction on non-mineralized connective tissue and bone, also can be observed following treatment with a RAGE isoform compared to a vehicle control.

4. Additional Angiogenesis-Related Treatments

CSR and ligand isoforms, including RTK isoforms such as VEGFR isoforms, for example, Fltl isoforms, and EphA isoforms also can be used to treat angiogenic and inflammatory-related conditions such as proliferation of synoviocytes, infiltration of inflammatory cells, inflammatory joint disease including cartilage destruction and pannus formation, such as are present in rheumatoid arthritis (RA). For example, an autoimmune model of collagen type-II induced arthritis, such as polyarticular arthritis induced in mice, can be used as a model for human RA. In such a model, mice can be treated with a CSR of ligand isoform, including but not limited to a HER2 isoform, FGFR isoform, VEGFR isoform, or other such isoform such as any described herein, such as by local injection of the protein or by gene therapy means. Following treatment, the mice can be observed for reduction of arthritic symptoms including paw swelling, erythema and ankylosis. Reduction in synovial angiogenesis and synovial inflammation also can be observed.

Other angiogenesis-related conditions amenable to treatment with VEGFR isoforms include hemangioma. One of the most frequent angiogenic diseases of childhood is hemangioma. In most cases, the tumors are benign and regress without intervention. In more severe cases, the tumors progress to large cavernous and infiltrative forms and create clinical complications. Systemic forms of hemangiomas, the hemangiomatoses, have a high mortality rate. Many cases of hemangiomas exist that cannot be treated or are difficult to treat with therapeutics currently in use.

VEGFR isoforms can be employed in the treatment of such diseases and conditions where angiogenesis is responsible for damage such as in Osler-Weber-Rendu disease, or hereditary hemorrhagic telangiectasia. This is an inherited disease characterized by multiple small angiomas, tumors of blood or lymph vessels. The angiomas are found in the skin and mucous membranes, often accompanied by epistaxis (nosebleeds) or gastrointestinal bleeding and sometimes with pulmonary or hepatic arteriovenous fistula. Diseases and disorders characterized by undesirable vascular permeability also can be treated by VEGFR isoforms. These include edema associated with brain tumors, ascites associated with malignancies, Meigs' syndrome, lung inflammation, nephrotic syndrome, pericardial effusion and pleural effusion.

Angiogenesis also is involved in normal physiological processes such as reproduction and wound healing. Angiogenesis is an important step in ovulation and also in implantation of the blastula after fertilization. Modulation of angiogenesis by VEGFR isoforms can be used to induce amenorrhea, to block ovulation or to prevent implantation by the blastula. VEGFR isoforms also can be used in surgical procedures. For example, in wound healing, excessive repair or fibroplasia can be a detrimental side effect of surgical procedures and may be caused or exacerbated by angiogenesis. Adhesions are a frequent complication of surgery and lead to problems such as small bowel obstruction.

PDGFR isoforms can be used in the regulation of neointima formation after arterial injury such as in arterial surgery. For example PDGFRB isoforms can be used to regulate PDGF-BB induced cell proliferation such as involved in neointima formation. PDGFR isoforms can be assessed for example, in a balloon-injured rooster femoral artery model. An adenovirus vector expressing a PDGFR isoform is constructed and transduced in vivo in the arterial model. Neointima-associated thrombosis is assessed in the transduced arteries to observe reduction compared with controls.

CSR and ligand isoforms useful in treatment of angiogenesis-related diseases and conditions also can be used in combination therapies such as with anti-angiogenesis drugs and molecules which interact with other signaling molecules in RTK-related pathways, including modulation of VEGFR ligands. For example, the known anti-rheumatic drug, bucillamine (BUC), was shown to include within its mechanism of action the inhibition of VEGF production by synovial cells. Anti-rheumatic effects of BUC are mediated by suppression of angiogenesis and synovial proliferation in the arthritic synovium through the inhibition of VEGF production by synovial cells. Combination therapy of such drugs with VEGFR isoforms can allow multiple mechanisms and sites of action for treatment.

5. Cancers

RTK isoforms such as isoforms of EGFR, TIE/TEK, VEGFR and FGFR can be used in treatment of cancers. RTK isoforms including, but not limited to, EGFR RTK isoforms, such as ErbB2 and ErbB3 isoforms, VEGFR isoforms such as Flt1 isoforms, FGFR isoforms such as FGFR4 isoforms, and EphA1 isoforms can be used to treat cancer. Examples of cancer to be treated herein include, but are not limited to, carcinoma, lymphoma, blastoma, sarcoma, and leukemia or lymphoid malignancies. Additional examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Combination therapies can be used with EGFR isoforms including anti-hormonal compounds, cardioprotectants, and anti-cancer agents such as chemotherapeutics and growth inhibitory agents.

Cancers treatable with EGFR isoforms generally are those that express an EGFR receptor or a receptor with which an EGF ligand interacts. Such cancers are known to those of skill in the art and/or can be identified by any means known in the art for detecting EGFR expression. An example of an ErbB2 expression diagnostic/prognostic assay available includes HERCEPTEST.RTM. (Dako). Paraffin embedded tissue sections from a tumor biopsy are subjected to the IHC assay and accorded an ErbB2 protein staining intensity criteria. Tumors accorded with less than a threshold score can be characterized as not overexpressing ErbB2, whereas those tumors with greater than or equal to a threshold score can be characterized as overexpressing ErbB2. In one example of treatment, ErbB2-overexpressing tumors are assessed as candidates for treatment with an EGFR isoform such as an ErbB2 isoform.

Isoforms provided herein can be used for treatment of cancers. For example, TIE/TEK isoforms can be used in the treatment of cancers such as by modulating tumor-related angiogenesis. Vascularization is involved in regulating cancer growth and spread.

For example, inhibition of angiogenesis and neovascularization inhibits solid tumor growth and expansion. TIE/TEK receptors such as Tie2 have been shown to influence vascular development in normal and cancerous tissues. TIE/TEK isoforms can be used as an inhibitor of tumor angiogenesis. A TIE/TEK isoform is produced such as by expression of the protein in cells. For example, secreted forms of TIE/TEK isoform can be expressed in cells and harvested from the media. Protein can be purified or partially-purified by biochemical means known in the art and by uses of antibody purification, such as antibodies raised against TIE/TEK isoform or a portion thereof or by use of a tagged TIE/TEK isoform and a corresponding antibody. Effects on angiogenesis can be monitored in an animal model such as by treating rat cornea with TIE/TEK isoform formulated as conditioned media in hydron pellets surgically implanted into a micropocket of a rat cornea or as or as purified protein (e.g. 100 μg/dose) administered to the window chamber. For example, rat models such as F344 rats with avascular corneas can be used in combination with tumor-cell conditioned media or by implanting a fragment of a tumor into the window chamber of an eye to induce angiogenesis. Corneas can be examined histologically to detect inhibition of angiogenesis induced by tumor-cell conditioned media. TIE/TEK isoforms also can be used to treat malignant and metastatic conditions such as solid tumors, including primary and metastatic sarcomas and carcinomas.

FGFR4 isoforms can be used to treat cancers, for example pituitary tumors.

Animal models can be used to mimic progression of human pituitary tumor progress. For example, an N-terminally shortened form of FGFR, ptd-FGFR4, expressed in transgenic mice recapitulates pituitary tumorigenesis (Ezzat et al. (2002) J. Clin. Invest. 109:69-78), including pituitary adenoma formation in the absence of prolonged and massive hyperplasia. FGFR4 isoforms can be administered to ptd-FGFR4 mice and the pituitary architecture and course of tumor progression compared with control mice.

6. Alzheimer's Disease

CSR receptor or ligand isoforms, such as EGFR isoforms, also can be used to 30 treat inflammatory conditions and other conditions involving such responses, such as Alzheimer's disease and related conditions. A variety of mouse models are available for human Alzheimer's disease including transgenic mice overexpressing mutant amyloid precursor protein and mice expressing familial autosomal dominant-linked PS1 and mice expressing both proteins (PS1 M146L/APPK670N:M671L). Alzheimer's models are treated such as by injection of ErbB isoforms. Plaque development can be assessed such as by observation of neuritic plaques in the hippocampus, entorhinal cortex, and cerebral cortex, using staining and antibody immunoreactivity assays.

Other neurodegenerative diseases, such as Creutzfeldt-Jakob disease and Huntington's disease, can be treated with CSR or ligand isoforms. For example, RAGE and its ligands are accumulated in prion protein plaques in Creutzfeldt-Jakob disease and in the caudate nucleus in Huntington's disease. Treatment of neurodegenerative diseases with CSR or ligand isoforms, such as for example, RAGE isoforms can limit inflammation and disease associated with sustained RAGE signaling.

7. Smooth Muscle Proliferative-Related Diseases and Conditions

CSR isoforms, including EGFR isoforms, such as ErbB isoforms, can be employed for the treatment of a variety of diseases and conditions involving smooth muscle cell proliferation in a mammal, such as a human. An example is treatment of cardiac diseases involving proliferation of vascular smooth muscle cells (VSMC) and leading to intimal hyperplasia such as vascular stenosis, restenosis resulting from angioplasty or surgery or stent implants, atherosclerosis and hypertension. In such conditions, an interplay of various cells and cytokines released act in autocrine, paracrine or juxtacrine manner, which result in migration of VSMCs from their normal location in media to the damaged intima. The migrated VSMCs proliferate excessively and lead to thickening of intima, which results in stenosis or occlusion of blood vessels. The problem is compounded by platelet aggregation and deposition at the site of lesion. Alpha-thrombin, a multifunctional serine protease, is concentrated at the site of vascular injury and stimulates VSMC proliferation. Following activation of this receptor, VSMCs produce and secrete various autocrine growth factors, including PDGF-AA, HB-EGF and TGF. EGFRs are involved in signal transduction cascades that ultimately result in migration and proliferation of fibroblasts and VSMCs, as well as stimulation of VSMCs to secrete various factors that are mitogenic for endothelial cells and induction of chemotactic response in endothelial cells. Treatment with EGFR isoforms can be used to modulate such signaling and responses.

EGFR isoforms such as ErbB2 and ErbB3 isoforms can be used to treat conditions where EGFRs such as ErbB2 and ErbB3 modulate bladder SMCs, such as bladder wall thickening that occurs in response to obstructive syndromes affecting the lower urinary tract. EGFR isoforms can be used in controlling proliferation of bladder smooth muscle cells, and consequently in the prevention or treatment of urinary obstructive syndromes.

EGFR isoforms can be used to treat obstructive airway diseases with underlying pathology involving smooth muscle cell proliferation. One example is asthma which manifests in airway inflammation and bronchoconstriction. EGF has been shown to stimulate proliferation of human airway SMCs and is likely to be one of the factors involved in the pathological proliferation of airway SMCs in obstructive airway diseases. EGFR isoforms can be used to modulate effects and responses to EGF by EGFRs.

8. Inflammatory Diseases

CSR and ligand isoforms, such as TNFR isoforms or RAGE isoforms, can be used in the treatment of inflammatory diseases including central nervous system diseases (CNS), autoimmune diseases, airway hyper-responsiveness conditions such as in asthma, rheumatoid arthritis and inflammatory bowel disease.

TNF-α and lymphotoxin (LT) are proinflammatory cytokines and critical mediators in inflammatory responses in diseases and conditions such as multiple sclerosis. TNF-α and LT-α are produced by infiltrating lymphocytes and macrophages and additionally by activated CNS parenchymal cells, microglial cells and astrocytes. In MS patients, TNF-α is overproduced in serum and cerebrospinal fluid. In lesions, TNF-α and TNFR are extensively expressed. TNF-α and LT-α can induce selective toxicity of primary oligodendrocytes and induce myelin damage in CNS tissues. Thus, these two cytokines have been implicated in demyelination.

Experimental autoimmune encephalomyelitis (EAE) can serve as a model for multiple sclerosis (MS) (see for example, Probert et al. (2000) Brain 123: 2005-2019). EAE can be induced in a number of genetically susceptible species by immunization with myelin and myelin components such as myelin basic protein, proteolipid protein and myelin oligodendrocyte glycoprotein (MOG). For example, MOG-induced EAE recapitulates essential features of human MS including the chronic, relapsing clinical disease course, the pathohistological triad of inflammation, reactive gliosis, and the formation of large confluent demyelinated plaques. Additional MS models include transgenic mice overexpressing TNF-α, which model nonauto-immune mediated MS. Transgenic mice are engineered to express TNF-α locally in glial cells; human and murine TNF-α trigger MS-like symptoms. TNFR isoforms can be assessed in EAE animal models. Isoforms are administered, such as by injection, and the course and progression of symptoms is monitored compared to control animals.

Cytokines such as TNF α also are involved in airway smooth muscle contractile properties. TNFR1 and TNFR2 play a role in modulating biological affects in airway smooth muscle. TNFR2 modulates calcium homeostasis and thereby modulates airway smooth muscle hyper-responsiveness. TNFR1 modulates effects of TNF-α in airway smooth muscle. Airway smooth muscle response can be assessed in murine tracheal rings induced with carbachol. Effects, such as carbachol-induced contraction, in the presence and absence of TNF-α can be monitored. TNFR isoforms can be added to tracheal rings to assess the effects of isoforms on airway smooth muscle.

CSRs, including TNFRs and other CSRs, modulate inflammation in diseases such as rheumatoid arthritis (RA) (Edwards et al. (2003) Adv Drug Deliv. Rev. 55(10):1315-36). TNFR isoforms, including TNFR1 or TNFR2 isoforms, can be used to treat RA. For example, TNFR isoforms can be injected locally or systemically. Isoforms can be dosed daily or weekly. Pegylated TNFR isoforms can be used to reduce immunogenicity. Primate models are available for RA treatments. Response of tender and swollen joints can be monitored in subjects treated with TNFR isoforms and controls to assess TNFR isoform treatment.

9. Cardiovascular Disease

CSR or ligand isoforms, including for example, RAGE isoforms, can be used in treatment of cardiovascular disease. RAGE and its ligands accumulate in ageing tissues including in the ageing human heart leading to sustained and chronic RAGE-mediated signaling. For example, RAGE signaling can mediate regulation of cell-matrix interactions through the activation of matrix metalloproteinases that has been observed, wfor example, in cardiac fibroblasts associated with cardiac fibrosis. Conversely, decreased levels of a soluble RAGE isoform in the plasma of patients with coronary artery disease, but not in control subjects, correlates with prognosis of athereosclerosis and vascular inflammation associated with coronary artery disease. Treatment of patients with cardiovascular disease and related conditions with RAGE isoforms may exert antiatherogenic effects by preventing ligand-mediated RAGE-dependent cellular activation.

10. Kidney Disease

CSR and ligand isoforms, including RAGE isoforms, can be used in treatment of chronic kidney disease. Kidney disease is characterized by chronic inflammation and elevated blood levels of proinflammatory cytokines such as TNF-α, IL-1′, and AGE, a ligand for RAGE. RAGE also is accumulated on peripheral blood monocytes from patients with chronic kidney disease, increasing as renal function deteriorates. RAGE/RAGE ligand signaling is associated with the chronic monocyte-mediated systemic inflammation associated with chronic kidney disease. Treatment with RAGE isoforms can diminish binding of RAGE ligands to cell surface RAGE and attenuate RAGE-mediated signaling such as the production of proinflammatory cytokines like TNF-α.

J. Combination Therapies

CSR or ligand isoforms, particularly those provided herein that are modified to include additional amino acids at their N-terminus following expression or secretion, can be used in combination with each other, with other cell surface receptor or ligand isoforms, such as a herstatin or any described, for example, in U.S. application Ser. Nos. 09/942,959, 09/234,208, 09/506,079; U.S. Provisional Application Ser. Nos. 60/571,289, 60/580,990 and 60/666,825; and U.S. Pat. No. 6,414,130, published International PCT application No WO 00/44403, WO 1/61356, WO 2005/016966, including but not limited to, those set forth in any of SEQ ID Nos. 32, 34, 36, 38, 40, 42, 44, 46, 48, 140, 142, 143, 145, 147, 149, 150, 152, 153, 155, 157, 159, 161-168, 170, 172, 174, 176, 178, 180, 181, 183, 185, 186, 188, 190, 192, 194, 196, 198, 200, 202, 204, 206, 208, 210, 212, 214, 216, 217, 219, 221, 223, 225, 227, 229, 230-233, 225, 237, 239, 241, 243, 245, 247, 248-251, 253, 255, 257, 259, 261, 263, 264-270, 272, 274-280, 282, 284, 286, 288, 289-303, or 319-333); and/or with other existing drugs and therapeutics to treat diseases and conditions, particularly those involving aberrant angiogenesis and/or neovascularization, including, but not limited to, cancers and other proliferative disorders, inflammatory diseases and autoimmune disorders, as set forth herein and known to those of skill in the art.

For example, as described herein a number of isoforms can be used to treat angiogenesis-related conditions and diseases and/or control tumor proliferation. Such treatments can be performed in conjunction with anti-angiogenic and/or anti-tumorigenic drugs and/or therapeutics. Examples of anti-angiogenic and anti-tumorigenic drugs and therapies useful for combination therapies include tyrosine kinase inhibitors and molecules capable of modulating tyrosine kinase signal transduction and can be used in combination therapies including, but not limited to, 4-aminopyrrolo[2,3-d]pyrimidines (see for example, U.S. Pat. No. 5,639,757), and quinazoline compounds and compositions (e.g., U.S. Pat. No. 5,792,771. Other compounds useful in combination therapies include steroids such as the angiostatic 4,9(11)-steroids and C21-oxygenated steroids, angiostatin, endostatin, vasculostatin, canstatin and maspin, angiopoietins, bacterial polysaccharide CM101 and the antibody LM609 (U.S. Pat. No. 5,753,230), thrombospondin (TSP-1), platelet factor 4 (PF4), interferons, metalloproteinase inhibitors, pharmacological agents including AGM-1470/TNP-470, thalidomide, and carboxyamidotriazole (CAI), cortisone such as in the presence of heparin or heparin fragments, anti-Invasive Factor, retinoic acids and paclitaxel (U.S. Pat. No. 5,716,981; incorporated herein by reference), shark cartilage extract, anionic polyamide or polyurea oligomers, oxindole derivatives, estradiol derivatives and thiazolopyrimidine derivatives.

In another example, a CSR or ligand isoform, such as a VEGF isoform, can be administered with an agent for treatment of diabetes. Such agents include agents for the treatment of any or all conditions such as diabetic periodontal disease, diabetic vascular disease, tubulointerstitial disease and diabetic neuropathy. In another example, a CSR isoform is administered with an agent that treats cancers such as an anti-cancer agent, a chemotherapeutic agent, and growth inhibitory agent, including coadministration of cocktails of different chemotherapeutic agents. Examples of chemotherapeutic agents include taxanes (such as paclitaxel and doxetaxel) and anthracycline antibiotics. Preparation and dosing schedules for such chemotherapeutic agents may be used according to manufacturers' instructions or as determined empirically by the skilled practitioner. Preparation and dosing schedules for such chemotherapy also are described in Chemotherapy Service Ed., M. C. Perry, Williams & Wilkins, Baltimore, Md. (1992). Examples of cancers to be treated include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Any of the CSR isoforms can be administered in combination with two or more agents for treatment of a disease or a condition.

Additional compounds can be used in combination therapy with CSR or ligand isoforms. Anti-hormonal compounds can be used in combination therapies, such as with EGFR isoforms. Examples of such compounds include an anti-estrogen compound such as tamoxifen; an anti-progesterone such as onapristone and an anti-androgen such as flutamide, in dosages known for such molecules. It also can be beneficial to also coadminister a cardioprotectant (to prevent or reduce myocardial dysfunction that can be associated with therapy) or one or more cytokines. In addition to the above therapeutic regimes, the patient may be subjected to surgical removal of cancer cells and/or radiation therapy.

Combinations of CSR or ligand isoforms, particularly those provided herein including modified forms of isoforms containing one or more additional amino acids at their N-terminus, with one or more different CSR or ligand isoforms including with herstatins and other agents, can be used for treating cancers and other disorders involving aberrant angiogenesis (see, e.g. copending and published applications U.S. application Ser. Nos. 09/942,959, 09/234,208, 09/506,079; U.S. Provisional Application Ser. Nos. 60/571,289, 60/580,990 and 60/666,825; and U.S. Pat. No. 6,414,130, published International PCT application No WO 00/44403, WO 01/61356, WO 2005/016966) are provided. The cell surface receptors include receptor tyrosine kinases, such as members of the VEGFR, FGFR, PDGFR (including Rα, Rβ, CSF1R, Kit), MET (including c-Met, c-RON), TIE and EPHA families. These can include ErbB2 (HER-2), ErbB3, ErbB4, EGFR, DDR1, DDR2, EphA1, EphB1, FGFR-2, FGFR-3, FGFR-4, MET, PDGFR-A, TEK, Tie-1, KIT, VEGFR-1, VEGFR-2, VEGFR-3, Flt1, Flt3, RON, or CSFIR, TNFR1, TNFR2, RON, CSFR1 and others. The cell surface receptors also can include isoforms of TNFRs or RAGE. Ligand isoforms also can be used in combination including HGF isoforms. Exemplary of such isoforms are the herstatins (see, SEQ ID NOS:290-303 and encoding nucleic acid sequences set forth in SEQ ID NOS:304-318), polypeptides that include the intron portion of a herstatin (see, SEQ ID NOS: 319-333 and encoding nucleic acid sequences set forth in SEQ ID NOS: 334-348), as well as any isoforms provided herein. The combinations of isoforms and/or drug agent selected is a function of the disease to be treated and is based upon consideration of the target tissues and cells and receptors expressed thereon.

The combinations, for example, can target two or more cell surface receptors or steps in the angiogenic and/or endothelial cell maintenance pathways or can target two or more cell surface receptors or steps in a disease process, such as any in which one or both of these pathways are implicated, such as inflammatory diseases, tumors and all other noted herein and known to those of skill in the art. The two or more agents can be administered as a single composition or can be administered as two or more compositions (where there are more than two agents) simultaneously, intermittently or sequentially. They can be packaged as a kit that contains two or more compositions separately or as a combined composition and optionally with instructions for administration and/or devices for administration, such as syringes.

Adjuvants and other immune modulators can be used in combination with CSR isoforms in treating cancers, for example to increase immune response to tumor cells. Combination therapy can increase the effectiveness of treatments and in some cases, create synergistic effects such that the combination is more effective than the additive effect of the treatments separately. Examples of adjuvants include, but are not limited to, bacterial DNA, nucleic acid fraction of attenuated mycobacterial cells (BCG; Bacillus-Calmette-Guerin), synthetic oligonucleotides from the BCG genome, and synthetic oligonucleotides containing CpG motifs (CpG ODN; Wooldridge et al. (1997) Blood 89:2994-2998), levamisole, aluminum hydroxide (alum), BCG, Incomplete Freud's Adjuvant (IFA), QS-21 (a plant derived immunostimulant), keyhole limpet hemocyanin (KLH), and dinitrophenyl (DNP). Examples of immune modulators include but are not limited to, cytokines such as interleukins (e.g., IL-2, IL-3, IL-4, IL-5, IL-6, IL-7, IL-9, IL-10, IL-11, IL-12, IL-13, IL-15, IL-16, IL-17, IL-18, IL-1α, IL-1β, and IL-1 RA), granulocyte colony stimulating factor (G-CSF), granulocyte-macrophage colony stimulating factor (GM-CSF), oncostatin M, erythropoietin, leukemia inhibitory factor (LIF), interferons, B7.1 (also known as CD80), B7.2 (also known as B70, CD86), TNF family members (TNF-α, TNF-β, LT-β, CD40 ligand, Fas ligand, CD27 ligand, CD30 ligand, 4-1BBL, Trail), and MIF, interferon, cytokines such as IL-2 and IL-12; and chemotherapy agents such as methotrexate and chlorambucil.

Preclinical Studies

Model animal studies can be used in preclinical evaluation of RTK isoforms that are candidate therapeutics. Parameters that can be assessed include, but are not limited to efficacy and concentration-response, safety, pharmnacokinetics, interspecies scaling and tissue distribution. Model animal studies include assays such as described herein as well as those known to one of skill in the art. Animal models can be used to obtain data that then can be extrapolated to human dosages for design of clinical trials and treatments with RTK isoforms. For example, efficacy and concentration-response VEGFR inhibitors in tumor-bearing mice can be extrapolated to human treatment (Mordenti et al., (1999) Toxicol Pathol. Jan-Feb; 27(1):14-21) in order to define clinical dosing regimens effective to maintain a therapeutic inhibitor, such as an antibody against VEGFR for human use in the required efficacious range. Similar models and dose studies can be applied to VEGFR isoform dosage determination and translated into appropriate human doses, as well as other techniques known to the skilled artisan. Preclinical safety studies and preclinical pharmacokinetics can be performed, for example in monkeys, mice, rats and rabbits. Pharmacokinetic data from mice, rats and monkeys has been used to predict the pharmacokinetics of the counterpart therapeutic in humans using allometric scaling. Accordingly, appropriate dosage information can be determined for the treatment of human pathological conditions, including rheumatoid arthritis, ocular neovascularization and cancer. A humanized version of the anti-VEGF antibody has been employed in clinical trials as an anti-cancer agent (Brem, (1998) Cancer Res. 58(13):2784-92; Presta et al., (1997) Cancer Res. 57(20):4593-9) and such clinical data also can be considered as a reference source when designing therapeutic doses for VEGFR isoforms.

### K EXAMPLES

The following examples are included for illustrative purposes only and are not intended to limit the scope of the invention.

### Example 1

### Method for Cloning CSR Isoforms

A. Preparation of Messenger RNA

MRNA isolated from major human tissue types from healthy or diseased tissues or cell lines were purchased from Clontech (BD Biosciences, Clontech, Palo Alto, Calif.) and Stratagene (La Jolla, Calif.). Equal amounts of mRNA were pooled and used as templates for reverse transcription-based PCR amplification (RT-PCR).

B. cDNA Synthesis

MRNA was denatured at 70° C. in the presence of 40% DMSO for 10 min and quenched on ice. First-strand cDNA was synthesized with either 200 ng oligo(dT) or 20 ng random hexamers in a 20 μl reaction containing 10% DMSO, 50 mM Tris-HCl (pH 8.3), 75 mM KCl, 3 mM MgCl₂, 10 mM DTT, 2 mM each dNTP, 5 μg mRNA, and 200 units of Stratascript reverse transcriptase (Stratagene, La Jolla, Calif.). After incubation at 37° C. for 1 h, the cDNA from both reactions were pooled and treated with 10 units of RNase H (Promega, Madison, Wis.).

C. PCR Amplification

Gene-specific PCR primers specific to a cell surface receptor (see e.g., Table 8 for exemplary cell surface receptors) were selected using the Oligo 6.6 software (Molecular Biology Insights, Inc., Cascade, Colo.) and synthesized by Qiagen-Operon (Richmond, Calif.). The forward primers (see e.g., Table 9) flank the start codon. The reverse primers flank the stop codon or were chosen from regions at least 1.5 kb downstream from the start codon (see Table 9). Each PCR reaction contained 10 ng of reverse-transcribed cDNA, 0.025 U/μl TaqPlus (Stratagene), 0.0035 U/μl PfuTurbo (Stratagene), 0.2 mM dNTP (Amersham, Piscataway, N.J.), and 0.2 μM forward and reverse primers in a total volume of 50 μl. PCR conditions were 35 cycles and 94.5° C. for 45 s, 58° C. for 50 s, and 72° C. for 5 min. The reaction was terminated with an elongation step of 72° C. for 10 min.

TABLE 8
LIST OF GENES FOR CLONING CSR Isoforms

| | | | | | | | SEQ |
|---|---|---|---|---|---|---|---|
| | | | Catalytic | SEQ ID | | | ID |
| Family | Member | nt ACC. # | Domain | NO: | ORF | prt ACC.# | NO: |
| DDR | DDR1 | NM_013993 | 2149-3057 | 355 | 337- | NP_054699 | 392 |
| | | | | | 3078 | | |
| | DDR2 | NM_006182 | 2022-2900 | 356 | 354- | NP_006173 | 393 |
| | | | | | 2921 | | |
| EPH | EPHA1 | NM-005232 | 1939-2736 | 357 | 88-3018 | NP_005223 | 394 |
| | EPHA2 | NM-004431 | 1956-2759 | 358 | 138- | NP_004422 | 395 |
| | | | | | 3068 | | |
| | EPHA3 | NM-005233 | 2086-2859 | 359 | 226- | NP_005224 | 396 |
| | | | | | 3177 | | |
| | EPHA4 | NM_004438 | 1885-2685 | 360 | 43-3003 | NP_004429 | 397 |
| | EPHA5 | L36644 | 1259-1460 | 361 | 1-2976 | AAA74245 | 398 |
| | EPHA6 | AL133666 | 691-1332 | 362 | 343- | CAB63775 | 399 |
| | | | | | 1347 | | |
| | EPHA7 | NM_004440 | 2092-2892 | 363 | 214- | NP_004431 | 400 |
| | | | | | 3210 | | |
| | EPHA8 | NM_020526 | 2028-2801 | 364 | 126- | NP_065387 | 401 |
| | | | | | 3143 | | |
| | EPHB1 | NM_004441 | 2051-2857 | 365 | 215- | NP_004432 | 402 |
| | | | | | 3169 | | |
| | EPHB2 | AF025304 | 1886-2681 | 366 | 26-3193 | AAB94602 | 403 |
| | EPHB3 | NM_004443 | 2316-3122 | 367 | 438- | NP_004434 | 404 |
| | | | | | 3434 | | |
| | EPHB4 | NM_004444 | 2200-3006 | 368 | 376- | NP_004435 | 405 |
| | | | | | 3339 | | |
| | EPHB6 | NM_004445 | 2761-3498 | 369 | 799- | NP_004436 | 406 |
| | | | | | 3819 | | |
| ERB | EGFR | NM_005228 | 2380-3148 | 370 | 247- | NP_005219 | 407 |
| | | | | | 3879 | | |
| | ERBB2 | NM_004448 | 2396-3164 | 371 | 239- | NP_004439 | 408 |
| | | | | | 4006 | | |
| | ERBB3 | NM_001982 | 2318-3086 | 372 | 194- | NP_001973 | 409 |
| | | | | | 4222 | | |
| FGFR | FGFR1 | M34641 | 1435-2263 | 373 | 10-2472 | AAA35835 | 410 |
| | FGFR2 | NM_000141 | 2009-2872 | 374 | 593- | NP_000132 | 411 |
| | | | | | 3058 | | |
| | FGFR3 | NM_000142 | 1429-2292 | 375 | 40-2460 | NP_000133 | 412 |
| | FGFR4 | NM_002011 | 1534-2394 | 376 | 157- | NP_002002 | 413 |
| | | | | | 2565 | | |
| MET | MET | NM_000245 | 3419-4198 | 377 | 188- | NP_000236 | 414 |
| | | | | | 4360 | | |
| | RON | NM_002447 | 3242-4260 | 378 | 29-4231 | NP_002438 | 415 |
| PDGFR | CSF1R | NM_005211 | 2012-3208 | 379 | 293- | NP_005202 | 416 |
| | | | | | 3211 | | |
| | FLT3 | NM_004119 | 1861-2886 | 380 | 58-3039 | NP_004110 | 417 |
| | KIT | NM_000222 | 1762-2799 | 381 | 22-2952 | NP_000213 | 418 |
| | PDGFRA | NM_006206 | 2147-3253 | 382 | 395- | NP_006197 | 419 |
| | | | | | 3664 | | |
| | PDGFRB | NM_002609 | 2133-3215 | 383 | 357- | NP_002600 | 420 |
| | | | | | 3677 | | |
| RAGE | RAGE | NM_001136 | | 384 | 25-1239 | NP_001127 | 421 |
| TEK | TEK | NM_000459 | 2603-3433 | 385 | 149- | NP_000450 | 422 |
| | | | | | 3523 | | |
| | TIE | NM_005424 | 2579-3409 | 386 | 80-3496 | NP_005415 | 423 |
| TNFR | TNFR1 | NM_001065 | 1323- | 387 | 282- | NP_001056 | 424 |
| | | | 1598(DD) | | 1649 | | |
| | TNFR2 | NM_001066 | n/a | 388 | 90-1475 | NP_001057 | 425 |
| VEGFR | VEGFR1 | NM_002019 | 2704-3702 | 389 | 250- | NP_002010 | 426 |
| | | | | | 4266 | | |
| | VEGFR2 | NM_002253 | 2779-3792 | 390 | 304- | NP_002244 | 427 |
| | | | | | 4374 | | |
| | VEGFR3 | NM_002020 | 2530-3525 | 391 | 22-3918 | NP_002011 | 428 |
| HGF | HGF | NM_000601 | | 460 | 166- | NP_000592 | 461 |
| | | | | | 2352 | | |

TABLE 9
PRIMERS FOR PCR CLONING.

| SEQ | | |
|---|---|---|
| ID | | |
| NO | Primer | Sequence |
| 463 | CSF1R_F1 | CTG CCA CTT CCC CAC CGA GG |
| 464 | DDR1_F1 | GGG ATC AGG AGC TAT GGG ACC A |
| 465 | DDR2_F1 | CTG AGA TGA TCC TGA TTC CCA GAA |
| 466 | EPHA1_F1 | GGA GCT ATG GAG CGG CGC TG |
| 467 | EPHA2_F1 | AGC GAG AAG CGC GGC ATG GA |
| 468 | EPHA3_F1 | CAC CAG CAA CAT GGA TTG TCA GC |
| 469 | EPHA4_F1 | CGA ACC ATG GCT GGG ATT TTC TA |
| 470 | EPHA7_F1 | ATA AAA CCT GCT CAT GCA CCA TG |
| 471 | EPHB1_F1 | GCG ATG GCC CTG GAT TAT CTA |
| 472 | EPHB2_F1 | CCC CGG GAA GCG CAG CCA |
| 473 | EPHB3_F1 | GCT CCT AGA GCT GCC ACG GC |
| 474 | EPHB4_F1 | GAT CCT ACC CGA GTG AGG CGG |
| 475 | CSF1R_R1 | GGG CTC CTG CAG AGA TGG GTA |
| 476 | DDR1_R1 | AGA GCC ATT GGG GAC ACA GGG A |
| 477 | DDR2_R1 | AGC CTG ACT CCT CCT CCC CTG |
| 478 | EPHA1_R1 | AGC TCT GTC AGC AAG ACC CTG G |
| 479 | EPHA2_R1 | AGG TGG TGT CTG GGG CCA GGT C |
| 480 | EPHA3_R1 | GTC AGG CTT GAG GCT ACT GAT GG |
| 481 | EPHA4_R1 | AAC ATA GGA AGT GAG AGG GTT CAG |
| | | G |
| 482 | EPHA7_R1 | ACT CCA TTG GGA TGC TCT GGT TC |
| 483 | EPHB1_R1 | AGC CCA TCA ATC CTT GCT GTG |
| 484 | EPHB2_R1 | GCG TGC CCG CAC CTG GAA GA |
| 485 | EPHB3_R1 | GCT GGT CAC TGT GGA GGC GA |
| 486 | EPHB4_R1 | GGT AGC TGG CTC CCC GCT TCA |
| 487 | CSF1R_R2 | CCG AGG GTC TTA CCA AAC TGC |
| 488 | DDR1_R2 | AAG CGG AGT CGA GAT CGA GGG A |
| 489 | DDR2_R2 | GGG GAA CTC CTC CAC AGC CA |
| 490 | EPHA1_R2 | CGG GTA AAG TCC AAG GCT CCC |
| 491 | EPHA2_R2 | GAC ACA GGA TGG ATG GAT CTC GG |
| 492 | EPHA3_R2 | ATC AAT GGA TAT GTT GGT GGC ATC |
| 493 | EPHA4_R2 | AGG ATG CGT CAA TTT CTT TGG CA |
| 494 | EPHA7_R2 | CTG CAC CAA TCA CAC GCT CAA |
| 495 | EPHB1_R2 | ATC AAT CTC CTT GGC AAA CTC C |
| 496 | EPHB2_R2 | GCC CAT GAT GGA GGC TTC GC |
| 497 | EPHB3_R2 | ACG CAG GAC ACG TCG ATC TCC |
| 498 | EPHB4_R2 | ACC TGC ACC AAT CAC CTC TTC AA |
| 499 | EPHB6_F1 | AGA GTG GCG GGC ATG GTG TG |
| 500 | EPHB6_R1 | GCG GAG CTG ATA GTC CAG GAT G |
| 501 | EPHB6_R2 | CCT GTC CCA ATG ACC TCC TCA A |
| 502 | EPHA6_F1 | GGA GAT GAA AGA CTC TCC ATT TCA |
| | | AG |
| 503 | FGFR1_F1 | ATT CGG GAT GTG GAG CTG GA |
| 504 | FGFR2_F1 | AGG ACC GGG GAT TGG TAC CG |
| 505 | FGFR3_F1 | CAT GGG CGC CCC TGC CTG |
| 506 | FGFR4_F1 | AGA AGG AGA TGC GGC TGC TG |
| 507 | TNFR1A(p55)_F1 | AGC TGT CTG GCA TGG GCC TCT C |
| 508 | TNFR1B(p55)_F1 | ACC GGA CCC CGC CCG CAC |
| 509 | EPHA6_R1 | ATCT TAG ACC GAC AGA AAA TTT |
| | | GGC |
| 510 | FGFR1_R1 | CAA GGG ACC ATC CTG CGT GC |
| 511 | FGFR2_R1 | AGG GGC TTG CCC AGT GTC AG |
| 512 | FGFR3_R1 | GCT CCC ATT TGG GGT CGG CA |
| 513 | FGFR4_R1 | CGG GGG AAC TCC CAT AGT GG |
| 514 | TNFR1A(p55)_R1 | GGC GCA GCC TCA TCT GAG AAG A |
| 515 | TNFR1B(p55)_R1 | CAC AGC CCA CAC CGG CCT GG |
| 516 | FLT3_F1 | GGA GGC CAT GCC GGC GTT G |
| 517 | KIT-F1 | CGC AGC TAC CGC GAT GAG AGG |
| 518 | MET_F1 | CTC ATA ATG AAG GCC CCC GC |
| 519 | PDGFRA_F1 | AAG TTT CCC AGA GCT ATG GGG A |
| 520 | PDGFRB_F1 | AGC AGC AAG GAC ACC ATG CG |
| 521 | RON_F1 | GGT CCC AGC TCG CCT CGA TG |
| 522 | TEK_F1 | AGA TTT GGG GAA GCA TGG ACT C |
| 523 | TIE_F1 | CGG CCT CTG GAG TAT GGT CTG |
| 524 | VEGFR1_F1 | CAT GGT CAG CTA CTG GGA CAC C |
| 525 | VEGFR2_F1 | AGG TGC AGG ATG CAG AGC AAG |
| 526 | VEGFR3_F1 | AGC GGC CGG AGA TGC AGC G |
| 527 | FLT3_R1 | CTG CTC GAC ACC CAC TGT CCA |
| 528 | KIT-R1 | GCA GAA GTC TTG CCC ACA TCG |
| 529 | MET_R1 | CTT CGT GAT CTT CTT CCC AGT GA |
| 530 | PDGFRA_R1 | AGA TTC TTA GCC AGG CAT CGC A |
| 531 | PDGFRB_R1 | AGC GCA CCG ACA GTG GCC GA |
| 532 | RON_R1 | GCA CGG GCT GCC CAC TGT CA |
| 533 | TEK_R1 | CTG TCC GAG GTT CCA AAT AGT TGA |
| 534 | TIE_R1 | CGT TCT CAC TGG GGT CCA CCA |
| 535 | VEGFR1_R1 | ATT ATT GCC ATG CGC TGA GTG A |
| 536 | VEOFR1_R1 | GCC GCT TGG ATA ACA AGG GTA |
| 537 | VEGFR3_R1 | AAC TCG GTC CAG GTG TCC AGG C |
| 538 | FLT3_R2 | CTT GGA AAC TCC CAT TTG AGA TCA |
| 539 | KIT-R2 | ACA ACC TTC CCG AAA GCT CCA |
| 540 | MET_R2 | ACT ACA TGC TGC ACT GCC TGG A |
| 541 | PDGFRA_R2 | CCC GAC CAA GCA CTA GTC CAT C |
| 542 | PDGFRB_R2 | CCA GAG CCG AGG GTG CGT CC |
| 543 | RON_R2 | CAG GTC ATT CAG GTT GGG AGG A |
| 544 | TEK_R2 | ATT TGA TGT CAT TCC AGT CAA GCA |
| 545 | TIE_R2 | AGC ACT GGG TAG CTC AGG GGC |
| 546 | VEGFR1_R2 | AAC TCC CAC TTG CTG GCA TCA |
| 547 | VEGFR2_R2 | AAT TCC CAT TTG CTG GCA TCA |
| 548 | VEGFR3_R2 | ATT CCC ACT GGC TGG CAT CGT A |
| 549 | RAGE_Fu | CAG GAC CCT GGA AGG AAG CA |
| 550 | RAGE_F1 | AGG ATG GCA GCC GGA ACA G |
| 551 | RAGE_f1R1 | CCC CTC AAG GCC CTC CAG TA |
| 552 | RAGE_Intron3R1 | GGA AGT CAG AGG CCC TCA TGG |
| 553 | RAGE_Intron4R1 | GGG AAA GAG TGG TGA CCT CAG A |
| 554 | RAGE_Intron5R1 | CTT GGG GGG CAC CTT AGG ACT C |
| 555 | RAGE_Intron6R1 | ACT CCC TCT TTC CCT AAG GGT CA |
| 556 | RAGE_Intron7R1 | GTT ATG GTT CAC CCT ACC TCC CA |
| 557 | RAGE_Intron8R1 | ATTT AGC TCA GAG GGA AGA AGG GA |
| 558 | HGF_F1 | AGG ATT CTT TCA CCC AGG CA |
| 559 | HGF_intron11R1 | GAA TAA ATG CCA GAC CAC CTA |
| 560 | HGF_F2 | ACC ATG TGG GTG ACC AAA CT |
| 561 | HGF_intron11R2 | TCA CAA GAC ACC AAT CCC TAA CT |
| 562 | HGF_intron13R1 | TCC ATA TTT CTG GGA ATA GGA GGA |
| | | C |

D. Cloning and Sequencing of PCR Products

PCR products were electrophoresed on a 1% agarose gel, and DNA from detectable bands was stained with Gelstar (BioWhitaker Molecular Application, Walkersville, Md.). The DNA bands were extracted with the QiaQuick gel extraction kit (Qiagen, Valencia, Calif.), ligated into the pDrive UA-cloning vector (Qiagen), and transformed into Escherichia coli. Recombinant plasmids were selected on LB agar plates containing 100 μg/ml carbenicillin. For each transfection, 192 colonies were randomly picked and their cDNA insert sizes were determined by PCR with M13 forward and reverse vector primers. Representative clones from PCR products with distinguishable molecular masses as visualized by fluorescence imaging (Alpha Innotech, San Leandro, Calif.) were then sequenced from both directions with vector primers (M13 forward and reverse). All clones were sequenced entirely using custom primers for directed sequencing completion across gapped regions.

E. Sequence Analysis

Computational analysis of alternative splicing was performed by alignment of each cDNA sequence to its respective genomic sequence using SIM4 (a computer program for analysis of splice variants). Only transcripts with canonical (e.g. GT-AG) donor-acceptor splicing sites were considered for analysis. Clones encoding CSR isoforms were studied further (see below, Table 10).

F. Exemplary CSR Isoforms

Exemplary CSR isoforms, prepared using the methods described herein, are set forth below in Table 10. Nucleic acid molecules encoding CSR isoforms are provided and include those that contain sequences of nucleotides or ribonucleotides or nucleotide or ribonuculeotide analogs. SEQ ID NOS for exemplary nucleic acid and amino acid sequences of exemplary CSR isoform polypeptides are depicted in Table 10.

TABLE 10
CSR Isoforms

| | | | | SEQ ID | SEQ ID |
|---|---|---|---|---|---|
| | | | | NO | NO |
| Gene | ID | Type | Length | (nucleotide) | (amino acid) |
| DDR1 | SR005_A11 | Exon deletion | 286 aa | 139 | 140 |
| DDR1 | SR005_A10 | Exon deletion | 243 aa | 141 | 142 |
| EPHA1 | SR004_G03 | Intron fusion | 474 aa | 144 | 145 |
| EPHA1 | SR004_G07 | Intron fusion, exon | 311 aa | 146 | 147 |
| | | deletion | | | |
| EPHA1 | SR004_H03 | Intron fusion | 490 aa | 148 | 149 |
| EPHA2 | SR016_E12 | Intron fusion | 497 aa | 151 | 152 |
| EPHB1 | SR005_D06 | Exon shorten | 242 aa | 154 | 155 |
| EPHB4 | SR012_C08 | Exon deletion | 306 aa | 156 | 157 |
| EPHB4 | SR012_D11 | Exon shorten | 516 aa | 158 | 159 |
| EPHB4 | SR012_E11 | Exon shorted | 414 aa | 160 | 161 |
| FGFR1 | SR001_E12 | Exon deletions | 228 aa | 169 | 170 |
| FGFR1 | SR022_C02 | Exon deletion, intron | 320 aa | 171 | 172 |
| | | fusion | | | |
| FGFR2 | SR022_C10 | Intron fusion | 266 aa | 173 | 174 |
| FGFR2 | SR022_C11 | Intron fusion | 317 aa | 175 | 176 |
| FGFR2 | SR022_D04 | Exon deletion, intron | 281 aa | 177 | 178 |
| | | fusion | | | |
| FGFR2 | SR022_D06 | Intron fusion | 396 aa | 179 | 180 |
| FGFR4 | SR002_A11 | Intron fusion | 72 aa | 182 | 183 |
| FGFR4 | SR002_A10 | Intron fusion | 446 aa | 184 | 185 |
| MET | SR020_C10 | Intron fusion | 413 aa | 187 | 188 |
| MET | SR020_C12 | Intron fusion | 468 aa | 189 | 190 |
| MET | SR020_D04 | Intron fusion | 518 aa | 191 | 192 |
| MET | SR020_D07 | Intron fusion | 596 aa | 193 | 194 |
| MET | SR020_D11 | Intron fusion | 408 aa | 195 | 196 |
| MET | SR020_E11 | Intron fusion | 621 aa | 197 | 198 |
| MET | SR020_F08 | Intron fusion | 664 aa | 199 | 200 |
| MET | SR020_F11 | Intron fusion | 719 aa | 201 | 202 |
| MET | SR020_F12 | Intron fusion | 697 aa | 203 | 204 |
| MET | SR020_G03 | Exon shorten, intron | 691 aa | 205 | 206 |
| | | fusion | | | |
| MET | SR020_G07 | Intron fusion | 661 aa | 207 | 208 |
| MET | SR020_H03 | Intron fusion | 755 aa | 209 | 210 |
| MET | SR020_H06 | Intron fusion | 823 aa | 211 | 212 |
| MET | SR020_H07 | Intron fusion | 877 aa | 213 | 214 |
| MET | SR020_H08 | Exon deletion, intron | 764 aa | 215 | 216 |
| | | fusion | | | |
| RON | SR004_C11 | Intron fusion | 495 aa | 218 | 219 |
| RON | SR014_C01 | Intron fusion | 541 aa | 220 | 221 |
| RON | SR014_C09 | Intron fusion | 908 aa | 222 | 223 |
| RON | SR014_E12 | Intron fusion | 647 aa | 224 | 225 |
| CSF1R | SR005_A06 | Exon deletion | 306 aa | 226 | 227 |
| KIT | SR002_H01 | Intron fusion | 413 aa | 228 | 229 |
| PDGFRB | SR007_C09 | Exon shorten (4 bp) | 336 aa | 232 | 233 |
| RAGE | SR021A05 | Intron fusion | 146 | 234 | 235 |
| RAGE | SR021C02 | Intron fusion | 266 | 236 | 237 |
| RAGE | SR021C06 | Intron fusion | 387 | 238 | 239 |
| RAGE | SR021C08 | Intron fusion | 173 | 240 | 241 |
| RAGE | SR021F06 | Intron fusion | 172 | 242 | 243 |
| TEK | SR007_G02 | Intron fusion, exon | 367 aa | 244 | 245 |
| | | shorten | | | |
| TEK | SR007_H03 | Exon deletion, Intron | 468 aa | 246 | 247 |
| | | fusion | | | |
| TIE | SR006_A04 | Intron fusion | 251 aa | 253 | 254 |
| TIE | SR006_B07 | Intron fusion | 379 aa | 255 | 256 |
| TIE | SR006_B06 | Intron fusion | 161 aa | 257 | 258 |
| TIE | SR006_B12 | Intron fusion | 414 aa | 259 | 260 |
| TIE | SR006_B10 | Exon deletion | 317 aa | 261 | 262 |
| TIE | SR016_G03 | Intron fusion | 751 aa | 263 | 264 |
| TNFR1B | SR003_H02 | Intron fusion | 155 aa | 272 | 273 |
| VEGFR1 | SR004_C05 | Intron fusion | 174 aa | 274 | 275 |
| VEGFR1 | SR01_C02 | Intron fusion | 541 aa | n/a | 280 |
| VEGFR2 | SR015_F01 | Exon shorten | 712 aa | 282 | 283 |
| VEGFR3 | SR007_E10 | Exon short | 227 aa | 284 | 285 |
| VEGFR3 | SR007_F05 | Exon deletion | 295 aa | 286 | 287 |
| VEGFR3 | SR015_G09 | Intron fusion | 765 aa | 288 | 289 |
| HGF | SR023A02 | Intron fusion | 467 aa | 349 | 350 |
| HGF | SR023A08 | Intron fusion | 472 aa | 351 | 352 |
| HGF | SR023E09 | Intron fusion | 514 aa | 353 | 354 |

### Example 2

Preparation and Expression of Intron Fusion Protein Constructs in Human Cells

A. Generation of tPA cDNA

In order to obtain human tissue plasminogen activator (tPA) cDNA, PCR primers specific for the 5′ portion of the human tissue plasminogen activator (tPA) including the tPA signal/pro sequence (based on the human tPA cDNA sequence as set forth in SEQ ID NO: 1) were selected based on the published information (Kohne et al (1999) J Cellular Biochem 75:446-461) and synthesized by Qiagen-Operon (Richmond, Calif.). The sequences of the primers are set forth in SEQ ID NO:7 and SEQ ID NO:8. Each PCR reaction contained 10 ng of reverse transcribed cDNA, 0.025 U/μl TaqPlus (Stratagene), 0.0035 U/μl PfuTurbo (Stratagene), 0.2 mM dNTP (Amersham, Piscataway, N.J.), and 0.2 μM forward and reverse primers in a total volume of 50 μl. PCR conditions were 35 cycles at 94.50° C. for 45 s, 58° C. for 50 s, and 72° C. for 5 min. The reaction was terminated with an elongation step of 720° C. for 10 min. PCR products were electrophoresed on a 1% agarose gel, and DNA from detectable bands was stained with Gelstar (BioWhitaker Molecular Application, Walkersville, Md.). The DNA bands were extracted with the QiaQuick gel extraction kit (Qiagen, Valencia, Calif.), ligated into the pDrive UA-cloning vector (Qiagen), and transformed into Escherichia coli for purification of the pDrive-tPA vector.

B. PCR Amplification and Expression cloning of the tPA Signal/Pro Sequence

In order to clone the portion of the nucleic acid that includes the nucleotides encoding the tPA signal/pro sequence (see Table 11) as set forth in SEQ ID NO: 1, PCR was performed using the primers as forth in SEQ ID NO:9 and SEQ ID NO:10 (see Table 12). The primers were generated to contain restriction enzyme cleavage sites for Nhe I and Xho I, as well as a myc-tag, to facilitate cloning of the amplified product into the pCI expression plasmid (Promega). Alternatively, restriction enzyme cleavage sites for EcoRI and Xba I were generated by running a PCR reaction with the primers as set forth in SEQ ID NO: 11 and SEQ ID NO: 12, and the amplified product was cloned into the pcDNA 3.1 expression plasmid (Invitrogen). The PCR reaction was performed as above with 10 ng pDrive-tPA. The PCR conditions included 35 cycles at 94.5° C. for 45 s, 580° C. for 50 s, and 72° C. for 5 min. The reaction was terminated with an elongation step of 72° C. for 10 min. The tPA encoded cDNA was digested with Nhe I and Xho I or with EcoRI and Xba I to generate the tPA signal/pro sequence fragment and subcloned into the pCI expression plasmid (Promega) at the Nhe I and Xho I sites to form the pCI-tPA:myc vector or subcloned into the pcDNA3.1 expression plasmid (Invitrogen) at the EcoR I and Xba I site to form the pcDNA3.1-tPA vector.

TABLE 11
LIST OF GENES FOR CLONING tPA-intron fusion protein
CONSTRUCTS

| | | SEQ | | | SEQ |
|---|---|---|---|---|---|
| | | ID | | | ID |
| nt ACC. # | Description | NO: | ORF | prt ACC.# | NO: |
| NM_000930 | tPA | 3 | | NP_000921 | 4 |
| | tPA pre/pro | 1 | | | 2 |
| | sequence | | | | |

C. Cloning of Intron Fusion Proteins into the pCI-tPA Vector

Intron fusion proteins were PCR amplified from their pDrive sequencing vector, respectively, and subsequently cloned into the pCI-tPA:myc vector. For the PCR amplification, the forward primers contain an Xho I site, and the reverse primers contain a Not I site. VEGFR1-intron fusion protein without a signal sequence (SEQ ID NO. 279) was PCR amplified using the primers as set forth in SEQ ID NOS:13 and 14. The Met-intron fusion protein without a signal sequence (SEQ ID NO. 214) was amplified using the primers as set forth in SEQ ID NOS:15 and 16. The FGFR2-intron fusion protein without a signal sequence (SEQ ID NO:180) was PCR amplified using the primers as set forth in SEQ ID NOS:17 and 18. The FGFR2-intron fusion protein without a signal sequence (SEQ ID NO:178) was PCR amplified using the primers as set forth in SEQ ID NOS:21 and 22. The FGFR-4-intron fusion protein without a signal sequence (SEQ ID NO: 185) was PCR amplified using the primers set forth in SEQ ID NO:23 and 24. The RAGE intron fusion protein without a signal sequence (see e.g., SEQ ID NO:237) was PCR amplified using primers set forth in SEQ ID NOS:25 and 26. The TEK intron fusion protein without a signal sequence (see e.g., SEQ ID NO:245) was PCR amplified using the primers set forth in SEQ ID NO:27 and 28. The RON intron fusion protein without a signal sequence (see e.g., SEQ ID NO:223) was PCR amplified using the primers set forth in SEQ ID NO:29 and 30. Each PCR reaction contained 10 ng of reverse transcribed cDNA, 0.025 U/μl TaqPlus (Stratagene), 0.0035 U/μl PfuTurbo (Stratagene), 0.2 mM dNTP (Amersham, Piscataway, N.J.), and 0.2 μM forward and reverse primers in a total volume of 50 μl. PCR conditions were 25 cycles and 94.5° C. for 45 s, 580° C. for 50 s, and 72° C. for 5 min. The reaction was terminated with an elongation step of 720° C. for 10 min. PCR products were electrophoresed on a 1% agarose gel, and DNA from detectable bands was stained with Gelstar (BioWhitaker Molecular Application, Walkersville, Md.). The DNA bands were extracted with the QiaQuick gel extraction kit (Qiagen, Valencia, Calif.), subcloned into the pCI-tPA:myc vector at the Xho I and Not I sites downstream of the tPA/pro sequence to generate tPA:myc-intron fusion protein constructs as set forth in SEQ ID NOS. 31-35, 39-47 (nucleotide) and 32-36, 40-48 (amino acid).

The nucleic acid encoding herstatin (Dimercept™)-intron fusion protein without a signal sequence, as set forth in SEQ ID NO:289, was PCR amplified from pcDNA3.1 His-Herstatin (provided by Gail Clinton (OHSU)) and subsequently cloned into the pcDNA3.1-tPA vector. For the PCR amplification, the forward primers were generated to contain an Xba I site, and the reverse primers to contain a Not I site. The cDNA encoding the herstatin-intron fusion protein was amplified using the primers as set forth in SEQ ID NOS:19 and 20. The PCR reaction was performed as described above. PCR products were purified and subcloned into the pcDNA3.1-tPA vector at the Xba I and Not I sites to generate tpA-HER2 intron fusion protein construct as set forth in SEQ ID NO. 37 (nucleotide) and SEQ ID NO. 38 (amino acid). Exemplary tPA-intron fusion protein fusion proteins are set forth in Table 13.

TABLE 12
PRIMERS FOR PCR CLONING.

| SEQ | | | |
|---|---|---|---|
| ID | | | |
| NO | Primer | ID | Sequence |
| 7 | tPA_F | | CTCTGCGAGGAAAGGGAAGGA |
| 8 | tPA_R | | CGTGCCCCTGTAGCTGATGCC |
| 9 | tPApre/pro_F1 | | ATTAGCTAGCCACCATGGATGCAA |
| | | | TGAAGAGAGGG |
| 10 | tPApre/pro_R1 | | ATTACTCGAGCAGATCCTCTTCTG |
| | | | AGATGAGTTTTTGTTCTGGCTCCT |
| | | | CTTCGAATCG |
| 11 | tPApre/pro_F2 | | ATTAGAATTCCACCATGGATGCAA |
| | | | TGAAGAGAGGG |
| 12 | tPApre/pro_R2 | | ATTATCTAGATCTGGCTCCTCTTC |
| | | | TGAATCG |
| 13 | VEGFR11FP_F | SR018_C02 | AAGGCTCGAGTCAAAATTAAAAGA |
| | | | TCCTGAAC |
| 14 | VEGFR11FP_R | SR018_C02 | AAGGAAAAAAGCGGCCGCTCACGG |
| | | | AAGGAAATGGAAG |
| 15 | METIFP_F | SR020_H07 | AAGGCTCGAGTGTAAAGAGGCA |
| | | | CTAGCAAAG |
| 16 | METIFP_R | SR020_H07 | AAGGAAAAAAGCGGCCGCTCACGG |
| | | | AAGGAAATGGAAG |
| 17 | FGFR2IFP_F | SR022_D06 | AAGGCTCGAGCCCTCCTTCAGTTT |
| | | | AGTTGA |
| 18 | FGFR2IFP_R | SR022_D06 | AAGGAAAAAAGCGGCCGCTTATGC |
| | | | AAGGATAAAAGGGG |
| 19 | DCPTIFP_F | Herstatin | AATTTCTAGACAAGTGTGCACCGG |
| | | | CACAGAC |
| 20 | DCPTIFP_R | Herstatin | AAGGAAAAGCGGCCGCTCAGCCTT |
| | | | CATACCGGGAC |
| 21 | FGFR2IFP_F2 | SR022_D04 | AATTCTCGAGCCCTCCTTCAGTTT |
| | | | AGTTGA |
| 22 | FGFR2IFP_R2 | SR022_D04 | AATTGAATTC TTATGCAAGGATA |
| | | | AAAGGGGC |
| 23 | FGFR4LFP_F | SR002_A10 | AATTCTCGAGGAGGAAGTGGAGCT |
| | | | TGAGCC |
| 24 | FGFR4IFP_R | SR002_A10 | AATTGAATTCCTAACTCAGTCCCT |
| | | | CCCAG |
| 25 | RAGEIFP_F | SR021_C02 | AATTCTCGAGCAAAACATCACAGC |
| | | | CCGGA |
| 26 | RAGEIFP_R | SR021_C02 | AATTGAATTCCTAAGGGTCAGACT |
| | | | TCCAGA |
| 27 | TEKIFP_F | SR007_G02 | AATTCTCGAGGTGGAAGGTGCCAT |
| | | | GGACT |
| 28 | TEKIFP_R | SR007_G02 | AATTGAATTCTTACCACTGTTTAC |
| | | | TTCTATATGA |
| 29 | RONIFP_F | SR014_C09 | AATTCTCGAGGACTGGCAGTGCCC |
| | | | GCG |
| 30 | RONIFP_R | SR014_C09 | AATTGAATTCTCATGAGGACCAGC |
| | | | CAGTAG |

TABLE 13
tPA-intron fusion protein Fusions

| | | SEQ ID NO | SEQ ID NO |
|---|---|---|---|
| ID | Isoform Type | (nucleotide) | (amino acid) |
| SR018C02 | tPA-myc-VEGFR-1 | 31 | 32 |
| SR02H07 | tPA-myc-MET | 33 | 34 |
| SR022D06 | tPA-myc-FGFR-2 | 35 | 36 |
| Herstatin | tPA_DCPT | 37 | 38 |
| SR022D04 | tPA-myc-FGFR-2 | 39 | 40 |
| SR002A10 | tPA-myc-FGFR-4 | 41 | 42 |
| SR021C02 | tPA-myc-RAGE | 43 | 44 |
| SR007G02 | tPA-myc-TEK | 45 | 46 |
| SR014C09 | tPA-myc-RON | 47 | 48 |

D. Protein Expression and Secretion

Medium from cultured human cells was assessed for secretion of each of the tPA-intron fusion proteins. To express the tPA-intron fusion proteins in human cells, human embryonic kidney 293T cells were seeded at 2×10⁶ cells/well in a 6-well plate and maintained in Dulbecco's modified Eagle's medium (DMEM) and 10% fetal bovine serum (Invitrogen). Cells were transfected using LipofectAMINE 2000 (Invitrogen) following the manufacturer's instructions. On the day of transfection, 5 μg plasmid DNA was mixed with 15 μl of LipofectAMINE 2000 in 0.5 ml of serum-free DMEM. The mixture was incubated for 20 minutes at room temperature before it was added to the cells. Cells were incubated at 37° C. in a CO₂ incubator for 48 hours. To study the protein secretion of intron fusion proteins, the conditioned media was collected 48 hours after transfection and expression levels were analyzed by Western blotting. Conditioned media was analyzed by separation on SDS-polyacrylamide gels followed by immunoblotting using an anti-Myc antibody (Invitrogen) or an anti-Herstatin antibody (Upstate). Antibodies were diluted 1:5000. To study the cellular protein expression of the intron fusion proteins, after cell culture media was removed, the transfected cells were harvested and lysed in a cell lysis buffer (PBS/0.25% Triton X-100). Lysates were clarified by centrifugation to remove insoluble cell debris. Typically, 10 μg protein from each sample was separated on an SDS-PAGE gel after protein concentrations were determined. Cell lysates were analyzed by Western blotting using an anti-Myc antibody (Invitrogen) or an anti-Herstatin antibody (Upstate). Expression and secretion of intron fusion proteins containing a tPA pre/prosequence were compared to intron fusion proteins containing the original or endogenous signal peptide. Comparisons of expression and secretion of intron fusion proteins are depicted in Table 14 and Table 15.

TABLE 14
Summary of intron fusion protein Protein Expression and Secretion

| intron | | Protein | Protein | Protein | Protein |
|---|---|---|---|---|---|
| fusion | | Expression w/ | Secretion w/ | Expression w/ | Secretion w/ |
| protein ID | Gene | Original sp | Original sp | tPA sp | tPA sp |
| SR018C02 | VEGFR1 | − | − | +++ | +++ |
| SR020H07 | MET | ++ | − | +++ | +++ |
| SR022D04 | FGFR2 | ++ | + | +++ | +++ |
| SR021C02 | RAGE | ++ | + | +++ | +++ |
| SR002A10 | FGFR4 | ++ | − | ++ | + |
| SR007G02 | TEK | ++ | − | ++ | + |
| SR014C09 | RON | ++ | − | ++ | + |
| Herstatin | HER2 | +++ | − | +++ | +++ |
| SR022D06 | FGFR2 | ++ | + | +++ | +++ |

TABLE 15
tPA-intron fusion protein fusion facilitates secretion of the
recombinant intron fusion proteins in 293T cells

| tPA-intron | | Fold increase in |
|---|---|---|
| fusion protein | Clone ID | Protein Secretion |
| tPA-FGFR-2 | SR022D06 | 5 |
| tPA-VEGFR-1 | SR018C02 | 10 |
| tPA-MET | SR020H07 | 30 |
| tPA-HER2 | Herstatin | 30 |

### Example 3

Herstatin (Dimercept™) Purification and Cell-Based Growth Inhibition Assays

A. Transient Expression of tPA-HER2 Using 293T Cells

293T cells (ATCC) were maintained in DMEM/10% fetal bovine serum. For transfection, cells were seeded at a density of 1×10⁷ per 100-mm cell culture plate. Transient transfection was carried out 24 hours later using LipofectAmine™ 2000 reagent (Invitrogen) following the manufacturer's recommendation. Briefly, 293T cells were fed with serum-free DMEM immediately before the transfection started. For transfection of each of the 293T cell plate, 75 μl of LipofectAmine 2000 and 25 μg of the tPA-HER2 expression construct (or a pcDNA control plasmid) were mixed in 2 ml of serum-free DMEM. The DNA-LipofectAmine mixture was incubated at room temperature for 20 min and then applied dropwise to the 293T cell plate. Supernatants from the transfected cells were collected 48 hours later, centrifuged, and filtered to remove remaining cells. Clarified supernatants were processed for protein purification.

B. Purification of a Partially Purified Herstatin (Dimercept™)

Transiently transfected conditioned cell culture medium containing the expressed herstatin protein product encoded by the construct was concentrated approximately 10 fold either using tangential flow membranes or using stirred cell system filters, exhibiting a 10,000 molecular weight separation cutoff. The materials retained by the membrane or filter were further processed. Following the aforementioned concentration/volume reduction, the sample was diluted with cold 50 mM sodium acetate, pH 5.5 (the sample was diluted with either one or two equal volumes of buffer) and the pH was monitored and adjusted using acetic acid or HCl, as required to achieve a final pH of 5.5. After pH adjustment, the conditioned medium was passed through a 0.45 micron filter to remove any particulates, prior to column chromatography.

The above mentioned concentrated/conditioned material was subsequently loaded (50-300 ml of feed per 5 ml bed volume; 1-3 ml/min flow rate) onto an SP-Sepharose ion exchange chromatography column, equilibrated in 50 mM sodium acetate, pH 5.5. The load was washed onto the column using column equilibration buffer, and the washed eluate monitored until the optical absorbance at 280 nm was minimal and constant. The resulting flow through and wash of the column was retained for later evaluation.

Column elution of bound protein was performed using an isocratic step elution approach employing, in serial sequence, the following buffers: 50 mM sodium acetate, pH 5.5, 200 mM sodium chloride; 50 mM sodium acetate, pH 5.5, 500 mM sodium chloride; 50 mM sodium acetate, pH 5.5, 1M sodium chloride; and, 50 mM sodium acetate, pH 5.5, 2M sodium chloride. At each elution stage, the 280 nm absorbance profile of the eluate was monitored and a baseline-to-baseline pool was made containing the materials eluted from the column under those respective conditions. Immediately upon pooling of the fractions, the pH was adjusted to between 7.0 and 7.5 using 1M Tris-HCl, pH 8 (˜10 μl/ml of fraction pool).

Most operations were carried out at 2-8° C. Materials thus prepared and aliquots of all fractions generated during the isolation process were stored either at 2-8° C. or −80° C. until further analysis.

C. Assay Purified Herstatin (Dimercept™) for Anti-Proliferative Activity

Bioassay Assessment—Alamar Blue Growth Inhibitory Assay for Herstatin

DU-145 cells were seeded in 96-well plate, 5000/well in DMEM containing 2% fetal bovine serum on the day before the assay. Cells were treated with 2-fold serial dilution of pooled fractions of purified herstatin (nDcp) and controls (representing 10%, 5%, 2.5%, 1.25%, and 0.75% if the assay volume) in 0.2% of FBS/DMEM. After 5 days of incubation at 37° C., cell density in the wells was measured by the Alamar Blue (Sigma Cat. # R7017) method. 100 μl of 2× Alamar Blue was added to each well containing 100 μl culture medium and fluorescence was measured of each treated and control wells at Ex.=530 mn /Em.=590 nm in 2-4 hours. DU145 growth inhibition was analyzed by dose-responsive curve based on fluorescence reading and compared to results from control treatments. The purified herstatin pooled fraction inhibited cell proliferation and growth by about 15% at a concentration of 0.75% of the assay volume with maximum inhibition observed (80% inhibition compared to a pcDNA control) at 1.25% of the assay volume.

Since modifications will be apparent to those of skill in this art, it is intended that this invention be limited only by the scope of the appended claims.

1. A polypeptide, comprising a receptor tyrosine kinase (RTK) isoform operatively linked directly or indirectly via a polypeptide linker to a heterologous precursor sequence or a sufficient portion thereof to effect secretion, processing and/or trafficking of the linked RTK intron fusion protein.

2. The polypeptide of claim 1, wherein the RTK isoform contains an endogenous signal sequence.

3. The polypeptide of claim 1, wherein the RTK isoform does not contain an endogenous signal sequence.

4. The polypeptide of claim 1, wherein the precursor sequence is selected from among a tissue plasminogen activator (tPA) pre/prosequence or a sufficient portion thereof to effect secretion, and allelic and species variants thereof.

5. The polypeptide of claim 4, wherein the tPA pre/prosequence is a mammalian tPA pre/prosequence.

6. The polypeptide of claim 4, wherein the tPA pre/prosequence comprises the sequence of amino acids set forth in SEQ ID NO:2 or allelic variants thereof or variants that have at least about 80%, 85%, 90%, 95%, 96%, 97%, 98%, 99% or more sequence identity, wherein and the tPA portion effects secretion, processing and/or trafficking of the linked RTK isoform.

7. The polypeptide of claim 1, wherein the RTK isoform is selected from among a VEGFR, FGFR, PDGFR, MET, EPH, TIE, DDR and HER fusion protein.

8. The polypeptide of claim 7, wherein the RTK isoform is selected from a DDR1, EphA1, EphA2, EphA8, EphB1, EphB4, EGFR, HER-2 (ErbB2), ErbB3, FGFR-1, FGFR-2, FGFR-4, MET, RON, CSF1R, KIT, PDGFR-A, PDGFR-B, TEK, Tie-1, VEGFR-1, VEGFR-2, VEGFR-3 and allelic variants thereof or variants thereof that have at least about 80%, 85%, 90%, 95%, 96%, 97%, 98%, 99% or more sequence identity with any of these RTK isoforms, wherein the variants possess at least one activity of the corresponding RTK isoform.

9. The polypeptide of claim 8, wherein the RTK isoform comprises a sequence of amino acids set forth in any one of SEQ ID NOS: 140, 142, 143, 145, 147, 149, 150, 152, 153, 155, 157, 159, 161-168, 170, 172, 174, 176, 178, 180, 181, 183, 185, 186, 188, 190, 192, 194, 196, 198, 200, 202, 204, 206, 208, 210, 212, 214, 216, 217, 219, 221, 223, 225, 227, 229-231, 233, 245, 247-251, 253, 255, 257, 259, 261, 263-270, 274-280, 282, 284, 286, 288, 289-303 or an active portion thereof.

10. The polypeptide of claim 1, wherein the RTK isoform is operatively linked via a linker to a tPA precursor sequence or a sufficient portion thereof to effect secretion.

11. The polypeptide of claim 10, wherein the linker is a restriction enzyme linker that is encoded by a sequence of nucleotides recognized by one or more restriction enzymes.

12. The polypeptide of claim 11, wherein the restriction enzyme linker is joined between an isoform and a tPA pre/prosequence or a sufficient portion thereof to effect secretion.

13. The polypeptide of claim 1, further comprising a multimerization domain.

14. The polypeptide of claim 1, wherein the tag is linked between the restriction enzyme linker and a tPA precursor sequence or a sufficient portion thereof to effect secretion.

15. The polypeptide of claim 14, wherein the tag is a myc tag.

16. The polypeptide of claim 15, wherein the RTK isoform is selected from a VEGFR-1, FGFR-2, FGFR-4, TEK, RON, MET and allelic variants thereof or variants thereof that have at least about 80%, 85%, 90%, 95%, 96%, 97%, 98%, 99% or more sequence identity with any of these RTK isoforms, wherein the variants possess at least one activity of the corresponding RTK isoform.

17. The polypeptide of claim 1, comprising a sequence of amino acids set forth in any one of SEQ ID NOS: 32, 34, 36, 40, 42, 46 and or 48.

18. The polypeptide of claim 13, wherein the construct includes a restriction enzyme linker, and the tag is located between the restriction enzyme linker and the isoform.

19. The polypeptide of claim 13, wherein the tag is a Poly-His tag.

20. The polypeptide of claim 1, wherein the RTK isoform is HER-2 or an allelic variant thereof.

21. The polypeptide of claim 20, comprising a sequence of amino acids set forth in SEQ ID NO: 38.

22. A polypeptide, comprising a Receptor for Advanced Glycation Endproducts (RAGE) isoform operatively linked directly or indirectly via a polypeptide linker to a heterologous precursor sequence or a sufficient portion thereof to effect secretion and/or trafficking of the RAGE isoform, wherein the polypeptide optionally includes a tag that facilitates polypeptide purification and/or detection.

23. The polypeptide of claim 22, wherein the RAGE isoform contains an endogenous signal sequence.

24. The polypeptide of claim 23, wherein the RAGE isoform protein does not contain an endogenous signal sequence.

25. The polypeptide of claim 22, wherein the precursor sequence is a tissue plasminogen activator (tPA) pre/prosequence or a sufficient portion thereof to effect secretion, or allelic variants thereof or variants thereof that have at least about 80%, 85%, 90%, 95%, 96%, 97%, 98%, 99% or more sequence identity with the precursor sequence to effect secretion and/or processing or trafficking of the RAGE isoform.

26. The polypeptide of claim 25, wherein the tPA pre/prosequence is a mammalian tPA pre/prosequence.

27. The polypeptide of claim 25, wherein the tPA pre/prosequence comprises the sequence of amino acids set forth in SEQ ID NO:2, or allelic variants thereof.

28. The polypeptide of claim 22, wherein the RAGE isoform comprises a sequence of amino acids set forth in any of SEQ ID NOS: 235, 237, 239, 241, 243, or an active portion thereof or a variant thereof that has at least about 80%, 85%, 90%, 95%, 96%, 97%, 98%, 99% or more sequence identity therewith.

29. The polypeptide of any claim 22, wherein the RAGE isoform is operatively linked by a linker to a tPA pre/prosequence or a sufficient portion thereof to effect secretion.

30. The polypeptide of claim 29, wherein the linker is a restriction enzyme linker that is encoded by a sequence of nucleotides recognized by one or more restriction enzymes.

31. The polypeptide of claim 30, wherein the restriction enzyme linker is joined between the RAGE isoform or an active portion thereof and a tPA pre/prosequence or a sufficient portion thereof to effect secretion.

32. The polypeptide of claim 22, further comprising a multimerization domain.

33. The polypeptide of claim 22, wherein the polypeptide contains a restriction enzyme linker, and the tag is linked between the restriction enzyme linker and a tPA pre/prosequence or a sufficient portion thereof to effect secretion.

34. The polypeptide of claim 33, wherein the tag is a myc tag.

35. The polypeptide of claim 22, comprising a sequence of amino acids set forth in SEQ ID NO: 44.

36. A polypeptide, comprising a tumor necrosis factor receptor (TNFR) isoform operatively linked directly or indirectly via a linker to a heterologous precursor sequence or a sufficient portion thereof to effect secretion, processing and/or trafficking of the TNFR isoform, wherein the polypeptide optionally includes a tag that facilitates polypeptide purification and/or detection.

37. The polypeptide of claim 36, wherein the TNFR isoform contains an endogenous signal sequence.

38. The polypeptide of claim 36, wherein the TNFR isoform does not contain an endogenous signal sequence.

39. The polypeptide of claim 36, wherein the precursor sequence is a tissue plasminogen activator (tPA) pre/prosequence or a sufficient portion thereof to effect secretion, or allelic variants thereof or a variant thereof that has at least about 80%, 85%, 90%, 95%, 96%, 97%, 98%, 99% or more sequence identity therewith.

40. The polypeptide of claim 39, wherein the tPA pre/prosequence is a mammalian tPA pre/prosequence.

41. The polypeptide of claim 39, wherein the tPA pre/prosequence comprises the sequence of amino acids set forth in SEQ ID NO:2.

42. The polypeptide of claim 36, wherein the TNFR isoform is a TNFR1 or a TNFR2.

43. The polypeptide of claim 42, wherein the TNFR isoform is a TNFR2 isoform.

44. The polypeptide of claim 43, wherein the TNFR isoform comprises a sequence of amino acids set forth in SEQ ID NO: 272 or an active portion thereof or a variant thereof that has at least about 80%, 85%, 90%, 95%, 96%, 97%, 98%, 99% or more sequence identity therewith.

45. The polypeptide of claim 36, wherein the TNFR isoform is operatively linked by a linker to a tPA pre/prosequence or to a sufficient portion thereof to effect secretion, processing or trafficking.

46. The polypeptide of claim 45, wherein the linker is a restriction enzyme linker that is encoded by a sequence of nucleotides recognized by one or more restriction enzymes.

47. The polypeptide of claim 46, wherein the restriction enzyme linker is joined between an isoform or an active portion thereof and a tPA pre/prosequence or a sufficient portion thereof to effect secretion.

48. The polypeptide of claim 36, further comprising a multimerization domain.

49. The polypeptide of claim 36, wherein the polypeptide includes a restriction enzyme linker, and the tag is linked between the restriction enzyme linker and a tPA precursor sequence or a sufficient portion thereof to effect secretion.

50. The polypeptide of claim 36, wherein the tag is a myc tag.

51. A nucleic acid molecule, comprising a sequence of nucleotides that encodes a polypeptide of claim 1.

52. A nucleic acid molecule, comprising a sequence of nucleotides set forth in any one of SEQ ID NOS: 31, 33, 35, 37, 39, 41, 43, 45 and 47.

53. A vector, comprising the DNA construct of claim 51.

54. The vector of claim 53 that is a mammalian expression vector.

55. The vector of claim 54 that is selected from among a pCI vector and a pcDNA3.1 vector.

56. The vector of claim 53 that is selected from among an adenovirus vector, an adeno-associated virus vector, EBV, SV40, cytomegalovirus vector, vaccinia virus vector, herpesvirus vector, a retrovirus vector, a lentivirus vector, or an artificial chromosome.

57. The vector of claim 53 that is episomal or that integrates into the chromosome of a cell into which it is introduced.

58. A cell, comprising the vector of claim 53.

59. A cell of claim 58, that is a mammalian cell.

60. A cell of claim 59, wherein the mammalian cell is selected from among a mouse, rat, human, monkey, chicken, and hamster cell.

61. A cell of claim 59, wherein the cell is selected from among a CHO, Balb/3T3, HeLa, MT2, mouse NS0 and other myeloma cell lines, hybridoma and heterohybridoma cell lines, lymphocytes, fibroblasts, Sp2/0, COS, NIH3T3, HEK293, 293T, 293S, 2B8, and HKB cells, and EBNA-1 cell.

62. A method of producing a CSR isoform or a ligand isoform, comprising culturing a cell of claim 58, whereby the isoform is secreted.

63. The method of claim 62, further comprising purifying the secreted isoform from the cell culture.

64. The method of claim 63, wherein:
the isoform comprises an epitope tag for facilitating purification; and
the epitope tag is expressed on the protein.

65. The method of claim 63, wherein the purified protein is treated with an exoprotease.

66. The method of claim 65, wherein the exoprotease is a plasmin-like protease.

67. A method of producing a CSR isoform or a ligand isoform, comprising introducing a nucleic acid molecule encoding the isoform and a signal sequence, whereby the isoform is secreted from the cell.

68. The method of claim 67, wherein the cell is a mammalian cell.

69. The method of claim 68, wherein the mammalian cell is selected from among a mouse, rat, human, monkey, chicken, and hamster cell.

70. The method of claim 68, wherein the cell is selected from among a CHO, Balb/3T3, HeLa, MT2, mouse NS0 and other myeloma cell lines, hybridoma and heterohybridoma cell lines, lymphocytes, fibroblasts, Sp2/0, COS, NIH3T3, HEK293, 293T, 293S, 2B8, and HKB cells and EBNA-1 cells.

71. The method of claim 67, wherein the nucleic acid molecule comprises a sequence of nucleotides that encodes a polypeptide of any of SEQ ID NOS: 31, 33, 35, 37, 39, 41, 43, 45and 47.

72. The method of claim 67, wherein the nucleic acid molecule is introduced into a cell by a method selected from among transfection, electroporation, and nuclear microinjection.

73. The method of claim 67, wherein the nucleic acid molecule is introduced into a cell by using calcium phosphate, a cationic lipid reagent, or a polycation.

74. The method of claim 73, wherein the cationic lipid reagent is selected from among: a 1:1 (w/w) formulation of the cationic lipid N-[1-(2,3-dioleyloxy)propyl]-N,N,N-trimethylammonium chloride (DOTMA) and dioleoyl-phosphatidyl-ethanol-amine (DOPE); a 3:1 (w/w) formulation of polycationic lipid 2,3-dioleyloxy-N-[2(spermine-carboxamido) ethyl]-N,N-dimethyl-1-propanaminiumtrifluoroacetate (DOSPA) and dioleoyl phosphatidyl-ethanolamine (DOPE) and other compositions comprising one or more of DOTMA, DOSPA and DOPE.

75. The method of claim 67, further comprising purifying the secreted isoform from the cell culture.

76. The method of claim 75, wherein purifying the isoform is facilitated by an epitope tag expressed by the protein.

77. The method of claim 75, wherein the purified protein is treated with an exoprotease.

78. The method of claim 77, wherein the exoprotease is a plasmin-like protease.

79. A polypeptide, comprising a cell surface receptor (CSR) or ligand isoform wherein:
the polypeptide lacks an endogenous precursor sequence; and
the polypeptide contains one or more additional amino acids at its N-terminus.

80. The polypeptide of claim 79, wherein the endogenous precursor sequence comprises a signal sequence.

81. The polypeptide of claim 79, wherein the endogenous precursor sequence comprises a signal sequence and one additional amino acid.

82. The polypeptide of claim 79, wherein the CSR isoform is an isoform selected from among an RTK, TNFR, and RAGE isoform.

83. The polypeptide of claim 79, wherein the ligand isoform is an isoform of HGF.

84. The polypeptide claim 79, comprising all or a portion of a sequence of amino acids set forth in any one of SEQ ID NOS: 140, 142, 143, 145, 147, 149, 150, 152, 153, 155, 157, 159, 161-168, 170, 172, 174, 176, 178, 180, 181, 183, 185, 186, 188, 190, 192, 194, 196, 198, 200, 202, 204, 206, 208, 210, 212, 214, 216, 217, 219, 221, 223, 225, 227, 229-231, 233, 235, 237, 239, 241, 243, 245, 247-251, 253, 255, 257, 259, 261, 263-270, 272, 274-280, 282, 284, 286, 288, 289-303, 350, 352 and 354, allelic or species variants thereof and variants thereof that have at least about 80%, 85%, 90%, 95%, 96%, 97%, 98%, 99% or more sequence identity therewith and possess at least one activity of the corresponding polypeptide set forth in any of SEQ ID NOS: 140, 142, 143, 145, 147, 149, 150, 152, 153, 155, 157, 159, 161-168, 170, 172, 174, 176, 178, 180, 181, 183, 185, 186, 188, 190, 192, 194, 196, 198, 200, 202, 204, 206, 208, 210, 212, 214, 216, 217, 219, 221, 223, 225, 227, 229-231, 233, 235, 237, 239, 241, 243, 245, 247-251, 253, 255, 257, 259, 261, 263-270, 272, 274-280, 282, 284, 286, 288, 289-303, 350, 352 and 354.

85. The polypeptide of claim 79, wherein the one or more additional amino acid at the N-terminus is one or more of a restriction enzyme linker sequence or a portion of a prosequence of tPA or an epitope tag, wherein a restriction enzyme linker is encoded by a sequence of nucleotides recognized by one or more restriction enzymes.

86. The polypeptide of claim 79, wherein the one or more additional amino acids at the N-terminus are GAR, SR, or LE.

87. The polypeptide of claim 79, wherein the one or more additional amino acids at the N-terminus are GARSR or GARLE.

88. The polypeptide of claim 79, comprising a multimerization domain.

89. A pharmaceutical composition, comprising a polypeptide of any one of claims 1, 22, 36 and 79.

90. A method of treating a disease or condition comprising, administering to a subject a pharmaceutical composition of claim 89, wherein the disease or condition is mediated by a cognate CSR or ligand.

91. The method of claim 90, wherein the disease or condition is an inflammatory disease, cancer, angiogenesis-mediated disease, or a hyperproliferative disease.

92. The method of claim 91, wherein the disease or condition is selected from among ocular disease, atherosclerosis, diabetes, rheumatoid arthritis, hemangioma, wound healing, Alzheimer's disease, Creutzfeldt-Jakob disease, Huntington's disease, smooth muscle proliferative-related disease, multiple sclerosis, cardiovascular disease, and kidney disease.

93. The method of claim 91, wherein the cancer is selected from among carcinoma, lymphoma, blastoma, sarcoma, leukemia, lymphoid malignancies, squamous cell cancer, lung cancer including small-cell lung cancer, non-small-cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, and head and neck cancer.

94. A polypeptide, comprising a hepatocyte growth factor (HGF) isoform operatively linked directly or indirectly to a heterologous precursor sequence or a sufficient portion thereof to effect secretion and/or trafficking of the HGF isoform, wherein the polypeptide optionally includes a tag that facilitates polypeptide purification and/or detection.

95. The polypeptide of claim 94, wherein the HGF isoform contains an endogenous signal sequence.

96. The polypeptide of claim 95, wherein the HGF isoform does not contain an endogenous signal sequence.

97. The polypeptide of claim 94, wherein the precursor sequence is a tissue plasminogen activator (tPA) pre/prosequence or a sufficient portion thereof to effect secretion, or allelic variants thereof or variants that have at least about 80%, 85%, 90%, 95%, 96%, 97%, 98%, 99% or more sequence identity, wherein the variants effect secretion, processing and/or trafficking of the linked isoform.

98. The polypeptide of claim 97, wherein the tPA pre/prosequence is a mammalian tPA pre/prosequence.

99. The polypeptide of claim 97, wherein the tPA pre/prosequence comprises the sequence of amino acids set forth in SEQ ID NO:2, or allelic variants thereof.

100. The polypeptide of claim 94, wherein the HGF isoform comprises a sequence of amino acids set forth in any one of SEQ ID NOS: 350, 352, or 354 or an active portion thereof.

101. The polypeptide of claim 95, wherein the HGF isoform is operatively linked by a linker to a tPA pre/prosequence or a sufficient portion thereof to effect secretion, processing and/or trafficking of the linked isoform.

102. The polypeptide of claim 101, wherein the linker is a restriction enzyme linker that is encoded by a sequence of nucleotides recognized by one or more restriction enzymes.

103. The polypeptide of claim 102, wherein the restriction enzyme linker is joined between an isoform or an active portion thereof and a tPA pre/prosequence or a sufficient portion thereof to effect secretion.

104. The polypeptide of claim 94, wherein:
the polypeptide comprises a restriction enzyme linker, wherein the linker is a restriction enzyme linker that is encoded by a sequence of nucleotides recognized by one or more restriction enzyme; and
the tag is linked between the restriction enzyme linker and a tPA precursor sequence or a sufficient portion thereof to effect secretion.

105. The polypeptide of claim 94, wherein the tag is a myc tag.

106. A pharmaceutical composition, comprising a nucleic acid molecule of claim 51, wherein the disease or condition is mediated by a CSR or ligand therefor.

107. A method of treating a disease or condition, comprising:administering to a subject a pharmaceutical composition of claim 106, wherein the disease or condition is mediated by a cognate CSR or ligand.

108. The method of claim 107, wherein the disease or condition is an inflammatory disease, cancer, angiogenesis-mediated disease, or a hyperproliferative disease.

109. The method of claim 108, wherein the disease or condition is selected from among ocular disease, atherosclerosis, diabetes, rheumatoid arthritis, hemangioma, wound healing, Alzheimer's disease, Creutzfeldt-Jakob disease, Huntington's disease, smooth muscle proliferative-related disease, multiple sclerosis, cardiovascular disease, and kidney disease.

110. The method of claim 109, wherein the cancer is selected from among carcinoma, lymphoma, blastoma, sarcoma, leukemia, lymphoid malignancies, squamous cell cancer, lung cancer including small-cell lung cancer, non-small-cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, and head and neck cancer.

111. A nucleic acid molecule, comprising a sequence of nucleotides that encodes a polypeptide of claim 22.

112. A pharmaceutical composition, comprising the nucleic acid molecule of claim 111.

113. A nucleic acid molecule, comprising a sequence of nucleotides that encodes a polypeptide of claim 36.

114. A pharmaceutical composition, comprising the nucleic acid molecule of claim 113.
